SEQUENCE LISTING (61 sequences)

SEQ ID NO: 1 - protein, 12 aa, Homo sapiens
  Spacer (IgG4hinge)
ESKYGPPCPPCP

SEQ ID NO: 2 - DNA, 36 bp, Homo sapiens
  Spacer (IgG4hinge)
gaatctaagtacggaccgccctgccccccttgccct

SEQ ID NO: 3 - protein, 119 aa, Homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, Artificial Sequence
  T2A
LEGGGEGRGSLLTCGDVEENPGPR

SEQ ID NO: 7 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  CD28
FWVLVVVGGVLACYSLLVTVAFIIFWV

SEQ ID NO: 9 - protein, 66 aa, Homo sapiens
  CD28
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  CD28
RSKRSRLLHSDYMNMTPRRPGPTRKHYQPYAPPRDFAAYRS

SEQ ID NO: 11 - protein, 41 aa, Homo sapiens
  CD28
RSKRSRGGHSDYMNMTPRRPGPTRKHYQPYAPPRDFAAYRS

SEQ ID NO: 12 - protein, 42 aa, Homo sapiens
  4-1BB
KRGRKKLLYIFKQPFMRPVQTTQEEDGCSCRFPEEEEGGCEL

SEQ ID NO: 13 - protein, 112 aa, Homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 17 - protein, 18 aa, Artificial Sequence
  T2A
EGRGSLLTCGDVEENPGP

SEQ ID NO: 18 - protein, 22 aa, Artificial Sequence
  P2A
GSGATNFSLLKQAGDVEENPGP

SEQ ID NO: 19 - protein, 19 aa, Artificial Sequence
  P2A
ATNFSLLKQAGDVEENPGP

SEQ ID NO: 20 - protein, 20 aa, Artificial Sequence
  E2A
QCTNYALLKLAGDVESNPGP

SEQ ID NO: 21 - protein, 22 aa, Artificial Sequence
  F2A
VKQTLNFDLLKLAGDVESNPGP

SEQ ID NO: 22 - protein, 9 aa, Artificial Sequence
  Linker
  REPEAT (5)...(9): SGGGG is repeated 5 times
PGGGSGGGG

SEQ ID NO: 23 - protein, 17 aa, Artificial Sequence
  Linker
GSADDAKKDAAKKDGKS

SEQ ID NO: 24 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 25 - protein, 22 aa, Artificial Sequence
  GMCSFR alpha chain signal sequence
MLLLVTSLLLCELPHPAFLLIP

SEQ ID NO: 26 - protein, 18 aa, Artificial Sequence
  CD8 alpha signal peptide
MALPVTALLLPLALLLHA

SEQ ID NO: 27 - protein, 15 aa, Artificial Sequence
  Hinge
EPKSCDKTHTCPPCP

SEQ ID NO: 28 - protein, 12 aa, Artificial Sequence
  Hinge
ERKCCVECPPCP

SEQ ID NO: 29 - protein, 61 aa, Artificial Sequence
  Hinge
ELKTPLGDTHTCPRCPEPKSCDTPPPCPRCPEPKSCDTPPPCPRCPEPKSCDTPPPCPRCP

SEQ ID NO: 30 - protein, 12 aa, Artificial Sequence
  Hinge
ESKYGPPCPSCP

SEQ ID NO: 31 - protein, 12 aa, Artificial Sequence
  Hinge
ESKYGPPCPPCP

SEQ ID NO: 32 - protein, 9 aa, Artificial Sequence
  Hinge
YGPPCPPCP

SEQ ID NO: 33 - protein, 10 aa, Artificial Sequence
  Hinge
KYGPPCPPCP

SEQ ID NO: 34 - protein, 14 aa, Artificial Sequence
  Hinge
EVVVKYGPPCPPCP

SEQ ID NO: 35 - protein, 11 aa, Artificial Sequence
  CDR L1
RASQDISKYLN

SEQ ID NO: 36 - protein, 7 aa, Artificial Sequence
  CDR L2
SRLHSGV

SEQ ID NO: 37 - protein, 9 aa, Artificial Sequence
  CDR L3
GNTLPYTFG

SEQ ID NO: 38 - protein, 5 aa, Artificial Sequence
  CDR H1
DYGVS

SEQ ID NO: 39 - protein, 16 aa, Artificial Sequence
  CDR H2
VIWGSETTYYNSALKS

SEQ ID NO: 40 - protein, 7 aa, Artificial Sequence
  CDR H3
YAMDYWG

SEQ ID NO: 41 - protein, 120 aa, Artificial Sequence
  VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 42 - protein, 107 aa, Artificial Sequence
  VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 43 - protein, 245 aa, Artificial Sequence
  scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 44 - protein, 11 aa, Artificial Sequence
  CDR L1
KASQNVGTNVA

SEQ ID NO: 45 - protein, 7 aa, Artificial Sequence
  CDR L2
SATYRNS

SEQ ID NO: 46 - protein, 9 aa, Artificial Sequence
  CDR L3
QQYNRYPYT

SEQ ID NO: 47 - protein, 5 aa, Artificial Sequence
  CDR H1
SYWMN

SEQ ID NO: 48 - protein, 17 aa, Artificial Sequence
  CDR H2
QIYPGDGDTNYNGKFKG

SEQ ID NO: 49 - protein, 13 aa, Artificial Sequence
  CDR H3
KTISSVVDFYFDY

SEQ ID NO: 50 - protein, 122 aa, Artificial Sequence
  VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 51 - protein, 108 aa, Artificial Sequence
  VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 52 - protein, 15 aa, Artificial Sequence
  Linker
GGGGSGGGGSGGGGS

SEQ ID NO: 53 - protein, 245 aa, Artificial Sequence
  scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 54 - protein, 12 aa, Artificial Sequence
  CDR H3
HYYYGGSYAMDY

SEQ ID NO: 55 - protein, 7 aa, Artificial Sequence
  CDR L2
HTSRLHS

SEQ ID NO: 56 - protein, 9 aa, Artificial Sequence
  CDR L3
QQGNTLPYT

SEQ ID NO: 57 - DNA, 735 bp, Artificial Sequence
  Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 58 - protein, 5 aa, Artificial Sequence
  Hinge
  VARIANT (1)...(1): Xaa = Gly, Cys or Arg
  VARIANT (4)...(4): Xaa = Cys or Thr
XPPXP

SEQ ID NO: 59 - protein, 18 aa, Artificial Sequence
  Linker
GSTSGSGKPGSGEGSTKG

SEQ ID NO: 60 - protein, 451 aa, Artificial Sequence
  Heavy Chain
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 215 aa, Artificial Sequence
  Light Chain
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2021/0069246 A1
(43) Pub. Date: Mar. 11, 2021

(54) COMBINATION THERAPY USING ADOPTIVE CELL THERAPY AND CHECKPOINT INHIBITOR

(71) Applicant: Celgene Corporation, Summit, NJ (US)

(72) Inventors: Stanley R. FRANKEL, Summit, NJ (US); Jens HASSKARL, Boudry (CH); Oliver MANZKE, Boudry (CH)

(73) Assignee: Celgene Corporation, Summit, NJ (US)

(21) Appl. No.: 16/965,287

(22) PCT Filed: Jan. 31, 2019
(86) PCT No.: PCT/US2019/016190

§ 371 Date: Jul. 27, 2020

Related U.S. Application Data
(60) Provisional application No. 62/624,802, filed on Jan. 31, 2018.

(51) Int. Cl. A61K 35/17; A61P 35/00; A61K 39/395; C07K 16/28; C07K 14/725; C07K 14/705; A61K 9/00; A61K 31/7076; A61K 31/664; A61K 38/17

(52) U.S. Cl. CPC A61K 35/17; A61P 35/00; A61K 39/39558; C07K 16/2803; C07K 14/7051; C07K 14/70521; A61K 2039/545; C07K 14/70517; A61K 9/0019; A61K 31/7076; A61K 31/664; A61K 38/1774; A61K 38/177; C07K 14/70578

(57) ABSTRACT

Provided are methods, compositions, uses and articles of manufacture of combination therapies involving immunotherapies, such as adoptive cell therapy, e.g., T cell therapy, and the use of a checkpoint inhibitor, such as an anti-PD-L1 antibody or antigen-binding fragment thereof for treating subjects with disease and conditions such as certain B cell malignancies, and related methods, compositions, uses and articles of manufacture. The cells generally express recombinant receptors such as chimeric antigen receptors (CARs). In some embodiments, the disease or condition is a non-Hodgkin lymphoma (NHL), such as relapsed or refractory NHL or specific NHL subtype.

### CROSS-REFERENCE TO RELATED APPLICATIONS

This application claims priority from U.S. provisional application No. 62/624,802, filed Jan. 31, 2018, entitled “COMBINATION THERAPY USING ADOPTIVE CELL THERAPY AND ANTI-PD-L1 ANTIBODY,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042015640SeqList.TXT, created Jan. 31, 2019 which is 40 kilobytes in size. The information in the electronic format of the Sequence Listing is incorporated by reference in its entirety.

### FIELD

The present disclosure relates in some aspects to combination therapies involving immunotherapies, such as adoptive cell therapy, e.g., T cell therapy, and the use of a checkpoint inhibitor, such as an anti-PD-L1 antibody or antigen-binding fragment thereof for treating subjects with disease and conditions such as certain B cell malignancies, and related methods, compositions, uses and articles of manufacture. The cells generally express recombinant receptors such as chimeric antigen receptors (CARs). In some embodiments, the disease or condition is a non-Hodgkin lymphoma (NHL), such as relapsed or refractory NHL or specific NHL subtype.

### BACKGROUND

Various strategies are available for immunotherapy, for example administering engineered T cells for adoptive therapy. For example, strategies are available for engineering T cells expressing genetically engineered antigen receptors, such as CARs, and administering compositions containing such cells to subjects. Improved strategies are needed to improve efficacy of the cells, for example, improving the persistence, activity and/or proliferation of the cells upon administration to subjects. Provided are methods, compositions, kits, and systems that meet such needs.

### SUMMARY

Provided herein are methods, compositions, uses, article of manufacture involving combination therapies involving administration of an immunotherapy involving a cell therapy, such as a T cell therapy, and subsequently administering to the subject a checkpoint inhibitor, such as an inhibitor of the PD-1/PD-L1 axis of the immune checkpoint pathway, such as an anti-PD-L1 antibody or antigen-binding fragment thereof to a subject having a B cell malignancy. In some aspects, the B cell malignancy is a non-Hodgkin lymphoma (NHL), such as relapsed or refractory NHL or specific NHL subtype. In some aspects, the provided methods, uses, and article of manufacture involve the administration of a T cell therapy such as CAR-expressing T cells comprises an antigen-binding domain that binds to an antigen expressed on B cells or an antigen expressed by or associated with cells of a B cell malignancy. In some aspects the antigen is CD19. In some aspects, the provided methods, uses, and article of manufacture involve the administration of a checkpoint inhibitor, such as an anti-PD-L1 antibody or antigen-binding fragment thereof, comprising administration of the checkpoint inhibitor, such as an anti-PD-L1 antibody or antigen binding fragment thereof, in at least two 28-day cycles. In some aspects, each of said at least two 28-day cycles comprises administration of a total dosage amount of at or about 400 mg to at or about 2000 mg, such as at or about 750 mg to at or about 2000 mg, or at or about 400 mg to at or about 600 mg, for example, for administering certain checkpoint inhibitors such as an anti-PD-L1 antibody or fragment thereof. In some embodiments, the first 28-day cycle is carried out by administering a greater number of individual doses of a checkpoint inhibitor, such as an anti-PD-L1 antibody or antigen binding fragment thereof. In some embodiments, the first 28-day cycle of the administration of checkpoint inhibitor, such as the anti-PD-L1 antibody or fragment thereof, is initiated at a time between day 22 and day 36 of initiation of the administration of the cell therapy.

Provided herein are methods of treatment that involve: (a) administering a T cell therapy to a subject having a B cell malignancy, said cell therapy comprising a dose of genetically engineered T cells expressing a chimeric antigen receptor, wherein the chimeric antigen receptor specifically binds to a target antigen expressed by the B cell malignancy; and (b) subsequently administering to the subject a checkpoint inhibitor that is an antibody or antigen-binding fragment thereof capable of blocking an immune checkpoint pathway protein, wherein a total dosage amount of the checkpoint inhibitor is administered in each of at least two dosage cycles, wherein the total dosage amount of the checkpoint inhibitor in the first of the at least two dosage cycles: is the same as or less than the total dosage amount administered in the second and/or a subsequent dosage cycle; and is administered in more than one individual dose over the course of the first dosage cycle, wherein the number of individual doses is greater than the number of individual doses administered in the second and/or a subsequent dosage cycle.

Provided herein are methods of treatment that involve administering, to a subject having a B cell malignancy a checkpoint inhibitor that is an antibody or antigen-binding fragment thereof capable of blocking an immune checkpoint pathway protein, said subject having been administered a T cell therapy comprising a dose of genetically engineered T cells expressing a chimeric antigen receptor that specifically binds to a target antigen expressed by the B cell malignancy, wherein a total dosage amount of the checkpoint inhibitor is administered in each of at least two dosage cycles, wherein the total dosage amount of the checkpoint inhibitor in the first of the at least two dosage cycles: is the same as or less than the total dosage amount administered in the second and/or a subsequent dosage cycle; and is administered in more than one individual dose over the course of the first dosage cycle, wherein the number of individual doses is greater than the number of individual doses administered in the second and/or a subsequent dosage cycle.

In some of any such embodiments, the dosage cycle is a 21-day cycle. In some of any such embodiments, the dosage cycle is a 28-day cycle.

In some of any such embodiments, the total dosage amount in the first of the at least two dosage cycles is the same as the total dosage amount in the second of the at least two dosage cycles.

In some of any such embodiments, the first of the at least two dosage cycles comprises 2, 3, 4 or more individual doses.

In some of any such embodiments, the dosage cycle is a 28-day cycle and the individual doses of the first of the at least two 28-day cycles are administered as four doses each once every week (Q1W), two doses each as Q1W doses for two consecutive weeks, or two doses each as Q1W doses for two consecutive weeks and followed by one dose once in two weeks (Q2W).

In some of any such embodiments, each of said at least two dosage cycles comprises administering independently a total dosage amount of at or about 400 mg to at or about 2000 mg of the checkpoint inhibitor.

In some of any such embodiments, the checkpoint inhibitor blocks an immune checkpoint pathway protein selected from among PD-L1, PD-L2, PD-1 and CTLA-4.

In some of any such embodiments, the checkpoint pathway is PD-1/PD-L1 and the checkpoint inhibitor is an anti-PD-1 antibody. In some of any such embodiments, the checkpoint inibitior is nivolumab, pembrolizumab, or cemiplimab. In some of any such embodiments, each of said at least two dosage cycle comprises administering independently a total dosage amount of at or about 400 mg to at or about 600 mg, optionally at or about 480 mg.

In some of any such embodiments, the checkpoint pathway is PD-1/PD-L1 and the checkpoint inhibitor is an anti-PD-L1 antibody. In some of any such embodiments, each of said at least two dosage cycle comprises administering independently a total dosage amount of 750 mg to 2000 mg, optionally at or about 1500 mg.

In some of any such embodiments, administration of the checkpoint inhibitor and/or the start of the first dosage cycle is initiated at a time at or after, optionally immediately after or within 1 to 3 days after: (i) peak or maximum level of the cells of the T cell therapy are detectable in the blood of the subject; (ii) the number of cells of the T cell therapy detectable in the blood, after having been detectable in the blood, is not detectable or is reduced, optionally reduced compared to a preceding time point after administration of the T cell therapy; (iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy; (iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject; (v) the subject exhibits disease progression and/or has relapsed following remission after treatment with the T cell therapy; and/or (iv) the subject exhibits increased tumor burden as compared to tumor burden at a time prior to or after administration of the cells and prior to initiation of administration of the checkpoint inhibitor.

In some of any such embodiments, administration of the checkpoint inhibitor and/or the start of the first dosage cycle is initiated at or within 29 days, 36 days, 43 days or 50 days after initiation of administration of the T cell therapy. In some of any such embodiments, administration of the checkpoint inhibitor and/or the start of the first dosage cycle is initiated from or from about 22 days to 36 days after initiation of administration of the T cell therapy. In some of any such embodiments, administration of the checkpoint inhibitor and/or the start of the first dosage cycle is initiated at or about 29 days after initiation of administration of the T cell therapy. In some of any such embodiments, administration of the checkpoint inhibitor and/or the start of the first dosage cycle is initiated at or about 43 days after initiation of administration of the T cell therapy.

In some of any such embodiments, at the time of administering the checkpoint inhibitor and/or the start of the first dosage cycle, the subject does not exhibit a severe toxicity following administration of the T cell therapy. In some of any such embodiments: the severe toxicity is severe cytokine release syndrome (CRS), optionally grade 3 or higher, prolonged grade 3 or higher or grade 4 or 5 CRS; and/or the severe toxicity is severe neurotoxicity, optionally grade 3 or higher, prolonged grade 3 or higher or grade 4 or 5 neurotoxicity.

Provided herein are methods of treatment that involve: (a) administering a T cell therapy to a subject having a B cell malignancy, said T cell therapy comprising a dose of genetically engineered T cells expressing a chimeric antigen receptor, wherein the chimeric antigen receptor specifically binds to a target antigen expressed by cells of the B cell malignancy; and (b) subsequently administering to the subject an anti-PD-L1 antibody or antigen-binding fragment thereof, wherein said administration comprises carrying out at least two 28-day cycles, wherein: the first 28-day cycle comprises administering the anti-PD-L1 antibody or antigen-binding fragment thereof as two individual doses each once-weekly (Q1W) for two consecutive weeks of the 28-day cycle, each of said individual doses in an amount of or about 375 mg, followed by one dose once in two weeks (Q2W) of the 28-day cycle in an amount of or about 750 mg; and the second and/or a subsequent 28-day cycle comprises administering the anti-PD-L1 antibody or antigen-binding fragment thereof as one dose every four weeks (Q4W) for in an amount of or about 1500 mg.

Provided herein are methods of treatment that involve administering an anti-PD-L1 antibody or antigen-binding fragment thereof to a subject having a B cell malignancy, said subject having been administered a T cell therapy comprising a dose of genetically engineered T cells expressing a chimeric antigen receptor that specifically binds to a target antigen expressed by the B cell malignancy wherein the administration of the anti-PD-L1 antibody or antigen-binding fragment thereof comprises carrying out at least two 28-day cycles, wherein: the first 28-day cycle comprises administering the anti-PD-L1 antibody or antigen-binding fragment thereof as two individual doses each once-weekly (Q1W) for two consecutive weeks of the 28-day cycle, each of said individual doses in an amount of or about 375 mg, followed by one dose once in two weeks (Q2W) of the 28-day cycle in an amount of or about 750 mg; and the second and/or a subsequent 28-day cycle comprises administering the anti-PD-L1 antibody or antigen-binding fragment thereof as one dose every four weeks (Q4W) for in an amount of or about 1500 mg.

Provided herein are methods of treatment that involve: (a) administering a T cell therapy to a subject having a B cell malignancy, said T cell therapy comprising a dose of genetically engineered T cells expressing a chimeric antigen receptor, wherein the chimeric antigen receptor specifically binds to a target antigen expressed by the B cell maligancy; and (b) subsequently administering to the subject an anti-PD-L1 antibody or antigen-binding fragment thereof, said administration comprises carrying out at least two 28-day cycles, wherein: the first 28-day cycle comprises administering the anti-PD-L1 antibody or antigen-binding fragment thereof as four individual doses each once-weekly (Q1W) for the 28-day cycle, wherein, said four doses comprises two consecutive Q1W doses each independently of or of about 225 mg followed by two consecutive Q1W doses each independently of or of about 375 mg; and the second and/or a subsequent 28-day cycle comprises administering the anti-PD-L1 antibody or antigen-binding fragment thereof as two doses each every two weeks (Q2W) of the 28-day cycle, wherein each Q2W administration is each independently in an amount of or of about 750 mg.

Provided herein are methods of treatment that involve administering an anti-PD-L1 antibody or antigen-binding fragment thereof to a subject having a B cell malignancy, said subject having been administered a T cell therapy comprising a dose of genetically engineered T cells expressing a chimeric antigen receptor that specifically binds to a target antigen expressed on the B cell malignancy, wherein the anti-PD-L1 antibody or antigen-binding fragment thereof comprises carrying out at least two 28-day cycles, wherein: the first 28-day cycle comprises administering the anti-PD-L1 antibody or antigen-binding fragment thereof as four individual doses each once-weekly (Q1W) for the 28-day cycle, wherein the four individual doses comprises two consecutive Q1W doses each independently of or of about 225 mg followed by two consecutive Q1W doses each independently of or about 375 mg; and the second and/or a subsequent 28-day cycle comprises administering the anti-PD-L1 antibody or antigen-binding fragment thereof as two individual doses every two weeks (Q2W) for the second and/or subsequent 28-day cycle, wherein each dose independently is in an amount of or about 750 mg.

Provided herein are methods of treatment that involve: (a) administering a T cell therapy to a subject having a B cell malignancy, said T cell therapy comprising a dose of genetically engineered T cells expressing a chimeric antigen receptor, wherein the chimeric antigen receptor specifically binds to a target antigen expressed by the B cell malignancy; and (b) subsequently administering to the subject an anti-PD-L1 antibody or antigen-binding fragment thereof, said administration comprises carrying out at least two 28-day cycles, wherein: the first 28-day cycle comprises administering the anti-PD-L1 antibody or antigen-binding fragment thereof as two individual doses each once-weekly (Q1W), wherein each of said two doses independently comprises an amount of or of about 375 mg, optionally wherein the two doses are consecutive Q1W doses, optionally wherein the two doses are administered on days 15 and 22 in the 28-day cycle; and the second and/or a subsequent 28-day cycle comprises administering the anti-PD-L1 antibody or antigen-binding fragment thereof Q4W for one dose in the second and/or subsequent 28-day cycle in an amount of or about 1500 mg.

Provided herein are methods of treatment that involve administering an anti-PD-L1 antibody or antigen-binding fragment thereof to a subject having a B cell malignancy, said subject having been administered a T cell therapy comprising a dose of genetically engineered T cells expressing a chimeric antigen receptor that specifically binds to a target antigen expressed on the B cell malignancy, wherein the administration of the anti-PD-L1 antibody or antigen-binding fragment comprises carrying out at least two 28-day cycles, wherein: the first 28-day cycle comprises administering the anti-PD-L1 antibody or antigen-binding fragment thereof as two individual doses each once-weekly (Q1W), wherein each of said two doses independently comprises an amount of or of about 375 mg, optionally wherein the two doses are consecutive Q1W doses, optionally wherein the two doses are administered on days 15 and 22 in the 28-day cycle; and the second and/or a subsequent 28-day cycle comprises administering the anti-PD-L1 antibody or antigen-binding fragment thereof Q4W for one dose in the second and/or subsequent 28-day cycle in an amount of or about 1500 mg.

Provided herein are methods of treatment that involve: (a) administering a T cell therapy to a subject having a B cell malignancy, said cell therapy comprising a dose of genetically engineered T cells expressing a chimeric antigen receptor, wherein the chimeric antigen receptor specifically binds to a target antigen expressed by the B cell malignancy; and (b) subsequently administering to the subject an anti-PD-L1 antibody or antigen-binding fragment thereof, wherein: the administration of the anti-PD-L1 antibody or antigen-binding fragment comprises carrying out at least two 28-day cycles, each of said at least two 28-day cycles, comprising administering a total dosage amount of 750 mg to 2000 mg of the antibody or antigen-binding fragment; and in at least one of said at least two 28-day cycles, the administration of the total dosage amount of the anti-PD-L1 antibody or antigen-binding fragment is carried out by administering more than one individual doses of the antibody or fragment over the course of the at least one 28-day cycle.

Provided herein are methods of treatment that involve administering an anti-PD-L1 antibody or antigen-binding fragment thereof to a subject having a B cell malignancy, said subject having been administered a T cell therapy comprising a dose of genetically engineered T cells expressing a chimeric antigen receptor, wherein the chimeric antigen receptor specifically binds to a target antigen expressed by the B cell malignancy, wherein: the administration of the anti-PD-L1 antibody or antigen-binding fragment comprises carrying out at least two 28-day cycles, each of said at least two 28-day cycles, independently, comprising administering a total dosage amount of 750 mg to 2000 mg of the antibody or antigen-binding fragment; and in at least one of said at least two 28-day cycles, the administration of the total dosage amount of the anti-PD-L1 antibody or antigen-binding fragment is carried out by administering more than one individual dose of the antibody or fragment over the course of the at least one 28-day cycle.

In some of any such embodiments, in a first of said at least two 28-day cycles, the administration of the total dosage amount of the anti-PD-L1 antibody or antigen-binding fragment is carried out by administering a greater number of individual doses of the antibody or fragment as compared to the administration in the second and/or a subsequent 28-day cycle.

In some of any such embodiments, the total dosage amount of the anti-PD-L1 antibody or antigen-binding fragment in each 28-day cycle independently is between at or about 750 mg and at or about 1500 mg. In some of any such embodiments, the total dosage amount of the anti-PD-L1 antibody or antigen-binding fragment in at least one of the 28-day cycles is at or about 750 mg. In some of any such embodiments, the total dosage amount of the anti-PD-L1 antibody or antigen-binding fragment in at least one of the 28-day cycles is at or about 1200 mg. In some of any such embodiments, the total dosage amount of the anti-PD-L1 antibody or antigen-binding fragment in at least one of the 28-day cycles is at or about 1500 mg.

In some of any such embodiments, the total dosage amount of the anti-PD-L1 antibody or antigen-binding fragment in each 28-day cycle, independently, is at or about 1500 mg.

In some of any such embodiments, the total dosage amount of the anti-PD-L1 antibody or antigen-binding fragment in at least two of said at least two, and optionally in said at least two, 28-day cycles is the same total dosage amount. In some of any such embodiments, the total dosage amount of the anti-PD-L1 antibody or antigen-binding fragment is different in at least two of, or is different in each of, said at least two 28-day cycles. In some of any such embodiments, the total dosage amount of the anti-PD-L1 antibody or antigen-binding fragment in the first of said at least two 28-day cycles is lower than the second and/or a subsequent of said at least two 28-day cycle.

In some of any such embodiments, the administration of the total dosage amount in the first of said at least two 28-day cycles comprises administering 2, 3 or 4 individual doses of the anti-PD-L1 antibody or antigen-binding fragment thereof.

In some of any such embodiments, the administration of the total dosage amount in the first of said at least two 28-day cycles comprises administering the anti-PD-L1 antibody or antigen-binding fragment thereof as individual doses according to a dosing schedule selected from (i) two individual doses each once-weekly (Q1W) within the 28-day cycle, optionally on days 15 and 22 of the 28-day cycle; (ii) four individual doses each once-weekly (Q1W) for the 28-day cycle, optionally on days 1, 8, 15 and 22 of the 28-day cycle; (iii) two individual doses each Q1W for two consecutive weeks of the 28-day cycle, optionally on days 1 and 8 of the cycle, followed by one dose once in two weeks (Q2W) of the 28-day cycle, optionally on day 15 of the cycle; or (iv) two individual doses each every two weeks (Q2W) for the 28-day cycle, optionally on days 1 and 15 of the 28-day cycle.

In some of any such embodiments, each Q1W dose administered in the first 28-day cycle is independently from or from at or about 18% to at or about 32% of the total dosage amount administered in the first 28-day cycle, optionally is at or about 25% of the total dosage amount administered in the first 28-day cycle; and/or each Q2W dose administered in the first 28-day cycle is independently from or from at or about 40% to at or about 62.5% of the total dosage amount in the first 28-day cycle, optionally is at or about 50% of the total dosage amount administered in the first 28-day cycle.

In some of any such embodiments, the administration of the total dosage amount in the first 28-day cycle comprises administering the anti-PD-L1 antibody or antigen-binding fragment thereof according to dosing schedule (iii), wherein each of the two individual doses Q1W for two consecutive weeks is, each independently, in an amount of or of about 375 mg followed by one dose once Q2W in an amount of or of about 750 mg; the administration of the total dosage amount in the first 28-day cycle comprises administering the anti-PD-L1 antibody or antigen-binding fragment thereof according to dosing schedule set forth in (ii), wherein the four individual doses Q1W comprise two consecutive Q1W doses in an amount of or of about 225 mg followed by two consecutive Q1W doses in an amount of or of about 375 mg; or the administration of the total dosage amount in the first 28-day cycle comprises administering the anti-PD-L1 antibody or antigen-binding fragment according to dosing schedule set forth in (i), wherein each of the two individual doses Q1W are carried out for two consecutive Q1W doses in an amount of or of about 375 mg.

In some of any such embodiments, the administration of the total dosage amount in the first of said at least two 28-day cycles comprises administering individual doses according to a dosing schedule selected from (i) two individual doses on or about day 15 and on or about day 22 of the 28-day cycle; (ii) four individual doses on or about day 1, on or about day 8, on or about day 15 and on or about day 22 of the 28-day cycle; (iii) two individual doses on or about day 1 and on or about day 8 of the 28-day cycle, followed by one dose on or about day 15 of the cycle; or (iv) two doses on or about day 1 of the 28-day cycle and on or about day 15 of the 28-day cycle.

In some of any such embodiments, the administration of the total dosage amount in the first 28-day cycle comprises administering the anti-PD-L1 antibody or antigen-binding fragment thereof according to dosing schedule (iii), wherein each of the two individual doses comprise an amount of or of about 375 mg on or about day 1 and on or about day 8 of the 28-day cycle, followed by one dose in an amount of or of about 750 mg on or about day 15 of the cycle; the administration of the total dosage amount in the first 28-day cycle comprises administering the anti-PD-L1 antibody or antigen-binding fragment thereof according to dosing schedule set forth in (ii), wherein the four individual doses comprise two consecutive doses in an amount of or of about 225 mg on or about day 1 and on or about day 8, followed by two consecutive doses in an amount of or of about 375 mg on or about day 15 and on or about day 22 of the 28-day cycle; or the administration of the total dosage amount in the first 28-day cycle comprises administering the anti-PD-L1 antibody or antigen-binding fragment according to dosing schedule set forth in (i), wherein each of the two individual doses comprise two consecutive in an amount of or of about 375 mg on or about day 15 and on or about day 22 of the 28-day cycle.

In some of any such embodiments, the administration of the total dosage amount in the second and/or a subsequent 28-day cycle, independently, comprises administering 1 or 2 doses of the anti-PD-L1 antibody or antigen-binding fragment thereof.

In some of any such embodiments, the administration of the total dosage amount in the second and/or a subsequent 28-day cycle, independently, comprises a dosing schedule selected from: (i) two individual doses each every two weeks (Q2W) for the second and/or subsequent 28-day cycle, optionally on days 1 and 15 of the second and/or subsequent cycle; or (ii) one dose every four weeks (Q4W) of the second and/or subsequent 28-day cycle, optionally on day 1 of the second and/or subsequent cycle.

In some of any such embodiments: each Q2W dose of the second and/or subsequent 28-day cycle is or is about 50% of the total dosage amount of the second and/or subsequent 28 day cycle; and/or the Q4W dose of the second and/or subsequent 28-day cycle is or is about the total dosage amount of the second and/or subsequent 28 day cycle.

In some of any such embodiments, the second and/or a subsequent dose comprises administering the anti-PD-L1 antibody or antigen-binding fragment thereof Q2W for two doses in an amount of or of about 750 mg; or the second and/or a subsequent dose comprises administering the anti-PD-L1 antibody or antigen-binding fragment thereof Q4W for one dose in an amount of or of about 1500 mg.

In some of any such embodiments, at least two 28-day cycles further comprises a third 28-day cycle and/or wherein the subsequent 28-day cycle is a third 28-day cycle. In some of any such embodiments, the total dosage amount of the anti-PD-L1 antibody or antigen-binding fragment in the third 28-day cycle is the same as the total dosage amount administered in the first and/or in the second 28-day cycle. In some of any such embodiments, the total dosage amount of the anti-PD-L1 antibody or antigen-binding fragment in the third 28-day cycle is or is about 1500 mg.

In some of any such embodiments, (a) in the third 28-day cycle, the administration of the total dosage amount of the anti-PD-L1 antibody or antigen-binding fragment is carried out by administering the antibody or fragment in a greater number of individual doses as compared to in the first and/or second 28-day cycle; or (b) in the third 28-day cycle, the administration of the total dosage amount of the anti-PD-L1 antibody or antigen-binding fragment is carried out by administering the same number of doses of the antibody or fragment as compared to the second 28-day cycle.

In some of any such embodiments, the administration of the total dosage amount in the third 28-day cycle comprises administration of one dose every four weeks (Q4W) of the third 28-day cycle, optionally on day 1 of the third 28-day cycle.

In some of any such embodiments, the first of said at least two 28-day cycles is initiated at a time: (a) between day 22 and day 36 of initiation of the administration of the T cell therapy; or (b) at or after, optionally immediately after or within 1 to 3 days after: (i) peak or maximum level of the cells of the T cell therapy are detectable in the blood of the subject; (ii) the number of cells of the T cell therapy detectable in the blood, after having been detectable in the blood, is not detectable or is reduced, optionally reduced compared to a preceding time point after administration of the T cell therapy; (iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy; (iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject; (v) the subject exhibits disease progression and/or has relapsed following remission after treatment with the T cell therapy; and/or (iv) the subject exhibits increased tumor burden as compared to tumor burden at a time prior to or after administration of the cells and prior to initiation of administration of the anti-PD-L1 antibody.

In some of any such embodiments, the first of said at least two 28-day cycles is initiated at a time between day 22 and day 36 of initiation of the administration of the T cell therapy. In some of any such embodiments, the at least two 28-day cycles comprise no more than three 28-day cycles, optionally wherein the first of said at least two 28-day cycles is initiated between at or about day 22 and at or about day 36. In some of any such embodiments, the first of said at least two 28-day cycle is initiated at or about day 29 after initiation of the administration of the T cell therapy. In some of any such embodiments, the first of said at least two 28-day cycle is initiated at or about day 43 after initiation of administration of the T cell therapy.

Provided herein are methods of treatment that involve: (a) administering a T cell therapy to a subject having a B cell malignancy, said T cell therapy comprising a dose of genetically engineered T cells expressing a chimeric antigen receptor, wherein the chimeric antigen receptor specifically binds to a target antigen expressed by the B cell malignancy; and (b) subsequently administering to the subject an anti-PD-L1 antibody or antigen-binding fragment thereof, wherein the administration of antibody or antigen-binding fragment comprises carrying out between one and three 28-day cycles, each cycle comprising administering a total dosage amount of 750 mg to 2000 mg of the antibody or fragment, optionally wherein the first of said between one and three 28-day cycle begins between at or about day 22 and at or about day 36, optionally at day 29, after initiation of the T cell therapy.

Provided herein are methods of treatment that involve administering an anti-PD-L1 antibody or antigen-binding fragment thereof to a subject having a B cell malignancy, said subject having been administered a T cell therapy comprising a dose of genetically engineered T cells expressing a chimeric antigen receptor, wherein the chimeric antigen receptor specifically binds to a target antigen expressed by the B cell malignancy, wherein the administration of the antibody or antigen-binding fragment comprises carrying out between one and three 28-day cycles, each cycle comprises administering a total dosage amount of 900 mg to 2000 mg of the antibody or fragment, optionally wherein the first of said between one and three 28-day cycles begins between at or about day 22 and at or about day 36, optionally at about day 29, after initiation of the T cell therapy.

Provided are methods of treatment that involve: (a) administering a T cell therapy to a subject having a B cell malignancy, said cell therapy comprising a dose of genetically engineered T cells expressing a recombinant receptor; and (b) subsequently administering to the subject an anti-PD-L1 antibody or antigen-binding fragment thereof, wherein: the administration of the anti-PD-L1 antibody or antigen-binding fragment comprises carrying out at least two 28-day cycles, each of said at least two 28-day cycles, independently, comprising administering a total dosage amount of 750 mg to 2000 mg of the antibody or antigen-binding fragment; and in at least the one of said at least two 28-day cycles, the administration of the total dosage amount of the anti-PD-L1 antibody or antigen-binding fragment is carried out by administering more than one individual doses of the antibody or fragment over the course of the at least one 28-day cycle.

Provided here are methods of treatment that involve: an anti-PD-L1 antibody or antigen-binding fragment thereof to a subject having a B cell malignancy, said subject having been administered a T cell therapy comprising a dose of genetically engineered T cells expressing a recombinant receptor, wherein: the administration of the anti-PD-L1 antibody or antigen-binding fragment comprises carrying out at least two 28-day cycles, each of said at least two 28-day cycles, independently, comprising administering a total dosage amount of 750 mg to 2000 mg of the antibody or antigen-binding fragment; and in at least the one of said at least two 28-day cycles, the administration of the total dosage amount of the anti-PD-L1 antibody or antigen-binding fragment is carried out by administering more than one individual dose of the antibody or fragment over the course of the at least one 28-day cycle.

In some embodiments of any one of the methods provided herein, in a first of said at least two 28-day cycles, the administration of the total dosage amount of the anti-PD-L1 antibody or antigen-binding fragment is carried out by administering a greater number of individual doses of the antibody or fragment as compared to the administration in a second and/or a subsequent 28-day cycle.

In some embodiments of any one of the methods provided herein, the total dosage amount of the anti-PD-L1 antibody or antigen-binding fragment in each 28-day cycle independently is between at or about 750 mg and at or about 1500 mg. In some embodiments, the total dosage amount of the anti-PD-L1 antibody or antigen-binding fragment in at least one of the 28-day cycles is at or about 750 mg. In some embodiments, the total dosage amount of the anti-PD-L1 antibody or antigen-binding fragment in at least one of the 28-day cycles is at or about 1200 mg. In some embodiments, the total dosage amount of the anti-PD-L1 antibody or antigen-binding fragment in at least one of the 28-day cycles is at or about 1500 mg.

In some embodiments of any one of the methods provided herein, the total dosage amount of the anti-PD-L1 antibody or antigen-binding fragment in each 28-day cycle, independently, is at or about 1500 mg.

In some embodiments of any one of the methods provided herein, in the total dosage amount of the anti-PD-L1 antibody or antigen-binding fragment in at least two of said at least two, and optionally in said at least two, 28-day cycles is the same total dosage amount. In some embodiments, the total dosage amount of the anti-PD-L1 antibody or antigen-binding fragment is different in at least two of, or is different in each of, said at least two 28-day cycles. In some embodiments, the total dosage amount of the anti-PD-L1 antibody or antigen-binding fragment in the first of said at least two 28-day cycles is lower than the second and/or a subsequent of said at least two 28-day cycle.

In some embodiments of any one of the methods provided herein, the administration of the total dosage amount in the first of said at least two 28-day cycles comprises administering 2, 3 or 4 individual doses of the anti-PD-L1 antibody or antigen-binding fragment thereof.

In some embodiments of any one of the methods provided herein, the administration of the total dosage amount in the first of said at least two 28-day cycles comprises administering individual doses according to a dosing schedule selected from (i) once-weekly (Q1W) for two individual doses, optionally on days 15 and 22 of the 28-day cycle; (ii) once-weekly (Q1W) for four individual doses, optionally on days 1, 8, 15 and 22 of the 28-day cycle; (iii) Q1W for two consecutive doses, optionally on days 1 and 8 of the cycle, followed by every two weeks (Q2W) for one dose, optionally on day 15 of the cycle; or (iv) every two weeks (Q2W) for two doses, optionally on days 1 and 15 of the 28-day cycle. In some embodiments, each Q1W dose administered in the first 28-day cycle is independently from or from at or about 18% to at or about 32% of the total dosage amount administered in the first 28-day cycle, optionally is at or about 25% of the total dosage amount administered in the first 28-day cycle; and/or each Q2W dose administered in the first 28-day cycle is independently from or from at or about 40% to at or about 62.5% of the total dosage amount, optionally is at or about 50% of the total dosage amount administered in the first 28-day cycle. In some embodiments, the administration of the total dosage amount in the first 28-day cycle comprises administering the anti-PD-L1 antibody or antigen-binding fragment thereof Q1W for two consecutive doses, each independently, in an amount of or of about 375 mg followed by Q2W for one dose in an amount of or of about 750 mg; the administration of the total dosage amount in the first 28-day cycle comprises administering the anti-PD-L1 antibody or antigen-binding fragment thereof Q1W for four doses, said four doses comprising two consecutive doses of or about 225 mg followed by two consecutive doses of or about 375 mg; or the administration of the total dosage amount in the first 28-day cycle comprises administering the anti-PD-L1 antibody or antigen-binding fragment thereof Q1W for two consecutive doses in an amount of or about 375 mg.

In some embodiments of any one of the methods provided herein, the administration of the total dosage amount in the second and/or a subsequent 28-day cycle, independently, comprises administering 1 or 2 does of the anti-PD-L1 antibody or antigen-binding fragment thereof.

In some embodiments of any one of the methods provided herein, the administration of the total dosage amount in the second and/or a subsequent 28-day cycle, independently, comprises a dosing schedule selected from (i) every two weeks (Q2W) for two doses, optionally on days 1 and 15 of the cycle; or (ii) every four weeks (Q4W) for one dose, optionally on day 1 of the cycle. In some embodiments, each Q2W dose of the second and/or subsequent 28-day cycle is or is about 50% of the total dosage amount; and/or the Q4W dose of the second and/or subsequent 28-day cycle is or is about the total dosage amount. In some embodiments, the second and/or a subsequent dose comprises administering the anti-PD-L1 antibody or antigen-binding fragment thereof Q2W for two doses in an amount of or of about 750 mg; or the second and/or a subsequent dose comprises administering the anti-PD-L1 antibody or antigen-binding fragment thereof Q4W for one dose in an amount of or of about 1500 mg.

Provided here are methods of treatment that involve: (a) administering a T cell therapy to a subject having a B cell malignancy, said T cell therapy comprising a dose of genetically engineered T cells expressing a recombinant receptor; and (b) subsequently administering to the subject an anti-PD-L1 antibody or antigen-binding fragment thereof, wherein said administration comprises carrying out at least two 28-day cycles, wherein: the first 28-day cycle comprises administering the anti-PD-L1 antibody or antigen-binding fragment thereof once-weekly (Q1W) for two consecutive doses in an amount of or about 375 mg followed by every two weeks (Q2W) for one dose in an amount of or about 750 mg; and the second and/or a subsequent 28-day cycle comprises administering the anti-PD-L1 antibody or antigen-binding fragment thereof Q4W for one dose in an amount of or about 1500 mg.

Provided here are methods of treatment that involve: administering an anti-PD-L1 antibody or antigen-binding fragment thereof to a subject having a B cell malignancy, said subject having been administered a T cell therapy comprising a dose of genetically engineered T cells expressing a recombinant receptor, wherein the administration of the anti-PD-L1 antibody or antigen-binding fragment thereof comprises carrying out at least two 28-day cycles, wherein: the first 28-day cycle comprises administering the anti-PD-L1 antibody or antigen-binding fragment thereof once-weekly (Q1W) for two consecutive doses independently in an amount of or of about 375 mg followed by every two weeks (Q2W) for one dose in an amount of or of about 750 mg; and the second and/or a subsequent 28-day cycle comprises administering the anti-PD-L1 antibody or antigen-binding fragment thereof every four weeks (Q4W) for one dose in an amount of or of about 1500 mg.

Provided here are methods of treatment that involve: (a) administering a T cell therapy to a subject having a B cell malignancy, said T cell therapy comprising a dose of genetically engineered T cells expressing a recombinant receptor; and (b) subsequently administering to the subject an anti-PD-L1 antibody or antigen-binding fragment thereof, said administration comprises carrying out at least two 28-day cycles, wherein: the first 28-day cycle comprises administering the anti-PD-L1 antibody or antigen-binding fragment thereof once-weekly (Q1W) for four doses, said four doses comprising two consecutive doses each independently of or of about 225 mg followed by two consecutive doses each independently of or of about 375 mg; and the second and/or a subsequent 28-day cycle comprises administering the anti-PD-L1 antibody or antigen-binding fragment thereof every two weeks (Q2W) for two doses each independently in an amount of or of about 750 mg.

Provided here are methods of treatment that involve: administering an anti-PD-L1 antibody or antigen-binding fragment thereof to a subject having a B cell malignancy, said subject having been administered a T cell therapy comprising a dose of genetically engineered T cells expressing a recombinant receptor, wherein the anti-PD-L1 antibody or antigen-binding fragment thereof comprises carrying out at least two 28-day cycles, wherein: the first 28-day cycle comprises administering the anti-PD-L1 antibody or antigen-binding fragment thereof once-weekly (Q1W) for four doses, said four doses comprising two consecutive doses each independently of or of about 225 mg followed by two consecutive doses of or about 375 mg; and the second and/or a subsequent 28-day cycle comprises administering the anti-PD-L1 antibody or antigen-binding fragment thereof every two weeks (Q2W) for two doses in an amount of or about 750 mg.

Provided here are methods of treatment that involve: (a) administering a T cell therapy to a subject having a B cell malignancy, said T cell therapy comprising a dose of genetically engineered T cells expressing a recombinant receptor; and (b) subsequently administering to the subject an anti-PD-L1 antibody or antigen-binding fragment thereof, said administration comprises carrying out at least two 28-day cycles, wherein: the first 28-day cycle comprises administering the anti-PD-L1 antibody or antigen-binding fragment thereof once-weekly (Q1W) for two doses, each of said two doses each independently comprising an amount of or of about 375 mg, optionally wherein the two doses are consecutive doses, optionally wherein the two doses are administered days 15 and 22 in the 28-day cycle; and the second and/or a subsequent 28-day cycle comprises administering the anti-PD-L1 antibody or antigen-binding fragment thereof Q4W for one dose in an amount of or about 1500 mg.

Provided here are methods of treatment that involve: administering an anti-PD-L1 antibody or antigen-binding fragment thereof to a subject having a B cell malignancy, said subject having been administered a T cell therapy comprising a dose of genetically engineered T cells expressing a recombinant receptor, wherein the administration of the anti-PD-L1 antibody or antigen-binding fragment comprises carrying out at least two 28-day cycles, wherein: the first 28-day cycle comprises administering the anti-PD-L1 antibody or antigen-binding fragment thereof once-weekly (Q1W) for two doses, each of said two doses independently comprising an amount of or about 375 mg, optionally wherein the two doses are consecutive doses, optionally wherein the two doses are administered on days 15 and 22 in the 28-day cycle; and the second and/or a subsequent 28-day cycle comprises administering the anti-PD-L1 antibody or antigen-binding fragment thereof Q4W for one dose in an amount of or about 1500 mg.

In some embodiments of any one of the methods provided herein, at least two 28-day cycles further comprises a third 28-day cycle and/or wherein the subsequent 28-day cycle is a third 28-day cycle. In some embodiments, the total dosage amount of the anti-PD-L1 antibody or antigen-binding fragment in the third 28-day cycle is the same as the total dosage amount administered in the first and/or in the second 28-day cycle. In some embodiments, the total dosage amount of the anti-PD-L1 antibody or antigen-binding fragment in the third 28-day cycle is or is about 1500 mg. In some embodiments, (a) in the third 28-day cycle, the administration of the total dosage amount of the anti-PD-L1 antibody or antigen-binding fragment is carried out by administering the antibody or fragment in a greater number of individual doses as compared to in the first and/or second 28-day cycle; or (b) in the third 28-day cycle, the administration of the total dosage amount of the anti-PD-L1 antibody or antigen-binding fragment is carried out by administering the same number of doses of the antibody or fragment as compared to the second 28-day cycle. In some embodiments, the administration of the total dosage amount in the third 28-day cycle comprises administration every four weeks (Q4W) for one dose, optionally on day 1 of the third 28-day cycle.

In some embodiments of any one of the methods provided herein, the first of said at least two 28-day cycles is initiated at a time: (a) between day 22 and day 36 of initiation of the administration of the T cell therapy; or (b) at or after, optionally immediately after or within 1 to 3 days after: (i) peak or maximum level of the cells of the T cell therapy are detectable in the blood of the subject; (ii) the number of cells of the T cell therapy detectable in the blood, after having been detectable in the blood, is not detectable or is reduced, optionally reduced compared to a preceding time point after administration of the T cell therapy; (iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy; (iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject; (v) the subject exhibits disease progression and/or has relapsed following remission after treatment with the T cell therapy; and/or (vi) the subject exhibits increased tumor burden as compared to tumor burden at a time prior to or after administration of the cells and prior to initiation of administration of the anti-PD-L1 antibody.

In some embodiments of any one of the methods provided herein, the at least two 28-day cycles comprise no more than three 28-day cycles, optionally wherein the first of said at least two 28-day cycles begins between at or about day 22 and at or about day 36, optionally at or about day 29, after initiation of the administration of the T cell therapy.

Provided here are methods of treatment that involve: (a) administering a T cell therapy to a subject having a B cell malignancy, said T cell therapy comprising a dose of genetically engineered T cells expressing a recombinant receptor; and (b) subsequently administering to the subject an anti-PD-L1 antibody or antigen-binding fragment thereof, wherein the administration of antibody or antigen-binding fragment comprises carrying out between one and three 28-day cycles, each cycle comprising administering a total dosage amount of 750 mg to 2000 mg of the antibody or fragment, optionally wherein the first of said between one and three 28-day cycle begins between at or about day 22 and at or about day 36, optionally at day 29, after initiation of the T cell therapy.

Provided here are methods of treatment that involve: administering an anti-PD-L1 antibody or antigen-binding fragment thereof to a subject having a B cell malignancy, said subject having been administered a T cell therapy comprising a dose of genetically engineered T cells expressing a recombinant receptor, wherein the administration of the antibody or antigen-binding fragment comprises carrying out between one and three 28-day cycles, each cycle comprises administering a total dosage amount of 900 mg to 2000 mg of the antibody or fragment, optionally wherein the first of said between one and three 28-day cycles begins between at or about day 22 and at or about day 36, optionally at about day 29, after initiation of the T cell therapy.

In some embodiments of any one of the methods provided herein, the total dosage amount of the anti-PD-L1 antibody or antigen-binding fragment in each 28-day cycle independently is or is about 1200 mg to 1500 mg. In some embodiments, the total dosage amount of the anti-PD-L1 antibody or antigen-binding fragment in at least one 28-day cycle is or is about 1200 mg. In some embodiments, the total dosage amount of the anti-PD-L1 antibody or antigen-binding fragment in at least one 28-day cycle is or is about 1500 mg. In some embodiments, the total dosage amount of the anti-PD-L1 antibody or antigen-binding fragment in each 28-day cycle is or is about 1500 mg. In some embodiments, the total dosage amount in each 28-day cycle comprises administering 1, 2, 3 or 4 doses of the anti-PD-L1 antibody or antigen-binding fragment thereof.

In some embodiments of any one of the methods provided herein, each 28-day cycle independently comprises a dosing schedule selected from (i) once-weekly (Q1W) for four doses, optionally on days 1, 8, 15 and 22; (ii) Q1W for two consecutive doses, optionally on days 1 and 8, followed by every two weeks (Q2W) for one dose, optionally on day 15; (iii) every two weeks (Q2W) for two doses, optionally on days 1 and 15; or (iv) every four weeks (Q4W) for one dose, optionally on day 1.

In some of any such embodiments, each 28-day cycle independently comprises a dosing schedule selected from (i) four doses each once-weekly (Q1W), optionally on days 1, 8, 15 and 22 of the 28-day cycle; (ii) two consecutive doses each Q1W, optionally on days 1 and 8, followed by one dose once in two weeks (Q2W) for one dose, optionally on day 15, of the 28-day cycle; (iii) two doses each every two weeks (Q2W), optionally on days 1 and 15 of the 28-day cycle; or (iv) one dose every four weeks (Q4W), optionally on day 1, of the 28-day cycle.

In some embodiments of any one of the methods provided herein, the anti-PD-L1 antibody or antigen-binding fragment is administered on day 1, 8 and 15 in a first 28-day cycle, on day 1 in a second 28-day cycle, and on day 1 in a third 28-day cycle. In some embodiments of any one of the methods provided herein, the anti-PD-L1 antibody or antigen-binding fragment is administered on day 1, 8, 15 and 22 in a first 28-day cycle, on day 1 and 15 in a second 28-day cycle, and on day 1 in a third 28-day cycle. In some embodiments of any one of the methods provided herein, the anti-PD-L1 antibody or antigen-binding fragment is administered on day 1 in each 28-day cycle.

In some embodiments of any one of the methods provided herein, further involve: administering the anti-PD-L1 antibody or antigen-binding fragment in one or more further 28-day cycle if the subject exhibits no more than a partial response (PR) following the treatment and/or exhibits no more than a PR at three-months following initiation of administration of the T cell therapy and/or of the anti-PD-L1 antibody or fragment. In some of any such embodiments, the anti-PD-L1 antibody or antigen-binding fragment is administered in a total dosage amount of 900 mg to 2000 mg in each of the one or more further 28-day cycle, optionally in a total dosage amount of at or about 1500 mg. In some embodiments, the anti-PD-L1 antibody or antigen-binding fragment is administered in a total dosage amount of 900 mg to 2000 mg in each of the one or more further 28-day cycle, optionally at or about 1500 mg.

In some embodiments of any one of the methods provided herein, the anti-PD-L1 antibody or antigen-binding fragment is administered for a total duration of no more than 12 months.

In some embodiments of any one of the methods provided herein, the administration of the anti-PD-L1 antibody or antigen-binding fragment and/or the start of the first 28-day cycle is initiated greater than 21 days after initiation of administration of the T cell therapy.

In some embodiments of any one of the methods provided herein, the administration of the anti-PD-L1 antibody or antigen-binding fragment and/or the start of the first 28-day cycle is initiated at a time at or after, optionally immediately after or within 1 to 3 days after: (i) peak or maximum level of the cells of the T cell therapy are detectable in the blood of the subject; (ii) the number of cells of the T cell therapy detectable in the blood, after having been detectable in the blood, is not detectable or is reduced, optionally reduced compared to a preceding time point after administration of the T cell therapy; (iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy; (iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject; (v) the subject exhibits disease progression and/or has relapsed following remission after treatment with the T cell therapy; and/or (vi) the subject exhibits increased tumor burden as compared to tumor burden at a time prior to or after administration of the cells and prior to initiation of administration of the anti-PD-L1 antibody.

In some embodiments of any one of the methods provided herein, the administration of the anti-PD-L1 antibody or antigen-binding fragment and/or the start of the first 28-day cycle is initiated at or within 29 days, 36 days, 43 days or 50 days after initiation of administration of the T cell therapy. In some embodiments of any one of the methods provided herein, the administration of the anti-PD-L1 antibody or antigen-binding fragment and/or the start of the first 28-day cycle is initiated from or from about 22 days to 36 days after initiation of administration of the T cell therapy. In some embodiments of any one of the methods provided herein, the administration of the anti-PD-L1 antibody or antigen-binding fragment and/or the start of the first 28-day cycle is initiated at or about 29 days after initiation of administration of the T cell therapy. In some of any such embodiments, administration of the checkpoint inhibitor and/or the start of the first dosage cycle is initiated at or about 43 days after initiation of administration of the T cell therapy.

In some embodiments of any one of the methods provided herein, at the time of administering the anti-PD-L1 antibody or antigen-binding fragment and/or the start of the first 28-day cycle, the subject does not exhibit a severe toxicity following administration of the T cell therapy. In some embodiments, the severe toxicity is severe cytokine release syndrome (CRS), optionally grade 3 or higher, prolonged grade 3 or higher or grade 4 or 5 CRS; and/or the severe toxicity is severe neurotoxicity, optionally grade 3 or higher, prolonged grade 3 or higher or grade 4 or 5 neurotoxicity.

In some embodiments of any one of the methods provided herein, the anti-PD-L1 antibody or antigen-binding fragment thereof specifically binds to an extracellular domain of PD-L1. In some embodiments of any one of the methods provided herein, the anti-PD-L1 antibody or antigen-binding fragment thereof is MEDI4736 (durvalumab), MDPL3280A (atezolizumab), YW243.55.S70, MDX-1105 (BMS-936559), LY3300054, or MSB0010718C (avelumab), or is or comprises an antigen-binding fragment or region of any of the foregoing. In some embodiments of any one of the methods provided herein, the anti-PD-L1 antibody or antigen-binding fragment thereof is MEDI4736 (durvalumab) or is or comprises an antigen-binding fragment or region thereof. In some of any such embodiments, the anti-PD-L1 antibody antibody or antigen binding fragment thereof of MEDI4736 (durvalumab).

In some embodiments of any one of the methods provided herein, the B cell malignancy is a non-Hodgkin lymphoma (NHL). In some embodiments, wherein, at or immediately prior to the time of the administration of the T cell therapy the subject has relapsed following remission after treatment with, or become refractory to, one or more prior therapies for the NHL, optionally one or two prior therapies other than another dose of cells expressing the CAR, optionally wherein the prior therapy is or comprises a CD20-targeted agent or anthracycline. In some embodiments, the NHL comprises aggressive NHL, diffuse large B cell lymphoma (DLBCL), DLBCL-NOS, optionally transformed indolent; EBV-positive DLBCL-NOS; T cell/histiocyte-rich large B-cell lymphoma; primary mediastinal large B cell lymphoma (PMBCL); follicular lymphoma (FL), optionally, follicular lymphoma Grade 3B (FL3B); and/or high-grade B-cell lymphoma, with MYC and BCL2 and/or BCL6 rearrangements with DLBCL histology (double/triple hit). In some of any such embodiments, the NHL comprises diffuse large B cell lymphoma (DLBCL); DLBCL-NOS; DLBCL-NOS transformed indolent; follicular lymphoma Grade 3B (FL3B); and/or high-grade B-cell lymphoma, with MYC and BCL2 and/or BCL6 rearrangements with DLBCL histology (double/triple hit).

In some embodiments of any one of the methods provided herein, the subject is or has been identified as having an Eastern Cooperative Oncology Group Performance Status (ECOG) status of less than or equal to 1.

In some embodiments of any one of the methods provided herein, the recombinant receptor specifically binds to a target antigen expressed by the B cell malignancy. In some embodiments, the target antigen is a B cell antigen, optionally CD19.

In some of any such embodiments, the target antigen is a B cell antigen. In some of any such embodiments, the target antigen is CD19.

In some of any such embodiments, the chimeric antigen receptor (CAR) comprises an extracellular antigen-recognition domain that specifically binds to a target antigen and an intracellular signaling domain comprising an ITAM. In some of any such embodiments, the intracellular signaling domain comprises a signaling domain of a CD3-zeta (CD3) chain.

In some of any such embodiments, the chimeric antigen receptor (CAR) further comprises a costimulatory signaling region comprising a cytoplasmic signaling domain of a costimulatory molecule. In some of any such embodiments, the costimulatory signaling region comprises a cytoplasmic signaling domain of CD28 or 4-1BB. In some of any such embodiments, the costimulatory domain is or comprises a cytoplasmic signaling domain of 4-1BB.

In some of any such embodiments, the CAR comprises an scFv specific for CD19, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is or comprises a CD3zeta signaling domain, and optionally further comprises a spacer between the transmembrane domain and the scFv.

In some of any such embodiments, the CAR comprises, in order, an scFv specific for CD19, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is a CD3zeta signaling domain.

In some of any such embodiments, the CAR comprises, in order, an scFv specific for CD19, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain.

In some of any such embodiments, the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof.

In some of any such embodiments, the spacer comprises or consists of the formula X₁PPX₂P (SEQ ID NO:58), where X₁ is glycine, cysteine or arginine and X₂ is cysteine or threonine. In some of any such embodiments, the spacer comprises or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto. In some of any such embodiments, the spacer comprises the sequence of SEQ ID NO: 1.

In some of any such embodiments, the cytoplasmic signaling domain of a costimulatory molecule comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto, In some of any such embodiments, the cytoplasmic signaling domain derived from a primary signaling ITAM-containing molecule comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto.

In some of any such embodiments, the extracellular antigen-recognition domain is an scFv and the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40). In some of any such embodiments, the extracellular antigen-recognition domain is an scFv and the scFv comprises a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63. In some of any such embodiments, the extracellular antigen-recognition domain is an scFv and the scFv comprises a variable heavy chain region of FMC63 and a variable light chain region of FMC63. In some of any such embodiments, the wherein the extracellular antigen-recognition domain is an scFv and the scFv comprises a V_{H} region comprising an amino acid sequence set forth in SEQ ID NO:41. In some of any such embodiments, the wherein the extracellular antigen-recognition domain is an scFv and the scFv comprises a V_{L} region comprising an amino acid sequence set forth in SEQ ID NO:42.

In some of any such embodiments, the extracellular antigen-recognition domain is an scFv and the scFv comprises, in order, a V_{H}, optionally comprising the amino acid sequence set forth in SEQ ID NO:41, a linker, optionally comprising SEQ ID NO: 59, and a V_{L}, optionally comprising the amino acid sequence set forth in SEQ ID NO:42, and/or the scFv comprises a flexible linker and/or comprises the amino acid sequence set forth as SEQ ID NO: 43. In some of any such embodiments, the wherein the extracellular antigen-recognition domain is an scFv and the scFv comprise an amino acid sequence set forth in SEQ ID NO:43.

In some embodiments of any one of the methods provided herein, the recombinant receptor is a chimeric antigen receptor (CAR). In some embodiments, the chimeric antigen receptor (CAR) comprises an extracellular antigen-recognition domain that specifically binds to the antigen and an intracellular signaling domain comprising an ITAM. In some embodiments, the intracellular signaling domain comprises an signaling domain of a CD3-zeta (CD3) chain. In some embodiments, the chimeric antigen receptor (CAR) further comprises a costimulatory signaling region. In some embodiments, the costimulatory signaling region comprises a signaling domain of CD28 or 4-1BB. In some embodiments, the costimulatory domain is or comprises a domain of 4-1BB. In some embodiments, the CAR comprises an scFv specific for the antigen, a transmembrane domain, a cytoplasmic signaling domain derived from a costimulatory molecule, which optionally is or comprises a 4-1BB, and a cytoplasmic signaling domain derived from a primary signaling ITAM-containing molecule, which optionally is or comprises a CD3zeta signaling domain and optionally further comprises a spacer between the transmembrane domain and the scFv; the CAR comprises, in order, an scFv specific for the antigen, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is a CD3zeta signaling domain; or the CAR comprises, in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain. In some embodiments, the spacer is optionally a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID O:N 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine; and/or the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or the primary signaling domain comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv comprises, in order, a VH, a linker, optionally comprising SEQ ID NO: 41, and a VL, and/or the scFv comprises a flexible linker and/or comprises the amino acid sequence set forth as SEQ ID NO: 42.

In some embodiments of any one of the methods provided herein, the dose of genetically engineered T cells comprises from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, 5×10⁷ to 1×10⁸ total CAR-expressing T cells, each inclusive.

In some embodiments of any one of the methods provided herein, the dose of genetically engineered T cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells. In some embodiments of any one of the methods provided herein, the dose of genetically engineered T cells comprises at or about 5×10⁷ CAR-expressing cells. In some embodiments of any one of the methods provided herein, the dose of genetically engineered T cells comprises at or about 1×10⁸ CAR-expressing cells. In some of any such embodiments, the dose of genetically engineered T cells comprises at or about 1.5×10⁸ CAR-expressing cells.

In some embodiments of any one of the methods provided herein, the dose of cells is administered parenterally, optionally intravenously. In some embodiments, the T cells are primary T cells obtained from a subject. In some embodiments of any one of the methods provided herein, the T cells are autologous to the subject. In some embodiments of any one of the methods provided herein, the T cells are allogeneic to the subject.

In some embodiments of any one of the methods provided herein, the dose of genetically engineered T cells comprises CD4+ T cells expressing the CAR and CD8+ T cells expressing the CAR and the administration of the dose comprises administering a plurality of separate compositions, said plurality of separate compositions comprising a first composition comprising one of the CD4+ T cells and the CD8+ T cells and the second composition comprising the other of the CD4+ T cells or the CD8+ T cells. In some embodiments, the first composition and second composition are administered 0 to 12 hours apart, 0 to 6 hours apart or 0 to 2 hours apart or wherein the administration of the first composition and the administration of the second composition are carried out on the same day, are carried out between about 0 and about 12 hours apart, between about 0 and about 6 hours apart or between about 0 and 2 hours apart; and/or the initiation of administration of the first composition and the initiation of administration of the second composition are carried out between about 1 minute and about 1 hour apart or between about 5 minutes and about 30 minutes apart. In some embodiments, the first composition and second composition are administered no more than 2 hours, no more than 1 hour, no more than 30 minutes, no more than 15 minutes, no more than 10 minutes or no more than 5 minutes apart. In some embodiments, the first composition comprises the CD4+ T cells. In some embodiments, the first composition comprises the CD8+ T cells. In some embodiments, the first composition is administered prior to the second composition.

In some embodiments of any one of the methods provided herein, prior to the administration, the subject has been preconditioned with a lymphodepleting therapy comprising the administration of fludarabine and/or cyclophosphamide. In some embodiments, immediately prior to the administration, administering a lymphodepleting therapy to the subject comprising the administration of fludarabine and/or cyclophosphamide. In some embodiments, the lymphodepleting therapy comprises administration of cyclophosphamide at about 200-400 mg/m², optionally at or about 300 mg/m², inclusive, and/or fludarabine at about 20-40 mg/m², optionally 30 mg/m², daily for 2-4 days, optionally for 3 days, or wherein the lymphodepleting therapy comprises administration of cyclophosphamide at about 500 mg/m². In some embodiments, the lymphodepleting therapy comprises administration of cyclophosphamide at or about 300 mg/m² and fludarabine at about 30 mg/m² daily for 3 days; and/or the lymphodepleting therapy comprises administration of cyclophosphamide at or about 500 mg/m² and fludarabine at about 30 mg/m² daily for 3 days.

In some embodiments of any one of the methods provided herein, the subject is a human.

Also provided herein are kits that include: (a) a T cell therapy comprising a dose of genetically engineered T cells expressing a chimeric antigen receptor, wherein the chimeric antigen receptor specifically binds to a target antigen expressed by the B cell malignancy; (b) a checkpoint inhibitor that is an antibody or antigen-binding fragment thereof capable of blocking an immune checkpoint pathway protein, optionally wherein the checkpoint inhibitor thereof is formulated in one or more individual doses; and (c) instructions for administering the T cell therapy and/or the checkpoint inhibitor to a subject having a B cell malignancy, wherein the instructions specify administration of the T cell therapy and/or the checkpoint inhibitor according to the any of the embodiments described herein.

Also provided herein are kits that include: (a) a T cell therapy comprising a dose of genetically engineered T cells expressing a chimeric antigen receptor, wherein the chimeric antigen receptor specifically binds to a target antigen expressed by the B cell malignancy; and (b) instructions for administering the T cell therapy to a subject having a B cell malignancy, wherein the instructions specify that the subject is to be administered a checkpoint inhibitor that is an antibody or antigen-binding fragment thereof capable of blocking an immune checkpoint pathway protein, after the administration of the T cell therapy, wherein the instructions specify administration of the T cell therapy and/or the checkpoint inhibitor according to any of the embodiments described herein.

Also provided herein are kits that include: (a) a checkpoint inhibitor that is an antibody or antigen-binding fragment thereof capable of blocking an immune checkpoint pathway protein, optionally wherein the checkpoint inhibitor thereof is formulated in one or more individual doses; and (b) instructions for administering the checkpoint inhibitor to a subject having a B cell malignancy, wherein the instructions specify that the checkpoint inhibitor is administered after initiation of administration of a T cell therapy, the T cell therapy comprising a dose of genetically engineered T cells expressing a chimeric antigen receptor, wherein the chimeric antigen receptor specifically binds to a target antigen expressed by the B cell malignancy, wherein the instructions specify administration of the T cell therapy and/or the checkpoint inhibitor according to any of the embodiments described herein.

Provided here are kits that involve: (a) a T cell therapy comprising a dose of genetically engineered T cells expressing a recombinant receptor; (b) an anti-PD-L1 antibody or antigen-binding fragment thereof, optionally wherein the anti-PD-L1 antibody or fragment thereof is formulated in one or more unit doses; and (c) instructions for administering the genetically engineered cells and/or the anti-PD-L1 antibody or antigen-binding fragment to a subject having a B cell malignancy, wherein the instructions comprising instructing: (i) the administration of the administration of the anti-PD-L1 antibody or antigen-binding fragment carries out at least two 28-day cycles, each of said at least two 28-day cycles, independently, comprising administering a total dosage amount of 750 mg to 2000 mg of the antibody or antigen-binding fragment; and (ii) in at least the first of said at least two 28-day cycles, the administration of the total dosage amount of the anti-PD-L1 antibody or antigen-binding fragment is carried out by administering the antibody or fragment more than one time.

Provided here are methods of treatment that involve: (a) a T cell therapy comprising a dose of genetically engineered T cells expressing a recombinant receptor; and (b) instructions for administering the T cell therapy to a subject having a B cell malignancy, wherein the instructions specify that the subject is to be administered an anti-PD-L1 antibody or antigen-binding fragment thereof after the administration of T cells, wherein the instructions comprising instructing: (i) the administration of the administration of the anti-PD-L1 antibody or antigen-binding fragment carries out at least two 28-day cycles, each of said at least two 28-day cycles, independently, comprising administering a total dosage amount of 750 mg to 2000 mg of the antibody or antigen-binding fragment; and (ii) in at least the first of said at least two 28-day cycles, the administration of the total dosage amount of the anti-PD-L1 antibody or antigen-binding fragment is carried out by administering the antibody or fragment more than one time.

Provided here are methods of treatment that involve: (a) an anti-PD-L1 antibody or antigen-binding fragment thereof, optionally wherein the anti-PD-L1 antibody or fragment thereof is formulated in one or more unit doses; and (b) instructions for administering the anti-PD-L1 antibody or antigen-binding fragment to a subject having a B cell malignancy, wherein the instructions specify that the anti-PD-L1 antibody or fragment is administered after initiation of administration of a T cell therapy, the T cell therapy comprising a dose of genetically engineered T cells expressing a recombinant receptor, wherein the instructions comprising instructing: (i) the administration of the administration of the anti-PD-L1 antibody or antigen-binding fragment carries out at least two 28-day cycles, each of said at least two 28-day cycles independently, comprising administering a total dosage amount of 750 mg to 2000 mg of the antibody or antigen-binding fragment thereof; and (ii) in at least the first of said at least two 28-day cycles, the administration of the total dosage amount of the anti-PD-L1 antibody or antigen-binding fragment is carried out by administering the antibody or fragment more than one time.

In some embodiments of any one of the kits provided herein, the instructions further specify that in a first of said at least two 28-day cycles, the administration of the total dosage amount of the anti-PD-L1 antibody or antigen-binding fragment is carried out by administering the antibody or fragment a greater number of times as compared to a second and/or a subsequent 28-day cycle.

In some embodiments of any one of the kits provided herein, the total amount of the anti-PD-L1 antibody or antigen-binding fragment is or is about 225 mg to 2000 mg. In some embodiments, the total amount of the anti-PD-L1 antibody or antigen-binding fragment is or is about 750-1500 mg. In some embodiments, the anti-PD-L1 antibody or antigen-binding fragment is formulated in two or more unit doses, wherein each unit dose is or is about 225 mg to 2000 mg. In some embodiments, each unit dose is or is about 225 mg to 1500 mg.

In some embodiments of any one of the kits provided herein, the anti-PD-L1 antibody or antigen-binding fragment thereof specifically binds to an extracellular domain of PD-L1. In some embodiments of any one of the kits provided herein, the anti-PD-L1 antibody or antigen-binding fragment thereof is MEDI4736 (durvalumab), MDPL3280A (atezolizumab), YW243.55.S70, MDX-1105 (BMS-936559), LY3300054, or MSB0010718C (avelumab), or is an antigen-binding fragment thereof. In some embodiments, the anti-PD-L1 antibody or antigen-binding fragment thereof is MEDI4736 (durvalumab) or is an antigen-binding fragment thereof.

In some embodiments of any one of the kits provided herein, the recombinant receptor specifically binds to a target antigen expressed by the B cell malignancy. In some embodiments, the target antigen is a B cell antigen, optionally CD19.

In some embodiments of any one of the kits provided herein, the recombinant receptor is a chimeric antigen receptor (CAR). In some embodiments, the chimeric antigen receptor (CAR) comprises an extracellular antigen-recognition domain that specifically binds to the antigen and an intracellular signaling domain comprising an ITAM. In some embodiments, the intracellular signaling domain comprises an intracellular domain of a CD3-zeta (CD3ζ) chain. In some embodiments, the chimeric antigen receptor (CAR) further comprises a costimulatory signaling region. In some embodiments, the costimulatory signaling region comprises a signaling domain of CD28 or 4-1BB. In some embodiments, the costimulatory domain is a domain of 4-1BB. In some embodiments, the CAR comprises an scFv specific for the antigen, a transmembrane domain, a cytoplasmic signaling domain derived from a costimulatory molecule, which optionally is or comprises a 4-1BB, and a cytoplasmic signaling domain derived from a primary signaling ITAM-containing molecule, which optionally is or comprises a CD3zeta signaling domain and optionally further comprises a spacer between the transmembrane domain and the scFv; the CAR comprises, in order, an scFv specific for the antigen, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is a CD3zeta signaling domain; or the CAR comprises, in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain. In some embodiments, the spacer is optionally a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID O:N 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine; and/or the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or the primary signaling domain comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv comprises, in order, a VH, a linker, optionally comprising SEQ ID NO: 41, and a VL, and/or the scFv comprises a flexible linker and/or comprises the amino acid sequence set forth as SEQ ID NO: 42.

In some embodiments of any one of the kits provided herein, the T cell therapy comprises from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, 5×10⁷ to 1×10⁸ total CAR-expressing T cells, each inclusive. In some embodiments of any one of the kits provided herein, the T cell therapy comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells. In some embodiments of any one of the kits provided herein, the T cell therapy comprises at, about, or at least about 5×10⁷ CAR-expressing cells. In some embodiments of any one of the kits provided herein, the T cell therapy comprises at, about, or at least about 1×10⁸ CAR-expressing cells. In some embodiments of any one of the kits provided herein, the T cell therapy comprises primary T cells obtained from the subject.

In some embodiments of any one of the kits provided herein, the T cell therapy comprises cells that are autologous to the subject. In some embodiments of any one of the kits provided herein, the T cell therapy comprises cells are allogeneic to the subject.

In some embodiments of any one of the kits provided herein, the T cell therapy comprise CD4+ T cells expressing the CAR and CD8+ T cells expressing the CAR and the administration comprises administering a plurality of separate compositions, said plurality of separate compositions comprising a first composition comprising one of the CD4+ T cells and the CD8+ T cells and the second composition comprising the other of the CD4+ T cells or the CD8+ T cells. In some embodiments, the first composition and second composition are administered 0 to 12 hours apart, 0 to 6 hours apart or 0 to 2 hours apart or wherein the administration of the first composition and the administration of the second composition are carried out on the same day, are carried out between about 0 and about 12 hours apart, between about 0 and about 6 hours apart or between about 0 and 2 hours apart; and/or the initiation of administration of the first composition and the initiation of administration of the second composition are carried out between about 1 minute and about 1 hour apart or between about 5 minutes and about 30 minutes apart. In some embodiments, the instructions specify that he first composition and second composition is administered no more than 2 hours, no more than 1 hour, no more than 30 minutes, no more than 15 minutes, no more than 10 minutes or no more than 5 minutes apart. In some embodiments, the first composition comprises the CD4+ T cells. In some embodiments, the first composition comprises the CD8+ T cells. In some embodiments, the instructions specify that the first composition is administered prior to the second composition.

In some embodiments of any one of the kits provided herein, further comprising a lymphodepleting therapy comprising fludarabine and/or cyclophosphamide.

In some embodiments of any one of the kits provided herein, the instructions specify that the lymphodepleting therapy is administered prior to the administration of the T cell therapy and/or the anti-PD-L1 antibody or fragment thereof.

In some embodiments of any one of the kits provided herein, the instructions specify that the total dosage amount of the anti-PD-L1 antibody or antigen-binding fragment in each 28-day cycle independently is a dosage or a range of dosage in the range of about 750 mg to about 1500 mg. In some embodiments of any one of the kits provided herein, the instructions specify that the total dosage amount of the anti-PD-L1 antibody or antigen-binding fragment in at least one 28-day cycle is or is about 750 mg. In some embodiments of any one of the kits provided herein, the instructions specify that the total dosage amount of the anti-PD-L1 antibody or antigen-binding fragment in at least one 28-day cycle is or is about 1200 mg. In some embodiments of any one of the kits provided herein, the instructions specify that the total dosage amount of the anti-PD-L1 antibody or antigen-binding fragment in at least one 28-day cycle is or is about 1500 mg. In some embodiments of any one of the kits provided herein, the instructions specify that the total dosage amount of the anti-PD-L1 antibody or antigen-binding fragment in each 28-day cycle is or is about 1500 mg.

In some embodiments of any one of the kits provided herein, the instructions specify that the total dosage amount of the anti-PD-L1 antibody or antigen-binding fragment in said at least two 28-day cycles is the same. In some embodiments of any one of the kits provided herein, the instructions specify that the total dosage amount of the anti-PD-L1 antibody or antigen-binding fragment in said at least two 28-day cycles is different. In some embodiments of any one of the kits provided herein, the instructions specify that the total dosage amount of the anti-PD-L1 antibody or antigen-binding fragment in the first 28-day cycle is lower than the second and/or a subsequent 28-day cycle.

In some embodiments of any one of the kits provided herein, the instructions specify that the first 28-day cycle is carried out by administering 2, 3 or 4 doses of the anti-PD-L1 antibody or antigen-binding fragment thereof.

In some embodiments of any one of the kits provided herein, the instructions specify that the first 28-day cycle is carried out by a dosing schedule selected from (i) once-weekly (Q1W) for two doses, optionally on days 15 and 22; (ii) once-weekly (Q1W) for four doses, optionally on days 1, 8, 15 and 22; (iii) Q1W for two consecutive doses, optionally on days 1 and 8, followed by every two weeks (Q2W) for one dose, optionally on day 15; or (iv) every two weeks (Q2W) for two doses, optionally on days 1 and 15. In some embodiments, the instructions specify that: each Q1W dose of the first 28-day cycle is independently from or from about 18% to 32% of the total dosage amount, optionally is or is about 25% of the total dosage amount in the cycle; and/or each Q2W dose of the first 28-day cycle is independently from or from about 40% to 62.5% of the total dosage amount, optionally is or is about 50% of the total dosage amount in the cycle. In some embodiments, the instructions specify that:the first 28-day cycle is carried out by administering the anti-PD-L1 antibody or antigen-binding fragment thereof Q1W for two consecutive doses in an amount of or about 375 mg followed by Q2W for one dose in an amount of or about 750 mg; the first 28-day cycle is carried out by administering the anti-PD-L1 antibody or antigen-binding fragment thereof Q1W for four doses, said four doses comprising two consecutive doses of or about 225 mg followed by two consecutive doses of or about 375 mg; or the first 28-day cycle is carried out by administering the anti-PD-L1 antibody or antigen-binding fragment thereof Q1W for two consecutive doses in an amount of or about 375 mg.

In some embodiments of any one of the kits provided herein, the instructions specify that the second and/or a subsequent 28-day cycle is carried out by administering 1 or 2 does of the anti-PD-L1 antibody or antigen-binding fragment thereof.

In some embodiments of any one of the kits provided herein, the instructions specify that the second and/or a subsequent 28-day cycle is carried out with a dosing schedule selected from (i) every two weeks (Q2W) for two doses, optionally on days 1 and 15; or (ii) every four weeks (Q4W) for one dose, optionally on day 1. In some embodiments, the instructions specify that: each Q2W dose of the second and/or a subsequent 28-day cycle is or is about 50% of the total dosage amount; and/or the Q4W dose of the second and/or a subsequent 28-day cycle is or is about the total dosage amount. In some embodiments, the instructions specify that: the second and/or a subsequent dose is carried out by administering the anti-PD-L1 antibody or antigen-binding fragment thereof Q2W for two doses in an amount of or about 750 mg; or the second and/or a subsequent dose is carried out by administering the anti-PD-L1 antibody or antigen-binding fragment thereof Q4W for one dose in an amount of or about 1500 mg.

In some embodiments of any one of the kits provided herein, the instructions specify that: the first 28-day cycle is carried out by administering the anti-PD-L1 antibody or antigen-binding fragment thereof once-weekly (Q1W) for two consecutive doses in an amount of or about 375 mg followed by every two weeks (Q2W) for one dose in an amount of or about 750 mg; and the second and/or a subsequent 28-day cycle is carried out by administering the anti-PD-L1 antibody or antigen-binding fragment thereof Q4W for one dose in an amount of or about 1500 mg.

In some embodiments of any one of the kits provided herein, the instructions specify that: the first 28-day cycle is carried out by administering the anti-PD-L1 antibody or antigen-binding fragment thereof once-weekly (Q1W) for four doses, said four doses have two consecutive doses of or about 225 mg followed by two consecutive doses of or about 375 mg; and the second and/or a subsequent 28-day cycle is carried out by administering the anti-PD-L1 antibody or antigen-binding fragment thereof every two weeks (Q2W) for two doses in an amount of or about 750 mg.

In some embodiments of any one of the kits provided herein, the instructions specify that: the first 28-day cycle is carried out by administering the anti-PD-L1 antibody or antigen-binding fragment thereof once-weekly (Q1W) for two doses, each of said doses is or is about an amount of or about 375 mg, optionally wherein the doses are consecutive doses, optionally wherein the doses are carried out on days 15 and 22 in the 28-day cycle; and the second and/or a subsequent 28-day cycle is carried out by administering the anti-PD-L1 antibody or antigen-binding fragment thereof Q4W for one dose in an amount of or about 1500 mg.

In some embodiments of any one of the kits provided herein, the instructions specify that the administration of the administration of the anti-PD-L1 antibody or antigen-binding fragment carries out at least three 28-day cycles. In some embodiments, the instructions specify that the total dosage amount of the anti-PD-L1 antibody or antigen-binding fragment in the third 28-day cycle is the same as the first and/or second 28-day cycle. In some embodiments, the instructions specify the total dosage amount of the anti-PD-L1 antibody or antigen-binding fragment in the third 28-day cycle is or is about 1500 mg, In some embodiments, the instructions specify that in the third 28-day cycle, the administration of the total dosage amount of the anti-PD-L1 antibody or antigen-binding fragment is carried out by administering the antibody or fragment a greater number of times as compared to the first and/or second 28-day cycle. In some embodiments, the instructions specify that in the third 28-day cycle, the administration of the total dosage amount of the anti-PD-L1 antibody or antigen-binding fragment is carried out by administering the antibody or fragment the same number of times as compared to the second 28-day cycle. In some embodiments, the instructions specify that the third 28-day cycle is carried out with a dosing schedule every four weeks (Q4W) for one dose, optionally on day 1.

In some embodiments of any one of the kits provided herein, the instructions specify that the administration of the anti-PD-L1 antibody or antigen-binding fragment is carried out by no more than three 28-day cycles after initiation of the T cell therapy.

In some embodiments of any one of the kits provided herein, the instructions specify that each 28-day cycle is independently carried out with a dosing schedule selected from (i) once-weekly (Q1W) for four doses, optionally on days 1, 8, 15 and 22; (ii) Q1W for two consecutive doses, optionally on days 1 and 8, followed by every two weeks (Q2W) for one dose, optionally on day 15; (iii) every two weeks (Q2W) for two doses, optionally on days 1 and 15; or (iv) every four weeks (Q4W) for one dose, optionally on day 1.

In some embodiments of any one of the kits provided herein, the instructions specify that the anti-PD-L1 antibody or antigen-binding fragment is administered on day 1, 8 and 15 in a first 28-day cycle, on day 1 in a second 28-day cycle, and on day 1 in a third 28-day cycle. In some embodiments of any one of the kits provided herein, the instructions specify that the anti-PD-L1 antibody or antigen-binding fragment is administered on day 1, 8, 15 and 22 in a first 28-day cycle, on day 1 and 15 in a second 28-day cycle, and on day 1 in a third 28-day cycle. In some embodiments of any one of the kits provided herein, the instructions specify that the anti-PD-L1 antibody or antigen-binding fragment is administered on day 1 in each 28-day cycle.

In some embodiments of any one of the kits provided herein, the instructions specify that the administration of the anti-PD-L1 antibody or antigen-binding fragment is carried out by one or more further 28-day cycle if the subject exhibits a partial response (PR) following the treatment.

In some embodiments of any one of the kits provided herein, the instructions specify that the administration of the anti-PD-L1 antibody or antigen-binding fragment is carried out for a total duration of about 12 months or less than about 12 months.

In some embodiments of any one of the kits provided herein, the instructions specify that the administration of the anti-PD-L1 antibody or antigen-binding fragment is initiated at a time greater than 21 days (e.g., at about 29 days, within 22-36 days) after initiation of administration of the T cell therapy. In some embodiments of any one of the kits provided herein, the instructions specify that the administration of the anti-PD-L1 antibody or antigen-binding fragment is initiated at or within about 29 days, 36 days, 43 days or 50 days after initiation of administration of the T cell therapy.

In some embodiments of any one of the kits provided herein, the instructions instructs that anti-PD-L1 antibody or antigen-binding fragment should not be administered when the subject exhibits a severe toxicity. In some embodiments, the instructions specify that: the severe toxicity is severe cytokine release syndrome (CRS), optionally grade 3 or higher, prolonged grade 3 or higher or grade 4 or 5 CRS; and/or the severe toxicity is severe neurotoxicity, optionally grade 3 or higher, prolonged grade 3 or higher or grade 4 or 5 neurotoxicity.

In some embodiments of any one of the kits provided herein, the instructions specify that: the administration of the anti-PD-L1 antibody or antigen-binding fragment is initiated at a time at or after, optionally immediately after or within 1 to 3 days after: (i) peak or maximum level of the cells of the T cell therapy are detectable in the blood of the subject; (ii) the number of cells of the T cell therapy detectable in the blood, after having been detectable in the blood, is not detectable or is reduced, optionally reduced compared to a preceding time point after administration of the T cell therapy; (iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy; (iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject; (v) the subject exhibits disease progression and/or has relapsed following remission after treatment with the T cell therapy; and/or (vi) the subject exhibits increased tumor burden as compared to tumor burden at a time prior to or after administration of the cells and prior to initiation of administration of the anti-PD-L1 antibody.

In some embodiments of any one of the kits provided herein, the subject is human.

In some embodiments of any one of the kits provided herein, the instructions specify that the administration of the T cell therapy or the anti-PD-L1 antibody or antigen-binding fragment is for treating a non-Hodgkin lymphoma (NHL). In some embodiments, the instructions specify that the administration of the T cell therapy or the anti-PD-L1 antibody or antigen-binding fragment is for treating a non-Hodgkin lymphoma (NHL) in the subject, wherein the subject has relapsed following remission after treatment with, or become refractory to, one or more prior therapies for the NHL, optionally one or two prior therapies other than another dose of cells expressing the CAR, optionally wherein the prior therapy is or comprises a CD20-targeted agent or anthracycline. In some embodiments, the instructions specify the NHL as any one of aggressive NHL, diffuse large B cell lymphoma (DLBCL), DLBCL-NOS, optionally transformed indolent; EBV-positive DLBCL-NOS; T cell/histiocyte-rich large B-cell lymphoma; primary mediastinal large B cell lymphoma (PMBCL); follicular lymphoma (FL), optionally, follicular lymphoma Grade 3B (FL3B); and/or high-grade B-cell lymphoma, with MYC and BCL2 and/or BCL6 rearrangements with DLBCL histology (double/triple hit).

In some embodiments of any one of the kits provided herein, the instructions specify that the subject must be identified as having an Eastern Cooperative Oncology Group Performance Status (ECOG) status of less than or equal to 1 to qualify for being a candidate who is or will be subject to the use of this kit.

In some embodiments of any one of the kits provided herein, the cells are suitable for being administered parenterally, optionally intravenously.

In some embodiments of any one of the kits provided herein, the anti-PD-L1 antibody or antigen-binding fragment is suitable for being administered parenterally, optionally intravenously.

### BRIEF DESCRIPTION OF THE DRAWINGS

FIG. 1 depicts surface expression, as detected by flow cytometry, of PD-1 on 1) a population of T cells gated for positive surface expression of both CD4 and an anti-CD19 chimeric antigen receptor (CAR) (see left), or 2) a population of T cells gated for positive surface expression of both CD8 and an anti-CD19 chimeric antigen receptor (CAR) (see right) immediately post thaw (Time 0), and following incubation for 24 hours in culture with K562.CD19 target cells at a effector:target (E:T) ratio of 1.25:1, compared with fluorescence-minus-one (FMO) control, as described in Example 1.

FIG. 2 depicts surface expression by median fluorescence intensity, as detected by flow cytometry, of PD-1 on 1) a population of T cells gated for positive surface expression of both CD4 and an anti-CD19 chimeric antigen receptor (CAR) (see top), or 2) a population of T cells gated for positive surface expression of both CD8 and an anti-CD19 chimeric antigen receptor (CAR) (see bottom) immediately post thaw (Time 0, TO), and following incubation for 24 hours in culture with K562.CD19 target cells at three different effector:target (E:T) ratio of 5:1, 2.5:1 or 1.25:1, as described in Example 1.

FIG. 3A depict the percentages of either CD4 positive anti-CD19 CAR-expressing cells (see FIG. 3A, top) or CD8 positive anti-CD19 CAR-expressing cells (see FIG. 3A, bottom) from 3 donors that were positive for PD-1 surface expression following stimulation of K562.CD19 target cells at a effector:target (E:T) ratio of 2.5:1, as described in Example 1. FIG. 3B depicts mean fluorescence intensity (MFI) of PD-1 on either CD4 positive anti-CD19 CAR-expressing cells (see FIG. 3B, top) or CD8 positive anti-CD19 CAR-expressing cells (see FIG. 3B, bottom) following stimulation of K562.CD19 target cells at a effector:target (E:T) ratio of 2.5:1, as described in Example 1.

FIGS. 4A-4C depicts cytokine (IFNγ, IL-2, and TNF-α) production levels in supernatants taken after incubation of anti-CD19 CAR-expressing cells from three different donors (FIGS. 4A, 4B and 4C) with (1) K562.CD19 target cells in the presence of durvalumab (see black inverted triangle), (2) K562.CD19 target cells in the presence of an isotype control (see black circle) (3) K562.CD19.PDL1 target cells in the presence of durvalumab (see grey inverted triangle) or (4) K562.CD19.PDL1 target cells in the presence of an isotype control (see grey circle) for 24 hours, as described in Example 2A.

FIGS. 5A-5C depict surface expression of CD25, CD69, and PD-1 on either CD4 positive anti-CD19 CAR-expressing cells or CD8 positive anti-CD19 CAR-expressing cells from Donor 1 (see FIG. 5A), Donor 2 (see FIG. 5B), and Donor 3 (see FIG. 5C), after the cells were separately co-cultured with (1) K562.CD19 target cells in the presence of durvalumab (see black inverted triangle), (2) K562.CD19 target cells in the presence of an isotype control (see black circle) (3) K562.CD19.PDL1 target cells in the presence of durvalumab (see grey inverted triangle) or (4) K562.CD19.PDL1 target cells in the presence of an isotype control (see grey circle) for 24 hours, as described in Example 2B.

FIG. 6 depicts the number of CD3 positive CAR-expressing T cells, CD4 positive CAR-expressing T cells, and CD8 positive CAR-expressing T cells in peripheral blood of a subject with chemorefractory transformed DLBCL measured at certain time points, as described in Example 3.

### DETAILED DESCRIPTION

Provided herein are combination therapies involving administration of a cell therapy, such as a T cell therapy, and a checkpoint inhibitor, such as an anti-PD-L1 antibody (or antigen-binding fragment thereof) for treating a disease or condition, e.g. a B cell malignancy. In some aspects, the checkpoint inhibitor is capable of inhibiting or blocking a protein or component of an immune checkpoint pathway, such as the PD-1/PD-L1 axis of the checkpoint pathway. In some embodiments, exemplary checkpoint inhibitors include an anti-PD-L1 antibody or an anti-PD-1 antibody. In some aspects, the T cell therapy is an adoptive T cell therapy comprising T cells that specifically recognize and/or target an antigen associated with a disease or disorder, e.g. a cancer or proliferative disease, such as a B cell malignancy, e.g. Non-Hodgkin Lymphoma (NHL) or a subtype thereof. Also provided are combinations and articles of manufacture, such as kits, that contain a composition comprising the T cell therapy and/or a composition comprising the immunomodulatory compound, and uses of such compositions and combinations to treat or prevent diseases, conditions, and disorders, including cancers.

Cell therapies, such as T cell-based therapies, for example, adoptive T cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be effective in the treatment of cancer and other diseases and disorders. The engineered expression of recombinant receptors, such as chimeric antigen receptors (CARs), on the surface of T cells enables the redirection of T cell specificity. In clinical studies, CAR-expressing T cells (CAR-T) cells, for example anti-CD19 CAR-T cells, have produced durable, complete responses in both leukemia and lymphoma patients (Porter et al. (2015) Sci Transl Med., 7:303ra139; Kochenderfer (2015) J. Clin. Oncol., 33: 540-9; Lee et al. (2015) Lancet, 385:517-28; Maude et al. (2014) N Engl J Med, 371:1507-17).

In certain contexts, available approaches to adoptive cell therapy may not always be entirely satisfactory. For example, although CAR T cell persistence can be detected in many subjects with lymphoma, fewer complete responses (CRs) have been observed in subjects with NHL compared to subjects with acute lymphoblastic leukemia (ALL). More specifically, while higher overall response rates of up to 80% (CR rate 47% to 60%) have been reported after CAR T cell infusion, responses in some are transient and subjects have been shown to relapse in the presence of persistent CAR T cells (Neelapu, 58th Annual Meeting of the American Society of Hematology (ASH): 2016; San Diego, Calif., USA. Abstract No. LBA-6.2016; Abramson, Blood. 2016 Dec. 1; 128(22):4192). Another study reported a long term CR rate of 40% (Schuster, Ann Hematol. 2016 October; 95(11):1805-10).

In some aspects, an explanation for this is the immunological exhaustion of circulation CAR-expressing T cells and/or changes in T lymphocyte populations. This is because, in some contexts, optimal efficacy can depend on the ability of the administered cells to have the capability to become activated, expand, to exert various effector functions, including cytotoxic killing and secretion of various factors such as cytokines, to persist, including long-term, to differentiate, transition or engage in reprogramming into certain phenotypic states (such as long-lived memory, less-differentiated, and effector states), to avoid or reduce immunosuppressive conditions in the local microenvironment of a disease, to provide effective and robust recall responses following clearance and re-exposure to target ligand or antigen, and avoid or reduce exhaustion, anergy, peripheral tolerance, terminal differentiation, and/or differentiation into a suppressive state.

In some embodiments, the exposure and persistence of engineered cells is reduced or declines after administration to the subject. Yet, observations indicate that, in some cases, the administered cells expressing the recombinant receptors (e.g., increased number of cells or duration over time) can re-expand in vivo to improve efficacy and therapeutic outcomes in adoptive cell therapy.

In some aspects, the provided embodiments permit such re-expansion or reduce or prevent exhaustion and/or suppression of the administered cells, for example by virtue of administering a checkpoint inhibitor, such as an inhibitor of the PD-1/PD-L1 immune checkpoint axis, e.g., an anti-PD-L1 antibody or an anti-PD-1 antibody.

Programmed cell death 1 (PD-1) is an immune checkpoint protein that is expressed in B cells, NK cells, and T cells (Shinohara et al., 1995, Genomics 23:704-6; Blank et al., 2007, Cancer Immunol Immunother 56:739-45; Finger et al., 1997, Gene 197:177-87; Pardoll (2012) Nature Reviews Cancer 12:252-264). The major role of PD-1 is to limit the activity of T cells in peripheral tissues during inflammation in response to infection, as well as to limit autoimmunity. PD-1 expression is induced in activated T cells and binding of PD-1 to one of its endogenous ligands acts to inhibit T-cell activation by inhibiting stimulatory kinases. PD-1 is also highly expressed on regulatory T cells (Treg) cells and may increase their proliferation in the presence of ligand (Pardoll (2012) Nature Reviews Cancer 12:252-264). The primary result of PD-1 ligation by its ligands is to inhibit signaling downstream of the T cell Receptor (TCR). PD-1 signaling is thought to require binding to a PD-1 ligand in close proximity to a peptide antigen presented by major histocompatibility complex (MHC), which is bound to the TCR (Freeman, Proc. Natl. Acad. Sci., U.S.A, 105:10275-10276 (2008)). PD-L1 is the predominant PD-1 ligand causing inhibitory signal transduction in T cells. PD-L2 is a different ligand of PD-1 that can inhibit T cell function or activity.

PD-L1 (Programmed Cell Death Ligand-1; also known as B7 homolog 1 (B7-H7), or cluster of differentiation encoded by the CD274 gene (CD274)) binds PD-1 (Programmed Cell Death Protein 1) and plays a role in the regulation of the immune system functions including immunity and self-tolerance. PD-L1 is expressed on T cells, e.g., regulatory T cells (Tregs), antigen presenting cells (APCs, e.g. dentritic cells (DCs), macrophages, and B cells), as well as non-hematopoeitic cells including pancreatic islet cells, vascular endothelial cells (placenta, testes, eye), and in tumors. The PD-L1/PD-1 pathway is involved in attenuation of self-reactive T cells, development of inducible Treg cells, suppression of CD4+ effector T cells and CD8+T cells. Thus, interfering with the inhibitory signal through the PD-L1/PD-1 pathway is a therapeutic option for enhancing anti-tumor immunity.

In some embodiments, activated CD8+ cytotoxic T cells can recognize their target antigen presented on tumor cells and initiate tumor-cell killing after formation of the immune synapse. In some contexts, tumor cells can dampen T cell activity by expressing the PD-L1 and/or PD-L2 that bind to the PD-1 on T cells, resulting in inhibitory checkpoint signaling that decreases cytotoxicity and leads to T cell exhaustion. PD-L1 is expressed in lymphoma, both on the tumor and in the tumor microenvironment (TME), and might play a role in tumor-associated immunosuppression of CAR T cells (Andorsky, Clin. Cancer. Res. 2011 Jul. 1; 17(13):4232-44).

In some contexts, antibodies interfering with the immune checkpoint produce tumor regression in multiple cancers by disrupting the PD-L1/PD-1 immune checkpoint. PD-1 blocking antibodies inhibit the interaction of PD-L1 and PD-L2 with PD-1, whereas anti-PD-L1 antibodies inhibit the interaction between PD-L1 and PD-1, in some contexts, resulting in enhanced T cell activity, increased cytokine production, and tumor cell directed cytotoxicity. Expression of PD-1 and PD-L1 has been detected in various lymphomas. High levels of PD-L1 promote T cell exhaustion, and PD-L1 blockade reinvigorates T cell function.

In some contexts, tumor-infiltrating lymphocytes (TILs) express PD-1, and preliminary data also suggest that CAR T cells, upon stimulation through the CAR, upregulate expression of PD-1 and PD-L1, but not PD-L2. (See PCT Application WO2016196388.) Thus, both TILs and CARs-expressing T cells may, in some cases, be targets of suppression by PD-1/PD-L1. With these data in mind, CAR T cells given in combination with agents that block T cell suppression through the PD-1 pathway may have enhanced antitumor activity due to improved expansion of CAR T cells and prolonged duration of CAR T cells persistence and function. In some contexts, PD-L1 is upregulated in response to interferon-gamma (IFNγ) (Chen, Immunobiology. 2012 April; 217(4):385-93; Abiko, Br J Cancer. 2015 Apr. 28; 112(9):1501-9). As such, PD-L1 expression may be induced or further upregulated on tumor cells and other infiltrating cells at the site of CAR T cell action due to secretion of IFNγ by activated CAR T cells. Expression of PD-L1 by lymphoma cells may play a role in tumor-associated immunosuppression within the tumor microenvironment.

Monoclonal blocking antibodies to PD-1 or PD-L1 have been shown to be safe and effective in subjects with various cancers, and may be useful in reversing the PD-L1 mediated immunosuppression in subjects treated with CAR T cells. Blockade of the PD-1/PD-L1 axis has been explored in NHL (Lesokhin, J Clin Oncol. 2016 Aug. 10; 34(23):2698-704). Results of a Phase 1 study of the PD-1 antagonist monoclonal antibody nivolumab in NHL showed that PD-1 inhibition had an acceptable safety profile at similar dose levels (1 and 3 mg/kg) used for the treatment of solid tumors. Among 31 treated subjects with B-cell NHL, 71% experienced drug-related adverse events (AEs), including two subjects (7%) with serious adverse events (SAEs) of pneumonitis. The clinical study included 11 subjects with diffuse large B-cell lymphoma (DLBCL), and evidence of clinical activity was observed. Two subjects achieved a CR and two additional subjects achieved a PR. Median progression-free survival (PFS) was 6 (range 6 to 29) weeks for the cohort of subjects with DLBCL. Studies evaluating the activity of durvalumab in B-cell malignancies are currently ongoing.

Preliminary results from a clinical trial investigating another CD19-directed CAR T cell product CTL019 (Schuster, Blood. 2016 Dec. 1; 128(22):3026) in DLBCL identified high PD-L1 expression in lymphoma before CAR T cell infusion as one possible mechanism of resistance. Treatment of a subject not responding to CAR T cell therapy with a PD-1 blocking antibody resulted in a clinically significant antitumor response and expansion of CAR T cells (Chong, Blood. 2017 Feb. 23; 129(8):1039-41).

In certain embodiments, it is found that the pharmacokinetics (PK) of the cell therapy in the blood of subjects following administration of the cell therapy is similar or not substantially different between subjects that respond (e.g. exhibit a CR or an objective response (OR)) versus subjects that do not respond (e.g. exhibit a PD) to the cell therapy. In some embodiments, such observations indicate that the cell therapy has or is expanding in the subject but may not exhibit optimal efficacy. In some embodiments, the anti-PD-L1 antibody (or antigen-binding fragment thereof) is administered to the subjects that do not respond (e.g., exhibit a progressive disease (PD)) to the cell therapy or the subjects in which the cell therapy has not exhibited optimal efficacy.

In some contexts, optimal efficacy of a cell therapy can depend on the ability of the administered cells to recognize and bind to a target, e.g., target antigen, to traffic, localize to and successfully enter appropriate sites within the subject, tumors, and environments thereof. In some contexts, optimal efficacy can depend on the ability of the administered cells to become activated, expand, to exert various effector functions, including cytotoxic killing and secretion of various factors such as cytokines, to persist, including long-term, to differentiate, transition or engage in reprogramming into certain phenotypic states (such as long-lived memory, less-differentiated, and effector states), to avoid or reduce immunosuppressive conditions in the local microenvironment of a disease, to provide effective and robust recall responses following clearance and re-exposure to target ligand or antigen, and avoid or reduce exhaustion, anergy, peripheral tolerance, terminal differentiation, and/or differentiation into a suppressive state.

In some aspects, the efficacy of the cell therapy, e.g., T cell therapy, may be limited by the immunosuppressive activity or factors present in the local microenvironment of the disease or disorder, e.g., the TME. In some aspects, the TME contains or produces factors or conditions that can suppress the activity, function, proliferation, survival and/or persistence of T cells administered for T cell therapy. Without being bound by theory, the provided methods and uses are based on the hypothesis that a cell therapy, e.g., a CAR-expressing cell therapy, may be functionally inhibited by components of the immune checkpoint pathways, such as PD-L1, encountered in the lymphoma tumor microenvironment and that subjects may derive benefit from a combination therapy comprising a cell therapy and a checkpoint inhibitor, such as an anti-PD-L1 antibody (or antigen-binding fragment thereof), to prolong the cell therapy, e.g., a CAR-expressing T cell effector function upon encounter with this immunosuppressive signal.

In some embodiments, administration of a checkpoint inhibitor, such as an anti-PD-L1 antibody (or antigen-binding fragment thereof), subsequently to initiation of administration of the cell therapy, e.g. dose of T cells (e.g. CAR+ T cells) can result in improved activity, efficacy and/or persistence of the cell therapy and/or improve responses of the treated subject. In some embodiments, the combination therapy enhances, boosts and/or promotes the efficacy and/or safety of the therapeutic effect of the cell therapy, e.g. engineered T cell therapy, such as CAR+ T cells. In some embodiments, the checkpoint inhibitor, such as the anti-PD-L1 antibody (or antigen-binding fragment thereof), enhances or improves the efficacy, survival or persistence of the administered cells, e.g., cells expressing the recombinant receptor, e.g. CAR.

In some embodiments, the provided embodiments are based on an observation that administering a checkpoint inhibitor, such as an anti-PD-L1 antibody, subsequently to initiation of administration of a cell therapy (e.g. CAR+ T cells) and with a fractionated dosing schedule to subjects having a B cell malignancy, was generally safe, and was associated with improved pharmacokinetic profiles, overall response and prolonged response, in certain subjects. In some aspects, the fractionated dosing schedule includes a dosing regimen involving a plurality of cycles of administration (e.g. administration of the checkpoint inhibitor, such as an anti-PD-L1 antibody, in at least two 28-day cycles) in which the first cycle is carried out by administering a greater number of individual doses of the checkpoint inhibitor, e.g., anti-PD-L1 antibody, compared to the number of individual doses in the second or subsequent cycles. In particular, improvement in expansion of CAR+ T cells and prolonged response, such as prolonged complete response, were observed in certain subjects after administration of the checkpoint inhibitor, e.g., anti-PD-L1 antibody, even after a time point in which an initial reduction of CAR+ T cells was observed. In some aspects, the observations are consistent with a re-expansion or reduction of exhaustion and/or reduction of suppression of the administered CAR+ T cells. The results as shown for an exemplary checkpoint inhibitor, e.g. an anti-PD-L1 antibody durvalumab, support that the provided dosing regimen is safe in combination with CAR+ T cell therapy and can result in a clinical response in patients, including prolonged remission, particularly in patients achieving re-expansion and/or an improved pharmacokinetic profile of CAR+ T cells after administration of the checkpoint inhibitor. In some aspects, such results support combination therapy of CAR+ T cells and a checkpoint inhibitor, such as an inhibitor targeting the PD-1/PD-L1 pathway, such as an anti-PD-L1 antibody or an anti-PD-1 antibody, to achieve improved activity, potency, expansion and/or persistence of the cell therapy and/or improve responses of the treated subject.

In some aspects, the provided methods and uses provide for or achieve improved or more durable responses or efficacy as compared to certain alternative methods, e.g. methods that include administration of the T cell therapy or a checkpoint inhibitor, such as anti-PD-L1 antibody, as a monotherapy or without administration as a combination therapy together as described herein, such as in particular groups of subjects treated or based on the particular dosing method or regimen. In some embodiments, the methods are advantageous by virtue of administering T cell therapy, such as a composition including cells for adoptive cell therapy, e.g., such as a T cell therapy (e.g. CAR-expressing T cells), and a checkpoint inhibitor, such as an anti-PD-L1 antibody (or antigen-binding fragment thereof).

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. COMBINATION THERAPY

Provided are methods and uses of engineered cells, such as T cells (e.g., CAR-T cells) and a checkpoint inhibitor, such as an inhibitor of the PD-1/PD-L1 axis of the immune checkpoint pathway, such as an anti-PD-L1 antibody (or antigen-binding fragment thereof) and compositions thereof, for the treatment of subjects having a disease or condition, which generally is or includes a cancer or a tumor, such as certain B cell malignancies. In some aspects, the methods are for treating a leukemia or a lymphoma, such as a non-Hodgkin lymphoma (NHL). In some aspects, the methods and uses provide for or achieve improved response and/or more durable responses or efficacy, e.g., in particular groups of subjects treated, as compared to certain alternative methods. Also provided are articles of manufacture and kits containing a T cell therapy containing the cells and/or the checkpoint inhibitor, e.g., anti-PD-L1 antibody or antigen binding fragment thereof, e.g., for use in the methods provided herein. In some embodiments, the articles of manufacture and kits optionally contain instructions for using, according to the methods provided herein.

The engineered cells and the checkpoint inhibitor, e.g., anti-PD-L1 antibody or an anti-PD-1 antibody or fragment thereof, are useful in a variety of therapeutic, diagnostic and prophylactic indications. For example, the engineered cells or compositions comprising the engineered cells and the checkpoint inhibitor are useful in treating a variety of diseases and disorders in a subject. Such methods and uses include therapeutic methods and uses, for example, involving administration of the engineered cells and the checkpoint inhibitor, or compositions containing the same, to a subject having a disease, condition, or disorder, such as a tumor or cancer. In some embodiments, the engineered cells or compositions comprising the same and the checkpoint inhibitor are administered in an effective amount to effect treatment of the disease or disorder. Uses include uses of the engineered cells or compositions and the checkpoint inhibitor in such methods and treatments, and in the preparation of a medicament in order to carry out such therapeutic methods. In some embodiments, the methods are carried out by administering the engineered cells or compositions comprising the same and the checkpoint inhibitor, individually or together, to the subject having or suspected of having the disease or condition. In some embodiments, the methods thereby treat the disease or condition or disorder in the subject.

In some embodiments, the methods and uses include 1) administering to the subject a T cell therapy involving cells expressing genetically engineered (recombinant) cell surface receptors, which generally are chimeric receptors such as chimeric antigen receptors (CARs), recognizing an antigen expressed by, associated with and/or specific to the a B cell malignancy, such as a leukemia or lymphoma (e.g. NHL) and/or cell type from which it is derived, and 2) administering to the subject a checkpoint inhibitor, e.g., an anti-PD-L1 antibody (or antigen-binding fragment thereof). In some embodiments, the checkpoint inhibitor, e.g., the anti-PD-L1 or antigen-binding fragment thereof is administered after (subsequently) to administering the T cell therapy. In some cases, the checkpoint inhibitor, e.g., the anti-PD-L1 antibody or antigen-binding fragment is administered to a subject that has received administration of a T cell therapy. The methods generally involve administering one or more doses of the cells and one or more doses of a checkpoint inhibitor, e.g., an anti-PD-L1 antibody (or antigen-binding fragment thereof) to the subject. In some embodiments, the combination therapy is administered to a subject having the particular disease or condition to be treated. The disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer). Exemplary antigens, which include antigens associated with various diseases and conditions that can be treated, are described above. In particular embodiments, the chimeric antigen receptor or transgenic TCR specifically binds to an antigen associated with the disease or condition. In some embodiments, antigens targeted by the receptors include antigens associated with and/or expressed by a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30. In some aspects, the antigen expressed by or associated with a B cell malignancy is CD19.

Among provided embodiments is a method for treating a B-cell malignancy. In some embodiments, the diseases or disorders to be treated include leukemia and lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma (MZL), Burkitt lymphoma (BL), Hodgkin lymphoma (HL), non-Hodgkin lymphoma (NHL), Anaplastic large cell lymphoma (ALCL), follicular lymphoma, refractory follicular lymphoma, diffuse large B-cell lymphoma (DLBCL) and multiple myeloma (MM). In some embodiments, disease or condition is a B cell malignancy selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL). In some embodiments, the disease or condition is NHL and the NHL is selected from the group consisting of aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B).

In some embodiments, the methods involve treating a subject having a lymphoma or a leukemia, such as a non-Hodgkin lymphoma (NHL) with a dose of antigen receptor-expressing cells (e.g. CAR-expressing cells) and a subsequent dose of a checkpoint inhibitor, e.g., an anti-PD-L1 antibody (or antigen-binding fragment thereof).

In some embodiments, NHL can be staged based on the Lugano classification (see, e.g., Cheson et al., (2014) JCO 32(27):3059-3067; Cheson, B. D. (2015) Chin Clin Oncol 4(1):5). In some cases, the stages are described by Roman numerals I through IV (1-4), and limited stage (I or II) lymphomas that affect an organ outside the lymph system (an extranodal organ) are indicated by an “E.” Stage I represents involvement in one node or a group of adjacent nodes, or a single extranodal lesions without nodal involvement (IE). Stage 2 represents involvement in two or more nodal groups on the same side of the diaphragm or stage I or II by nodal extent with limited contiguous extranodal involvement (IIE). Stage III represents involvement in nodes on both sides of the diaphragm or nodes above the diaphragm with spleen involvement. Stage IV represents involvement in additional non-contiguous extralymphatic involvement. In addition, “bulky disease” can be used to describe large tumors in the chest, in particular for stage II. The extent of disease is determined by positron emission tomography (PET)-computed tomography (CT) for avid lymphomas, and CT for non-avid histologies.

In some embodiments, the Eastern Cooperative Oncology Group (ECOG) performance status indicator can be used to assess or select subjects for treatment, e.g., subjects who have had poor performance from prior therapies (see, e.g., Oken et al. (1982) Am J Clin Oncol. 5:649-655). In some embodiments, the subject has an ECOG status of less than or equal to 1. The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of self-care; see e.g., Sorensen et al., (1993) Br J Cancer 67(4) 773-775. The criteria reflective of the ECOG performance status are described in Table 1 below:

TABLE 1
ECOG Performance Status Criteria

| Grade | ECOG performance status |
|---|---|
| 0 | Fully active, able to carry on all pre-disease performance |
| | without restriction |
| 1 | Restricted in physically strenuous activity but ambulatory |
| | and able to carry out work of a light or sedentary nature, e.g., |
| | light house work, office work |
| 2 | Ambulatory and capable of all selfcare but unable to carry out |
| | any work activities; up and about more than 50% of |
| | waking hours |
| 3 | Capable of only limited selfcare; confined to bed or chair more |
| | than 50% of waking hours |
| 4 | Completely disabled; cannot carry on any selfcare; totally |
| | confined to bed or chair |
| 5 | Dead |

In some embodiments, the subject has or has been identified as having as having a double/triple hit lymphoma or a lymphoma of the double/triple hit molecular subtypes. In some embodiments, the lymphoma is a double hit lymphoma characterized by the presence of MYC (myelocytomatosis oncogene), BCL2 (B-cell lymphoma 2), and/or BCL6 (B-cell lymphoma 6) gene rearrangements (e.g., translocations). In some embodiments, the gene rearrangement affects the MYC/8q24 locus in combination with another gene rearrangement. For example, the other gene rearrangement includes t(14; 18)(q32; q21) involving BCL2. In some embodiments, the gene rearrangements affect the MYC/8q24 locus in combination with BCL6/3q27. In some embodiments, the lymphoma is a triple hit lymphoma characterized by the presence of MYC, BCL2, and BCL6 gene rearrangements; see, e.g., Aukema et al., (2011) Blood 117:2319-2331. In some aspects of such embodiments the subject is ECOG 0-1 or does not have or is not suspected or characterized as having DLBCL transformed from MZL or CLL. In aspects, the therapy is indicated for such subjects and/or the instructions indicate administration to a subject within such population. In some embodiments, based on the 2016 WHO criteria (Swerdlow et al., (2016) Blood 127(20):2375-2390), double/triple hit lymphoma can be considered high-grade B-cell lymphoma, with MYC and BCL2 and/or BCL6 rearrangements with DLBCL histology (double/triple hit).

In some embodiments, the combination therapy is administered to subjects who are or are likely to be or who are predicted to be poor responders and/or who do not, are likely not to and/or who are predicted not to respond or do not respond within a certain time and/or to a certain extent to treatment with the cell therapy, e.g. dose of T cells (e.g. CAR+ T cells). In some embodiments, the combination therapy is administered to subjects who do not or are not likely to or are not predicted to exhibit a complete response or overall response, such as within 1 month, within two months or within three months after initiation of administration of the cell therapy. In some embodiments, the combination therapy is administered to subjects who exhibit or are likely to exhibit or who are predicted to exhibit progressive disease (PD), such as within 1 month, two months or three months, following administration of the cell therapy. In some embodiments, a subject is likely or predicted not to exhibit a response or a certain response based on a plurality of similarly situated subjects so treated or previously treated with the cell therapy.

In some embodiments, the provided methods involve treating a specific group or subset of subjects, e.g., subjects identified as having high-risk disease, e.g., high-risk NHL. In some aspects, the methods treat subjects having a form of aggressive and/or poor prognosis B-cell non-Hodgkin lymphoma (NHL), such as NHL that has relapsed or is refractory (R/R) to standard therapy has a poor prognosis. In some cases, the overall response rate (ORR) to available therapies, to a standard of care, or to a reference therapy for the disease and/or patient population for which the therapy is indicated, is less than 40% and/or the complete response (CR) is less than 20%. In some embodiments, in chemorefractory DLBCL, the ORR with a reference or available treatment or standard-of-care therapy is about 26% and the CR is about 8% (Crump et al. Outcomes in refractory aggressive diffuse large B-cell lymphoma (DLBCL): Results from the international SCHOLAR study. ASCO 2016 [Abstract 7516]). In some aspects, the provided methods, compositions, uses and articles of manufacture achieve improved and superior responses to available therapies.

In some embodiments, the methods, uses and articles of manufacture involve, or are used for treatment of subjects involving, selecting or identifying a particular group or subset of subjects, e.g., based on specific types of disease, diagnostic criteria, prior treatments and/or response to prior treatments. In some embodiments, the methods involve treating a subject having relapsed following remission after treatment with, or become refractory to, one or more prior therapies; or a subject that has relapsed or is refractory (R/R) to one or more prior therapies, e.g., one or more lines of standard therapy. In some embodiments, the methods involve treating subjects having diffuse large B-cell lymphoma (DLBCL), not otherwise specified (NOS; de novo and transformed from indolent), primary mediastinal (thymic) large B-cell lymphoma (PMBCL) or follicular lymphoma grade 3B (FL3B), Epstein-Barr virus (EBV) positive DLBCL, or EBV positive NOS. In some embodiments, the methods involve treating a subject that has an Eastern Cooperative Oncology Group Performance Status (ECOG) of less than 1, such as 0-1. In some embodiments, the methods treat a poor-prognosis population or of DLBCL patients or subject thereof that generally responds poorly to therapies or particular reference therapies, such as one having one or more, such as two or three, chromosomal translocations (such as so-called “double-hit” or “triple-hit” lymphoma, which is high grade B-cell lymphoma with MYC and BCL2 and/or BCL6 rearrangements with DLBCL histology; having translocations MYC/8q24 loci, usually in combination with the t (14; 18) (q32; q21) bcl-2 gene or/and BCL6/3q27 chromosomal translocation; see, e.g., Xu et al. (2013) Int J Clin Exp Pathol. 6(4): 788-794), and/or one having relapsed, optionally relapsed within 12 months, following administration of an autologous stem cell transplant (ASCT), and/or one having been deemed chemorefractory.

In some embodiments, the antigen receptor (e.g. CAR) specifically binds to a target antigen associated with the disease or condition, such as associated with NHL. In some embodiments, the antigen associated with the disease or disorder is selected from CD20, CD19, CD22, ROR1, CD45, CD21, CD5, CD33, Igkappa, Iglambda, CD79a, CD79b or CD30.

In some embodiments, the methods include administration of the cell therapy and a checkpoint inhibitor, e.g., an anti-PD-L1 antibody (or antigen-binding fragment thereof) to a subject, which is, at risk for, or suspected of having the disease, condition or disorder. In some embodiments, the subject is the subject is an adult. In some embodiments, the subject is over at or about 30, 40, 50, 60, or 70 years of age.

In some embodiments, the methods include administration of cells to a subject selected or identified as having a certain prognosis or risk of NHL. Non-Hodgkin lymphoma (NHL) can be a variable disease. Some subjects with NHL may survive without treatment while others may require immediate intervention. In some cases, subjects with NHL may be classified into groups that may inform disease prognosis and/or recommended treatment strategy. In some cases, these groups may be “low risk,” “intermediate risk,” “high risk,” and/or “very high risk” and patients may be classified as such depending on a number of factors including, but not limited to, genetic abnormalities and/or morphological or physical characteristics. In some embodiments, subjects treated in accord with the methods, and/or with the articles of manufacture or compositions, are classified or identified based on the risk of NHL. In some embodiments, the subject is one that has high risk NHL.

In some embodiments, the subject has been previously treated with a therapy or a therapeutic agent targeting the disease or condition, e.g., NHL, prior to administration of the cells expressing the recombinant receptor. In some embodiments, the subject has been previously treated with a hematopoietic stem cell transplantation (HSCT), e.g., allogenic HSCT or autogenic HSCT. In some embodiments, the subject has had poor prognosis after treatment with standard therapy and/or has failed one or more lines of previous therapy. In some embodiments, the subject has been treated or has previously received at least or about at least or about 1, 2, 3, 4, 5, 6, or 7 other therapies for treating the NHL other than a lymphodepleting therapy. In some embodiments, the subject has been previously treated with chemotherapy or radiation therapy. In some aspects, the subject is refractory or non-responsive to the other therapy or therapeutic agent. In some embodiments, the subject has persistent or relapsed disease, e.g., following treatment with another therapy or therapeutic intervention, including chemotherapy or radiation.

In some embodiments, the methods include administration of cells to a subject selected or identified as having high-risk NHL. In some embodiments, the subject exhibits one or more cytogenetic abnormalities, such as associated with high-risk NHL. In some embodiments, the subject is selected or identified based on having a disease or condition characterized or determined to be aggressive NHL, diffuse large B cell lymphoma (DLBCL), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL). In particular embodiments, the subject to be treated using the methods provided herein include subjects with aggressive NHL, in particular, with diffuse large B-cell lymphoma (DLBCL), not otherwise specified (NOS; de novo and transformed from indolent), primary mediastinal (thymic) large B-cell lymphoma (PMBCL) or follicular lymphoma grade 3B (FL3B), Epstein-Barr virus (EBV) positive DLBCL, EBV positive NOS, or high grade B-cell lymphoma with MYC and BCL2 and/or BCL6 rearrangements with DLBCL histology (“double-hit” or “triple-hit” lymphoma). In some embodiments, the subject has poor performance status. In some aspects, the population to be treated includes subjects having an Eastern Cooperative Oncology Group Performance Status (ECOG) that is anywhere from 0-2. In other aspects of any of the embodiments, the subjects to be treated included ECOG 0-1 or do not include ECOG 2 subjects. In some aspects of any of the embodiments, the subjects to be treated have failed two or more prior therapies. In some embodiments, the subject does not have DLBCL transformed from marginal zone lymphoma (MZL) and chronic lymphocytic leukemia

(CLL; Richter's). In some embodiments, a subject with CLL can exhibit Richter's syndrome (RS), defined as the transformation of CLL into an aggressive lymphoma, most commonly diffuse large B-cell lymphoma (DLBCL) (see, e.g., Parikh et al. Blood 2014 123:1647-1657). In some embodiments, the subject has mantle cell lymphoma (MCL). In some embodiments, the subject has features that correlate with poor overall survival. In some embodiments, the subject has never achieved a complete response (CR), never received autologous stem cell transplant (ASCT), refractory to 1 or more second line therapy, has primary refractory disease, and/or has an ECOG performance score of 2.

In some embodiments, the subject to be treated includes a group of subjects with aggressive NHL, in particular, with diffuse large B-cell lymphoma (DLBCL), not otherwise specified (NOS; de novo and transformed from indolent), primary mediastinal (thymic) large B-cell lymphoma (PMBCL) or follicular lymphoma grade 3B (FL3B), Epstein-Barr virus (EBV) positive DLBCL, EBV positive NOS, or high grade B-cell lymphoma with MYC and BCL2 and/or BCL6 rearrangements with DLBCL histology (“double-hit” or “triple-hit” lymphoma). In some embodiments, the subject's disease has relapsed or been refractory to at least two prior lines of therapy. In some embodiments, the prior therapy comprises a CD20-targeted agent and/or an anthracycline. In some embodiments, the subjects have a ECOG score of 0-2 or 0-1 at screening. In some embodiments, the subjects have positron emission tomography (PET)-positive disease as per Lugano Classification (Cheson, 2014). In some embodiments, the subject may optionally have previously been treated with allogenic stem cell transplantation (SCT).

In some embodiments, the subjects are at least 40 years old at the time they are administered the combination therapy (e.g., at the time they are administered the cell therapy). In some embodiments, the subjects are less than 40 years old at the time they are administered the combination therapy (e.g., at the time they are administered the cell therapy). In some embodiments, the subjects are about 40-65 years old at the time they are administered the combination therapy (e.g., at the time they are administered the cell therapy). In some embodiments, the subjects are at least 65 years old at the time they are administered the combination therapy (e.g., at the time they are administered the cell therapy).

In some embodiments, the subjects are male. In some embodiments, the subjects are female.

In some embodiments, the subjects are refractory to last prior therapy. In some embodiments, the subjects have a relapse to last prior therapy. The status is refractory if a subject achieved less than a partial response to last prior therapy. In some embodiments, the subjects have a prior chemotherapy. In some embodiments, the subjects are chemorefractory to the prior chemotherapy. In some embodiments, the subjects are chemosensitive to the prior therapy. The status is chemorefractory is a subject achieved stable disease (SD) or progressive disease (PD) to last chemotherapy-containing regimen or relapsed less than 12 months after autologous stem cell transplant. Otherwise the status is chemosensitive.

In some embodiments, the methods, cells and compositions can provide high rate of durable response to high risk patients with poor prognosis, with a reduced risk of adverse effects or toxicities. In some embodiments, the methods and uses provide for or achieve a higher response rate and/or more durable responses or efficacy and/or a reduced risk of toxicity or other side effects that can be associated with the combination therapy including the cell therapy, such as neurotoxicity (NT) or cytokine release syndrome (CRS).

In some embodiments, at least 35%, at least 40%, at least 50%, at least 55%, at least 60%, at least 65%, at least 70%, or at least 75% or more of the subjects treated according to the provided methods, and/or with the provided articles of manufacture or compositions, achieve a CR. In some embodiments, at least 50%, at least 60%, at least 70%, at least 80%, or at least 90% of the subjects treated according to the provided methods, and/or with the provided articles of manufacture or compositions, achieve an objective response of a partial response (PR). In some embodiments, at least 60%, at least 70%, at least 80%, at least 90%, at least 95% or more of the subjects treated according to the provided methods, and/or with the provided articles of manufacture or compositions, achieve a CR or PR by one month, by two months or by three months.

In some embodiments, by three months, four months, five months, six months or more after initiation of administration of the cell therapy, at least 60%, at least 70%, at least 80%, at least 90%, at least 95% or more of the subjects treated according to the provided methods, and/or with the provided articles of manufacture or compositions, remain in response, such as remain in CR or OR. In some embodiments, such response, such as CR or OR, is durable for at least three months, four months, five months, six months, seven months, eight months or nine months, such as in at least or about at least 60%, at least 70%, at least 80%, at least 90%, at least 95% or more of the subjects treated according to the provided methods or in such subjects who achieve a CR by one month or by three months. In some embodiments, at least 60%, at least 70%, at least 80%, at least 90%, at least 95% or more of the subjects treated according to the provided methods, and/or with the provided articles of manufacture or compositions, or such subjects who achieve a CR by one month or by three months, survive or survive without progression for greater than or greater than about three months, four months, five months, six months, seven months, eight months or nine months.

In some aspects, the provided methods can achieve a high or a particular rate of response (such as a rate of response among a population as assessed after a certain period post-administration, such as three months or six months), e.g., ORR (such as a 6-month or 3-month ORR) of 75% or 80% or 81%, 82%, 83%, 84% or 85% or more and CR rate (such as a 6-month or 3-month CR rate) of 50% or more, 55% or more, 60% or more, 65% or more, 70% or more, 71%, 72%, 73% or more or approximately 75% or more, which also is durable such as for a particular period of time or at least a particular period of time, e.g., is sustained for more than 1, 3 or 6 months or more or 9 months or more after initiation of therapy. In some embodiments, such rates of response and durability are received following about 1, 2, 3, 4, 5, 6, 7, 8, 9, or 10 administration or dose of the checkpoint inhibitor, e.g., anti-PD-L1 antibody or antigen-binding fragment.

A. Administration of Cell Therapy

Methods for administration of cells for adoptive cell therapy are known and may be used in connection with the provided methods, compositions and articles of manufacture and kits. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cells for use in or administered in connection with the provided methods contain or are engineered to contain an engineered receptor, e.g., an engineered antigen receptor, such as a chimeric antigen receptor (CAR), or a T cell receptor (TCR). Among the compositions are pharmaceutical compositions and formulations for administration, such as for adoptive cell therapy. Also provided are therapeutic methods for administering the cells and compositions to subjects, e.g., patients, in accord with the provided methods, and/or with the provided articles of manufacture or compositions.

The cells generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors. Exemplary engineered cells for administering as a cell therapy in the provided methods are described in Section II.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells of the T cell therapy can be administered in a composition formulated for administration, or alternatively, in more than one composition (e.g., two compositions) formulated for separate administration. The dose(s) of the cells may include a particular number or relative number of cells or of the engineered cells, and/or a defined ratio or compositions of two or more sub-types within the composition, such as CD4 vs. CD8 T cells.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the treatment of disease, the appropriate dosage may depend on the type of disease to be treated, the type of cells or recombinant receptors, the severity and course of the disease, previous therapy, the subject's clinical history and response to the cells, and the discretion of the attending physician. The compositions and cells are in some embodiments suitably administered to the subject at one time or over a series of treatments.

Preconditioning subjects with immunodepleting (e.g., lymphodepleting) therapies in some aspects can improve the effects of adoptive cell therapy (ACT).

Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the first or subsequent dose.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

I. Compositions and Formulations

In some embodiments, the dose of cells of the cell therapy, such as a T cell therapy comprising cells engineered with a recombinant antigen receptor, e.g. CAR or TCR, is provided as a composition or formulation, such as a pharmaceutical composition or formulation. Such compositions can be used in accord with the provided methods and/or with the provided articles of manufacture or compositions, such as in the treatment of diseases, conditions, and disorders.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some embodiments, the cell therapy, such as engineered T cells (e.g. CAR T cells), are formulated with a pharmaceutically acceptable carrier. In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some embodiments contains cells in amounts effective to treat the disease or condition, such as a therapeutically effective or prophylactically effective amount. Therapeutic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

2. Dosing

In some embodiments, a dose of cells is administered to subjects in accord with the provided methods, and/or with the provided articles of manufacture or compositions. In some embodiments, the size or timing of the doses is determined as a function of the particular disease or condition in the subject. In some cases, the size or timing of the doses for a particular disease in view of the provided description may be empirically determined.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells, or individual populations of sub-types of cells, are administered to the subject at a range of about one million to about 100 billion cells and/or that amount of cells per kilogram of body weight, such as, e.g., at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 450 million cells, at or about 650 million cells, at or about 800 million cells, at or about 900 million cells, at or about 3 billion cells, at or about 30 billion cells, at or about 45 billion cells) or any value in between these ranges and/or per kilogram of body weight. Dosages may vary depending on attributes particular to the disease or disorder and/or patient and/or other treatments.

In some embodiments, the dose of cells is a flat dose of cells or fixed dose of cells such that the dose of cells is not tied to or based on the body surface area or weight of a subject.

In some embodiments, the dose of genetically engineered cells comprises from or from about 1×10⁵ to 5×10⁸ total CAR-expressing T cells, 1×10⁵ to 2.5×10⁸ total CAR-expressing T cells, 1×10⁵ to 1×10⁸ total CAR-expressing T cells, 1×10⁵ to 5×10⁷ total CAR-expressing T cells, 1×10⁵ to 2.5×10⁷ total CAR-expressing T cells, 1×10⁵ to 1×10⁷ total CAR-expressing T cells, 1×10⁵ to 5×10⁶ total CAR-expressing T cells, 1×10⁵ to 2.5×10⁶ total CAR-expressing T cells, 1×10⁵ to 1×10⁶ total CAR-expressing T cells, 1×10⁶ to 5×10⁸ total CAR-expressing T cells, 1×10⁶ to 2.5×10⁸ total CAR-expressing T cells, 1×10⁶ to 1×10⁸ total CAR-expressing T cells, 1×10⁶ to 5×10⁷ total CAR-expressing T cells, 1×10⁶ to 2.5×10⁷ total CAR-expressing T cells, 1×10⁶ to 1×10⁷ total CAR-expressing T cells, 1×10⁶ to 5×10⁶ total CAR-expressing T cells, 1×10⁶ to 2.5×10⁶ total CAR-expressing T cells, 2.5×10⁶ to 5×10⁸ total CAR-expressing T cells, 2.5×10⁶ to 2.5×10⁸ total CAR-expressing T cells, 2.5×10⁶ to 1×10⁸ total CAR-expressing T cells, 2.5×10⁶ to 5×10⁷ total CAR-expressing T cells, 2.5×10⁶ to 2.5×10⁷ total CAR-expressing T cells, 2.5×10⁶ to 1×10⁷ total CAR-expressing T cells, 2.5×10⁶ to 5×10⁶ total CAR-expressing T cells, 5×10⁶ to 5×10⁸ total CAR-expressing T cells, 5×10⁶ to 2.5×10⁸ total CAR-expressing T cells, 5×10⁶ to 1×10⁸ total CAR-expressing T cells, 5×10⁶ to 5×10⁷ total CAR-expressing T cells, 5×10⁶ to 2.5×10⁷ total CAR-expressing T cells, 5×10⁶ to 1×10⁷ total CAR-expressing T cells, 1×10⁷ to 5×10⁸ total CAR-expressing T cells, 1×10⁷ to 2.5×10⁸ total CAR-expressing T cells, 1×10⁷ to 1×10⁸ total CAR-expressing T cells, 1×10⁷ to 5×10⁷ total CAR-expressing T cells, 1×10⁷ to 2.5×10⁷ total CAR-expressing T cells, 2.5×10⁷ to 5×10⁸ total CAR-expressing T cells, 2.5×10⁷ to 2.5×10⁸ total CAR-expressing T cells, 2.5×10⁷ to 1×10⁸ total CAR-expressing T cells, 2.5×10⁷ to 5×10⁷ total CAR-expressing T cells, 5×10⁷ to 5×10⁸ total CAR-expressing T cells, 5×10⁷ to 2.5×10⁸ total CAR-expressing T cells, 5×10⁷ to 1×10⁸ total CAR-expressing T cells, 1×10⁸ to 5×10⁸ total CAR-expressing T cells, 1×10⁸ to 2.5×10⁸ total CAR-expressing T cells, or 2.5×10⁸ to 5×10⁸ total CAR-expressing T cells.

In some embodiments, the dose of genetically engineered cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

In some embodiments, the cell therapy comprises administration of a dose comprising a number of cell from or from about 1×10⁵ to 5×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3⁺ or CD8⁺, in some cases also recombinant receptor-expressing (e.g. CAR⁺) cells. In some embodiments, the cell therapy comprises administration of a dose comprising a number of cell from or from about 1×10⁵ to 5×10⁸ CD3⁺ or CD8⁺ total T cells or CD3⁺ or CD8⁺ recombinant receptor-expressing cells, from or from about 5×10⁵ to 1×10⁷ CD3⁺ or CD8⁺ total T cells or CD3⁺ or CD8⁺ recombinant receptor-expressing cells, or from or from about 1×10⁶ to 1×10⁷ CD3⁺ or CD8⁺ total T cells or CD3⁺ or CD8⁺ recombinant receptor-expressing cells, each inclusive. In some embodiments, the cell therapy comprises administration of a dose comprising a number of cell from or from about 1×10⁵ to 5×10⁸ total CD3⁺/CAR⁺ or CD8⁺/CAR⁺ cells, from or from about 5×10⁵ to 1×10⁷ total CD3⁺/CAR⁺ or CD8⁺/CAR⁺ cells, or from or from about 1×10⁶ to 1×10⁷ total CD3⁺/CAR⁺ or CD8⁺/CAR⁺ cells, each inclusive.

In some embodiments, the dose of T cells comprises: at or about 5×10⁷ recombinant receptor-expressing T cells or at or about 2.5×10⁷ recombinant receptor-expressing CD8⁺ T cells. In some embodiments, the dose of T cells comprises: at or about 1×10⁸ recombinant receptor-expressing T cells or at or about 5×10⁷ recombinant receptor-expressing CD8⁺ T cells. In some embodiments, the dose of T cells comprises: at or about 1.5×10⁸ recombinant receptor-expressing T cells or at or about 0.75×10⁸ recombinant receptor-expressing CD8⁺ T cells.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of about 1×10⁶ to 5×10⁸ such cells, such as 2×10⁶, 5×10⁶, 1×10⁷, 5×10⁷, 1×10⁸, 2×10⁸, 3×10⁸, or 4×10⁸ total such cells, or the range between any two of the foregoing values. In some embodiments, where the subject is a human, the dose includes between about 1×10⁶ and 3×10⁸ total recombinant receptor (e.g., CAR)-expressing cells, e.g., in the range of about 1×10⁷ to 2×10⁸ such cells, such as 1×10⁷, 5×10⁷, 1×10⁸ or 1.5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁵ to 5×10⁸ total recombinant receptor (e.g. CAR)-expressing T cells or total T cells, 1×10⁵ to 1.5×10⁸ total recombinant receptor (e.g. CAR)-expressing T cells or total T cells, 1×10⁵ to 1×10⁸ total recombinant receptor (e.g. CAR)-expressing T cells or total T cells, from or from about 5×10⁵ to 1×10⁷ total recombinant receptor (e.g. CAR)r-expressing T cells or total T cells, or from or from about 1×10⁶ to 1×10⁷ total recombinant receptor (e.g. CAR)-expressing T cells or total T cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between about 1×10⁶ and 1×10⁸ total recombinant receptor (e.g., CAR)-expressing CD8+ cells, e.g., in the range of about 5×10⁶ to 1×10⁸ such cells, such cells 1×10⁷, 2.5×10⁷, 5×10⁷, 7.5×10⁷ or 1×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD8+ T cells, 1×10⁷ to 2.5×10⁷ total recombinant receptor-expressing CD8+ T cells, from or from about 1×10⁷ to 0.75×10⁸ total recombinant receptor-expressing CD8+ T cells, each inclusive. In some embodiments, the dose of cells comprises the administration of or about 1×10⁷, 2.5×10⁷, 5×10⁷, 7.5×10⁷ or 1×10⁸ total recombinant receptor-expressing CD8+ T cells.

In some embodiments, for example, where the subject is human, the CD4+ T cells of the dose, including in a dose including CD4+ and CD8+ T cells, includes between about 1×10⁶ and 1×10⁸ total recombinant receptor (e.g., CAR)-expressing CD4+ cells, e.g., in the range of about 5×10⁶ to 1×10⁸ such cells, such as 1×10⁷, 2.5×10⁷, 5×10⁷, 7.5×10⁷ or 1×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD4+ T cells, 1×10⁷ to 2.5×10⁷ total recombinant receptor-expressing CD4+ T cells, from or from about 1×10⁷ to 0.75×10⁸ total recombinant receptor-expressing CD4+ T cells, each inclusive. In some embodiments, the dose of cells comprises the administration of or about 1×10⁷, 2.5×10⁷, 5×10⁷, 7.5×10⁷ or 1×10⁸ total recombinant receptor-expressing CD4+ T cells.

In some embodiments, the dose of cells, e.g., recombinant receptor-expressing T cells, is administered to the subject as a single dose or is administered only one time within a period of two weeks, one month, three months, six months, 1 year or more.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8+− and CD4+− enriched populations, respectively, e.g., CD4+ and/or CD8+ T cells each individually including cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, a composition containing cells to be administered, or a dose or a population of cells to be administered, such as a composition, a population or a dose of engineered T cells, are enriched for or contain at least a certain percentage or proportion of particular sub-types of cells. In some embodiments, a composition or a population of cells for administration is enriched for or contain at least a certain percentage or proportion of CD8+ and/or CD4+ T cells.

In some embodiments, a composition or a population of cells for administration is enriched for or contain at least a certain percentage or proportion of CD8+ T cells. In some of any embodiments, a composition or a population of cells for administration comprises at least at or about 50%, at or about 60%, at or about 70%, at or about 80%, at or about 90%, at or about 91%, at or about 92%, at or about 93%, at or about 94%, at or about 95%, at or about 96%, at or about 97%, at or about 98% or at or about 99% CD8+ T cells. In some embodiments, a composition or a population of cells for administration comprises at least at or about 95% CD8+ T cells. In some embodiments, a composition or a population of cells for administration comprises at least at or about 96% CD8+ T cells. In some embodiments, a composition or a population of cells for administration comprises at least at or about 97% CD8+ T cells. In some embodiments, a composition or a population of cells for administration comprises at least at or about 98% CD8+ T cells. In some embodiments, a composition or a population of cells for administration comprises at least at or about 99% CD8+ T cells.

In some embodiments, a composition or a population of cells for administration is enriched for or contain at least a certain percentage or proportion of CD4+ T cells. In some of any embodiments, a composition or a population of cells for administration comprises at least at or about 50%, at or about 60%, at or about 70%, at or about 80%, at or about 90%, at or about 91%, at or about 92%, at or about 93%, at or about 94%, at or about 95%, at or about 96%, at or about 97%, at or about 98% or at or about 99% CD4+ T cells. In some embodiments, a composition or a population of cells for administration comprises at least at or about 95% CD4+ T cells. In some embodiments, a composition or a population of cells for administration comprises at least at or about 96% CD4+ T cells. In some embodiments, a composition or a population of cells for administration comprises at least at or about 97% CD4+ T cells. In some embodiments, a composition or a population of cells for administration comprises at least at or about 98% CD4+ T cells. In some embodiments, a composition or a population of cells for administration comprises at least at or about 99% CD4+ T cells.

In some embodiments, a composition or a population of cells for administration is enriched for or contain at least a certain combined percentage or proportion of CD8+ and CD4+ T cells. In some of any embodiments, in a composition or a population of cells for administration, the percentage or proportion of CD8+ T cells and CD4+ T cells together is at least at or about 50%, at or about 60%, at or about 70%, at or about 80%, at or about 90%, at or about 91%, at or about 92%, at or about 93%, at or about 94%, at or about 95%, at or about 96%, at or about 97%, at or about 98% or at or about 99%. In some of any embodiments, in a composition or a population of cells for administration, the percentage or proportion of CD8+ T cells and CD4+ T cells together is at least at or about 95%. In some of any embodiments, in a composition or a population of cells for administration, the percentage or proportion of CD8+ T cells and CD4+ T cells together is at least at or about 96%. In some embodiments, In some of any embodiments, in a composition or a population of cells for administration, the percentage or proportion of CD8+ T cells and CD4+ T cells together is at least at or about 97%. In some of any embodiments, in a composition or a population of cells for administration, the percentage or proportion of CD8+ T cells and CD4+ T cells together is at least at or about 98%. In some of any embodiments, in a composition or a population of cells for administration, the percentage or proportion of CD8+ T cells and CD4+ T cells together is at least at or about 99%.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered within 48 hours of each other, such as no more than 36 hours of each other or not more than 24 hours of each other. In some embodiments, the first composition and second composition are administered 0 to 12 hours apart, 0 to 6 hours apart or 0 to 2 hours apart. In some embodiments, the initiation of administration of the first composition and the initiation of administration of the second composition are carried out no more than 2 hours, no more than 1 hour, or no more than 30 minutes apart, no more than 15 minutes, no more than 10 minutes or no more than 5 minutes apart. In some embodiments, the initiation and/or completion of administration of the first composition and the completion and/or initiation of administration of the second composition are carried out no more than 2 hours, no more than 1 hour, or no more than 30 minutes apart, no more than 15 minutes, no more than 10 minutes or no more than 5 minutes apart.

In some embodiments, the first composition and the second composition is mixed prior to the administration into the subject. In some embodiments, the first composition and the second composition is mixed shortly (e.g., within 6 hours, 5 hours, 4 hours, 3 hours, 2 hours, 1.5 hours, 1 hour, or 0.5 hour) before the administration, In some embodiments, the first composition and the second composition is mixed immediately before the administration.

In some composition, the first composition, e.g., first composition of the dose, comprises CD4+ T cells. In some composition, the first composition, e.g., first composition of the dose, comprises CD8+ T cells. In some embodiments, the first composition is administered prior to the second composition.

In some embodiments, the dose or composition of cells includes a defined or target ratio of CD4+ cells expressing a recombinant receptor to CD8+ cells expressing a recombinant receptor and/or of CD4+ cells to CD8+ cells, which ratio optionally is approximately 1:1 or is between approximately 1:3 and approximately 3:1, such as approximately 1:1. In some aspects, the administration of a composition or dose with the target or desired ratio of different cell populations (such as CD4+:CD8+ ratio or CAR+CD4+:CAR+CD8+ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

B. Administration of a Checkpoint Inhibitor, e.g., an Anti-PD-L1 Antibody or Fragment Thereof

In some embodiments of the methods, compositions, combinations, kits or articles of manufacture provided herein, the combination therapy comprises administering a checkpoint inhibitor, such as an anti-PD-L1 antibody (or antigen-binding fragment thereof), e.g., a pharmaceutical composition containing such checkpoint inhibitors, e.g., an anti-PD-L1 antibody (or antigen-binding fragment thereof). In some aspects, the checkpoint inhibitor is capable of inhibiting or blocking a protein or component of an immune checkpoint pathway, such as the PD-1/PD-L1 axis of the checkpoint pathway. In some embodiments, exemplary checkpoint inhibitors include an anti-PD-L1 antibody or an anti-PD-1 antibody.

1. Anti-PD-L1 Antibody or Fragment Thereof

In some embodiments of the methods, compositions, combinations, kits or articles of manufacture provided herein, the combination therapy comprises administering an anti-PD-L1 antibody (or antigen-binding fragment thereof), e.g., a pharmaceutical composition containing an anti-PD-L1 antibody (or antigen-binding fragment thereof). In some of any of the embodiments, the checkpoint inhibitor is or comprises an anti-PD-1 antibody or an antigen-binding fragment thereof.

In some embodiments, the anti-PD-L1 antibody specifically binds to PD-L1, such as to the extracellular region of PD-L1. In some embodiments, the anti-PD-L1 antibody (or antigen-binding fragment thereof) binds to PD-L1 with a binding affinity (K_{D}) of less than about 5, 4, 3, 2.5, 2, or 1 nanomolar (nM). In some embodiments, the anti-PD-L1 antibody (or antigen-binding fragment thereof) binds to the target (i.e., PD-L1) with a binding affinity (K_{D}) of about 5 nM to about 1 nM; or about 5 nM to about 2 nM; or about 5 nM to about 3 nM; or about 5 nM to about 4 nM; or about 3 nM to about 1 nM; or about 2 nM to about 1 nM. In some embodiments, the anti-PD-L1 antibody (or antigen-binding fragment thereof) binds to the target (i.e., PD-L1) with a binding affinity (K_{D}) of less than about 950 picomolar (pM). In some embodiments, the anti-PD-L1 antibody (or antigen-binding fragment thereof) binds to the target (i.e., PD-L1) with a binding affinity (K_{D}) of less than about 900, 800, 700, 600, 500, 400, 300, 200, or 100 pM. In some embodiments, the anti-PD-L1 antibody (or antigen-binding fragment thereof) binds to the target (i.e., PD-L1) with a binding affinity (K_{D}) of about 900 pM to about 100 pM; or about 900 pM to about 200 pM; or about 900 pM to about 300 pM; or about 900 pM to about 400 pM; or about 900 pM to about 500 pM; or about 900 pM to about 600 pM; or about 900 pM to about 700 pM; or about 200 pM to about 100 pM; or about 300 pM to about 200 pM; or about 400 pM to about 300 pM. In some embodiments, the anti-PD-L1 antibody (or antigen-binding fragment thereof) binds to the target (i.e., PD-L1) with a binding affinity (K_{D}) of less than about 90 pM, 80 pM, 70 pM, 60 pM, 55 pM or 50 pM. In some embodiments, the anti-PD-L1 antibody (or antigen-binding fragment thereof) binds to the target (i.e., PD-L1) with a binding affinity (K_{D}) of about 100 pM to about 50 pM; or about 100 pM to about 70 pM; or about 100 pM to about 80 pM; or about 100 pM to about 90 pM; or about 70 pM to about 50 pM; or about 60 pM to about 50 pM; or about 55 pM to about 50 pM. The K_{D} may be assessed using a method known to one of skill in the art (e.g., a BIAcore assay, ELISA) (Biacore International AB, Uppsala, Sweden).

In some embodiments, the binding properties of the anti-PD-L1 antibody (or antigen-binding fragment thereof) with reference to binding PD-L1 may also be measured by reference to the dissociation or association rates (k_{off} and kₒₙ respectively). In some embodiments, the anti-PD-L1 antibody (or antigen-binding fragment thereof) has a kₒₙ rate (antibody (Ab)+antigen (Ag)^{kon}→Ab-Ag) of at least about 10⁴ M⁻¹ s⁻¹, at least about 5×10⁴ M⁻¹ s⁻¹ at least about 10⁵ M⁻¹ s⁻¹, at least about 2×10⁵ M⁻¹ s⁻¹, at least about 5×10⁵ M⁻¹ s⁻¹, at least about 10⁶ M⁻¹ s⁻¹, at least about 5×10⁶ M⁻¹ S⁻¹, at least about 10⁷ M⁻¹ S⁻¹, at least about 5×10⁷ M⁻¹ S⁻¹, or at least about 10⁸ M⁻¹ s−1. In some embodiments, the kₒₙ rate is measured by a BIAcore assay.

In some embodiments, the binding properties of the anti-PD-L1 antibody (or antigen-binding fragment thereof) has a k_{off} rate (antibody (Ab)+antigen (Ag)^{koff}→Ab-Ag) of less than about 5×10⁻¹ s⁻¹, less than about 10⁻¹ s⁻¹, less than about 5×10⁻² s⁻¹, less than about 10⁻² s⁻¹, less than about 5×10⁻³ s⁻¹, less than about 10⁻³ s⁻¹, less than about 5×10⁻⁴ s⁻¹, less than about 10⁻⁴ s⁻¹, less than about 5×10⁻⁵ s⁻¹, less than about 10⁻⁵ s⁻¹, less than about 5×10⁻⁶ s⁻¹, less than about 10⁻⁶ s⁻¹, less than about 5×10⁻⁷ s⁻¹, less than about 10⁻⁷ s⁻¹ less than about 5×10⁻⁸ s⁻¹, less than about 10⁻⁸ s⁻¹ less than about 5×10⁻⁹ s⁻¹, less than about 10⁻⁹ s⁻¹, or less than about 10⁻¹° s⁻¹. In some embodiments, the k_{off} rate is measured by a BIAcore assay.

In some embodiments, the anti-PD-L1 antibody (or antigen-binding fragment thereof) does not bind to PD-L2. In some embodiments, the PD-L2 is human PD-L2. In some embodiments, the anti-PD-L1 antibody (or antigen-binding fragment thereof) does not bind to B7-H2 (e.g., human B7-H2). In some embodiments, the anti-PD-L1 antibody (or antigen-binding fragment thereof) does not bind to B7-H3 (e.g., human B7-H3). In some embodiments, the anti-PD-L1 antibody (or antigen-binding fragment thereof) does not bind to CD28 (e.g., human CD28). In some embodiments, the anti-PD-L1 antibody (or antigen-binding fragment thereof) does not bind to a CTLA-4 (e.g., human CTLA-4). In some embodiments, the anti-PD-L1 antibody (or antigen-binding fragment thereof) does not bind to PD-1 (e.g., human PD-1).

In some embodiments, the anti-PD-L1 antibody (or antigen-binding fragment thereof) is cross-reactive with PD-L1 from species other than human. In some embodiments, the anti-PD-L1 antibody (or antigen-binding fragment thereof) is cross-reactive with cynomolgus monkey PD-L1. In some embodiments, the anti-PD-L1 antibody (or antigen-binding fragment thereof) is cross-reactive with mouse PD-L1, e.g., 2.7A4. In some embodiments, the anti-PD-L1 antibody (or antigen-binding fragment thereof) is cross-reactive with both cynomolgus monkey PD-L1 and with mouse PD-L1, e.g., 2.7A4. IN some embodiments, the anti-PD-L1 antibody (or antigen-binding fragment thereof) is cross-reactive with cynomolgus monkey PD-L1 but not with mouse PD-L1, e.g., 2.9D10 and 2.14H9.

In some embodiments, the anti-PD-L1 antibody (or antigen-binding fragment thereof) is a monoclonal antibody. In some embodiments, the anti-PD-L1 antibody (or antigen-binding fragment thereof) is a fully human monoclonal antibody or a fragment thereof. In some embodiments, the anti-PD-L1 antibody (or antigen-binding fragment thereof) is an engineered antibody. In some embodiments, the anti-PD-L1 antibody (or antigen-binding fragment thereof) is a chimeric or humanized antibody. In some embodiments, the anti-PD-L1 antibody (or antigen-binding fragment thereof) comprises at least one mutation in the Fc region.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 June 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 2, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 2
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32 . . . 34 | H26--H35B | H30--H35B |
| (Kabat | | | | |
| Numbering¹) | | | | |
| CDR-H1 | H31--H35 | H26--H32 | H26--H35 | H30--H35 |
| (Chothia | | | | |
| Numbering²⁾ | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. In some embodiments, specific CDR sequences are specified.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM or Contact method. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the provided antibodies are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region; and multispecific antibodies formed from antibody fragments. In particular embodiments, the antibodies are single-chain antibody fragments comprising a heavy chain variable (V_{H}) region and/or a light chain variable (V_{L}) region, such as scFvs.

Single-domain antibodies (sdAbs) are antibody fragments comprising all or a portion of the heavy chain variable region or all or a portion of the light chain variable region of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the anti-PD-L1 antibodies in accord with the provided embodiments are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

In some embodiments, the anti-PD-L1 antibody (or antigen-binding fragment thereof) is of the IgG isotype. In some embodiments, the anti-PD-L1 antibody (or antigen-binding fragment thereof) is of the IgG1, IgG2, IgG3, or IgG4 isotype. In some embodiments, the anti-PD-L1 antibody (or antigen-binding fragment thereof) is a variant of the IgG1, IgG2, IgG3, or IgG4 isotype. In some embodiments, the anti-PD-L1 antibody (or antigen-binding fragment thereof) is of IgG2 isotype. In some embodiments, the anti-PD-L1 antibody (or antigen-binding fragment thereof) has reduced potential to elicit effector function. In some embodiments, the anti-PD-L1 antibody (or antigen-binding fragment thereof) is a fully human monoclonal antibody of the IgG1 isotype. In some embodiments, the anti-PD-L1 antibody (or antigen-binding fragment thereof) has increased potential to elicit Antibody Directed Cell-mediated Cytotoxicity (ADCC). In some embodiments, the anti-PD-L1 antibody (or antigen-binding fragment thereof) is of the z, za or f allotype. In some embodiments, the anti-PD-L1 antibody (or antigen-binding fragment thereof) is not an IgG antibody or fragment thereof. In some embodiments, the anti-PD-L1 antibody (or antigen-binding fragment thereof) is an IgM or IgD antibody or fragment thereof, or a variant of an IgM or IgD antibody or fragment thereof. In some embodiments, the anti-PD-L1 antibody (or antigen-binding fragment thereof) is a single chain variable fragment (scFv). In some embodiments, the anti-PD-L1 antibody (or antigen-binding fragment thereof) is a non-IgG-like fragment (e.g., nanobody, DARPin).

Exemplary PD-L1 antibodies include those disclosed in: U.S. Pat. No. 8,217,149; Ser. No. 12/633,339; U.S. Pat. No. 8,383,796; Ser. No. 13/091,936; U.S. Pat. No. 8,552,154; Ser. No. 13/120,406; US patent publication No. 20110280877; Ser. No. 13/068,337; US Patent Publication No. 20130309250; Ser. No. 13/892,671; WO2013019906; WO2013079174; U.S. application Ser. No. 13/511,538 (filed Aug. 7, 2012), which is the US National Phase of International Application No. PCT/US10/58007 (filed 2010); and U.S. application Ser. No. 13/478,511 (filed May 23, 2012).

Exemplary anti-PD-L1 antibodies include MDX-1105 (Medarex), MEDI4736 (Durvalumab, Medimmune, see U.S. Pat. No. 8,779,108) MPDL3280A (Genentech), AMP224 (GlaxoSmithKline), MSB0010718C (Avelumab, Pfizer), and BMS-935559 (Bristol-Myers Squibb). MEDI4736 (Durvalumab) is a human monoclonal antibody that binds to PD-L1, and inhibits interaction of the ligand with PD-1. MDPL3280A (Genentech/Roche) is a human Fc optimized IgG1 monoclonal antibody that binds to PD-L1. MDPL3280A and other human monoclonal antibodies to PD-L1 are described in U.S. Pat. No. 7,943,743 and U.S Publication No. 20120039906. Other anti-PD-L1 binding agents include YW243.55.570 (see WO2010/077634) and MDX-1105 (also referred to as BMS-936559, and, e.g., anti-PD-L1 binding agents described in WO2007/005874), or antigen-binding fragment of any of the foregoing.

In some embodiments, the anti-PD-L1 antibody (or antigen-binding fragment thereof) is an antibody (or antigen-binding fragment thereof) disclosed in U.S. Pat. No. 8,779,108. In some embodiments, the anti-PD-L1 antibody (or antigen-binding fragment thereof) is an antibody or fragment thereof comprising the CDRs sequences of an antibody or targeting binding agent disclosed in U.S. Pat. No. 8,799,108. In some embodiments, the anti-PD-L1 antibody (or antigen-binding fragment thereof) is an antibody or fragment thereof comprising the VH/VL sequences of an antibody or targeting binding agent disclosed in U.S. Pat. No. 8,799,108. In some embodiments, the anti-PD-L1 antibody (or antigen-binding fragment thereof) comprises sequences set forth in SEQ ID NO:60-61. In some embodiments, the anti-PD-L1 antibody or antigen-binding fragment is MEDI4736 (Durvalumab).

In some embodiments, the anti-PD-L1 antibody (or antigen-binding fragment thereof) is an antibody or fragment thereof comprising the CDRs of any antibody described herein. In some embodiments, the anti-PD-L1 antibody (or antigen-binding fragment thereof) is an antibody or fragment thereof that competes with any antibody described herein. In some embodiments, the anti-PD-L1 antibody (or antigen-binding fragment thereof) is an antibody or fragment thereof that binds to the same epitope as any antibody described herein binds to.

2. Anti-PD-1 Antibody or Fragment Thereof

In some embodiments of the methods, compositions, combinations, kits or articles of manufacture provided herein, the combination therapy comprises administering an anti-PD-1 antibody (or antigen-binding fragment thereof), e.g., a pharmaceutical composition containing an anti-PD-1 antibody (or antigen-binding fragment thereof). In some of any of the embodiments, the checkpoint inhibitor is or comprises an anti-PD-1 antibody or an antigen-binding fragment thereof.

In some embodiments, the anti-PD-1 antibody specifically binds to PD-1, such as to the extracellular region of PD-1. In some embodiments, the anti-PD-1 antibody (or antigen-binding fragment thereof) binds to PD-1 with a binding affinity (K_{D}) of less than about 5, 4, 3, 2.5, 2, or 1 nanomolar (nM). In some embodiments, the anti-PD-1 antibody (or antigen-binding fragment thereof) binds to the target (i.e., PD-1) with a binding affinity (K_{D}) of about 5 nM to about 1 nM; or about 5 nM to about 2 nM; or about 5 nM to about 3 nM; or about 5 nM to about 4 nM; or about 3 nM to about 1 nM; or about 2 nM to about 1 nM. In some embodiments, the anti-PD-1 antibody (or antigen-binding fragment thereof) binds to the target (i.e., PD-1) with a binding affinity (K_{D}) of less than about 950 picomolar (pM). In some embodiments, the anti-PD-1 antibody (or antigen-binding fragment thereof) binds to the target (i.e., PD-1) with a binding affinity (K_{D}) of less than about 900, 800, 700, 600, 500, 400, 300, 200, or 100 pM. In some embodiments, the anti-PD-1 antibody (or antigen-binding fragment thereof) binds to the target (i.e., PD-1) with a binding affinity (K_{D}) of about 900 pM to about 100 pM; or about 900 pM to about 200 pM; or about 900 pM to about 300 pM; or about 900 pM to about 400 pM; or about 900 pM to about 500 pM; or about 900 pM to about 600 pM; or about 900 pM to about 700 pM; or about 200 pM to about 100 pM; or about 300 pM to about 200 pM; or about 400 pM to about 300 pM. In some embodiments, the anti-PD-1 antibody (or antigen-binding fragment thereof) binds to the target (i.e., PD-1) with a binding affinity (K_{D}) of less than about 90 pM, 80 pM, 70 pM, 60 pM, 55 pM or 50 pM. In some embodiments, the anti-PD-1 antibody (or antigen-binding fragment thereof) binds to the target (i.e., PD-1) with a binding affinity (K_{D}) of about 100 pM to about 50 pM; or about 100 pM to about 70 pM; or about 100 pM to about 80 pM; or about 100 pM to about 90 pM; or about 70 pM to about 50 pM; or about 60 pM to about 50 pM; or about 55 pM to about 50 pM. The K_{D} may be assessed using a method known to one of skill in the art (e.g., a BIAcore assay, ELISA) (Biacore International AB, Uppsala, Sweden).

In some embodiments, the binding properties of the anti-PD-1 antibody (or antigen-binding fragment thereof) with reference to binding PD-1 may also be measured by reference to the dissociation or association rates (k_{off} and kₒₙ respectively). In some embodiments, the anti-PD-1 antibody (or antigen-binding fragment thereof) has a icₒₙ rate (antibody (Ab)+antigen (Ag)^{kon}→-Ab-Ag) of at least about 10⁴ M⁻¹ s⁻¹, at least about 5×10⁴ M⁻¹ s⁻¹ at least about 10⁵ M⁻¹ s⁻¹, at least about 2×10⁵ M⁻¹ s⁻¹, at least about 5×10⁵ M⁻¹ s⁻¹, at least about 10⁶ M⁻¹ s⁻¹, at least about 5×10⁶ M⁻¹ s⁻¹, at least about 10⁷ M⁻¹ s⁻¹, at least about 5×10⁷ M⁻¹ s⁻¹, or at least about 10⁸ M⁻¹ s−1. In some embodiments, the kₒₙ rate is measured by a BIAcore assay.

In some embodiments, the binding properties of the anti-PD-1 antibody (or antigen-binding fragment thereof) has a k_{off} rate (antibody (Ab)+antigen (Ag)^{koff}→Ab-Ag) of less than about 5×10⁻¹ s⁻¹, less than about 10⁻¹ s⁻¹, less than about 5×10⁻² s⁻¹, less than about 10⁻² s⁻¹, less than about 5×10⁻³ s⁻¹, less than about 10⁻³ s⁻¹, less than about 5×10⁻⁴ s⁻¹, less than about 10⁻⁴ s⁻¹, less than about 5×10⁻⁵ s⁻¹, less than about 10⁻⁵ s⁻¹, less than about 5×10⁻⁶ s⁻¹, less than about 10⁻⁶ s⁻¹, less than about 5×10⁻⁷ s⁻¹, less than about 10⁻⁷ s⁻¹ less than about 5×10⁻⁸ s⁻¹, less than about 10⁻⁸ s⁻¹ less than about 5×10⁻⁹ s⁻¹, less than about 10⁻⁹ s⁻¹, or less than about 10⁻¹⁰ s⁻¹. In some embodiments, the k_{off} rate is measured by a BIAcore assay.

In some embodiments, the anti-PD-1 antibody (or antigen-binding fragment thereof) is a monoclonal antibody. In some embodiments, the anti-PD-1 antibody (or antigen-binding fragment thereof) is a fully human monoclonal antibody or a fragment thereof. In some embodiments, the anti-PD-1 antibody (or antigen-binding fragment thereof) is an engineered antibody. In some embodiments, the anti-PD-1 antibody (or antigen-binding fragment thereof) is a chimeric or humanized antibody. In some embodiments, the anti-PD-1 antibody (or antigen-binding fragment thereof) comprises at least one mutation in the Fc region.

In some embodiments, the anti-PD-1 antibody (or antigen-binding fragment thereof) is of the IgG isotype. In some embodiments, the anti-PD-1 antibody (or antigen-binding fragment thereof) is of the IgG1, IgG2, IgG3, or IgG4 isotype. In some embodiments, the anti-PD-1 antibody (or antigen-binding fragment thereof) is a variant of the IgG1, IgG2, IgG3, or IgG4 isotype. In some embodiments, the anti-PD-1 antibody (or antigen-binding fragment thereof) is of IgG2 isotype. In some embodiments, the anti-PD-1 antibody (or antigen-binding fragment thereof) has reduced potential to elicit effector function. In some embodiments, the anti-PD-1 antibody (or antigen-binding fragment thereof) is a fully human monoclonal antibody of the IgG1 isotype. In some embodiments, the anti-PD-1 antibody (or antigen-binding fragment thereof) has increased potential to elicit Antibody Directed Cell-mediated Cytotoxicity (ADCC). In some embodiments, the anti-PD-1 antibody (or antigen-binding fragment thereof) is of the z, za or f allotype. In some embodiments, the anti-PD-1 antibody (or antigen-binding fragment thereof) is not an IgG antibody or fragment thereof. In some embodiments, the anti-PD-1 antibody (or antigen-binding fragment thereof) is an IgM or IgD antibody or fragment thereof, or a variant of an IgM or IgD antibody or fragment thereof. In some embodiments, the anti-PD-1 antibody (or antigen-binding fragment thereof) is a single chain variable fragment (scFv). In some embodiments, the anti-PD-1 antibody (or antigen-binding fragment thereof) is a non-IgG-like fragment (e.g., nanobody, DARPin).

Programmed cell death 1 (PD-1) is an immune checkpoint protein that is expressed in B cells, NK cells, and T cells (Shinohara et al., 1995, Genomics 23:704-6; Blank et al., 2007, Cancer Immunol Immunother 56:739-45; Finger et al., 1997, Gene 197:177-87; Pardoll (2012) Nature Reviews Cancer 12:252-264). The major role of PD-1 is to limit the activity of T cells in peripheral tissues during inflammation in response to infection, as well as to limit autoimmunity. PD-1 expression is induced in activated T cells and binding of PD-1 to one of its endogenous ligands acts to inhibit T-cell activation by inhibiting stimulatory kinases. PD-1 also acts to inhibit the TCR “stop signal”. PD-1 is highly expressed on Treg cells and may increase their proliferation in the presence of ligand (Pardoll (2012) Nature Reviews Cancer 12:252-264). Anti-PD 1 antibodies have been used for treatment of melanoma, non-small-cell lung cancer, bladder cancer, prostate cancer, colorectal cancer, head and neck cancer, triple-negative breast cancer, leukemia, lymphoma and renal cell cancer (Topalian et al., 2012, N Engl J Med 366:2443-54; Lipson et al., 2013, Clin Cancer Res 19:462-8; Berger et al., 2008, Clin Cancer Res 14:3044-51; Gildener-Leapman et al., 2013, Oral Oncol 49:1089-96; Menzies & Long, 2013, Ther Adv Med Oncol 5:278-85). Exemplary anti-PD-1 antibodies include nivolumab (Opdivo by BMS), pembrolizumab (Keytruda by Merck), pidilizumab (CT-011 by Cure Tech), lambrolizumab (MK-3475 by Merck), and AMP-224 (Merck), nivolumab (also referred to as Opdivo, BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are described in U.S. Pat. No. 8,008,449 and WO2006/121168. Pidilizumab (CT-011; Cure Tech) is a humanized IgGlk monoclonal antibody that binds to PD-1. Pidilizumab and other humanized anti-PD-1 monoclonal antibodies are described in WO2009/101611. Pembrolizumab (formerly known as lambrolizumab, and also referred to as Keytruda, MK03475; Merck) is a humanized IgG4 monoclonal antibody that binds to PD-1. Pembrolizumab and other humanized anti-PD-1 antibodies are described in U.S. Pat. No. 8,354,509 and WO2009/114335. Other anti-PD-1 antibodies include AMP 514 (Amplimmune), among others, e.g., anti-PD-1 antibodies described in U.S. Pat. No. 8,609,089, US 2010028330, US 20120114649 and/or US 20150210769. AMP-224 (B7-DCIg; Amplimmune; e.g., described in WO2010/027827 and WO2011/066342), is a PD-L2 Fc fusion soluble receptor that blocks the interaction between PD-1 and B7-H1.

In some of any of the embodiments, the checkpoint inhibitor is or comprises an anti-PD-1 antibody or an antigen-binding fragment thereof. In some embodiments, the checkpoint inhibitor is an anti-PD-1 antibody, such as nivolumab, pembrolizumab, or cemiplimab, or an antigen-binding fragment thereof. In some embodiments, the checkpoint inhibitor is nivolumab.

3. Other Immune Checkpoint Inhibitors

As used herein, the term “immune checkpoint inhibitor” refers to molecules that totally or partially reduce, inhibit, interfere with or modulate one or more checkpoint proteins. Checkpoint proteins regulate T-cell activation or function. These proteins are responsible for co-stimulatory or inhibitory interactions of T-cell responses. Immune checkpoint proteins regulate and maintain self-tolerance and the duration and amplitude of physiological immune responses. In some embodiments, the subject can be administered an additional agent that can enhance or boost the immune response, e.g., immune response effected by the binding molecules (e.g., BCMA-binding molecules), recombinant receptors, cells and/or compositions provided herein, against a disease or condition, e.g., a cancer, such as any described herein.

Immune checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors, ligands and/or receptor-ligand interaction. In some embodiments, modulation, enhancement and/or stimulation of particular receptors can overcome immune checkpoint pathway components. Illustrative immune checkpoint molecules that may be targeted for blocking, inhibition, modulation, enhancement and/or stimulation include, but are not limited to, PD-1 (CD279), PD-L1 (CD274, B7-H1), PDL2 (CD273, B7-DC), CTLA-4, LAG-3 (CD223), TIM-3, 4-1BB (CD137), 4-1BBL (CD137L), GITR (TNFRSF18, AITR), CD40, OX40 (CD134, TNFRSF4), CXCR2, tumor associated antigens (TAA), B7-H3, B7-H4, BTLA, HVEM, GAL9, B7H3, B7H4, VISTA, KIR, 2B4 (belongs to the CD2 family of molecules and is expressed on all NK, γδ, and memory CD8+ (αβ) T cells), CD160 (also referred to as BY55), CGEN-15049, CEACAM (e.g., CEACAM-1, CEACAM-3 and/or CEACAM-5), TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and a transforming growth factor receptor (TGFR; e.g., TGFR beta). Immune checkpoint inhibitors include antibodies, or antigen binding fragments thereof, or other binding proteins that bind to and block or inhibit and/or enhance or stimulate the activity of one or more of any of the said molecules.

Exemplary immune checkpoint inhibitors include Tremelimumab (CTLA-4 blocking antibody, also known as ticilimumab, CP-675,206), anti-OX40, PD-L1 monoclonal antibody (Anti-B7-H1; MEDI4736), MK-3475 (PD-1 blocker), nivolumab (anti-PD-1 antibody), CT-011 (anti-PD-1 antibody), BY55 monoclonal antibody, AMP224 (anti-PD-L1 antibody), BMS-936559 (anti-PD-L1 antibody), MPLDL3280A (anti-PD-L1 antibody), MSB0010718C (anti-PD-L1 antibody) and ipilimumab (anti-CTLA-4 antibody, also known as Yervoy®, MDX-010 and MDX-101). Exemplary of immunomodulatory antibodies include, but are not limited to, Daclizumab (Zenapax), Bevacizumab (Avastin®), Basiliximab, Ipilimumab, Nivolumab, pembrolizumab, MPDL3280A, Pidilizumab (CT-011), MK-3475, BMS-936559, MPDL3280A (Atezolizumab), tremelimumab, IMP321, BMS-986016, LAG525, urelumab, PF-05082566, TRX518, MK-4166, dacetuzumab (SGN-40), lucatumumab (HCD122), SEA-CD40, CP-870, CP-893, MEDI6469, MEDI6383, MOXR0916, AMP-224, MSB0010718C (Avelumab), MEDI4736, PDR001, rHIgM12B7, Ulocuplumab, BKT140, Varlilumab (CDX-1127), ARGX-110, MGA271, lirilumab (BMS-986015, IPH2101), 1PH2201, ARGX-115, Emactuzumab, CC-90002 and MNRP1685A or an antibody-binding fragment thereof. Other exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon .gamma., CAS 951209-71-5, available from IRX Therapeutics).

Cytotoxic T-lymphocyte-associated antigen (CTLA-4), also known as CD152, is a co-inhibitory molecule that functions to regulate T-cell activation. CTLA-4 is a member of the immunoglobulin superfamily that is expressed exclusively on T-cells. CTLA-4 acts to inhibit T-cell activation and is reported to inhibit helper T-cell activity and enhance regulatory T-cell immunosuppressive activity. Although the precise mechanism of action of CTLA-4 remains under investigation, it has been suggested that it inhibits T cell activation by outcompeting CD28 in binding to CD80 and CD86, as well as actively delivering inhibitor signals to the T cell (Pardoll (2012) Nature Reviews Cancer 12:252-264). Anti-CTLA-4 antibodies have been used in clinical trials for the treatment of melanoma, prostate cancer, small cell lung cancer, non-small cell lung cancer (Robert & Ghiringhelli, 2009, Oncologist 14:848-61; Ott et al., 2013, Clin Cancer Res 19:5300; Weber, 2007, Oncologist 12:864-72; Wada et al., 2013, J Transl Med 11:89). A significant feature of anti-CTLA-4 is the kinetics of anti-tumor effect, with a lag period of up to 6 months after initial treatment required for physiologic response. In some cases, tumors may actually increase in size after treatment initiation, before a reduction is seen (Pardoll (2012) Nature Reviews Cancer 12:252-264). Exemplary anti-CTLA-4 antibodies include ipilimumab (Bristol-Myers Squibb) and tremelimumab (Pfizer). Ipilimumab has recently received FDA approval for treatment of metastatic melanoma (Wada et al., 2013, J Transl Med 11:89).

Lymphocyte activation gene-3 (LAG-3), also known as CD223, is another immune checkpoint protein. LAG-3 has been associated with the inhibition of lymphocyte activity and in some cases the induction of lymphocyte anergy. LAG-3 is expressed on various cells in the immune system including B cells, NK cells, and dendritic cells. LAG-3 is a natural ligand for the MHC class II receptor, which is substantially expressed on melanoma-infiltrating T cells including those endowed with potent immune-suppressive activity. Exemplary anti-LAG-3 antibodies include BMS-986016 (Bristol-Myers Squib), which is a monoclonal antibody that targets LAG-3. IMP701 (Immutep) is an antagonist LAG-3 antibody and IMP731 (Immutep and GlaxoSmithKline) is a depleting LAG-3 antibody. Other LAG-3 inhibitors include IMP321 (Immutep), which is a recombinant fusion protein of a soluble portion of LAG-3 and Ig that binds to MHC class II molecules and activates antigen presenting cells (APC). Other antibodies are described, e.g., in WO2010/019570 and US 2015/0259420

T-cell immunoglobulin domain and mucin domain-3 (TIM-3), initially identified on activated Th1 cells, has been shown to be a negative regulator of the immune response. Blockade of TIM-3 promotes T-cell mediated anti-tumor immunity and has anti-tumor activity in a range of mouse tumor models. Combinations of TIM-3 blockade with other immunotherapeutic agents such as TSR-042, anti-CD137 antibodies and others, can be additive or synergistic in increasing anti-tumor effects. TIM-3 expression has been associated with a number of different tumor types including melanoma, NSCLC and renal cancer, and additionally, expression of intratumoral TIM-3 has been shown to correlate with poor prognosis across a range of tumor types including NSCLC, cervical, and gastric cancers. Blockade of TIM-3 is also of interest in promoting increased immunity to a number of chronic viral diseases. TIM-3 has also been shown to interact with a number of ligands including galectin-9, phosphatidylserine and HMGB1, although which of these, if any, are relevant in regulation of anti-tumor responses is not clear at present. In some embodiments, antibodies, antibody fragments, small molecules, or peptide inhibitors that target TIM-3 can bind to the IgV domain of TIM-3 to inhibit interaction with its ligands. Exemplary antibodies and peptides that inhibit TIM-3 are described in US 2015/0218274, WO2013/006490 and US 2010/0247521. Other anti-TIM-3 antibodies include humanized versions of RMT3-23 (Ngiow et al., 2011, Cancer Res, 71:3540-3551), and clone 8B.2C12 (Monney et al., 2002, Nature, 415:536-541). Bi-specific antibodies that inhibit TIM-3 and PD-1 are described in US 2013/0156774.

In some embodiments, the additional agent is a CEACAM inhibitor (e.g., CEACAM-1, CEACAM-3, and/or CEACAM-5 inhibitor). In some embodiments, the inhibitor of CEACAM is an anti-CEACAM antibody molecule. Exemplary anti-CEACAM-1 antibodies are described in WO 2010/125571, WO 2013/082366 WO 2014/059251 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In some embodiments, the anti-CEACAM antibody binds to CEACAM-5 as described in, e.g., Zheng et al. PLoS One. (2011) 6(6): e21146), or cross-reacts with CEACAM-1 and CEACAM-5 as described in, e.g., WO 2013/054331 and US 2014/0271618.

4. Compositions and Formulations

In some embodiments of the combination therapy methods, compositions, combinations, kits and uses provided herein, the combination therapy can be administered in one or more compositions, e.g., a pharmaceutical composition containing a checkpoint inhibitor, e.g., anti-PD-L1 antibody (or antigen-binding fragment thereof), or a pharmaceutically acceptable salt of hydrate thereof.

In some embodiments, the composition, e.g., a pharmaceutical composition containing the checkpoint inhibitor, e.g., anti-PD-L1 antibody (or antigen-binding fragment thereof), or a pharmaceutically acceptable salt of hydrate thereof, can include carriers such as a diluent, adjuvant, excipient, or vehicle with which the checkpoint inhibitor, e.g., anti-PD-L1 antibody (or antigen-binding fragment thereof), or a pharmaceutically acceptable salt of hydrate thereof, and/or the cells are administered. Examples of suitable pharmaceutical carriers are described in “Remington's Pharmaceutical Sciences” by E. W. Martin. Such compositions will contain a therapeutically effective amount of the checkpoint inhibitor, e.g., anti-PD-L1 antibody (or antigen-binding fragment thereof), or a pharmaceutically acceptable salt of hydrate thereof, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Saline solutions and aqueous dextrose and glycerol solutions also can be employed as liquid carriers, particularly for injectable solutions. The pharmaceutical compositions can contain any one or more of a diluents(s), adjuvant(s), antiadherent(s), binder(s), coating(s), filler(s), flavor(s), color(s), lubricant(s), glidant(s), preservative(s), detergent(s), sorbent(s), emulsifying agent(s), pharmaceutical excipient(s), pH buffering agent(s), or sweetener(s) and a combination thereof. In some embodiments, the pharmaceutical composition can be liquid, solid, a lyophilized powder, in gel form, and/or combination thereof. In some aspects, the choice of carrier is determined in part by the particular inhibitor and/or by the method of administration.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), stabilizers and/or preservatives. The compositions containing the checkpoint inhibitor, e.g., anti-PD-L1 antibody (or antigen-binding fragment thereof) or a pharmaceutically acceptable salt of hydrate thereof can also be lyophilized.

In some embodiments, the pharmaceutical compositions can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g., sublingual), and transdermal administration or any route. In some embodiments, other modes of administration also are contemplated. In some embodiments, the administration is by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, administration is by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration. In some embodiments, it is administered by multiple bolus administrations, for example, over a period of no more than 3 days, or by continuous infusion administration.

In some embodiments, the administration can be local, topical or systemic depending upon the locus of treatment. In some embodiments local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. In some embodiments, compositions also can be administered with other biologically active agents, either sequentially, intermittently or in the same composition. In some embodiments, administration also can include controlled release systems including controlled release formulations and device controlled release, such as by means of a pump. In some embodiments, the administration is oral.

In some embodiments, pharmaceutically and therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. In some embodiments, unit dosage forms, include, but are not limited to, tablets, capsules, pills, powders, granules, sterile parenteral solutions or suspensions, and oral solutions or suspensions, and oil water emulsions containing suitable quantities of the compounds or pharmaceutically acceptable derivatives thereof. Unit dose forms can be contained ampoules and syringes or individually packaged tablets or capsules. Unit dose forms can be administered in fractions or multiples thereof. In some embodiments, a multiple dose form is a plurality of identical unit dosage forms packaged in a single container to be administered in segregated unit dose form. Examples of multiple dose forms include vials, bottles of tablets or capsules or bottles of pints or gallons.

5. Dosing

In some embodiments, the provided combination therapy method involves administering to the subject an checkpoint inhibitor, such as anti-PD-L1 antibody or antigen binding fragment thereof, e.g. durvalumab, and the cell therapy, such as a T cell therapy (e.g. CAR-expressing T cells).

In some embodiments, the administration of the checkpoint inhibitor, such as anti-PD-L1 antibody or antigen binding fragment thereof, e.g. durvalumab, is initiated prior to, subsequently to, during, during the course of, simultaneously, near simultaneously, sequentially and/or intermittently with the administration of the cell therapy, such as a T cell therapy (e.g. CAR-expressing T cells). In some embodiments, the method involves initiating the administration of the checkpoint inhibitor, such as anti-PD-L1 antibody or antigen binding fragment thereof, after (subsequently to) administration of the T cell therapy.

In some embodiments, the checkpoint inhibitor, such as anti-PD-L1 antibody or antigen binding fragment thereof, e.g. durvalumab is administered, greater than or greater than about 21 days after initiation of administration of the cell therapy. In certain aspects, the initiation of administration of the checkpoint inhibitor, such as anti-PD-L1 antibody or antigen binding fragment thereof, e.g. durvalumab, in the provided combination therapy is from or from about 22 days to 50 days after initiation of administration of the T cell therapy, e.g. at or about at 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days or 50 days after initiation of administration of the T cell therapy. In some embodiments, the initiation of administration of the checkpoint inhibitor, such as anti-PD-L1 antibody or antigen binding fragment thereof, e.g. durvalumab, is from or from about 22 days to 43 days, such as 22 days to 36 days after initiation of administration of the T cell therapy. In some embodiments, the initiation of administration of the checkpoint inhibitor, such as anti-PD-L1 antibody or antigen binding fragment thereof, e.g. durvalumab, is at or about 29 days after initiation of administration of the T cell therapy.

In certain aspects, the provided methods can enhance, increase or potentiate T cell therapy in subjects in which a peak response to the T cell therapy has been observed but in which the response, e.g. presence of T cells and/or reduction in tumor burden, has become reduced or is no longer detectable. In some cases, initiation of administration the checkpoint inhibitor, such as anti-PD-L1 antibody or antigen binding fragment thereof, e.g. durvalumab, is carried out at or within a week, such as within 1, 2 or 3 days after (i) a time in which peak or maximum level of the cells of the T cell therapy are detectable in the blood of the subject; (ii) the number of cells of the T cell therapy detectable in the blood, after having been detectable in the blood, is not detectable or is reduced, optionally reduced compared to a preceding time point after administration of the T cell therapy; (iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy; (iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject; (v) the subject exhibits disease progression and/or has relapsed following remission after treatment with the T cell therapy; and/or (iv) the subject exhibits increased tumor burden as compared to tumor burden at a time prior to or after administration of the cells and prior to initiation of administration of the checkpoint inhibitor, such as anti-PD-L1 antibody

In some embodiments, at the time at which the subject is first administered the checkpoint inhibitor, such as anti-PD-L1 antibody or antigen binding fragment thereof, e.g. durvalumab, and/or at any subsequent time after initiation of the administration, the subject does not exhibit a sign or symptom of a severe toxicity, such as severe cytokine release syndrome (CRS) or severe toxicity. In some embodiments, the administration of the checkpoint inhibitor, such as anti-PD-L1 antibody or antigen binding fragment thereof, e.g. durvalumab s at a time at which the subject does not exhibit a sign or symptom of severe CRS and/or does not exhibit grade 3 or higher CRS, such as prolonged grade 3 CRS or grade 4 or 5 CRS. In some embodiments, the administration of the checkpoint inhibitor, such as anti-PD-L1 antibody or antigen binding fragment thereof, e.g. durvalumab is at a time at which the subject does not exhibit a sign or symptom of severe neurotoxicity and/or does not exhibit grade 3 or higher neurotoxicity, such as prolonged grade 3 neurotoxicity or grade 4 or grade 5 neurotoxicity. In some aspects, between the time of the initiation of the administration of the T cell therapy and the time of the administration of the checkpoint inhibitor, such as anti-PD-L1 antibody or antigen binding fragment thereof, e.g. durvalumab the subject has not exhibited severe CRS and/or has not exhibited grade 3 or higher CRS, such as prolonged grade 3 CRS or grade 4 or 5 CRS. In some instances, between the time of the initiation of the administration of the T cell therapy and the time of the administration of the checkpoint inhibitor, such as anti-PD-L1 antibody or antigen binding fragment thereof, e.g. durvalumab, the subject has not exhibited severe neurotoxicity and/or does not exhibit grade 3 or higher neurotoxicity, such as prolonged grade 3 neurotoxicity or grade 4 or 5 neurotoxicity.

In some embodiments, the anti-PD-L1 antibody or antigen-binding fragment thereof, e.g. durvalumab, is administered in a therapeutically effective amount. In one embodiment, the anti-PD-L1 antibody or antigen-binding fragment, e.g. durvalumab, is administered in an amount, e.g. total dosage amount in a cycle, of from or from about 750 mg to at or about 2000 mg, such as from or from about 1200 mg to at or about 1500 mg. In some cases the amount is or is at least or at least at or about 750 mg, 800 mg, 900 mg, 1000 mg, 1100 mg, 1200 mg, 1300 mg, 1400 mg, 1500 mg, 1600 mg, 1700 mg, 1800 mg, 1900 mg or 2000 mg. In some cases the amount is or is at least or at least at or about 1500 mg. In some aspects, the anti-PD-L1 antibody or antigen-binding fragment thereof is durvalumab.

In some embodiments, such amounts are administered per kilogram of the subjects body weight. In some embodiments, when referencing dosage based on mg/kg of the subject, an average human subject is considered to have a mass of about 70 kg-75 kg, such as at or about 75 kg. In some embodiments, the anti-PD-L1 antibody or antigen-binding fragment, e.g. durvalumab, is administered in an amount, e.g. total dosage amount in a cycle, of from or from about 0.5 mg per kilogram of a subject's mass to at or about 30 mg/kg, such as from or from about 1 mg/kg to at or about 20 mg/kg. In some cases the amount is or is at least or at least about 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg.

In some embodiments, the anti-PD-1 antibody or antigen-binding fragment thereof, e.g. nivolumab, is administered in a therapeutically effective amount. In one embodiment, the anti-PD-1 antibody or antigen-binding fragment, e.g. nivolumab, is administered in an amount, e.g. total dosage amount in a cycle, of from or from about 200 mg to at or about 2000 mg, such as from or from about 400 mg to at or about 1000 mg, or from or from about 400 mg to at or about 600 mg. In some cases the amount is or is at least or at least at or about 200 mg, 225 mg, 240 mg, 200 mg, 300 mg, 400 mg, 450 mg, 480 mg, 500 mg, 600 mg, 700 mg, 800 mg, 900 mg, 1000 mg, 1600 mg, 1700 mg, 1800 mg, 1900 mg or 2000 mg. In some cases the amount is or is at least or at least at or about 480 mg. In some aspects, the anti-PD-1 antibody or antigen-binding fragment thereof is nivolumab.

In some embodiments, such amounts are administered per kilogram of the subjects body weight. In some embodiments, when referencing dosage based on mg/kg of the subject, an average human subject is considered to have a mass of about 70 kg-75 kg, such as at or about 75 kg. In some embodiments, the anti-PD-1 antibody or antigen-binding fragment, e.g. nivolumab, is administered in an amount, e.g. total dosage amount in a cycle, of from or from about 0.1 mg per kilogram of a subject's mass to at or about 10 mg/kg, such as from or from about 1 mg/kg to at or about 5 mg/kg. In some cases the amount is or is at least or at least about 0.1 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg or 10 mg/kg. In some embodiments, the provided methods involve continued administration, such as at regular intervals, of the checkpoint inhibitor, such as anti-PD-L1 antibody or antigen binding fragment thereof, e.g. durvalumab, after initiation of administration of the cell therapy.

Administration can be performed using cyclic administration as described herein. In some embodiments, cycling therapy involves the administration of an active agent for a period of time, optionally followed by a rest for a period of time, and repeating this sequential administration. Cycling therapy can be performed independently for each active agent (e.g., checkpoint inhibitor, such as anti-PD-L1 antibody or antigen-binding fragment thereof) over a prescribed duration of time. In some embodiments, the provided methods are carried out in a 28-day cycle. In some embodiments, the cycle, such as a 28-day cycle, is repeated a plurality of times. In certain embodiments, the cycle is repeated up to or up to about 2, 3, 4, 5, 6, 7, 8, 9, 10, 11, 12 or more times, such as in consecutive cycles. In some embodiments, the total duration of consecutive cycles does not exceed 12 months, or is carried out for a total duration of 12 months or less.

In some embodiments, administering the checkpoint inhibitor, such as anti-PD-L1 antibody or antigen-binding fragment thereof, e.g. durvalumab, such as in more than one cycle, is discontinued if the subject exhibits progressive disease, e.g. after the first, second or third cycle of the administration. In some embodiments, the combination therapy involves administering the checkpoint inhibitor, such as anti-PD-L1 antibody or antigen-binding fragment thereof for 3 months, e.g. three 28-day cycles, after initiation of the cell therapy, at which time the subject is re-evaluated for further treatment of the checkpoint inhibitor, such as anti-PD-L1 antibody or antigen-binding fragment thereof, e.g. durvalumab. In some embodiments, a subject exhibiting a partial response (PR) after having received the provided combination therapy with up to three 28-day cycles of the checkpoint inhibitor, such as anti-PD-L1 antibody or antigen-binding fragment thereof, e.g. durvalumab, may receive one or more further cycle, e.g. 28-day cycle, of the checkpoint inhibitor, such as anti-PD-L1 antibody or antigen-binding fragment thereof.

In some embodiments, the amount, e.g. total dosage amount in a cycle, is administered as one dose or is administered in more than one dose, e.g. 2, 3, 4 or more doses, such as during the course of a cycle of administration. In some embodiments, the frequency of administration is in the range of about a daily dose to about a once monthly dose. In certain embodiments, administration is once a day, twice a day, three times a day, four times a day, once every other day, twice a week, once every week, once every two weeks, once every three weeks, or once every four weeks. In some embodiments, an checkpoint inhibitor, such as anti-PD-L1 antibody or antigen-binding fragment is administered once a week (Q1W), e.g. four times a month or weekly. In another embodiment, an checkpoint inhibitor, such as anti-PD-L1 antibody or antigen-binding fragment is administered once every two weeks (Q2W), e.g. twice a month. In yet another embodiment, an checkpoint inhibitor, such as anti-PD-L1 antibody or antigen-binding fragment is administered once every 4 weeks (Q4W), e.g. once a month.

In certain embodiments, the anti-PD-L1 antibody or antigen-binding fragment is administered on days 1, 8, 15 and 22 in a 28 day cycle. In certain embodiments, the anti-PD-L1 antibody or antigen-binding fragment is administered on days 15 and 22 in a 28 day cycle. In certain embodiments, the anti-PD-L1 antibody or antigen-binding fragment is administered on day 1 and 15 in a 28 day cycle. In certain embodiments, the anti-PD-L1 antibody or antigen-binding fragment is administered on day 1 in a 28 day cycle.

In some embodiments, the first or a preceding cycle of administering the total dosage amount in two or more cycles involves administering a total dosage amount of the anti-PD-L1 antibody or antigen-binding fragment, e.g. durvalumab that is lower than a second or a subsequent cycle. In some embodiments, the lower dosage amount is 50% to 95% of the total dosage amount in a second or a subsequent cycle. In some embodiments, the total dosage amount of the anti-PD-L1 antibody or antigen-binding fragment, e.g. durvalumab in a first or a preceding cycle of administration is the same or similar as in a second or a subsequent cycle of administration.

In some embodiments, in the first cycle of two or more cycles, e.g. 28-day cycles, the administration of the total dosage amount of the anti-PD-L1 antibody or antigen-binding fragment, e.g. durvalumab, is carried out for more than one time in the cycle, e.g. for 2, 3, 4 or more days, such as at day 1, 8, 15 and 22 or days 15 and 22 of the first cycle. In some embodiments, in at least the first two cycles of two or more or three or more cycles, e.g. 28-days cycles, the administration of the total dosage amount of the anti-PD-L1 antibody or antigen-binding fragment, e.g. durvalumab, is carried out more than one time in each of the first two cycles, e.g. independently 2, 3 or 4 time in each of the at least first two cycles, such as independently at days 1, 8, 15 and 22 in at least one of the first two cycles and/or days 15 and 22 in at least one of the first two cycles. In some embodiments, the first or a preceding cycle of administering the total dosage amount in two or more cycles is carried out by administering the anti-PD-L1 antibody or antigen-binding fragment, e.g. durvalumab a greater number of times in the first or a preceding cycle as compared to a second or a subsequent cycle, e.g. 28-day cycle.

In such embodiments, a lower dosage amount of the anti-PD-L1 antibody or antigen-binding fragment, e.g. durvalumab is administered by each administration in a cycle when it is given at a lower total dosage amount and/or a greater number of times but at the same or similar total dosage amount, e.g. in a first or a preceding cycle compared to a second or a subsequent cycle. In some embodiments, it is contemplated that administering a lower dosage amount a greater number of times in a first or preceding cycle can result in the same or similar biological PD-L1 occupancy but a shorter half-life at the lower doses, compared to a cycle in which a similar total dosage amount is administered less times in the cycle. In some embodiments, a dosing regimen that provides an checkpoint inhibitor, such as anti-PD-L1 antibody or antigen-binding fragment with a shorter half-life may reduce the risk of developing a toxicity following the combination therapy.

In some embodiments, the combination therapy involves administering the anti-PD-L1 antibody or antigen-binding fragment, e.g. durvalumab in a 28-day cycle, such as in a first 28-day cycle of treatment regimen involving at least two 28-day cycles, (i) once-weekly (Q1W) for two doses, optionally on days 15 and 22; (ii) once-weekly (Q1W) for four doses, optionally on days 1, 8, 15 and 22; (iii) Q1W for two consecutive doses, optionally on days 1 and 8, followed by every two weeks (Q2W) for one dose, optionally on day 15; or (iv) every two weeks (Q2W) for two doses, optionally on days 1 and 15. In some embodiments, the weekly (Q1W) or every other week (Q2W) dose is a fraction or a portion of the total dosage amount administered in a cycle, e.g. a 28-day cycle. In some embodiments, each Q1W dose of the first 28 day cycle is independently from or from about 18% to 32% of the total dosage amount, such as is or is about 25% of the total dosage amount in the cycle. In some embodiments, each Q2W dose of the first 28 day cycle is independently from or from about 40% to 62.5% of the total dosage amount, optionally is or is about 50% of the total dosage amount in the cycle.

In some embodiments, in a 28-day cycle, such as in a first 28-day cycle of treatment regimen involving at least two 28-day cycles, the anti-PD-L1 antibody or antigen-binding fragment, e.g. durvalumab, is administered once weekly (Q1W) for two consecutive doses in an amount of or about 375 mg followed by once every two weeks (Q2W) for one dose in an amount of or about 750 mg. In some embodiments, in a 28-day cycle, such as in a first 28-day cycle of treatment regimen involving at least two 28-day cycles, the anti-PD-L1 antibody or antigen-binding fragment, e.g. durvalumab, is administered once weekly (Q1W) for four doses. In some embodiments, the four doses include administering two consecutive doses of or about 225 mg followed by two consecutive doses of or about 375 mg.

In some embodiments, in a 28-day cycle, such as in a first 28-day cycle of treatment regimen involving at least two 28-day cycles, the anti-PD-L1 antibody or antigen-binding fragment, e.g. durvalumab, is administered once weekly (Q1W) for two consecutive doses in an amount of or about 375 mg.

In some embodiments, in a 28-day cycle, such as in a second or a subsequent 28-day cycle of treatment regimen involving at least two 28-day cycles, the anti-PD-L1 antibody or antigen-binding fragment, e.g. durvalumab, is administered in 1 or 2 doses in the cycle. In some embodiment, in a 28-day cycle, such as in a second or a subsequent 28-day cycle of treatment regimen involving at least two 28-day cycles, the anti-PD-L1 antibody or antigen-binding fragment, e.g. durvalumab, is administered (i) once every two weeks (Q2W) for two doses, optionally on days 1 and 15; or (ii) once every four weeks (Q4W) for one dose, optionally on day 1. In some embodiments, each Q2W dose of such a 28 day cycle is independently from or from about 50% of the total dosage amount in the cycle. In some embodiments, each Q4W dose of the such a 28 day cycle is independently from or from about 100% of the total dosage amount.

In some embodiments, in a 28-day cycle, such as in a second or a subsequent 28-day cycle of treatment regimen involving at least two 28-day cycles, the anti-PD-L1 antibody or antigen-binding fragment, e.g. durvalumab, is administered every two weeks (Q2W) for two doses in an amount of or about 750 mg.

In some embodiments, in a 28-day cycle, such as in a second or a subsequent 28-day cycle of treatment regimen involving at least two 28-day cycles, the anti-PD-L1 antibody or antigen-binding fragment, e.g. durvalumab, is administered once a month (Q4W) for one dose in an amount of or about 1500 mg.

In some embodiments, the administration of an anti-PD-L1 antibody (or antigen-binding fragment thereof) comprises carrying out at least two 28-day cycles. In some embodiments, each of the at least two 28-day cycles comprises administrating a total dosage amount of at or about 750 mg to at or about 2000 mg, such as 750 mg to 2000 mg of the anti-PD-L1 antibody or antigen-binding fragment. In some embodiments, in at least the first of the at least two 28-day cycles, the administration of the total dosage amount of the anti-PD-L1 antibody or antigen-binding fragment is carried out by administering the antibody or antigen-binding fragment thereof is carried out by administering the antibody or fragment more than one time. In some embodiments, in a first of said at least two 28-day cycles, the administration of the total dosage amount of the anti-PD-L1 antibody or antigen-binding fragment is carried out by administering the antibody or fragment a greater number of times as compared to a second and/or a subsequent 28 day cycle. In some embodiments, the administration of the anti-PD-L1 antibody or antigen-binding fragment thereof is initiated greater than 21 days (e.g., 22-50 days) after initiation of the administration of the cell therapy. In some embodiments, at the time of administering the anti-PD-L1 antibody or antigen-binding fragment thereof, the subject does not exhibit a severe toxicity following administration of the cell therapy. In some embodiments, the anti-PD-L1 antibody or antigen-binding fragment thereof is durvalumab.

In some of any of the embodiments, the checkpoint inhibitor is or comprises an anti-PD-1 antibody or an antigen-binding fragment thereof. In some embodiments, the checkpoint inhibitor is an anti-PD-1 antibody, such as nivolumab, pembrolizumab, or cemiplimab, or an antigen-binding fragment thereof. In some embodiments, the checkpoint inhibitor is nivolumab. In some embodiments, the anti-PD-1 antibody is administered in a total dosage amount of at or about 400 mg to at or about 600 mg, such as 400 mg to 600 mg, e.g., for each dosage cycle. In some embodiments, the anti-PD-1 antibody is optionally at or about 480 mg, e.g., for each dosage cycle.

In some embodiments, in a 28-day cycle, such as in a first 28-day cycle of treatment regimen involving at least two 28-day cycles, the anti-PD-1 antibody or antigen-binding fragment, e.g. nivolumab, is administered once weekly (Q1W) for two consecutive doses in an amount of or about 120 mg followed by once every two weeks (Q2W) for one dose in an amount of or about 240 mg. In some embodiments, in a 28-day cycle, such as in a first 28-day cycle of treatment regimen involving at least two 28-day cycles, the anti-PD-1 antibody or antigen-binding fragment, e.g. nivolumab, is administered once weekly (Q1W) for four doses. In some embodiments, the four doses include administering two consecutive doses of or about 225 mg followed by two consecutive doses of or about 120 mg.

In some embodiments, in a 28-day cycle, such as in a first 28-day cycle of treatment regimen involving at least two 28-day cycles, the anti-PD-1 antibody or antigen-binding fragment, e.g. nivolumab, is administered once weekly (Q1W) for two consecutive doses in an amount of or about 120 mg.

In some embodiments, in a 28-day cycle, such as in a second or a subsequent 28-day cycle of treatment regimen involving at least two 28-day cycles, the anti-PD-1 antibody or antigen-binding fragment, e.g. nivolumab, is administered in 1 or 2 doses in the cycle. In some embodiment, in a 28-day cycle, such as in a second or a subsequent 28-day cycle of treatment regimen involving at least two 28-day cycles, the anti-PD-1 antibody or antigen-binding fragment, e.g. nivolumab, is administered (i) once every two weeks (Q2W) for two doses, optionally on days 1 and 15; or (ii) once every four weeks (Q4W) for one dose, optionally on day 1. In some embodiments, each Q2W dose of such a 28 day cycle is independently from or from about 50% of the total dosage amount in the cycle. In some embodiments, each Q4W dose of the such a 28 day cycle is independently from or from about 100% of the total dosage amount.

In some embodiments, in a 28-day cycle, such as in a second or a subsequent 28-day cycle of treatment regimen involving at least two 28-day cycles, the anti-PD-1 antibody or antigen-binding fragment, e.g. nivolumab, is administered every two weeks (Q2W) for two doses in an amount of or about 240 mg.

In some embodiments, in a 28-day cycle, such as in a second or a subsequent 28-day cycle of treatment regimen involving at least two 28-day cycles, the anti-PD-1 antibody or antigen-binding fragment, e.g. nivolumab, is administered once a month (Q4W) for one dose in an amount of or about 480 mg.

In some embodiments, the administration of an anti-PD-1 antibody (or antigen-binding fragment thereof) comprises carrying out at least two 28-day cycles. In some embodiments, each of the at least two 28-day cycles comprises administrating a total dosage amount of at or about 240 mg to at or about 1000 mg of the anti-PD-1 antibody or antigen-binding fragment. In some embodiments, in at least the first of the at least two 28-day cycles, the administration of the total dosage amount of the anti-PD-1 antibody or antigen-binding fragment is carried out by administering the antibody or antigen-binding fragment thereof is carried out by administering the antibody or fragment more than one time. In some embodiments, in a first of said at least two 28-day cycles, the administration of the total dosage amount of the anti-PD-1 antibody or antigen-binding fragment is carried out by administering the antibody or fragment a greater number of times as compared to a second and/or a subsequent 28 day cycle. In some embodiments, the administration of the anti-PD-1 antibody or antigen-binding fragment thereof is initiated greater than 21 days (e.g., 22-50 days) after initiation of the administration of the cell therapy. In some embodiments, at the time of administering the anti-PD-1 antibody or antigen-binding fragment thereof, the subject does not exhibit a severe toxicity following administration of the cell therapy. In some embodiments, the anti-PD-1 antibody or antigen-binding fragment thereof is nivolumab.

In some embodiments, the B cell malignancy is NHL, such as relapsing/refractory aggressive NHL. In some embodiments, the cell therapy, such as CAR-expressing T cells, comprise a chimeric antigen receptor specifically binding to a B cell antigen. In some embodiments, the antigen expressed by the B cell malignancy, such as the B cell antigen, is CD19.

### II. CELL THERAPY AND ENGINEERING CELLS

In some embodiments, the cell therapy (e.g., T cell therapy) for use in accord with the provided combination therapy methods includes administering engineered cells expressing recombinant receptors designed to recognize and/or specifically bind to molecules associated with the disease or condition and result in a response, such as an immune response against such molecules upon binding to such molecules. The receptors may include chimeric receptors, e.g., chimeric antigen receptors (CARs), and other transgenic antigen receptors including transgenic T cell receptors (TCRs). Also provided are populations of such cells, compositions containing such cells and/or enriched for such cells, such as in which cells of a certain type such as T cells or CD8+ or CD4+ cells are enriched or selected.

In some embodiments, the cells contain or are engineered to contain an engineered receptor, e.g., an engineered antigen receptor, such as a chimeric antigen receptor (CAR), or a T cell receptor (TCR). Also provided are populations of such cells, compositions containing such cells and/or enriched for such cells, such as in which cells of a certain type such as T cells or CD8⁺ or CD4⁺ cells are enriched or selected. Among the compositions are pharmaceutical compositions and formulations for administration, such as for adoptive cell therapy. Also provided are therapeutic methods for administering the cells and compositions to subjects, e.g., patients.

In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

A. Recombinant Receptors

In some embodiments, provided are engineered cells, such as immune cells, such as T cells, that express a recombinant receptor. Among the receptors are antigen receptors and receptors containing one or more component thereof. The recombinant receptors may include chimeric receptors, such as those containing ligand-binding domains or binding fragments thereof and intracellular signaling domains or regions, functional non-TCR antigen receptors, chimeric antigen receptors (CARs), and T cell receptors (TCRs), such as recombinant or transgenic TCRs, chimeric autoantibody receptor (CAAR) and components of any of the foregoing. The recombinant receptor, such as a CAR, generally includes the extracellular antigen (or ligand) binding domain linked to one or more intracellular signaling components, in some aspects via linkers and/or transmembrane domain(s).

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating a molecule such as a recombinant receptor, e.g., the CAR or TCR, to targeting moieties is known. See, for instance, Wadwa et al., J. Drug Targeting 3: 1 1 1 (1995), and U.S. Pat. No. 5,087,616.

1. Chimeric Antigen Receptors (CARs)

In some embodiments, engineered cells, such as T cells, are provided that express a CAR with specificity for a particular antigen (or marker or ligand), such as an antigen expressed on the surface of a particular cell type. In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In particular embodiments, the recombinant receptor, such as chimeric receptor, contains an intracellular signaling region, which includes a cytoplasmic signaling domain or region (also interchangeably called an intracellular signaling domain or region), such as a cytoplasmic (intracellular) region capable of inducing a primary activation signal in a T cell, for example, a cytoplasmic signaling domain or region of a T cell receptor (TCR) component (e.g. a cytoplasmic signaling domain or region of a zeta chain of a CD3-zeta (CD3) chain or a functional variant or signaling portion thereof) and/or that comprises an immunoreceptor tyrosine-based activation motif (ITAM).

In some embodiments, the chimeric receptor further contains an extracellular ligand-binding domain that specifically binds to a ligand (e.g. antigen) antigen. In some embodiments, the chimeric receptor is a CAR that contains an extracellular antigen-recognition domain that specifically binds to an antigen. In some embodiments, the ligand, such as an antigen, is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the antigen receptors include a CAR as described in U.S. Pat. No. 7,446,190, and those described in International Patent Application Publication No.: WO/2014055668 Al. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (V_{H}) and variable light (V_{L}) chains of a monoclonal antibody (mAb).

In some embodiments, the antibody or antigen-binding portion thereof is expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR. In some embodiments, the extracellular antigen binding domain specific for an MHC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

In some embodiments, the recombinant receptor, such as a chimeric receptor (e.g. CAR), includes a ligand-binding domain that binds, such as specifically binds, to an antigen (or a ligand). Among the antigens targeted by the chimeric receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

In some embodiments, the antigen (or a ligand) is a polypeptide. In some embodiments, it is a carbohydrate or other molecule. In some embodiments, the antigen (or a ligand) is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the CAR contains an antibody or an antigen-binding fragment (e.g. scFv) that specifically recognizes an antigen, such as an intact antigen, expressed on the surface of a cell.

Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

In some embodiments, the antibody or an antigen-binding fragment (e.g. scFv) that specifically recognizes an antigen, such as CD19.

In some embodiments, the antigen is CD19. In some embodiments, the scFv contains a V_{H} and a V_{L} derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments, the scFv and/or V_{H} is derived from FMC63. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the FMC63 antibody comprises CDR-H1 and CDR-H2 set forth in SEQ ID NOS: 38 and 39, respectively, and CDR-H3 set forth in SEQ ID NO: 40 or 54; and CDR-L1 set forth in SEQ ID NO: 35 and CDR-L2 set forth in SEQ ID NO: 36 or 55 and CDR-L3 set forth in SEQ ID NO: 37 or 34. In some embodiments, the FMC63 antibody comprises the heavy chain variable region (V_{H}) comprising the amino acid sequence of SEQ ID NO: 41 and the light chain variable region (V_{L}) comprising the amino acid sequence of SEQ ID NO: 42.

In some embodiments, the scFv comprises a variable light chain containing the CDR-L1 sequence of SEQ ID NO:35, a CDR-L2 sequence of SEQ ID NO:36, and a CDR-L3 sequence of SEQ ID NO:37 and/or a variable heavy chain containing a CDR-H1 sequence of SEQ ID NO:38, a CDR-H2 sequence of SEQ ID NO:39, and a CDR-H3 sequence of SEQ ID NO:40. In some embodiments, the scFv comprises a variable heavy chain region set forth in SEQ ID NO:41 and a variable light chain region set forth in SEQ ID NO:42. In some embodiments, the variable heavy and variable light chains are connected by a linker. In some embodiments, the linker is set forth in SEQ ID NO:56. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:57 or a sequence that exhibits at least 85%, 86%, 87%, 88%, 89%, 90%, 91%, 92%, 93%, 94%, 95%, 96%, 97%, 98%, or 99% sequence identity to SEQ ID NO:57. In some embodiments, the scFv comprises the sequence of amino acids set forth in SEQ ID NO:43 or a sequence that exhibits at least 85%, 86%, 87%, 88%, 89%, 90%, 91%, 92%, 93%, 94%, 95%, 96%, 97%, 98%, or 99% sequence identity to SEQ ID NO:43.

In some embodiments the scFv is derived from SJ25C1. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises CDR-H1, CDR-H2 and CDR-H3 set forth in SEQ ID NOS: 47-49, respectively, and CDR-L1, CDR-L2 and CDR-L3 sequences set forth in SEQ ID NOS: 44-46, respectively. In some embodiments, the SJ25C1 antibody comprises the heavy chain variable region (V_{H}) comprising the amino acid sequence of SEQ ID NO: 50 and the light chain variable region (V_{L}) comprising the amino acid sequence of SEQ ID NO: 51.

In some embodiments, the scFv comprises a variable light chain containing a CDR-L1 sequence of SEQ ID NO:44, a CDR-L2 sequence of SEQ ID NO: 45, and a CDR-L3 sequence of SEQ ID NO:46 and/or a variable heavy chain containing a CDR-H1 sequence of SEQ ID NO:47, a CDR-H2 sequence of SEQ ID NO:48, and a CDR-H3 sequence of SEQ ID NO:49. In some embodiments, the scFv comprises a variable heavy chain region set forth in SEQ ID NO:50 and a variable light chain region set forth in SEQ ID NO:51. In some embodiments, the variable heavy and variable light chain are connected by a linker. In some embodiments, the linker is set forth in SEQ ID NO:52. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:53 or a sequence that exhibits at least 85%, 86%, 87%, 88%, 89%, 90%, 91%, 92%, 93%, 94%, 95%, 96%, 97%, 98%, or 99% sequence identity to SEQ ID NO:53.

In some embodiments, the antigen is CD20. In some embodiments, the scFv contains a V_{H} and a V_{L} derived from an antibody or an antibody fragment specific to CD20. In some embodiments, the antibody or antibody fragment that binds CD20 is an antibody that is or is derived from Rituximab, such as is Rituximab scFv.

In some embodiments, the antigen is CD22. In some embodiments, the scFv contains a V_{H} and a V_{L} derived from an antibody or an antibody fragment specific to CD22. In some embodiments, the antibody or antibody fragment that binds CD22 is an antibody that is or is derived from m971, such as is m971 scFv.

In some embodiments, the CAR contains a TCR-like antibody, such as an antibody or an antigen-binding fragment (e.g. scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR.

Reference to “Major histocompatibility complex” (MHC) refers to a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e. MHC-peptide complex, for presentation of an antigen in a conformation recognizable by an antigen receptor on T cells, such as a TCRs or TCR-like antibody. Generally, MHC class I molecules are heterodimers having a membrane spanning a chain, in some cases with three a domains, and a non-covalently associated (32 microglobulin. Generally, MHC class II molecules are composed of two transmembrane glycoproteins, a and (3, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8⁺ T cells, but in some cases CD4+ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, a TCR-like antibody or antigen-binding portion, are known or can be produced by known methods (see e.g. US Published Application Nos. US 2002/0150914; US 2003/0223994; US 2004/0191260; US 2006/0034850; US 2007/00992530; US20090226474; US20090304679; and International PCT Publication No. WO 03/068201).

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen or other antigen as described below. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See e.g. US published application No. US20020150914, US2014/0294841; and Cohen C J. et al. (2003) J Mol. Recogn. 16:324-332.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Thus, in some embodiments, the chimeric antigen receptor, including TCR-like CARs, includes an extracellular portion containing an antibody or antibody fragment. In some embodiments, the antibody or fragment includes an scFv. In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the recombinant receptor such as the CAR, such as the antibody portion thereof, further includes a spacer, which may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, Hudecek et al. (2015) Cancer Immunol Res. 3(2): 125-135 or international patent application publication number WO2014031687. In some embodiments, the spacer has the sequence set forth in SEQ ID NO: 1, and is encoded by the sequence set forth in SEQ ID NO: 2. In some embodiments, the spacer has the sequence set forth in SEQ ID NO: 3. In some embodiments, the spacer has the sequence set forth in SEQ ID NO: 4.

In some aspects, the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) consists or comprises the sequence of amino acids set forth in SEQ ID NOS: 1, 3-5, 27-34 or 58, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine.

In some embodiments, the constant region or portion is of IgD. In some embodiments, the spacer has the sequence set forth in SEQ ID NO: 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 1, 3, 4 and 5.

The antigen recognition domain generally is linked to one or more intracellular signaling components, such as signaling components that mimic activation through an antigen receptor complex, such as a TCR complex, in the case of a CAR, and/or signal via another cell surface receptor. Thus, in some embodiments, the antigen binding component (e.g., antibody) is linked to one or more transmembrane and intracellular signaling regions. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling region are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding or antigen-recognition domain is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR includes a chimeric molecule between CD3-zeta (CD3-ζ) or Fc receptor γ and CD8, CD4, CD25 or CD16.

In some embodiments, upon ligation of the CAR, the cytoplasmic domain or intracellular signaling region of the CAR activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling region of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma or FcR beta. In some embodiments, cytoplasmic signaling molecule(s) in the CAR contain(s) a cytoplasmic signaling domain, portion thereof, or sequence derived from CD3 zeta.

In some embodiments, the CAR includes a signaling region and/or transmembrane portion of a costimulatory receptor, such as CD28, 4-1BB, OX40, DAP10, and ICOS. In some aspects, the same CAR includes both the signaling region and costimulatory components.

In some embodiments, the signaling region is included within one CAR, whereas the costimulatory component is provided by another CAR recognizing another antigen. In some embodiments, the CARs include activating or stimulatory CARs, and costimulatory CARs, both expressed on the same cell (see WO2014/055668).

In certain embodiments, the intracellular signaling region comprises a CD28 transmembrane and signaling domain linked to a CD3 (e.g., CD3-zeta) intracellular domain. In some embodiments, the intracellular signaling region comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular portion containing the antibody or fragment described herein. In some aspects, the chimeric antigen receptor includes an extracellular portion containing the antibody or fragment described herein and an intracellular signaling domain. In some embodiments, the antibody or fragment includes an scFv or a single-domain V_{H} antibody and the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain disposed between the extracellular domain and the intracellular signaling region.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 4-1BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:8; in some embodiments, the transmembrane-domain containing portion of the recombinant receptor comprises the sequence of amino acids set forth in SEQ ID NO: 9 or a sequence of amino acids having at least or at least about 85%, 86%, 87%, 88%, 89%, 90%, 91%, 92%, 93%, 94%, 95%, 96%, 97%, 98%, 99% or more sequence identity thereto.

In some embodiments, the chimeric antigen receptor contains an intracellular domain of a T cell costimulatory molecule. In some aspects, the T cell costimulatory molecule is CD28 or 4-1BB.

In some embodiments, the intracellular signaling region comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 10 or 11 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular region comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 12 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 12.

In some embodiments, the intracellular signaling region comprises a human CD3 chain, optionally a CD3 zeta stimulatory signaling domain or functional variant thereof, such as an 112 AA cytoplasmic domain of isoform 3 of human CD3 (Accession No.: P20963.2) or a CD3 zeta signaling domain as described in U.S. Pat. No. 7,446,190 or 8,911,993. In some embodiments, the intracellular signaling region comprises the sequence of amino acids set forth in SEQ ID NO: 13, 14 or 15 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 13, 14 or 15.

In some aspects, the spacer contains only a hinge region of an IgG, such as only a hinge of IgG4 or IgG1, such as the hinge only spacer set forth in SEQ ID NO:1. In other embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO:3. In some embodiments, the spacer is an Ig hinge, e.g., an IgG4 hinge, linked to a C_{H}3 domain only, such as set forth in SEQ ID NO:4. In some embodiments, the spacer is or comprises a glycine-serine rich sequence or other flexible linker such as known flexible linkers.

2. T Cell Receptors (TCRs)

In some embodiments, engineered cells, such as T cells, are provided that express a T cell receptor (TCR) or antigen-binding portion thereof that recognizes an peptide epitope or T cell epitope of a target polypeptide, such as an antigen of a tumor, viral or autoimmune protein.

In some embodiments, a “T cell receptor” or “TCR” is a molecule that contains a variable α and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or C_{β}, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g. present on the antigen-specific T cells, may be selected, such as by binding activity, e.g., particular affinity or avidity for the antigen.

In some embodiments, the genetically engineered antigen receptors include recombinant T cell receptors (TCRs) and/or TCRs cloned from naturally occurring T cells. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified by a skilled artisan. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007).

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known, See e.g., Soo Hoo, W. F. et al. PNAS (USA) 89, 4759 (1992); Wülfing, C. and Plückthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see e.g. International published PCT No. WO 03/020763). In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via peptide linker (see e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an a chain variable region sequence fused to the N terminus of a sequence a chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)₅-P- wherein P is proline, G is glycine and S is serine (SEQ ID NO:22). In some embodiments, the linker has the sequence GSADDAKKDAAKKDGKS (SEQ ID NO:23)

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10-5 and 10-12 M and all individual values and ranges therein. In some embodiments, the target antigen is an MHC-peptide complex or ligand.

In some embodiments, nucleic acid or nucleic acids encoding a TCR, such as α and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can be a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λ610, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, after the T-cell clone is obtained, the TCR alpha and beta chains are isolated and cloned into a gene expression vector. In some embodiments, the TCR alpha and beta genes are linked via picornavirus 2A ribosomal skip peptide so that both chains are coexpression. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; and Hackett et al. (2010) Molecular Therapy: The Journal of the American Society of Gene Therapy. 18:674-683.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g. lentiviral, vector.

3. Multi-Targeting

In some embodiments, the cells and methods include multi-targeting strategies, such as expression of two or more genetically engineered receptors on the cell, each recognizing the same of a different antigen and typically each including a different intracellular signaling component. Such multi-targeting strategies are described, for example, in International Patent Application Publication No: WO 2014055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (December, 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

For example, in some embodiments, the cells include a receptor expressing a first genetically engineered antigen receptor (e.g., CAR or TCR) which is capable of inducing an activating or stimulating signal to the cell, generally upon specific binding to the antigen recognized by the first receptor, e.g., the first antigen. In some embodiments, the cell further includes a second genetically engineered antigen receptor (e.g., CAR or TCR), e.g., a chimeric costimulatory receptor, which is capable of inducing a costimulatory signal to the immune cell, generally upon specific binding to a second antigen recognized by the second receptor. In some embodiments, the first antigen and second antigen are the same. In some embodiments, the first antigen and second antigen are different.

In some embodiments, the first and/or second genetically engineered antigen receptor (e.g. CAR or TCR) is capable of inducing an activating or stimulating signal to the cell. In some embodiments, the receptor includes an intracellular signaling component containing ITAM or ITAM-like motifs. In some embodiments, the activation induced by the first receptor involves a signal transduction or change in protein expression in the cell resulting in initiation of an immune response, such as ITAM phosphorylation and/or initiation of ITAM-mediated signal transduction cascade, formation of an immunological synapse and/or clustering of molecules near the bound receptor (e.g. CD4 or CD8, etc.), activation of one or more transcription factors, such as NF—KB and/or AP-1, and/or induction of gene expression of factors such as cytokines, proliferation, and/or survival.

In some embodiments, the first and/or second receptor includes intracellular signaling domains of costimulatory receptors such as CD28, CD137 (4-1BB), OX40, and/or ICOS. In some embodiments, the first and second receptor include an intracellular signaling domain of a costimulatory receptor that are different. In one embodiment, the first receptor contains a CD28 costimulatory signaling region and the second receptor contain a 4-1BB co-stimulatory signaling region or vice versa.

In some embodiments, the first and/or second receptor includes both an intracellular signaling domain containing ITAM or ITAM-like motifs and an intracellular signaling domain of a costimulatory receptor.

In some embodiments, the first receptor contains an intracellular signaling domain containing ITAM or ITAM-like motifs and the second receptor contains an intracellular signaling domain of a costimulatory receptor. The costimulatory signal in combination with the activating or stimulating signal induced in the same cell is one that results in an immune response, such as a robust and sustained immune response, such as increased gene expression, secretion of cytokines and other factors, and T cell mediated effector functions such as cell killing.

In some embodiments, neither ligation of the first receptor alone nor ligation of the second receptor alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the plurality of receptors are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a target cell.

In some embodiments, the two receptors induce, respectively, an activating and an inhibitory signal to the cell, such that binding by one of the receptor to its antigen activates the cell or induces a response, but binding by the second inhibitory receptor to its antigen induces a signal that suppresses or dampens that response. Examples are combinations of activating CARs and inhibitory CARs or iCARs. Such a strategy may be used, for example, in which the activating CAR binds an antigen expressed in a disease or condition but which is also expressed on normal cells, and the inhibitory receptor binds to a separate antigen which is expressed on the normal cells but not cells of the disease or condition.

In some embodiments, the multi-targeting strategy is employed in a case where an antigen associated with a particular disease or condition is expressed on a non-diseased cell and/or is expressed on the engineered cell itself, either transiently (e.g., upon stimulation in association with genetic engineering) or permanently. In such cases, by requiring ligation of two separate and individually specific antigen receptors, specificity, selectivity, and/or efficacy may be improved.

In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

B. Nucleic Acids, Vectors and Methods for Genetic Engineering

In some embodiments, the cells, e.g., T cells, are genetically engineered to express a recombinant receptor. In some embodiments, the engineering is carried out by introducing polynucleotides that encode the recombinant receptor. Also provided are polynucleotides encoding a recombinant receptor, and vectors or constructs containing such nucleic acids and/or polynucleotides.

In some cases, the nucleic acid sequence encoding the recombinant receptor contains a signal sequence that encodes a signal peptide. In some aspects, the signal sequence may encode a signal peptide derived from a native polypeptide. In other aspects, the signal sequence may encode a heterologous or non-native signal peptide, such as the exemplary signal peptide of the GMCSFR alpha chain set forth in SEQ ID NO:25 and encoded by the nucleotide sequence set forth in SEQ ID NO:24. In some cases, the nucleic acid sequence encoding the recombinant receptor, e.g., chimeric antigen receptor (CAR) contains a signal sequence that encodes a signal peptide. Non-limiting exemplary examples of signal peptides include, for example, the GMCSFR alpha chain signal peptide set forth in SEQ ID NO: 25 and encoded by the nucleotide sequence set forth in SEQ ID NO:24, or the CD8 alpha signal peptide set forth in SEQ ID NO:26.

In some embodiments, the polynucleotide encoding the recombinant receptor contains at least one promoter that is operatively linked to control expression of the recombinant receptor. In some examples, the polynucleotide contains two, three, or more promoters operatively linked to control expression of the recombinant receptor.

In certain cases where nucleic acid molecules encode two or more different polypeptide chains, e.g., a recombinant receptor and a marker, each of the polypeptide chains can be encoded by a separate nucleic acid molecule. For example, two separate nucleic acids are provided, and each can be individually transferred or introduced into the cell for expression in the cell. In some embodiments, the nucleic acid encoding the recombinant receptor and the nucleic acid encoding the marker are operably linked to the same promoter and are optionally separated by an internal ribosome entry site (IRES), or a nucleic acid encoding a self-cleaving peptide or a peptide that causes ribosome skipping, which optionally is a T2A, a P2A, an E2A or an F2A. In some embodiments, the nucleic acids encoding the marker and the nucleic acid encoding the recombinant receptor are operably linked to two different promoters. In some embodiments, the nucleic acid encoding the marker and the nucleic acid encoding the recombinant receptor are present or inserted at different locations within the genome of the cell. In some embodiments, the polynucleotide encoding the recombinant receptor is introduced into a composition containing cultured cells, such as by retroviral transduction, transfection, or transformation.

In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, transcription units can be engineered as a bicistronic unit containing an IRES (internal ribosome entry site), which allows coexpression of gene products ((e.g. encoding the marker and encoding the recombinant receptor) by a message from a single promoter. Alternatively, in some cases, a single promoter may direct expression of an RNA that contains, in a single open reading frame (ORF), two or three genes (e.g. encoding the marker and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004)). Various 2A elements are known. Examples of 2A sequences that can be used in the methods and system disclosed herein, without limitation, 2A sequences from the foot-and-mouth disease virus (F2A, e.g., SEQ ID NO: 21), equine rhinitis A virus (E2A, e.g., SEQ ID NO: 20), Thosea asigna virus (T2A, e.g., SEQ ID NO: 6 or 17), and porcine teschovirus-1 (P2A, e.g., SEQ ID NO: 18 or 19) as described in U.S. Patent Publication No. 20070116690.

Any of the recombinant receptors described herein can be encoded by polynucleotides containing one or more nucleic acid sequences encoding recombinant receptors, in any combinations or arrangements. For example, one, two, three or more polynucleotides can encode one, two, three or more different polypeptides, e.g., recombinant receptors. In some embodiments, one vector or construct contains a nucleic acid sequence encoding marker, and a separate vector or construct contains a nucleic acid sequence encoding a recombinant receptor, e.g., CAR. In some embodiments, the nucleic acid encoding the marker and the nucleic acid encoding the recombinant receptor are operably linked to two different promoters. In some embodiments, the nucleic acid encoding the recombinant receptor is present downstream of the nucleic acid encoding the marker.

In some embodiments, the vector backbone contains a nucleic acid sequence encoding one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:7 or 16) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR).

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 7 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16.

In some embodiments, the marker is or comprises a fluorescent protein, such as green fluorescent protein (GFP), enhanced green fluorescent protein (EGFR), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy, 2014 April 3. doi: 10.1038/gt.2014.25; Carlens et al. (2000) Exp. Hematol., 28(10): 1137-46; Alonso-Camino et al. (2013) Mol. Ther. Nucl. Acids., 2, e93; Park et al., Trends Biotechnol., 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV) or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the anti-CD3/anti-CD28 stimulus, for example) and subsequently be stimulated with a second type of stimulus e.g. via de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

C. Cells and Preparation of Cells for Genetic Engineering

In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood, 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or subpopulation, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° C. per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of stimulating or activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

### III. EXEMPLARY TREATMENT OUTCOMES AND METHODS FOR ASSESSING Same

In some embodiments of the methods, compositions, combinations, uses, kits and Articles of manufacture provided herein, the provided combination therapy results in one or more treatment outcomes, such as a feature associated with any one or more of the parameters associated with the therapy or treatment, as described below. In some embodiments, the method includes assessment of the exposure, persistence and proliferation of the T cells, e.g., T cells administered for the T cell based therapy. In some embodiments, the exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the characteristics of the T cells in vitro or ex vivo. In some embodiments, such assays can be used to determine or confirm the function of the T cells, e.g. T cell therapy, before, during, or after administering the combination therapy provided herein.

In some embodiments, the combination therapy can further include one or more screening steps to identify subjects for treatment with the combination therapy and/or continuing the combination therapy, and/or a step for assessment of treatment outcomes and/or monitoring treatment outcomes. In some embodiments, the step for assessment of treatment outcomes can include steps to evaluate and/or to monitor treatment and/or to identify subjects for administration of further or remaining steps of the therapy and/or for repeat therapy. In some embodiments, the screening step and/or assessment of treatment outcomes can be used to determine the dose, frequency, duration, timing and/or order of the combination therapy provided herein.

In some embodiments, any of the screening steps and/or assessment of treatment of outcomes described herein can be used prior to, during, during the course of, or subsequent to administration of one or more steps of the provided combination therapy, e.g., administration of the T cell therapy (e.g. CAR-expressing T cells), and/or a checkpoint inhibitor, e.g., anti-PD-L1 antibody (or antigen-binding fragment thereof). In some embodiments, assessment is made prior to, during, during the course of, or after performing any of the methods provided herein. In some embodiments, the assessment is made prior to performing the methods provided herein. In some embodiments, assessment is made after performing one or more steps of the methods provided herein. In some embodiments, the assessment is performed prior to administration of administration of one or more steps of the provided combination therapy, for example, to screen and identify patients suitable and/or susceptible to receive the combination therapy. In some embodiments, the assessment is performed during, during the course of, or subsequent to administration of one or more steps of the provided combination therapy, for example, to assess the intermediate or final treatment outcome, e.g., to determine the efficacy of the treatment and/or to determine whether to continue or repeat the treatments and/or to determine whether to administer the remaining steps of the combination therapy.

In some embodiments, treatment of outcomes includes improved immune function, e.g., immune function of the T cells administered for cell based therapy and/or of the endogenous T cells in the body. In some embodiments, exemplary treatment outcomes include, but are not limited to, enhanced T cell proliferation, enhanced T cell functional activity, changes in immune cell phenotypic marker expression, such as such features being associated with the engineered T cells, e.g. CAR-T cells, administered to the subject. In some embodiments, exemplary treatment outcomes include decreased disease burden, e.g., tumor burden, improved clinical outcomes and/or enhanced efficacy of therapy.

In some embodiments, the screening step and/or assessment of treatment of outcomes includes assessing the survival and/or function of the T cells administered for cell based therapy. In some embodiments, the screening step and/or assessment of treatment of outcomes includes assessing the levels of cytokines or growth factors. In some embodiments, the screening step and/or assessment of treatment of outcomes includes assessing disease burden and/or improvements, e.g., assessing tumor burden and/or clinical outcomes. In some embodiments, either of the screening step and/or assessment of treatment of outcomes can include any of the assessment methods and/or assays described herein and/or known in the art, and can be performed one or more times, e.g., prior to, during, during the course of, or subsequently to administration of one or more steps of the combination therapy. Exemplary sets of parameters associated with a treatment outcome, which can be assessed in some embodiments of the methods provided herein, include peripheral blood immune cell population profile and/or tumor burden.

In some embodiments, the methods affect efficacy of the cell therapy in the subject. In some embodiments, the persistence, expansion, and/or presence of recombinant receptor-expressing, e.g., CAR-expressing, cells in the subject following administration of the dose of cells in the method with a checkpoint inhibitor, e.g., anti-PD-L1 antibody (or antigen-binding fragment thereof) is greater as compared to that achieved via method without the administration of a checkpoint inhibitor, e.g., anti-PD-L1 antibody (or antigen-binding fragment thereof). In some embodiments of the immunotherapy methods provided herein, such as a T cell therapy (e.g. CAR-expressing T cells), assessment of the parameter includes assessing the expansion and/or persistence in the subject of the administered T cells for the immunotherapy, e.g., T cell therapy, as compared to a method in which the immunotherapy is administered to the subject in the absence of a checkpoint inhibitor, e.g., anti-PD-L1 antibody (or antigen-binding fragment thereof). In some embodiments, the methods result in the administered T cells exhibiting increased or prolonged expansion and/or persistence in the subject as compared to a method in which the T cell therapy is administered to the subject in the absence of a checkpoint inhibitor, e.g., anti-PD-L1 antibody (or antigen-binding fragment thereof).

In some embodiments, the administration of the checkpoint inhibitor, e.g., anti-PD-L1 antibody (or antigen-binding fragment thereof) decreases disease burden, e.g., tumor burden, in the subject as compared to a method in which the dose of cells expressing the recombinant receptor is administered to the subject in the absence of the checkpoint inhibitor, e.g., anti-PD-L1 antibody (or antigen-binding fragment thereof). In some embodiments, the administration of the checkpoint inhibitor, e.g., anti-PD-L1 antibody (or antigen-binding fragment thereof) decreases blast marrow in the subject as compared to a method in which the dose of cells expressing the recombinant receptor is administered to the subject in the absence of the checkpoint inhibitor, e.g., anti-PD-L1 antibody (or antigen-binding fragment thereof). In some embodiments, the administration of the checkpoint inhibitor, e.g., anti-PD-L1 antibody (or antigen-binding fragment thereof) results in improved clinical outcomes, e.g., objective response rate (ORR), progression-free survival (PFS) and overall survival (OS), compared to a method in which the dose of cells expressing the recombinant receptor is administered to the subject in the absence of the checkpoint inhibitor, e.g., anti-PD-L1 antibody (or antigen-binding fragment thereof).

In some embodiments, the subject can be screened prior to the administration of one or more steps of the combination therapy. For example, the subject can be screened for characteristics of the disease and/or disease burden, e.g., tumor burden, prior to administration of the combination therapy, to determine suitability, responsiveness and/or susceptibility to administering the combination therapy. In some embodiments, the screening step and/or assessment of treatment outcomes can be used to determine the dose, frequency, duration, timing and/or order of the combination therapy provided herein.

In some embodiments, the subject can be screened after administration of one of the steps of the combination therapy, to determine and identify subjects to receive the remaining steps of the combination therapy and/or to monitor efficacy of the therapy. In some embodiments, the number, level or amount of administered T cells and/or proliferation and/or activity of the administered T cells is assessed prior to administration and/or after administration of the checkpoint inhibitor, e.g., anti-PD-L1 antibody (or antigen-binding fragment thereof).

In some embodiments, the checkpoint inhibitor, e.g., anti-PD-L1 antibody (or antigen-binding fragment thereof) is administered after the cell therapy administration. In some embodiments, at least one cycle (e.g., a 28-day cycle) of the checkpoint inhibitor, e.g., anti-PD-L1 antibody (or antigen-binding fragment thereof) is not administered until at least about 22 to 50 days after the administration of the cell therapy. In some embodiments, the first cycle of the checkpoint inhibitor, e.g., anti-PD-L1 antibody (or antigen-binding fragment thereof) is not administered until at least about 22 to 50 days after the administration of the cell therapy. In some embodiments, at least one cycle (e.g., a first cycle) of the checkpoint inhibitor, e.g., anti-PD-L1 antibody (or antigen-binding fragment thereof) is administered at a time point of about 22 to 50 days after the administration of the cell therapy. In some embodiments, the checkpoint inhibitor, e.g., anti-PD-L1 antibody (or antigen-binding fragment thereof) is administered more than once in the cycle. In some embodiments, the cycle is a 28-day cycle.

In some embodiments, a change and/or an alteration, e.g., an increase, an elevation, a decrease or a reduction, in levels, values or measurements of a parameter or outcome compared to the levels, values or measurements of the same parameter or outcome in a different time point of assessment, a different condition, a reference point and/or a different subject is determined or assessed. For example, in some embodiments, a fold change, e.g., an increase or decrease, in particular parameters, e.g., number of engineered T cells in a sample, compared to the same parameter in a different condition, e.g., before administration of the checkpoint inhibitor, e.g., anti-PD-L1 antibody (or antigen-binding fragment thereof) can be determined. In some embodiments, the levels, values or measurements of two or more parameters are determined, and relative levels are compared. In some embodiments, the determined levels, values or measurements of parameters are compared to the levels, values or measurements from a control sample or an untreated sample. In some embodiments, the determined levels, values or measurements of parameters are compared to the levels from a sample from the same subject but at a different time point. The values obtained in the quantification of individual parameter can be combined for the purpose of disease assessment, e.g., by forming an arithmetical or logical operation on the levels, values or measurements of parameters by using multi-parametric analysis. In some embodiments, a ratio of two or more specific parameters can be calculated.

A. T Cell Exposure, Persistence and Proliferation

In some embodiments, the parameter associated with therapy or a treatment outcome, which include parameters that can be assessed for the screening steps and/or assessment of treatment of outcomes and/or monitoring treatment outcomes, is or includes assessment of the exposure, persistence and proliferation of the T cells, e.g., T cells administered for the T cell based therapy. In some embodiments, the increased exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the characteristics of the T cells in vitro or ex vivo. In some embodiments, such assays can be used to determine or confirm the function of the T cells used for the immunotherapy, e.g. T cell therapy, before or after administering one or more steps of the combination therapy provided herein.

In some embodiments, the administration of the checkpoint inhibitor, e.g., anti-PD-L1 antibody (or antigen-binding fragment thereof) are designed to promote exposure of the subject to the cells, e.g., T cells administered for T cell based therapy, such as by promoting their expansion and/or persistence over time. In some embodiments, the T cell therapy exhibits increased or prolonged expansion and/or persistence in the subject as compared to a method in which the T cell therapy is administered to the subject in the absence of the checkpoint inhibitor, e.g., anti-PD-L1 antibody (or antigen-binding fragment thereof).

In some embodiments, the provided methods increase exposure of the subject to the administered cells (e.g., increased number of cells or duration over time) and/or improve efficacy and therapeutic outcomes of the immunotherapy, e.g. T cell therapy. In some aspects, the methods are advantageous in that a greater and/or longer degree of exposure to the cells expressing the recombinant receptors, e.g., CAR-expressing cells, improves treatment outcomes as compared with other methods. Such outcomes may include patient survival and remission, even in individuals with severe tumor burden.

In some embodiments, the administration of the checkpoint inhibitor, e.g., anti-PD-L1 antibody (or antigen-binding fragment thereof) can increase the maximum, total, and/or duration of exposure to the cells, e.g. T cells administered for the T cell based therapy, in the subject as compared to administration of the T cells alone in the absence of the checkpoint inhibitor, e.g., anti-PD-L1 antibody (or antigen-binding fragment thereof). In some aspects, administration of the checkpoint inhibitor, e.g., anti-PD-L1 antibody (or antigen-binding fragment thereof), in the context of high disease burden (and thus higher amounts of antigen) and/or a more aggressive or resistant cancer enhances efficacy as compared with administration of the T cells alone in the absence of the checkpoint inhibitor, e.g., anti-PD-L1 antibody (or antigen-binding fragment thereof) in the same context, which may result in immunosuppression, anergy and/or exhaustion which may prevent expansion and/or persistence of the cells.

In some embodiments, the presence and/or amount of cells expressing the recombinant receptor (e.g., CAR-expressing cells administered for T cell based therapy) in the subject following the administration of the T cells and before, during and/or after the administration of the checkpoint inhibitor, e.g., anti-PD-L1 antibody (or antigen-binding fragment thereof) is detected. In some aspects, quantitative PCR (qPCR) is used to assess the quantity of cells expressing the recombinant receptor (e.g., CAR-expressing cells administered for T cell based therapy) in the blood or serum or organ or tissue sample (e.g., disease site, e.g., tumor sample) of the subject. In some aspects, persistence is quantified as copies of DNA or plasmid encoding the receptor, e.g., CAR, per microgram of DNA, or as the number of receptor-expressing, e.g., CAR-expressing, cells per microliter of the sample, e.g., of blood or serum, or per total number of peripheral blood mononuclear cells (PBMCs) or white blood cells or T cells per microliter of the sample.

In some embodiments, the cells are detected in the subject at or at least at 4, 7, 10, 14, 18, 21, 24, 27, or 28 days following the administration of the T cells, e.g., CAR-expressing T cells. In some aspects, the cells are detected at or at least at 2, 4, or 6 weeks following, or 3, 6, or 12, 18, or 24, or 30 or 36 months, or 1, 2, 3, 4, 5, or more years, following the administration of the T cells.

In some embodiments, the persistence of receptor-expressing cells (e.g. CAR-expressing cells) in the subject by the methods, following the administration of the T cells, e.g., CAR-expressing T cells and/or the checkpoint inhibitor, e.g., anti-PD-L1 antibody (or antigen-binding fragment thereof), is greater as compared to that which would be achieved by alternative methods such as those involving the administration of the immunotherapy alone, e.g., administration the T cells, e.g., CAR-expressing T cells, in the absence of the checkpoint inhibitor, e.g., anti-PD-L1 antibody (or antigen-binding fragment thereof).

The exposure, e.g., number of cells, e.g. T cells administered for T cell therapy, indicative of expansion and/or persistence, may be stated in terms of maximum numbers of the cells to which the subject is exposed, duration of detectable cells or cells above a certain number or percentage, area under the curve for number of cells over time, and/or combinations thereof and indicators thereof. Such outcomes may be assessed using known methods, such as qPCR to detect copy number of nucleic acid encoding the recombinant receptor compared to total amount of nucleic acid or DNA in the particular sample, e.g., blood, serum, plasma or tissue, such as a tumor sample, and/or flow cytometric assays detecting cells expressing the receptor generally using antibodies specific for the receptors. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor.

In some aspects, increased exposure of the subject to the cells includes increased expansion of the cells. In some embodiments, the receptor expressing cells, e.g. CAR-expressing cells, expand in the subject following administration of the T cells, e.g., CAR-expressing T cells, and/or following administration of the checkpoint inhibitor, e.g., anti-PD-L1 antibody (or antigen-binding fragment thereof). In some aspects, the methods result in greater expansion of the cells compared with other methods, such as those involving the administration of the T cells, e.g., CAR-expressing T cells, in the absence of administering the checkpoint inhibitor, e.g., anti-PD-L1 antibody (or antigen-binding fragment thereof).

In some aspects, the method results in high in vivo proliferation of the administered cells, for example, as measured by flow cytometry. In some aspects, high peak proportions of the cells are detected. For example, in some embodiments, at a peak or maximum level following the administration of the T cells, e.g., CAR-expressing T cells and/or the checkpoint inhibitor, e.g., anti-PD-L1 antibody (or antigen-binding fragment thereof), in the blood or disease-site of the subject or white blood cell fraction thereof, e.g., PBMC fraction or T cell fraction, at least about 10%, at least about 20%, at least about 30%, at least about 40%, at least about 50%, at least about 60%, at least about 70%, at least about 80%, or at least about 90% of the cells express the recombinant receptor, e.g., the CAR.

In some embodiments, the method results in a maximum concentration, in the blood or serum or other bodily fluid or organ or tissue of the subject, of at least 100, 500, 1000, 1500, 2000, 5000, 10,000 or 15,000 copies of or nucleic acid encoding the receptor, e.g., the CAR, per microgram of DNA, or at least 0.1, 0.2, 0.3, 0.4, 0.5, 0.6, 0.7, 0.8, or 0.9 receptor-expressing, e.g., CAR,-expressing cells per total number of peripheral blood mononuclear cells (PBMCs), total number of mononuclear cells, total number of T cells, or total number of microliters. In some embodiments, the cells expressing the receptor are detected as at least 10, 20, 30, 40, 50, or 60% of total PBMCs in the blood of the subject, and/or at such a level for at least 1, 2, 3, 4, 5, 6, 7, 8, 9, 10, 11, 12, 24, 36, 48, or 52 weeks following the T cells, e.g., CAR-expressing T cells and/or the checkpoint inhibitor, e.g., anti-PD-L1 antibody (or antigen-binding fragment thereof), or for 1, 2, 3, 4, or 5, or more years following such administration.

In some aspects, the method results in at least a 2-fold, at least a 4-fold, at least a 10-fold, or at least a 20-fold increase in copies of nucleic acid encoding the recombinant receptor, e.g., CAR, per microgram of DNA, e.g., in the serum, plasma, blood or tissue, e.g., tumor sample, of the subject.

In some embodiments, cells expressing the receptor are detectable in the serum, plasma, blood or tissue, e.g., tumor sample, of the subject, e.g., by a specified method, such as qPCR or flow cytometry-based detection method, at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 or more days following administration of the T cells, e.g., CAR-expressing T cells, or after administration of the checkpoint inhibitor, e.g., anti-PD-L1 antibody (or antigen-binding fragment thereof), for at least at or about 2, 3, 4, 5, 6, 7, 8, 9, 10, 11, 12, 13, 14, 15, 16, 17, 18, 19, 20, 21, 22, 23, or 24 or more weeks following the administration of the T cells, e.g., CAR-expressing T cells, and/or the checkpoint inhibitor, e.g., anti-PD-L1 antibody (or antigen-binding fragment thereof).

In some aspects, at least about 1×10², at least about 1×10³, at least about 1×10⁴, at least about 1×10⁵, or at least about 1×10⁶ or at least about 5×10⁶ or at least about 1×10⁷ or at least about 5×10⁷ or at least about 1×10⁸ recombinant receptor-expressing, e.g., CAR-expressing cells, and/or at least 10, 25, 50, 100, 200, 300, 400, or 500, or 1000 receptor-expressing cells per microliter, e.g., at least 10 per microliter, are detectable or are present in the subject or fluid, plasma, serum, tissue, or compartment thereof, such as in the blood, e.g., peripheral blood, or disease site, e.g., tumor, thereof. In some embodiments, such a number or concentration of cells is detectable in the subject for at least about 20 days, at least about 40 days, or at least about 60 days, or at least about 3, 4, 5, 6, 7, 8, 9, 10, 11, or 12 months, or at least 2 or 3 years, following administration of the T cells, e.g., CAR-expressing T cells, and/or following the administration of the checkpoint inhibitor, e.g., anti-PD-L1 antibody (or antigen-binding fragment thereof). Such cell numbers may be as detected by flow cytometry-based or quantitative PCR-based methods and extrapolation to total cell numbers using known methods. See, e.g., Brentjens et al., Sci Transl Med. 2013 5(177), Park et al, Molecular Therapy 15(4):825-833 (2007), Savoldo et al., JCI 121(5):1822-1826 (2011), Davila et al., (2013) PLoS ONE 8(4):e61338, Davila et al., Oncoimmunology 1(9):1577-1583 (2012), Lamers, Blood 2011 117:72-82, Jensen et al., Biol Blood Marrow Transplant 2010 September; 16(9): 1245-1256, Brentjens et al., Blood 2011 118(18):4817-4828.

In some aspects, the copy number of nucleic acid encoding the recombinant receptor, e.g., vector copy number, per 100 cells, for example in the peripheral blood or bone marrow or other compartment, as measured by immunohistochemistry, PCR, and/or flow cytometry, is at least 0.01, at least 0.1, at least 1, or at least 10, at about 1 week, about 2 weeks, about 3 weeks, about 4 weeks, about 5 weeks, or at least about 6 weeks, or at least about 2, 3, 4, 5, 6, 7, 8. 9, 10, 11, or 12 months or at least 2 or 3 years following administration of the cells, e.g., CAR-expressing T cells, and/or the checkpoint inhibitor, e.g., anti-PD-L1 antibody (or antigen-binding fragment thereof). In some embodiments, the copy number of the vector expressing the receptor, e.g. CAR, per microgram of genomic DNA is at least 100, at least 1000, at least 5000, or at least 10,000, or at least 15,000 or at least 20,000 at a time about 1 week, about 2 weeks, about 3 weeks, or at least about 4 weeks following administration of the T cells, e.g., CAR-expressing T cells, a checkpoint inhibitor, e.g., anti-PD-L1 antibody (or antigen-binding fragment thereof), or at least 2, 3, 4, 5, 6, 7, 8, 9, 10, 11, or 12 months or at least 2 or 3 years following such administration.

In some aspects, the receptor, e.g. CAR, expressed by the cells, is detectable by quantitative PCR (qPCR) or by flow cytometry in the subject, plasma, serum, blood, tissue and/or disease site thereof, e.g., tumor site, at a time that is at least about 3 months, at least about 6 months, at least about 12 months, at least about 1 year, at least about 2 years, at least about 3 years, or more than 3 years, following the administration of the cells, e.g., following the initiation of the administration of the T cells, e.g., CAR-expressing T cells, and/or the checkpoint inhibitor, e.g., anti-PD-L1 antibody (or antigen-binding fragment thereof).

In some embodiments, the area under the curve (AUC) for concentration of receptor- (e.g., CAR-) expressing cells in a fluid, plasma, serum, blood, tissue, organ and/or disease site, e.g. tumor site, of the subject over time following the administration of the T cells, e.g., CAR-expressing T cells and/a checkpoint inhibitor, e.g., anti-PD-L1 antibody (or antigen-binding fragment thereof), is greater as compared to that achieved via an alternative dosing regimen where the subject is administered the T cells, e.g., CAR-expressing T cells, in the absence of administering the checkpoint inhibitor, e.g., anti-PD-L1 antibody (or antigen-binding fragment thereof).

In some aspects, the method results in high in vivo proliferation of the administered cells, for example, as measured by flow cytometry. In some aspects, high peak proportions of the cells are detected. For example, in some embodiments, at a peak or maximum level following the T cells, e.g., CAR-expressing T cells and/a checkpoint inhibitor, e.g., anti-PD-L1 antibody (or antigen-binding fragment thereof), in the blood, plasma, serum, tissue or disease site of the subject or white blood cell fraction thereof, e.g., PBMC fraction or T cell fraction, at least about 10%, at least about 20%, at least about 30%, at least about 40%, at least about 50%, at least about 60%, at least about 70%, at least about 80%, or at least about 90% of the cells express the recombinant receptor, e.g., the CAR.

In some aspects, the increased or prolonged expansion and/or persistence of the dose of cells in the subject administered with the checkpoint inhibitor, e.g., anti-PD-L1 antibody (or antigen-binding fragment thereof) is associated with a benefit in tumor related outcomes in the subject. In some embodiments, the tumor related outcome includes a decrease in tumor burden or a decrease in blast marrow in the subject. In some embodiments, the tumor burden is decreased by or by at least at or about 10, 20, 30, 40, 50, 60, 70, 80, 90, or 100 percent after administration of the method. In some embodiments, disease burden, tumor size, tumor volume, tumor mass, and/or tumor load or bulk is reduced following the dose of cells by at least at or about 50%, 60%, 70%, 80%, 90% or more compared a subject that has been treated with a method that does not involve the administration of a checkpoint inhibitor, e.g., an anti-PD-L1 antibody (or antigen-binding fragment thereof).

B. T Cell Functional Activity

In some embodiments, parameters associated with therapy or a treatment outcome, which include parameters that can be assessed for the screening steps and/or assessment of treatment of outcomes and/or monitoring treatment outcomes, includes one or more of activity, phenotype, proliferation or function of T cells. In some embodiments, any of the known assays in the art for assessing the activity, phenotypes, proliferation and/or function of the T cells, e.g., T cells administered for T cell therapy, can be used. Prior to and/or subsequent to administration of the cells and/a checkpoint inhibitor, e.g., anti-PD-L1 antibody (or antigen-binding fragment thereof), the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al., J. Immunological Methods, 285(1): 25-40 (2004). In certain embodiments, the biological activity of the cells is measured by assaying expression and/or secretion of one or more cytokines, such as CD107a, IFNγ, IL-2, GM-CSF and TNFα, and/or by assessing cytolytic activity.

In some embodiments, assays for the activity, phenotypes, proliferation and/or function of the T cells, e.g., T cells administered for T cell therapy include, but are not limited to, ELISPOT, ELISA, cellular proliferation, cytotoxic lymphocyte (CTL) assay, binding to the T cell epitope, antigen or ligand, or intracellular cytokine staining, proliferation assays, lymphokine secretion assays, direct cytotoxicity assays, and limiting dilution assays. In some embodiments, proliferative responses of the T cells can be measured, e.g. by incorporation of ³H-thymidine, BrdU (5-Bromo-2′-Deoxyuridine) or 2′-deoxy-5-ethynyluridine (Edu) into their DNA or dye dilution assays, using dyes such as carboxyfluorescein diacetate succinimidyl ester (CFSE), CellTrace Violet, or membrane dye PKH26.

In some embodiments, assessing the activity, phenotypes, proliferation and/or function of the T cells, e.g., T cells administered for T cell therapy, include measuring cytokine production from T cells, and/or measuring cytokine production in a biological sample from the subject, e.g., plasma, serum, blood, and/or tissue samples, e.g., tumor samples. In some cases, such measured cytokines can include, without limitation, interlekukin-2 (IL-2), interferon-gamma (IFNγ), interleukin-4 (IL-4), TNF-alpha (TNFα), interleukin-6 (IL-6), interleukin-10 (IL-10), interleukin-12 (IL-12), granulocyte-macrophage colony-stimulating factor (GM-CSF), CD107a, and/or THF-beta (TGFβ). Assays to measure cytokines are well known in the art, and include but are not limited to, ELISA, intracellular cytokine staining, cytometric bead array, RT-PCR, ELISPOT, flow cytometry and bio-assays in which cells responsive to the relevant cytokine are tested for responsiveness (e.g. proliferation) in the presence of a test sample.

In some embodiments, assessing the activity, phenotypes, proliferation and/or function of the T cells, e.g., T cells administered for T cell therapy, include assessing cell phenotypes, e.g., expression of particular cell surface markers. In some embodiments, the T cells, e.g., T cells administered for T cell therapy, are assessed for expression of T cell activation markers, T cell exhaustion markers, and/or T cell differentiation markers. In some embodiments, the cell phenotype is assessed before administration. In some embodiments, the cell phenotype is assessed during, or after administration of cell therapy and/or the checkpoint inhibitor, e.g., anti-PD-L1 antibody (or antigen-binding fragment thereof). T cell activation markers, T cell exhaustion markers, and/or T cell differentiation markers for assessment include any markers known in the art for particular subsets of T cells, e.g., CD25, CD38, human leukocyte antigen-DR (HLA-DR), CD69, CD44, CD137, KLRG1, CD62L^{low}, CCR7^{low}, CD71, CD2, CD54, CD58, CD244, CD160, programmed cell death protein 1 (PD-1), lymphocyte activation gene 3 protein (LAG-3), T-cell immunoglobulin domain and mucin domain protein 3 (TIM-3), cytotoxic T lymphocyte antigen-4 (CTLA-4), band T lymphocyte attenuator (BTLA) and/or T-cell immunoglobulin and immunoreceptor tyrosine-based inhibitory motif domain (TIGIT) (see, e.g., Liu et al., Cell Death and Disease (2015) 6, e1792). In some embodiments, the assessed cell surface marker is CD25, PD-1 and/or TIM-3. In some embodiments, the assessed cell surface marker is CD25.

In some aspects, detecting the expression levels includes performing an in vitro assay. In some embodiments, the in vitro assay is an immunoassay, an aptamer-based assay, a histological or cytological assay, or an mRNA expression level assay. In some embodiments, the parameter or parameters for one or more of each of the one or more factors, effectors, enzymes and/or surface markers are detected by an enzyme linked immunosorbent assay (ELISA), immunoblotting, immunoprecipitation, radioimmunoassay (RIA), immuno staining, flow cytometry assay, surface plasmon resonance (SPR), chemiluminescence assay, lateral flow immunoassay, inhibition assay or avidity assay. In some embodiments, detection of cytokines and/or surface markers is determined using a binding reagent that specifically binds to at least one biomarker. In some cases, the binding reagent is an antibody or antigen-binding fragment thereof, an aptamer or a nucleic acid probe.

In some embodiments, the administration of the checkpoint inhibitor, e.g., anti-PD-L1 antibody (or antigen-binding fragment thereof) increases the level of circulating CAR T cells.

C. Response, Efficacy and Survival

In some embodiments, parameters associated with therapy or a treatment outcome, which include parameters that can be assessed for the screening steps and/or assessment of treatment of outcomes and/or monitoring treatment outcomes, includes tumor or disease burden. The administration of the immunotherapy, such as a T cell therapy (e.g. CAR-expressing T cells) and/or the checkpoint inhibitor, e.g., anti-PD-L1 antibody (or antigen-binding fragment thereof), can reduce or prevent the expansion or burden of the disease or condition in the subject. For example, where the disease or condition is a tumor, the methods generally reduce tumor size, bulk, metastasis, percentage of blasts in the bone marrow or molecularly detectable cancer and/or improve prognosis or survival or other symptom associated with tumor burden.

In some aspects, the administration in accord with the provided methods, and/or with the provided articles of manufacture or compositions, generally reduces or prevents the expansion or burden of the disease or condition in the subject. For example, where the disease or condition is a tumor, the methods generally reduce tumor size, bulk, metastasis, percentage of blasts in the bone marrow or molecularly detectable cancer and/or improve prognosis or survival or other symptom associated with tumor burden.

In some embodiments, the provided methods result in a decreased tumor burden in treated subjects compared to alternative methods in which the immunotherapy, such as a T cell therapy (e.g. CAR-expressing T cells) is given without administration of the checkpoint inhibitor, e.g., anti-PD-L1 antibody (or antigen-binding fragment thereof). It is not necessary that the tumor burden actually be reduced in all subjects receiving the combination therapy, but that tumor burden is reduced on average in subjects treated, such as based on clinical data, in which a majority of subjects treated with such a combination therapy exhibit a reduced tumor burden, such as at least 50%, 60%, 70%, 80%, 90%, 95% or more of subjects treated with the combination therapy, exhibit a reduced tumor burden.

Disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood, lymph or bone marrow in the context of certain hematological malignancies. Disease burden can include, in some embodiments, the mass of a tumor, the number or extent of metastases and/or the percentage of blast cells present in the bone marrow.

In some embodiments, the subject has a myeloma, a lymphoma or a leukemia. The extent of disease burden can be determined by assessment of residual leukemia in blood or bone marrow. In some embodiments, the subject has a non-Hodgkin lymphoma (NHL), an acute lymphoblastic leukemia (ALL), a chronic lymphocytic leukemia (CLL), a diffuse large B-cell lymphoma (DLBCL) or a myeloma, e.g., a multiple myeloma (MM). In some embodiments, the subject has a MM or a DBCBL.

In some aspects, response rates in subjects, such as subjects with NHL, are based on the Lugano criteria. (Cheson et al., (2014) JCO., 32(27):3059-3067; Johnson et al., (2015) Radiology 2:323-338; Cheson, B. D. (2015) Chin. Clin. Oncol. 4(1):5). In some aspects, response assessment utilizes any of clinical, hematologic, and/or molecular methods. In some aspects, response assessed using the Lugano criteria involves the use of positron emission tomography (PET)-computed tomography (CT) and/or CT as appropriate. PET-CT evaluations may further comprise the use of fluorodeoxyglucose (FDG) for FDG-avid lymphomas. In some aspects, where PET-CT will be used to assess response in FDG-avid histologies, a 5-point scale may be used. In some respects, the 5-point scale comprises the following criteria: 1, no uptake above background; 2, uptake ≤mediastinum; 3, uptake >mediastinum but ≤liver; 4, uptake moderately >liver; 5, uptake markedly higher than liver and/or new lesions; X, new areas of uptake unlikely to be related to lymphoma.

In some aspects, a complete response as described using the Lugano criteria involves a complete metabolic response and a complete radiologic response at various measureable sites. In some aspects, these sites include lymph nodes and extralymphatic sites, wherein a CR is described as a score of 1, 2, or 3 with or without a residual mass on the 5-point scale, when PET-CT is used. In some aspects, in Waldeyer's ring or extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake. In some aspects, response is assessed in the lymph nodes using CT, wherein a CR is described as no extralymphatic sites of disease and target nodes/nodal masses must regress to ≤1.5 cm in longest transverse diameter of a lesion (LDi). Further sites of assessment include the bone marrow wherein PET-CT-based assessment should indicate a lack of evidence of FDG-avid disease in marrow and a CT-based assessment should indicate a normal morphology, which if indeterminate should be IHC negative. Further sites may include assessment of organ enlargement, which should regress to normal. In some aspects, nonmeasured lesions and new lesions are assessed, which in the case of CR should be absent (Cheson et al., (2014) JCO., 32(27):3059-3067; Johnson et al., (2015) Radiology 2:323-338; Cheson, B. D. (2015) Chin. Clin. Oncol. 4(1):5).

In some aspects, a partial response (PR) as described using the Lugano criteria involves a partial metabolic and/or radiological response at various measureable sites. In some aspects, these sites include lymph nodes and extralymphatic sites, wherein a PR is described as a score of 4 or 5 with reduced uptake compared with baseline and residual mass(es) of any size, when PET-CT is used. At interim, such findings can indicate responding disease. At the end of treatment, such findings can indicate residual disease. In some aspects, response is assessed in the lymph nodes using CT, wherein a PR is described as ≥50% decrease in SPD of up to 6 target measureable nodes and extranodal sites. If a lesion is too small to measure on CT, 5 mm×5 mm is assigned as the default value; if the lesion is no longer visible, the value is 0 mm×0 mm; for a node >5 mm×5 mm, but smaller than normal, actual measurements are used for calculation. Further sites of assessment include the bone marrow wherein PET-CT-based assessment should indicate residual uptake higher than uptake in normal marrow but reduced compared with baseline (diffuse uptake compatible with reactive changes from chemotherapy allowed). In some aspects, if there are persistent focal changes in the marrow in the context of a nodal response, consideration should be given to further evaluation with MRI or biopsy, or an interval scan. In some aspects, further sites may include assessment of organ enlargement, where the spleen must have regressed by >50% in length beyond normal. In some aspects, nonmeasured lesions and new lesions are assessed, which in the case of PR should be absent/normal, regressed, but no increase. No response/stable disease (SD) or progressive disease (PD) can also be measured using PET-CT and/or CT based assessments. (Cheson et al., (2014) JCO., 32(27):3059-3067; Johnson et al., (2015) Radiology 2:323-338; Cheson, B. D. (2015) Chin. Clin. Oncol., 4(1):5).

In some respects, progression-free survival (PFS) is described as the length of time during and after the treatment of a disease, such as cancer, that a subject lives with the disease but it does not get worse. In some aspects, objective response (OR) is described as a measurable response. In some aspects, objective response rate (ORR) is described as the proportion of patients who achieved CR or PR. In some aspects, overall survival (OS) is described as the length of time from either the date of diagnosis or the start of treatment for a disease, such as cancer, that subjects diagnosed with the disease are still alive. In some aspects, event-free survival (EFS) is described as the length of time after treatment for a cancer ends that the subject remains free of certain complications or events that the treatment was intended to prevent or delay. These events may include the return of the cancer or the onset of certain symptoms, such as bone pain from cancer that has spread to the bone, or death.

In some embodiments, the measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response is durable for greater than 3 months or greater than 6 months.

In some aspects, the RECIST criteria is used to determine objective tumor response. (Eisenhauer et al., European Journal of Cancer 45 (2009) 228-247.) In some aspects, the RECIST criteria is used to determine objective tumor response for target lesions. In some respects, a complete response as determined using RECIST criteria is described as the disappearance of all target lesions and any pathological lymph nodes (whether target or non-target) must have reduction in short axis to <10 mm. In other aspects, a partial response as determined using RECIST criteria is described as at least a 30% decrease in the sum of diameters of target lesions, taking as reference the baseline sum diameters. In other aspects, progressive disease (PD) is described as at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm (in some aspects the appearance of one or more new lesions is also considered progression). In other aspects, stable disease (SD) is described as neither sufficient shrinkage to qualify for PR nor sufficient increase to qualify for PD, taking as reference the smallest sum diameters while on study.

In the case of MM, exemplary parameters to assess the extent of disease burden include such parameters as number of clonal plasma cells (e.g., >10% on bone marrow biopsy or in any quantity in a biopsy from other tissues; plasmacytoma), presence of monoclonal protein (paraprotein) in either serum or urine, evidence of end-organ damage felt related to the plasma cell disorder (e.g., hypercalcemia (corrected calcium >2.75 mmol/1); renal insufficiency attributable to myeloma; anemia (hemoglobin <10 g/dl); and/or bone lesions (lytic lesions or osteoporosis with compression fractures)).

In the case of DLBCL, exemplary parameters to assess the extent of disease burden include such parameters as cellular morphology (e.g., centroblastic, immunoblastic, and anaplastic cells), gene expression, miRNA expression and protein expression (e.g., expression of BCL2, BCL6, MUM1, LMO2, MYC, and p21).

In some aspects, response rates in subjects, such as subjects with CLL, are based on the International Workshop on Chronic Lymphocytic Leukemia (IWCLL) response criteria (Hallek, et al., Blood 2008, June 15; 111(12): 5446-5456). In some aspects, these criteria are described as follows: complete remission (CR), which in some aspects requires the absence of peripheral blood clonal lymphocytes by immunophenotyping, absence of lymphadenopathy, absence of hepatomegaly or splenomegaly, absence of constitutional symptoms and satisfactory blood counts; complete remission with incomplete marrow recovery (CRi), which in some aspects is described as CR above, but without normal blood counts; partial remission (PR), which in some aspects is described as ≥50% fall in lymphocyte count, ≥50% reduction in lymphadenopathy or ≥50% reduction in liver or spleen, together with improvement in peripheral blood counts; progressive disease (PD), which in some aspects is described as ≥50% rise in lymphocyte count to ≥5×10⁹/L, ≥50% increase in lymphadenopathy, ≥50% increase in liver or spleen size, Richter's transformation, or new cytopenias due to CLL; and stable disease, which in some aspects is described as not meeting criteria for CR, CRi, PR or PD.

In some embodiments, the subjects exhibits a CR or OR if, within 1 month of the administration of the dose of cells, lymph nodes in the subject are less than at or about 20 mm in size, less than at or about 10 mm in size or less than at or about 10 mm in size.

In some embodiments, an index clone of the CLL is not detected in the bone marrow of the subject (or in the bone marrow of greater than 50%, 60%, 70%, 80%, 90% or more of the subjects treated according to the methods. In some embodiments, an index clone of the CLL is assessed by IgH deep sequencing. In some embodiments, the index clone is not detected at a time that is at or about or at least at or about 1, 2, 3, 4, 5, 6, 12, 18 or 24 months following the administration of the cells.

In some embodiments, a subject exhibits morphologic disease if there are greater than or equal to 5% blasts in the bone marrow, for example, as detected by light microscopy, such as greater than or equal to 10% blasts in the bone marrow, greater than or equal to 20% blasts in the bone marrow, greater than or equal to 30% blasts in the bone marrow, greater than or equal to 40% blasts in the bone marrow or greater than or equal to 50% blasts in the bone marrow. In some embodiments, a subject exhibits complete or clinical remission if there are less than 5% blasts in the bone marrow.

In some embodiments, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable cancer. In some embodiments, molecularly detectable cancer can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify cancer cell based on leukemia-specific immunophenotypes. In some embodiments, molecular detection of cancer can detect as few as 1 leukemia cell in 100,000 normal cells. In some embodiments, a subject exhibits MRD that is molecularly detectable if at least or greater than 1 leukemia cell in 100,000 cells is detected, such as by PCR or flow cytometry. In some embodiments, the disease burden of a subject is molecularly undetectable or MRD⁻, such that, in some cases, no leukemia cells are able to be detected in the subject using PCR or flow cytometry techniques.

In the case of leukemia, the extent of disease burden can be determined by assessment of residual leukemia in blood or bone marrow. In some embodiments, a subject exhibits morphologic disease if there are greater than or equal to 5% blasts in the bone marrow, for example, as detected by light microscopy. In some embodiments, a subject exhibits complete or clinical remission if there are less than 5% blasts in the bone marrow.

In some embodiments, for leukemia, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable cancer. In some embodiments, molecularly detectable cancer can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify cancer cell based on leukemia-specific immunophenotypes. In some embodiments, molecular detection of cancer can detect as few as 1 leukemia cell in 100,000 normal cells. In some embodiments, a subject exhibits MRD that is molecularly detectable if at least or greater than 1 leukemia cell in 100,000 cells is detected, such as by PCR or flow cytometry. In some embodiments, the disease burden of a subject is molecularly undetectable or MRD⁻, such that, in some cases, no leukemia cells are able to be detected in the subject using PCR or flow cytometry techniques.

In some embodiments, the methods and/or administration of a cell therapy, such as a T cell therapy (e.g. CAR-expressing T cells) and/a checkpoint inhibitor, e.g., anti-PD-L1 antibody (or antigen-binding fragment thereof) decrease(s) disease burden as compared with disease burden at a time immediately prior to the administration of the immunotherapy, e.g., T cell therapy and/a checkpoint inhibitor, e.g., anti-PD-L1 antibody (or antigen-binding fragment thereof).

In some aspects, administration of the immunotherapy, e.g. T cell therapy and/a checkpoint inhibitor, e.g., anti-PD-L1 antibody (or antigen-binding fragment thereof), may prevent an increase in disease burden, and this may be evidenced by no change in disease burden.

In some embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative therapy, such as one in which the subject receives immunotherapy, e.g. T cell therapy alone, in the absence of administration of the checkpoint inhibitor, e.g., anti-PD-L1 antibody (or antigen-binding fragment thereof). In some embodiments, disease burden is reduced to a greater extent or for a greater duration following the combination therapy of administration of the immunotherapy, e.g., T cell therapy, and the checkpoint inhibitor, e.g., anti-PD-L1 antibody (or antigen-binding fragment thereof), compared to the reduction that would be effected by administering each of the agent alone, e.g., administering the checkpoint inhibitor, e.g., anti-PD-L1 antibody (or antigen-binding fragment thereof) to a subject having not received the immunotherapy, e.g. T cell therapy; or administering the immunotherapy, e.g. T cell therapy, to a subject having not received the checkpoint inhibitor, e.g., anti-PD-L1 antibody (or antigen-binding fragment thereof).

In some embodiments, the burden of a disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some embodiments, disease burden, e.g. tumor burden, is assessed by measuring the number or extent of metastases. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified. In some embodiments, exemplary parameters for determination include particular clinical outcomes indicative of amelioration or improvement in the disease or condition, e.g., tumor. Such parameters include: duration of disease control, including complete response (CR), partial response (PR) or stable disease (SD) (see, e.g., Response Evaluation Criteria In Solid Tumors (RECIST) guidelines), objective response rate (ORR), progression-free survival (PFS) and overall survival (OS). Specific thresholds for the parameters can be set to determine the efficacy of the method of combination therapy provided herein.

In some aspects, disease burden is measured or detected prior to administration of the immunotherapy, e.g. T cell therapy, following the administration of the immunotherapy, e.g. T cell therapy but prior to administration of the checkpoint inhibitor, e.g., anti-PD-L1 antibody (or antigen-binding fragment thereof), and/or following the administration of both the immunotherapy, e.g. T cell therapy and the checkpoint inhibitor, e.g., anti-PD-L1 antibody (or antigen-binding fragment thereof). In the context of multiple administration of one or more steps of the combination therapy, disease burden in some embodiments may be measured prior to, or following administration of any of the steps, doses and/or cycles of administration, or at a time between administration of any of the steps, doses and/or cycles of administration. In some embodiments, the administration of the checkpoint inhibitor, e.g., anti-PD-L1 antibody (or antigen-binding fragment thereof) is carried out at least two cycles (e.g., 28-day cycle), and disease burden is measured or detected prior to, during, and/or after each cycle.

In some embodiments, the burden is decreased by or by at least at or about 10, 20, 30, 40, 50, 60, 70, 80, 90, or 100 percent by the provided methods compared to immediately prior to the administration of the checkpoint inhibitor, e.g., anti-PD-L1 antibody (or antigen-binding fragment thereof) and the immunotherapy, e.g. T cell therapy. In some embodiments, disease burden, tumor size, tumor volume, tumor mass, and/or tumor load or bulk is reduced following administration of the immunotherapy, e.g. T cell therapy and the checkpoint inhibitor, e.g., anti-PD-L1 antibody (or antigen-binding fragment thereof), by at least at or about 10, 20, 30, 40, 50, 60, 70, 80, 90% or more compared to that immediately prior to the administration of the immunotherapy, e.g. T cell therapy and/or the checkpoint inhibitor, e.g., anti-PD-L1 antibody (or antigen-binding fragment thereof).

In some embodiments, reduction of disease burden by the method comprises an induction in morphologic complete remission, for example, as assessed at 1 month, 2 months, 3 months, 4 months, 5 months, 6 months, or more than 6 months, after administration of, e.g., initiation of, the combination therapy.

In some aspects, an assay for minimal residual disease, for example, as measured by multiparametric flow cytometry, is negative, or the level of minimal residual disease is less than about 0.3%, less than about 0.2%, less than about 0.1%, or less than about 0.05%.

In some embodiments, the event-free survival rate or overall survival rate of the subject is improved by the methods, as compared with other methods. For example, in some embodiments, event-free survival rate or probability for subjects treated by the methods at 6 months following the method of combination therapy provided herein, is greater than about 40%, greater than about 50%, greater than about 60%, greater than about 70%, greater than about 80%, greater than about 90%, or greater than about 95%. In some aspects, overall survival rate is greater than about 40%, greater than about 50%, greater than about 60%, greater than about 70%, greater than about 80%, greater than about 90%, or greater than about 95%. In some embodiments, the subject treated with the methods exhibits event-free survival, relapse-free survival, or survival to at least 6 months, or at least 1, 2, 3, 4, 5, 6, 7, 8, 9, or 10 years. In some embodiments, the time to progression is improved, such as a time to progression of greater than at or about 6 months, or at least 1, 2, 3, 4, 5, 6, 7, 8, 9, or 10 years.

In some embodiments, following treatment by the method, the probability of relapse is reduced as compared to other methods. For example, in some embodiments, the probability of relapse at 6 months following the method of combination therapy, is less than about 80%, less than about 70%, less than about 60%, less than about 50%, less than about 40%, less than about 30%, less than about 20%, or less than about 10%.

In some cases, the pharmacokinetics of administered cells, e.g., adoptively transferred cells are determined to assess the availability, e.g., bioavailability of the administered cells. Methods for determining the pharmacokinetics of adoptively transferred cells may include drawing peripheral blood from subjects that have been administered engineered cells, and determining the number or ratio of the engineered cells in the peripheral blood. Approaches for selecting and/or isolating cells may include use of chimeric antigen receptor (CAR)-specific antibodies (e.g., Brentjens et al., Sci. Transl. Med. 2013 March; 5(177): 177ra38) Protein L (Zheng et al., J. Transl. Med. 2012 February; 10:29), epitope tags, such as Strep-Tag sequences, introduced directly into specific sites in the CAR, whereby binding reagents for Strep-Tag are used to directly assess the CAR (Liu et al. (2016) Nature Biotechnology, 34:430; international patent application Pub. No. WO2015095895) and monoclonal antibodies that specifically bind to a CAR polypeptide (see international patent application Pub. No. WO2014190273). Extrinsic marker genes may in some cases be utilized in connection with engineered cell therapies to permit detection or selection of cells and, in some cases, also to promote cell suicide. A truncated epidermal growth factor receptor (EGFRt) in some cases can be co-expressed with a transgene of interest (a CAR or TCR) in transduced cells (see e.g. U.S. Pat. No. 8,802,374). EGFRt may contain an epitope recognized by the antibody cetuximab (Erbitux®) or other therapeutic anti-EGFR antibody or binding molecule, which can be used to identify or select cells that have been engineered with the EGFRt construct and another recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434).

In some embodiments, the number of CAR+ T cells in a biological sample obtained from the patient, e.g., blood, can be determined at a period of time after administration of the cell therapy, e.g., to determine the pharmacokinetics of the cells. In some embodiments, number of CAR+ T cells, optionally CAR+CD8+ T cells and/or CAR+CD4+ T cells, detectable in the blood of the subject, or in a majority of subjects so treated by the method, is greater than 1 cells per μL, greater than 5 cells per μL or greater than per 10 cells per μL.

### IV. TOXICITY

In embodiments of the provided methods, the subject is monitored for toxicity, e.g., development of cytokine release syndrome (CRS) or neurotoxicity (NT), in subjects administered a cell therapy (e.g., a T cell therapy). In some embodiments, the provided methods are carried out to reduce the risk of a toxic outcome or symptom, toxicity-promoting profile, factor, or property, such as a symptom or outcome associated with or indicative of severe cytokine release syndrome (CRS) or severe neurotoxicity.

In some embodiments, the provided methods do not result in a high rate or likelihood of toxicity or toxic outcomes, or reduces the rate or likelihood of toxicity or toxic outcomes, such as severe neurotoxicity (NT) or severe cytokine release syndrome (CRS), such as compared to certain other cell therapies. In some embodiments, the methods do not result in, or do not increase the risk of, severe NT (sNT), severe CRS (sCRS), macrophage activation syndrome, tumor lysis syndrome, fever of at least at or about 38 degrees Celsius for three or more days and a plasma level of CRP of at least at or about 20 mg/dL. In some embodiments, greater than or greater than about 30%, 35%, 40%, 50%, 55%, 60% or more of the subjects treated according to the provided methods do not exhibit any grade of CRS or any grade of neurotoxcity. In some embodiments, no more than 50% of subjects treated (e.g. at least 60%, at least 70%, at least 80%, at least 90% or more of the subjects treated) exhibit a cytokine release syndrome (CRS) higher than grade 2 and/or a neurotoxicity higher than grade 2. In some embodiments, at least 50% of subjects treated according to the method (e.g. at least 60%, at least 70%, at least 80%, at least 90% or more of the subjects treated) do not exhibit a severe toxic outcome (e.g. severe CRS or severe neurotoxicity), such as do not exhibit grade 3 or higher neurotoxicity and/or does not exhibit severe CRS, or does not do so within a certain period of time following the treatment, such as within a week, two weeks, or one month of the administration of the cells. In some embodiments, parameters assessed to determine certain toxicities include adverse events (AEs), dose-limiting toxicities (DLTs), CRS and NT.

In some aspects, the toxic outcome is or is associated with or indicative of cytokine release syndrome (CRS) or severe CRS (sCRS). CRS, e.g., sCRS, can occur in some cases following adoptive T cell therapy and administration to subjects of other biological products. See Davila et al., Sci Transl Med 6, 224ra25 (2014); Brentjens et al., Sci. Transl. Med. 5, 177ra38 (2013); Grupp et al., N. Engl. J. Med. 368, 1509-1518 (2013); and Kochenderfer et al., Blood 119, 2709-2720 (2012); Xu et al., Cancer Letters 343 (2014) 172-78.

Typically, CRS is caused by an exaggerated systemic immune response mediated by, for example, T cells, B cells, NK cells, monocytes, and/or macrophages. Such cells may release a large amount of inflammatory mediators such as cytokines and chemokines. Cytokines may trigger an acute inflammatory response and/or induce endothelial organ damage, which may result in microvascular leakage, heart failure, or death. Severe, life-threatening CRS can lead to pulmonary infiltration and lung injury, renal failure, or disseminated intravascular coagulation. Other severe, life-threatening toxicities can include cardiac toxicity, respiratory distress, neurologic toxicity and/or hepatic failure. CRS may be treated using anti-inflammatory therapy such as an anti-IL-6 therapy, e.g., anti-IL-6 antibody, e.g., tocilizumab, or antibiotics or other agents as described.

Outcomes, signs and symptoms of CRS are known and include those described herein. In some embodiments, where a particular dosage regimen or administration effects or does not effect a given CRS-associated outcome, sign, or symptom, particular outcomes, signs, and symptoms and/or quantities or degrees thereof may be specified.

In the context of administering CAR-expressing cells, CRS typically occurs 6-20 days after infusion of cells that express a CAR. See Xu et al., Cancer Letters 343 (2014) 172-78. In some cases, CRS occurs less than 6 days or more than 20 days after CAR T cell infusion. The incidence and timing of CRS may be related to baseline cytokine levels or tumor burden at the time of infusion. Commonly, CRS involves elevated serum levels of interferon (IFN)-γ, tumor necrosis factor (TNF)-α, and/or interleukin (IL)-2. Other cytokines that may be rapidly induced in CRS are IL-1β, 1L-6, IL-8, and IL-10.

Exemplary outcomes associated with CRS include fever, rigors, chills, hypotension, dyspnea, acute respiratory distress syndrome (ARDS), encephalopathy, ALT/AST elevation, renal failure, cardiac disorders, hypoxia, neurologic disturbances, and death. Neurological complications include delirium, seizure-like activity, confusion, word-finding difficulty, aphasia, and/or becoming obtunded. Other CRS-related outcomes include fatigue, nausea, headache, seizure, tachycardia, myalgias, rash, acute vascular leak syndrome, liver function impairment, and renal failure. In some aspects, CRS is associated with an increase in one or more factors such as serum-ferritin, d-dimer, aminotransferases, lactate dehydrogenase and triglycerides, or with hypofibrinogenemia or hepatosplenomegaly.

In some embodiments, outcomes associated with CRS include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures).

Exemplary CRS-related outcomes include increased or high serum levels of one or more factors, including cytokines and chemokines and other factors associated with CRS. Exemplary outcomes further include increases in synthesis or secretion of one or more of such factors. Such synthesis or secretion can be by the T cell or a cell that interacts with the T cell, such as an innate immune cell or B cell.

In some embodiments, the CRS-associated serum factors or CRS-related outcomes include inflammatory cytokines and/or chemokines, including interferon gamma (IFN-γ), TNF-a, IL-1β, IL-2, IL-6, IL-7, IL-8, IL-10, IL-12, sIL-2Ra, granulocyte macrophage colony stimulating factor (GM-CSF), macrophage inflammatory protein (MIP)-1, tumor necrosis factor alpha (TNFα), IL-6, and IL-10, IL-1β, IL-8, IL-2, MIP-1, Flt-3L, fracktalkine, and/or IL-5. In some embodiments, the factor or outcome includes C reactive protein (CRP). In addition to being an early and easily measurable risk factor for CRS, CRP also is a marker for cell expansion. In some embodiments, subjects that are measured to have high levels of CRP, such as ≥15 mg/dL, have CRS. In some embodiments, subjects that are measured to have high levels of CRP do not have CRS. In some embodiments, a measure of CRS includes a measure of CRP and another factor indicative of CRS.

In some embodiments, one or more inflammatory cytokines or chemokines are monitored before, during, or after CAR treatment. In some aspects, the one or more cytokines or chemokines include IFN-γ, TNF-α, IL-2, IL-1β, IL-6, IL-7, IL-8, IL-10, IL-12, sIL-2Rα, granulocyte macrophage colony stimulating factor (GM-CSF), or macrophage inflammatory protein (MIP). In some embodiments, IFN-γ, TNF-α, and IL-6 are monitored.

CRS criteria that appear to correlate with the onset of CRS to predict which patients are more likely to be at risk for developing sCRS have been developed (see Davilla et al. Science translational medicine. 2014; 6(224):224ra25). Factors include fevers, hypoxia, hypotension, neurologic changes, elevated serum levels of inflammatory cytokines, such as a set of seven cytokines (IFNγ, IL-5, IL-6, IL-10, Flt-3L, fractalkine, and GM-CSF) whose treatment-induced elevation can correlate well with both pretreatment tumor burden and sCRS symptoms. Other guidelines on the diagnosis and management of CRS are known (see e.g., Lee et al, Blood. 2014; 124(2):188-95). In some embodiments, the criteria reflective of CRS grade are those detailed in Table 3 below.

TABLE 3
Exemplary Grading Criteria for CRS

| Grade | Description of Symptoms |
|---|---|
| 1 | Not life-threatening, require only symptomatic treatment |
| Mild | such as antipyretics and anti-emetics (e.g., fever, nausea, |
| | fatigue, headache, myalgias, malaise) |
| 2 | Require and respond to moderate intervention: |
| Moderate | Oxygen requirement ≥40%, or |
| | Hypotension responsive to fluids or low dose of a |
| | single vasopressor, or |
| | Grade 2 organ toxicity (by CTCAE v4.0) |
| 3 | Require and respond to aggressive intervention: |
| Severe | Oxygen requirement ≥40%, or |
| | Hypotension requiring high dose of a single |
| | vasopressor (e.g., |
| | norepinephrine ≥20 μg/kg/min, dopamine ≥10 μg/kg/min, |
| | phenylephrine ≥200 μg/kg/min, or |
| | epinephrine ≥10 μg/kg/min), or |
| | Hypotension requiring multiple vasopressors (e.g., |
| | vasopressin + one of the above agents, or combination |
| | vasopressors equivalent to ≥20 μg/kg/min |
| | norepinephrine), or |
| | Grade 3 organ toxicity or Grade 4 transaminitis |
| | (by CTCAE v4.0) |
| 4 | Life-threatening: |
| Life- | Requirement for ventilator support, or |
| threatening | Grade 4 organ toxicity (excluding transaminitis) |
| 5 | Death |
| Fatal | |

In some embodiments, a subject is deemed to develop “severe CRS” (“sCRS”) in response to or secondary to administration of a cell therapy or dose of cells thereof, if, following administration, the subject displays: (1) fever of at least 38 degrees Celsius for at least three days; (2) cytokine elevation that includes either (a) a max fold change of at least 75 for at least two of the following group of seven cytokines compared to the level immediately following the administration: interferon gamma (IFNγ), GM-CSF, IL-6, IL-10, Flt-3L, fracktalkine, and IL-5 and/or (b) a max fold change of at least 250 for at least one of the following group of seven cytokines compared to the level immediately following the administration: interferon gamma (IFNγ), GM-CSF, IL-6, IL-10, Flt-3L, fracktalkine, and IL-5; and (c) at least one clinical sign of toxicity such as hypotension (requiring at least one intravenous vasoactive pressor) or hypoxia (PO₂<90%) or one or more neurologic disorder(s) (including mental status changes, obtundation, and/or seizures). In some embodiments, severe CRS includes CRS with a grade of 3 or greater, such as set forth in Table 3.

In some embodiments, outcomes associated with severe CRS or grade 3 CRS or greater, such as grade 4 or greater, include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures). In some embodiments, severe CRS includes CRS that requires management or care in the intensive care unit (ICU).

In some embodiments, the CRS, such as severe CRS, encompasses a combination of (1) persistent fever (fever of at least 38 degrees Celsius for at least three days) and (2) a serum level of CRP of at least at or about 20 mg/dL. In some embodiments, the CRS encompasses hypotension requiring the use of two or more vasopressors or respiratory failure requiring mechanical ventilation. In some embodiments, the dosage of vasopressors is increased in a second or subsequent administration.

In some embodiments, severe CRS or grade 3 CRS encompasses an increase in alanine aminotransferase, an increase in aspartate aminotransferase, chills, febrile neutropenia, headache, left ventricular dysfunction, encephalopathy, hydrocephalus, and/or tremor.

The method of measuring or detecting the various outcomes may be specified.

In some aspects, the toxic outcome of a therapy, such as a cell therapy, is or is associated with or indicative of neurotoxicity or severe neurotoxicity. In some embodiments, symptoms associated with a clinical risk of neurotoxicity include confusion, delirium, expressive aphasia, obtundation, myoclonus, lethargy, altered mental status, convulsions, seizure-like activity, seizures (optionally as confirmed by electroencephalogram [EEG]), elevated levels of beta amyloid (Aβ), elevated levels of glutamate, and elevated levels of oxygen radicals. In some embodiments, neurotoxicity is graded based on severity (e.g., using a Grade 1-5 scale (see, e.g., Guido Cavaletti & Paola Marmiroli Nature Reviews Neurology 6, 657-666 (December 2010); National Cancer Institute—Common Toxicity Criteria version 4.03 (NCI-CTCAE v4.03).

In some instances, neurologic symptoms may be the earliest symptoms of sCRS. In some embodiments, neurologic symptoms are seen to begin 5 to 7 days after cell therapy infusion. In some embodiments, duration of neurologic changes may range from 3 to 19 days. In some cases, recovery of neurologic changes occurs after other symptoms of sCRS have resolved. In some embodiments, time or degree of resolution of neurologic changes is not hastened by treatment with anti-IL-6 and/or steroid(s).

In some embodiments, a subject is deemed to develop “severe neurotoxicity” in response to or secondary to administration of a cell therapy or dose of cells thereof, if, following administration, the subject displays symptoms that limit self-care (e.g. bathing, dressing and undressing, feeding, using the toilet, taking medications) from among: 1) symptoms of peripheral motor neuropathy, including inflammation or degeneration of the peripheral motor nerves; 2) symptoms of peripheral sensory neuropathy, including inflammation or degeneration of the peripheral sensory nerves, dysesthesia, such as distortion of sensory perception, resulting in an abnormal and unpleasant sensation, neuralgia, such as intense painful sensation along a nerve or a group of nerves, and/or paresthesia, such as functional disturbances of sensory neurons resulting in abnormal cutaneous sensations of tingling, numbness, pressure, cold and warmth in the absence of stimulus. In some embodiments, severe neurotoxicity includes neurotoxicity with a grade of 3 or greater, such as set forth in Table 4. In some embodiments, a severe neurotoxicity is deemed to be a prolonged grade 3 if symptoms or grade 3 neurotoxicity last for 10 days or longer.

TABLE 4
Exemplary Grading Criteria for neurotoxicity

| Grade | Description of Symptoms |
|---|---|
| 1 | Mild or asymptomatic symptoms |
| Asymptomatic | |
| or Mild | |
| 2 | Presence of symptoms that limit instrumental |
| Moderate | activities of daily living (ADL), such as preparing |
| | meals, shopping for groceries or clothes, using |
| | the telephone, managing money |
| 3 | Presence of symptoms that limit self-care ADL, such |
| Severe | as bathing, dressing and undressing, feeding self, |
| | using the toilet, taking medications |
| 4 | Symptoms that are life-threatening, requiring urgent |
| Life-threatening | intervention |
| 5 | Death |
| Fatal | |

In some embodiments, the methods reduce symptoms associated with CRS or neurotoxicity compared to other methods. In some aspects, the provided methods reduce symptoms, outcomes or factors associated with CRS, including symptoms, outcomes or factors associated with severe CRS or grade 3 or higher CRS, compared to other methods. For example, subjects treated according to the present methods may lack detectable and/or have reduced symptoms, outcomes or factors of CRS, e.g. severe CRS or grade 3 or higher CRS, such as any described, e.g. set forth in Table 3. In some embodiments, subjects treated according to the present methods may have reduced symptoms of neurotoxicity, such as limb weakness or numbness, loss of memory, vision, and/or intellect, uncontrollable obsessive and/or compulsive behaviors, delusions, headache, cognitive and behavioral problems including loss of motor control, cognitive deterioration, and autonomic nervous system dysfunction, and sexual dysfunction, compared to subjects treated by other methods. In some embodiments, subjects treated according to the present methods may have reduced symptoms associated with peripheral motor neuropathy, peripheral sensory neuropathy, dysethesia, neuralgia or paresthesia.

In some embodiments, the methods reduce outcomes associated with neurotoxicity including damages to the nervous system and/or brain, such as the death of neurons. In some aspects, the methods reduce the level of factors associated with neurotoxicity such as beta amyloid (Aβ), glutamate, and oxygen radicals.

In some embodiments, the toxicity outcome is a dose-limiting toxicity (DLT). In some embodiments, the toxic outcome is the absence of a dose-limiting toxicity. In some embodiments, a dose-limiting toxicity (DLT) is defined as any grade 3 or higher toxicity as described or assessed by any known or published guidelines for assessing the particular toxicity, such as any described above and including the National Cancer Institute (NCI) Common Terminology Criteria for Adverse Events (CTCAE) version 4.0.

In some embodiments, the provided embodiments result in a low rate or risk of developing a toxicity, e.g. CRS or neurotoxicity or severe CRS or neurotoxicity, e.g. grade 3 or higher CRS or neurotoxicity, such as observed with administering a dose of T cells in accord with the provided combination therapy, and/or with the provided articles of manufacture or compositions. In some casesm this permits administration of the cell therapy on an outpatient basis. In some embodiments, the administration of the cell therapy, e.g. dose of T cells (e.g. CAR+ T cells) in accord with the provided methods, and/or with the provided articles of manufacture or compositions, is performed on an outpatient basis or does not require admission to the subject to the hospital, such as admission to the hospital requiring an overnight stay.

In some aspects, subjects administered the cell therapy, e.g. dose of T cells (e.g. CAR+ T cells) in accord with the provided methods, and/or with the provided articles of manufacture or compositions, including subjects treated on an outpatient basis, are not administered an intervention for treating any toxicity prior to or with administration of the cell dose, unless or until the subject exhibits a sign or symptom of a toxicity, such as of a neurotoxicity or CRS.

In some embodiments, if a subject administered the cell therapy, e.g. dose of T cells (e.g. CAR+ T cells), including subjects treated on an outpatient basis, exhibits a fever the subject is given or is instructed to receive or administer a treatment to reduce the fever. In some embodiments, the fever in the subject is characterized as a body temperature of the subject that is (or is measured at) at or above a certain threshold temperature or level. In some aspects, the threshold temperature is that associated with at least a low-grade fever, with at least a moderate fever, and/or with at least a high-grade fever. In some embodiments, the threshold temperature is a particular temperature or range. For example, the threshold temperature may be at or about or at least at or about 38, 39, 40, 41, or 42 degrees Celsius, and/or may be a range of at or about 38 degrees Celsius to at or about 39 degrees Celsius, a range of at or about 39 degrees Celsius to at or about 40 degrees Celsius, a range of at or about 40 degrees Celsius to at or about 41 degrees, or a range of at or about 41 degrees Celsius to at or about 42 degrees Celsius.

In some embodiments, the treatment designed to reduce fever includes treatment with an antipyretic. An antipyretic may include any agent, e.g., compound, composition, or ingredient, that reduces fever, such as one of any number of agents known to have antipyretic effects, such as NSAIDs (such as ibuprofen, naproxen, ketoprofen, and nimesulide), salicylates, such as aspirin, choline salicylate, magnesium salicylate, and sodium salicylate, paracetamol, acetaminophen, Metamizole, Nabumetone, Phenaxone, antipyrine, febrifuges. In some embodiments, the antipyretic is acetaminophen. In some embodiments, acetaminophen can be administered at a dose of 12.5 mg/kg orally or intravenously up to every four hours. In some embodiments, it is or comprises ibuprofen or aspirin.

In some embodiments, if the fever is a sustained fever, the subject is administered an alternative treatment for treating the toxicity. For subjects treated on an outpatient basis, the subject is instructed to return to the hospital if the subject has and/or is determined to or to have a sustained fever. In some embodiments, the subject has, and/or is determined to or considered to have, a sustained fever if he or she exhibits a fever at or above the relevant threshold temperature, and where the fever or body temperature of the subject is not reduced, or is not reduced by or by more than a specified amount (e.g., by more than 1° C., and generally does not fluctuate by about, or by more than about, 0.5° C., 0.4° C., 0.3° C., or 0.2° C.), following a specified treatment, such as a treatment designed to reduce fever such as treatment with an antipyreticm, e.g. NSAID or salicylates, e.g. ibuprofen, acetaminophen or aspirin. For example, a subject is considered to have a sustained fever if he or she exhibits or is determined to exhibit a fever of at least at or about 38 or 39 degrees Celsius, which is not reduced by or is not reduced by more than at or about 0.5° C., 0.4° C., 0.3° C., or 0.2° C., or by at or about 1%, 2%, 3%, 4%, or 5%, over a period of 6 hours, over a period of 8 hours, or over a period of 12 hours, or over a period of 24 hours, even following treatment with the antipyretic such as acetaminophen. In some embodiments, the dosage of the antipyretic is a dosage ordinarily effective in such as subject to reduce fever or fever of a particular type such as fever associated with a bacterial or viral infection, e.g., a localized or systemic infection.

In some embodiments, the subject has, and/or is determined to or considered to have, a sustained fever if he or she exhibits a fever at or above the relevant threshold temperature, and where the fever or body temperature of the subject does not fluctuate by about, or by more than about, 1° C., and generally does not fluctuate by about, or by more than about, 0.5° C., 0.4° C., 0.3° C., or 0.2° C. Such absence of fluctuation above or at a certain amount generally is measured over a given period of time (such as over a 24-hour, 12-hour, 8-hour, 6-hour, 3-hour, or 1-hour period of time, which may be measured from the first sign of fever or the first temperature above the indicated threshold). For example, in some embodiments, a subject is considered to or is determined to exhibit sustained fever if he or she exhibits a fever of at least at or about or at least at or about 38 or 39 degrees Celsius, which does not fluctuate in temperature by more than at or about 0.5° C., 0.4° C., 0.3° C., or 0.2° C., over a period of 6 hours, over a period of 8 hours, or over a period of 12 hours, or over a period of 24 hours.

In some embodiments, the fever is a sustained fever; in some aspects, the subject is treated at a time at which a subject has been determined to have a sustained fever, such as within one, two, three, four, five six, or fewer hours of such determination or of the first such determination following the initial therapy having the potential to induce the toxicity, such as the cell therapy, such as dose of T cells, e.g. CAR+ T cells.

In some embodiments, one or more interventions or agents for treating the toxicity, such as a toxicity-targeting therapies, is administered at a time at which or immediately after which the subject is determined to or confirmed to (such as is first determined or confirmed to) exhibit sustained fever, for example, as measured according to any of the aforementioned embodiments. In some embodiments, the one or more toxicity-targeting therapies is administered within a certain period of time of such confirmation or determination, such as within 30 minutes, 1 hour, 2 hours, 3 hours, 4 hours, 6 hours, or 8 hours thereof.

### V. ARTICLES OF MANUFACTURE AND KITS

Also provided are articles of manufacture containing a checkpoint inhibitor, e.g., an anti-PD-L1 antibody (or antigen-binding fragment thereof), and components for the immunotherapy, e.g., antibody or antigen binding fragment thereof or T cell therapy, e.g. engineered cells, and/or compositions thereof. The articles of manufacture may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition.

The article of manufacture may include (a) a first container with a composition contained therein, wherein the composition includes the engineered cells used for the immunotherapy, e.g. T cell therapy; and (b) a second container with a composition contained therein, wherein the composition includes the checkpoint inhibitor, e.g., anti-PD-L1 antibody (or antigen-binding fragment thereof). In some embodiments, the first container comprises a first composition and a second composition, wherein the first composition comprises a first population of the engineered cells used for the immunotherapy, e.g., CD4+ T cell therapy, and the second composition comprises a second population of the engineered cells, wherein the second population may be engineered separately from the first population, e.g., CD8+ T cell therapy. In some embodiments, the first and second cell compositions contain a defined ratio of the engineered cells, e.g., CD4+ and CD8+ cells (e.g., 1:1 ratio of CD4+:CD8+ CAR+ T cells). The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VI. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, a “subject” is a mammal, such as a human or other animal, and typically is human. In some embodiments, the subject, e.g., patient, to whom the checkpoint inhibitor, e.g., anti-PD-L1 antibody or antigen-binding fragment, engineered cells, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., engineered cells a checkpoint inhibitor, e.g., anti-PD-L1 or antigen-binding fragment, or a pharmaceutical formulation or composition thereof, in the context of administration, refers to an amount effective, at dosages/amounts and for periods of time necessary, to achieve a desired result, such as a therapeutic or prophylactic result.

A “therapeutically effective amount” of an agent, e.g., engineered cells a checkpoint inhibitor, e.g., anti-PD-L1 or antigen-binding fragment, or a pharmaceutical formulation or composition thereof, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the immunomodulatory polypeptides or engineered cells administered. In some embodiments, the provided methods involve administering the checkpoint inhibitor, e.g., anti-PD-L1 antibody or antigen-binding fragment, engineered cells (e.g. cell therapy), or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New. Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compounds, including cells. It may be a solution, a suspension, liquid, powder, a paste, aqueous, non-aqueous or any combination thereof.

As used herein, a “subject” is a mammal, such as a human or other animal, and typically is human.

### VII. EXEMPLARY EMBODIMENTS

Among the provided embodiments are:

1. A method of treatment, the method comprising:

(a) administering a T cell therapy to a subject having a B cell malignancy, said cell therapy comprising a dose of genetically engineered T cells expressing a recombinant receptor; and

(b) subsequently administering to the subject an anti-PD-L1 antibody or antigen-binding fragment thereof, wherein:
the administration of the anti-PD-L1 antibody or antigen-binding fragment comprises carrying out at least two 28-day cycles, each of said at least two 28-day cycles comprising administering a total dosage amount of 750 mg to 2000 mg of the antibody or antigen-binding fragment; and
in at least the one of said at least two 28-day cycles, the administration of the total dosage amount of the anti-PD-L1 antibody or antigen-binding fragment is carried out by administering more than one individual doses of the antibody or fragment over the course of the at least one 28-day cycle.

2. A method of treatment, the method comprising administering an anti-PD-L1 antibody or antigen-binding fragment thereof to a subject having a B cell malignancy, said subject having been administered a T cell therapy comprising a dose of genetically engineered T cells expressing a recombinant receptor, wherein:

the administration of the anti-PD-L1 antibody or antigen-binding fragment comprises carrying out at least two 28-day cycles, each of said at least two 28-day cycles, independently, comprising administering a total dosage amount of 750 mg to 2000 mg of the antibody or antigen-binding fragment; and
in at least the one of said at least two 28-day cycles, the administration of the total dosage amount of the anti-PD-L1 antibody or antigen-binding fragment is carried out by administering more than one individual dose of the antibody or fragment over the course of the at least one 28-day cycle.

3. The method of embodiment 1 or embodiment 2, wherein in a first of said at least two 28-day cycles, the administration of the total dosage amount of the anti-PD-L1 antibody or antigen-binding fragment is carried out by administering a greater number of individual doses of the antibody or fragment as compared to the administration in a second and/or a subsequent 28-day cycle.

4. The method of any of embodiments 1-3, wherein the total dosage amount of the anti-PD-L1 antibody or antigen-binding fragment in each 28-day cycle independently is between at or about 750 mg and at or about 1500 mg.

5. The method of any of embodiments 1-4, wherein the total dosage amount of the anti-PD-L1 antibody or antigen-binding fragment in at least one of the 28-day cycles is at or about 750 mg.

6. The method of any of embodiments 1-4, wherein the total dosage amount of the anti-PD-L1 antibody or antigen-binding fragment in at least one of the 28-day cycles is at or about 1200 mg.

7. The method of any of embodiments 1-4, wherein the total dosage amount of the anti-PD-L1 antibody or antigen-binding fragment in at least one of the 28-day cycles is at or about 1500 mg.

8. The method of any of embodiments 1-4 and 7, wherein the total dosage amount of the anti-PD-L1 antibody or antigen-binding fragment in each 28-day cycle, independently, is at or about 1500 mg.

9. The method of any of embodiments 1-8, wherein the total dosage amount of the anti-PD-L1 antibody or antigen-binding fragment in at least two of said at least two, and optionally in said at least two, 28-day cycles is the same total dosage amount.

10. The method of any of embodiments 1-8, wherein the total dosage amount of the anti-PD-L1 antibody or antigen-binding fragment is different in at least two of, or is different in each of, said at least two 28-day cycles.

11. The method of any of embodiments 2-8 and 10, wherein the total dosage amount of the anti-PD-L1 antibody or antigen-binding fragment in the first of said at least two 28-day cycles is lower than the second and/or a subsequent of said at least two 28-day cycle.

12. The method of any of embodiments 1-11, wherein the administration of the total dosage amount in the first of said at least two 28-day cycles comprises administering 2, 3 or 4 individual doses of the anti-PD-L1 antibody or antigen-binding fragment thereof.

13. The method of any of embodiments 1-12, wherein the administration of the total dosage amount in the first of said at least two 28-day cycles comprises administering individual doses according to a dosing schedule selected from (i) once-weekly (Q1W) for two individual doses, optionally on days 15 and 22 of the 28-day cycle; (ii) once-weekly (Q1W) for four individual doses, optionally on days 1, 8, 15 and 22 of the 28-day cycle; (iii) Q1W for two consecutive doses, optionally on days 1 and 8 of the cycle, followed by every two weeks (Q2W) for one dose, optionally on day 15 of the cycle; or (iv) every two weeks (Q2W) for two doses, optionally on days 1 and 15 of the 28-day cycle.

14. The method of embodiment 13, wherein:

each Q1W dose administered in the first 28-day cycle is independently from or from at or about 18% to at or about 32% of the total dosage amount administered in the first 28-day cycle, optionally is at or about 25% of the total dosage amount administered in the first 28-day cycle; and/or

each Q2W dose administered in the first 28-day cycle is independently from or from at or about 40% to at or about 62.5% of the total dosage amount, optionally is at or about 50% of the total dosage amount administered in the first 28-day cycle.

15. The method of embodiment 13 or embodiment 14, wherein:

the administration of the total dosage amount in the first 28-day cycle comprises administering the anti-PD-L1 antibody or antigen-binding fragment thereof Q1W for two consecutive doses, each independently, in an amount of or of about 375 mg followed by Q2W for one dose in an amount of or of about 750 mg;

the administration of the total dosage amount in the first 28-day cycle comprises administering the anti-PD-L1 antibody or antigen-binding fragment thereof Q1W for four doses, said four doses comprising two consecutive doses of or about 225 mg followed by two consecutive doses of or about 375 mg; or

the administration of the total dosage amount in the first 28-day cycle comprises administering the anti-PD-L1 antibody or antigen-binding fragment thereof Q1W for two consecutive doses in an amount of or about 375 mg.

16. The method of any of embodiments 3-15, wherein the administration of the total dosage amount in the second and/or a subsequent 28-day cycle, independently, comprises administering 1 or 2 does of the anti-PD-L1 antibody or antigen-binding fragment thereof.

17. The method of any of embodiments 3-16, wherein the administration of the total dosage amount in the second and/or a subsequent 28-day cycle, independently, comprises a dosing schedule selected from (i) every two weeks (Q2W) for two doses, optionally on days 1 and 15 of the cycle; or (ii) every four weeks (Q4W) for one dose, optionally on day 1 of the cycle.

18. The method of embodiment 17, wherein:

each Q2W dose of the second and/or subsequent 28-day cycle is or is about 50% of the total dosage amount; and/or

the Q4W dose of the second and/or subsequent 28-day cycle is or is about the total dosage amount.

19. The method of embodiment 17 or embodiment 18, wherein:

the second and/or a subsequent dose comprises administering the anti-PD-L1 antibody or antigen-binding fragment thereof Q2W for two doses in an amount of or of about 750 mg; or

the second and/or a subsequent dose comprises administering the anti-PD-L1 antibody or antigen-binding fragment thereof Q4W for one dose in an amount of or of about 1500 mg.

20. A method of treatment, the method comprising:

(a) administering a T cell therapy to a subject having a B cell malignancy, said T cell therapy comprising a dose of genetically engineered T cells expressing a recombinant receptor; and

(b) subsequently administering to the subject an anti-PD-L1 antibody or antigen-binding fragment thereof, wherein said administration comprises carrying out at least two 28-day cycles, wherein:
the first 28-day cycle comprises administering the anti-PD-L1 antibody or antigen-binding fragment thereof once-weekly (Q1W) for two consecutive doses in an amount of or about 375 mg followed by every two weeks (Q2W) for one dose in an amount of or about 750 mg; and
the second and/or a subsequent 28-day cycle comprises administering the anti-PD-L1 antibody or antigen-binding fragment thereof Q4W for one dose in an amount of or about 1500 mg.

21. A method of treatment, the method comprising administering an anti-PD-L1 antibody or antigen-binding fragment thereof to a subject having a B cell malignancy, said subject having been administered a T cell therapy comprising a dose of genetically engineered T cells expressing a recombinant receptor, wherein the administration of the anti-PD-L1 antibody or antigen-binding fragment thereof comprises carrying out at least two 28-day cycles, wherein:

the first 28-day cycle comprises administering the anti-PD-L1 antibody or antigen-binding fragment thereof once-weekly (Q1W) for two consecutive doses independently in an amount of or of about 375 mg followed by every two weeks (Q2W) for one dose in an amount of or of about 750 mg; and
the second and/or a subsequent 28-day cycle comprises administering the anti-PD-L1 antibody or antigen-binding fragment thereof every four weeks (Q4W) for one dose in an amount of or of about 1500 mg.

22. A method of treatment, the method comprising:

(a) administering a T cell therapy to a subject having a B cell malignancy, said T cell therapy comprising a dose of genetically engineered T cells expressing a recombinant receptor; and

(b) subsequently administering to the subject an anti-PD-L1 antibody or antigen-binding fragment thereof, said administration comprises carrying out at least two 28-day cycles, wherein:
the first 28-day cycle comprises administering the anti-PD-L1 antibody or antigen-binding fragment thereof once-weekly (Q1W) for four doses, said four doses comprising two consecutive doses each independently of or of about 225 mg followed by two consecutive doses each independently of or of about 375 mg; and
the second and/or a subsequent 28-day cycle comprises administering the anti-PD-L1 antibody or antigen-binding fragment thereof every two weeks (Q2W) for two doses each independently in an amount of or of about 750 mg.

23. A method of treatment, the method comprising administering an anti-PD-L1 antibody or antigen-binding fragment thereof to a subject having a B cell malignancy, said subject having been administered a T cell therapy comprising a dose of genetically engineered T cells expressing a recombinant receptor, wherein the anti-PD-L1 antibody or antigen-binding fragment thereof comprises carrying out at least two 28-day cycles, wherein:

the first 28-day cycle comprises administering the anti-PD-L1 antibody or antigen-binding fragment thereof once-weekly (Q1W) for four doses, said four doses comprising two consecutive doses each independently of or of about 225 mg followed by two consecutive doses of or about 375 mg; and

the second and/or a subsequent 28-day cycle comprises administering the anti-PD-L1 antibody or antigen-binding fragment thereof every two weeks (Q2W) for two doses in an amount of or about 750 mg.

24. A method of treatment, the method comprising:

(a) administering a T cell therapy to a subject having a B cell malignancy, said T cell therapy comprising a dose of genetically engineered T cells expressing a recombinant receptor; and

(b) subsequently administering to the subject an anti-PD-L1 antibody or antigen-binding fragment thereof, said administration comprises carrying out at least two 28-day cycles, wherein:
the first 28-day cycle comprises administering the anti-PD-L1 antibody or antigen-binding fragment thereof once-weekly (Q1W) for two doses, each of said two doses each independently comprising an amount of or of about 375 mg, optionally wherein the two doses are consecutive doses, optionally wherein the two doses are administered days 15 and 22 in the 28-day cycle; and
the second and/or a subsequent 28-day cycle comprises administering the anti-PD-L1 antibody or antigen-binding fragment thereof Q4W for one dose in an amount of or about 1500 mg.

25. A method of treatment, the method comprising administering an anti-PD-L1 antibody or antigen-binding fragment thereof to a subject having a B cell malignancy, said subject having been administered a T cell therapy comprising a dose of genetically engineered T cells expressing a recombinant receptor, wherein the administration of the anti-PD-L1 antibody or antigen-binding fragment comprises carrying out at least two 28-day cycles, wherein:
the first 28-day cycle comprises administering the anti-PD-L1 antibody or antigen-binding fragment thereof once-weekly (Q1W) for two doses, each of said two doses independently comprising an amount of or about 375 mg, optionally wherein the two doses are consecutive doses, optionally wherein the two doses are administered on days 15 and 22 in the 28-day cycle; and
the second and/or a subsequent 28-day cycle comprises administering the anti-PD-L1 antibody or antigen-binding fragment thereof Q4W for one dose in an amount of or about 1500 mg.

26. The method of any of embodiments 1-25, wherein at least two 28-day cycles further comprises a third 28-day cycle and/or wherein the subsequent 28-day cycle is a third 28-day cycle.

27. The method of embodiment 26, wherein the total dosage amount of the anti-PD-L1 antibody or antigen-binding fragment in the third 28-day cycle is the same as the total dosage amount administered in the first and/or in the second 28-day cycle.

28. The method of embodiment 26 or embodiment 27, wherein the total dosage amount of the anti-PD-L1 antibody or antigen-binding fragment in the third 28-day cycle is or is about 1500 mg.

29. The method of any of embodiments 26-28, wherein:

(a) in the third 28-day cycle, the administration of the total dosage amount of the anti-PD-L1 antibody or antigen-binding fragment is carried out by administering the antibody or fragment in a greater number of individual doses as compared to in the first and/or second 28-day cycle; or

(b) in the third 28-day cycle, the administration of the total dosage amount of the anti-PD-L1 antibody or antigen-binding fragment is carried out by administering the same number of doses of the antibody or fragment as compared to the second 28-day cycle.

30. The method of any of embodiments 26-29, wherein the administration of the total dosage amount in the third 28-day cycle comprises administration every four weeks (Q4W) for one dose, optionally on day 1 of the third 28-day cycle.

31. The method of any one of embodiments 1-30, wherein the start or day 1 of the first of said at least two 28-day cycles is initiated at a time:

(a) between day 22 and day 36 of initiation of the administration of the T cell therapy; or

(b) at or after, optionally immediately after or within 1 to 3 days after:
(i) peak or maximum level of the cells of the T cell therapy are detectable in the blood of the subject;
(ii) the number of cells of the T cell therapy detectable in the blood, after having been detectable in the blood, is not detectable or is reduced, optionally reduced compared to a preceding time point after administration of the T cell therapy;
(iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy;
(iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject;
(v) the subject exhibits disease progression and/or has relapsed following remission after treatment with the T cell therapy; and/or
(iv) the subject exhibits increased tumor burden as compared to tumor burden at a time prior to or after administration of the cells and prior to initiation of administration of the anti-PD-L1 antibody.

32. The method of any of embodiments 1-31, wherein the at least two 28-day cycles comprise no more than three 28-day cycles, optionally wherein the first of said at least two 28-day cycles is initiated between at or about day 22 and at or about day 36, optionally at or about day 29, after initiation of the administration of the T cell therapy.

33. A method of treatment, the method comprising:

(a) administering a T cell therapy to a subject having a B cell malignancy, said T cell therapy comprising a dose of genetically engineered T cells expressing a recombinant receptor; and

(b) subsequently administering to the subject an anti-PD-L1 antibody or antigen-binding fragment thereof, wherein the administration of antibody or antigen-binding fragment comprises carrying out between one and three 28-day cycles, each cycle comprising administering a total dosage amount of 750 mg to 2000 mg of the antibody or fragment, optionally wherein the first of said between one and three 28-day cycle begins between at or about day 22 and at or about day 36, optionally at day 29, after initiation of the T cell therapy.

34. A method of treatment, the method comprising administering an anti-PD-L1 antibody or antigen-binding fragment thereof to a subject having a B cell malignancy, said subject having been administered a T cell therapy comprising a dose of genetically engineered T cells expressing a recombinant receptor, wherein the administration of the antibody or antigen-binding fragment comprises carrying out between one and three 28-day cycles, each cycle comprises administering a total dosage amount of 900 mg to 2000 mg of the antibody or fragment, optionally wherein the first of said between one and three 28-day cycles begins between at or about day 22 and at or about day 36, optionally at about day 29, after initiation of the T cell therapy.

35. The method of embodiment 33 or embodiment 34, wherein the total dosage amount of the anti-PD-L1 antibody or antigen-binding fragment in each 28-day cycle independently is or is about 1200 mg to 1500 mg.

36. The method of any of embodiments 33-35, wherein the total dosage amount of the anti-PD-L1 antibody or antigen-binding fragment in at least one 28-day cycle is or is about 1200 mg.

37. The method of any of embodiments 33-35, wherein the total dosage amount of the anti-PD-L1 antibody or antigen-binding fragment in at least one 28-day cycle is or is about 1500 mg.

38. The method of any of embodiments 33-35 and 37, wherein the total dosage amount of the anti-PD-L1 antibody or antigen-binding fragment in each 28-day cycle is or is about 1500 mg.

39. The method of any of embodiments 33-38, wherein the total dosage amount in each 28-day cycle comprises administering 1, 2, 3 or 4 doses of the anti-PD-L1 antibody or antigen-binding fragment thereof.

40. The method of any of embodiments 33-39, wherein each 28-day cycle independently comprises a dosing schedule selected from (i) once-weekly (Q1W) for four doses, optionally on days 1, 8, 15 and 22; (ii) Q1W for two consecutive doses, optionally on days 1 and 8, followed by every two weeks (Q2W) for one dose, optionally on day 15; (iii) every two weeks (Q2W) for two doses, optionally on days 1 and 15; or (iv) every four weeks (Q4W) for one dose, optionally on day 1.

41. The method of any of embodiments 33-40, wherein the anti-PD-L1 antibody or antigen-binding fragment is administered on day 1, 8 and 15 in a first 28-day cycle, on day 1 in a second 28-day cycle, and on day 1 in a third 28-day cycle.

42. The method of any of embodiments 33-40, wherein the anti-PD-L1 antibody or antigen-binding fragment is administered on day 1, 8, 15 and 22 in a first 28-day cycle, on day 1 and 15 in a second 28-day cycle, and on day 1 in a third 28-day cycle.

43. The method of any of embodiments 33-40, wherein the anti-PD-L1 antibody or antigen-binding fragment is administered on day 1 in each 28-day cycle.

44. The method of any of embodiments 32-43, further comprising administering the anti-PD-L1 antibody or antigen-binding fragment in one or more further 28-day cycle if the subject exhibits no more than a partial response (PR) following the treatment and/or exhibits no more than a PR at three-months following initiation of administration of the T cell therapy and/or of the anti-PD-L1 antibody or fragment.

45. The method of embodiment 44, wherein the anti-PD-L1 antibody or antigen-binding fragment is administered in a total dosage amount of 900 mg to 2000 mg in each of the one or more further 28-day cycle, optionally at or about 1500 mg.

46. The method of any of embodiments 1-45, wherein the anti-PD-L1 antibody or antigen-binding fragment is administered for a total duration of no more than 12 months.

47. The method of embodiment any of embodiments 1-46, wherein the administration of the anti-PD-L1 antibody or antigen-binding fragment and/or the start of the first 28-day cycle is initiated greater than 21 days after initiation of administration of the T cell therapy.

48. The method of any of embodiments 1-47, wherein administration of the anti-PD-L1 antibody or antigen-binding fragment and/or the start of the first 28-day cycle is initiated at a time at or after, optionally immediately after or within 1 to 3 days after:

(i) peak or maximum level of the cells of the T cell therapy are detectable in the blood of the subject;

(ii) the number of cells of the T cell therapy detectable in the blood, after having been detectable in the blood, is not detectable or is reduced, optionally reduced compared to a preceding time point after administration of the T cell therapy;

(iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy;

(iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject;

(v) the subject exhibits disease progression and/or has relapsed following remission after treatment with the T cell therapy; and/or

(iv) the subject exhibits increased tumor burden as compared to tumor burden at a time prior to or after administration of the cells and prior to initiation of administration of the anti-PD-L1 antibody.

49. The method of any of embodiments 1-48, wherein administration of the anti-PD-L1 antibody or antigen-binding fragment and/or the start of the first 28-day cycle is initiated at or within 29 days, 36 days, 43 days or 50 days after initiation of administration of the T cell therapy.

50. The method of any of embodiments 1-49, wherein administration of the anti-PD-L1 antibody or antigen-binding fragment and/or the start of the first 28-day cycle is initiated from or from about 22 days to 36 days after initiation of administration of the T cell therapy.

51. The method of any of embodiments 1-50, wherein administration of the anti-PD-L1 antibody or antigen-binding fragment and/or the start of the first 28-day cycle is initiated at or about 29 days after initiation of administration of the T cell therapy.

52. The method of any of embodiments 1-51, wherein at the time of administering the anti-PD-L1 antibody or antigen-binding fragment and/or the start of the first 28-day cycle, the subject does not exhibit a severe toxicity following administration of the T cell therapy.

53. The method of embodiment 52, wherein:

the severe toxicity is severe cytokine release syndrome (CRS), optionally grade 3 or higher, prolonged grade 3 or higher or grade 4 or 5 CRS; and/or

the severe toxicity is severe neurotoxicity, optionally grade 3 or higher, prolonged grade 3 or higher or grade 4 or 5 neurotoxicity.

54. The method of any of embodiments 1-53, wherein the anti-PD-L1 antibody or antigen-binding fragment thereof specifically binds to an extracellular domain of PD-L1.

55. The method of any of embodiments 1-54, wherein the anti-PD-L1 antibody or antigen-binding fragment thereof is MEDI4736 (durvalumab), MDPL3280A (atezolizumab), YW243.55.S70, MDX-1105 (BMS-936559), LY3300054, or MSB0010718C (avelumab), or is or comprises an antigen-binding fragment or region of any of the foregoing.

56. The method of any of embodiments 1-55, wherein the anti-PD-L1 antibody or antigen-binding fragment thereof is MEDI4736 (durvalumab) or is or comprises an antigen-binding fragment or region thereof.

57. The method of any of embodiments 1-56, wherein the B cell malignancy is a non-Hodgkin lymphoma (NHL).

58. The method of embodiment 57, wherein, at or immediately prior to the time of the administration of the T cell therapy the subject has relapsed following remission after treatment with, or become refractory to, one or more prior therapies for the NHL, optionally one or two prior therapies other than another dose of cells expressing the CAR, optionally wherein the prior therapy is or comprises a CD20-targeted agent or anthracycline.

59. The method of embodiment 57 or embodiment 58, wherein the NHL comprises aggressive NHL, diffuse large B cell lymphoma (DLBCL), DLBCL-NOS, optionally transformed indolent; EBV-positive DLBCL-NOS; T cell/histiocyte-rich large B-cell lymphoma; primary mediastinal large B cell lymphoma (PMBCL); follicular lymphoma (FL), optionally, follicular lymphoma Grade 3B (FL3B); and/or high-grade B-cell lymphoma, with MYC and BCL2 and/or BCL6 rearrangements with DLBCL histology (double/triple hit).

60. The method of any of embodiments 1-59, wherein the subject is or has been identified as having an Eastern Cooperative Oncology Group Performance Status (ECOG) status of less than or equal to 1.

61. The method of any of embodiments 1-60, wherein the recombinant receptor specifically binds to a target antigen expressed by the B cell malignancy.

62. The method of embodiment 61, wherein the target antigen is a B cell antigen, optionally CD19.

63. The method of any of embodiments 1-62, wherein the recombinant receptor is a chimeric antigen receptor (CAR).

64. The method of embodiment 63, wherein the chimeric antigen receptor (CAR) comprises an extracellular antigen-recognition domain that specifically binds to the antigen and an intracellular signaling domain comprising an ITAM.

65. The method of embodiment 64, wherein the intracellular signaling domain comprises an signaling domain of a CD3-zeta (CD3) chain.

66. The method of embodiment 64 or embodiment 65, wherein the chimeric antigen receptor (CAR) further comprises a costimulatory signaling region.

67. The method of embodiment 66, wherein the costimulatory signaling region comprises a signaling domain of CD28 or 4-1BB.

68. The method of embodiment 66 or embodiment 67, wherein the costimulatory domain is or comprises a domain of 4-1BB.

69. The method of any of embodiments 63-68, wherein:

the CAR comprises an scFv specific for the antigen, a transmembrane domain, a cytoplasmic signaling domain derived from a costimulatory molecule, which optionally is or comprises a 4-1BB, and a cytoplasmic signaling domain derived from a primary signaling ITAM-containing molecule, which optionally is or comprises a CD3zeta signaling domain and optionally further comprises a spacer between the transmembrane domain and the scFv;

the CAR comprises, in order, an scFv specific for the antigen, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is a CD3zeta signaling domain; or

the CAR comprises, in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain.

70. The method of embodiment 69, wherein:

the spacer is optionally a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID O:N 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine; and/or

the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or

the primary signaling domain comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or

the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv comprises, in order, a VH, a linker, optionally comprising SEQ ID NO: 41, and a VL, and/or the scFv comprises a flexible linker and/or comprises the amino acid sequence set forth as SEQ ID NO: 42.

71. The method of any of embodiments 1-70, wherein the dose of genetically engineered T cells comprises from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, 5×10⁷ to 1×10⁸ total CAR-expressing T cells, each inclusive.

72. The method of any of embodiments 1-71, wherein the dose of genetically engineered T cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

73. The method of any of embodiments 1-72, wherein the dose of genetically engineered T cells comprises at or about 5×10⁷ CAR-expressing cells.

74. The method of any of embodiments 1-72, wherein the dose of genetically engineered T cells comprises at or about 1×10⁸ CAR-expressing cells.

75. The method of any of embodiments 1-74, wherein the dose of cells is administered parenterally, optionally intravenously.

76. The method of embodiment 75, wherein the T cells are primary T cells obtained from a subject.

77. The method of any of embodiments 1-76, wherein the T cells are autologous to the subject.

78. The method of any of embodiments 1-77, wherein the T cells are allogeneic to the subject.

79. The method of any of embodiments 1-78, wherein the dose of genetically engineered T cells comprises CD4+ T cells expressing the CAR and CD8+ T cells expressing the CAR and the administration of the dose comprises administering a plurality of separate compositions, said plurality of separate compositions comprising a first composition comprising one of the CD4+ T cells and the CD8+ T cells and the second composition comprising the other of the CD4+ T cells or the CD8+ T cells.

80. The method of embodiment 79, wherein:

the first composition and second composition are administered 0 to 12 hours apart, 0 to 6 hours apart or 0 to 2 hours apart or wherein the administration of the first composition and the administration of the second composition are carried out on the same day, are carried out between about 0 and about 12 hours apart, between about 0 and about 6 hours apart or between about 0 and 2 hours apart; and/or

the initiation of administration of the first composition and the initiation of administration of the second composition are carried out between about 1 minute and about 1 hour apart or between about 5 minutes and about 30 minutes apart.

81. The method of embodiment 79 or embodiment 80, wherein the first composition and second composition are administered no more than 2 hours, no more than 1 hour, no more than 30 minutes, no more than 15 minutes, no more than 10 minutes or no more than 5 minutes apart.

82. The method of any of embodiments 79-81, wherein the first composition comprises the CD4+ T cells.

83. The method of any of embodiments 79-82, wherein the first composition comprises the CD8+ T cells.

84. The method of any of embodiments 79-83, wherein the first composition is administered prior to the second composition.

85. The method of any of embodiments 1-84, wherein, prior to the administration, the subject has been preconditioned with a lymphodepleting therapy comprising the administration of fludarabine and/or cyclophosphamide. 86. The method of any of embodiments 1-85, further comprising, immediately prior to the administration, administering a lymphodepleting therapy to the subject comprising the administration of fludarabine and/or cyclophosphamide.

87. The method of embodiment 85 or embodiment 86, wherein the lymphodepleting therapy comprises administration of cyclophosphamide at about 200-400 mg/m², optionally at or about 300 mg/m², inclusive, and/or fludarabine at about 20-40 mg/m², optionally 30 mg/m², daily for 2-4 days, optionally for 3 days, or wherein the lymphodepleting therapy comprises administration of cyclophosphamide at about 500 mg/m².

88. The method of any of embodiments 85-87, wherein:

the lymphodepleting therapy comprises administration of cyclophosphamide at or about 300 mg/m² and fludarabine at about 30 mg/m² daily for 3 days; and/or

the lymphodepleting therapy comprises administration of cyclophosphamide at or about 500 mg/m² and fludarabine at about 30 mg/m² daily for 3 days.

89. The method of any of embodiments 1-88, wherein the subject is a human. 90. A kit comprising:

(a) a T cell therapy comprising a dose of genetically engineered T cells expressing a recombinant receptor;

(b) an anti-PD-L1 antibody or antigen-binding fragment thereof, optionally wherein the anti-PD-L1 antibody or fragment thereof is formulated in one or more unit doses; and

(c) instructions for administering the genetically engineered cells and/or the anti-PD-L1 antibody or antigen-binding fragment to a subject having a B cell malignancy, wherein the instructions specify:
(i) the administration of the anti-PD-L1 antibody or antigen-binding fragment is to be carried out for at least two 28-day cycles, each of said at least two 28-day cycles comprising administering a total dosage amount of 750 mg to 2000 mg of the antibody or antigen-binding fragment; and
(ii) in at least the first of said at least two 28-day cycles, the administration of the total dosage amount of the anti-PD-L1 antibody or antigen-binding fragment is carried out by administering the antibody or fragment more than one time.

91. A kit comprising:

(a) a T cell therapy comprising a dose of genetically engineered T cells expressing a recombinant receptor; and

(b) instructions for administering the T cell therapy to a subject having a B cell malignancy, wherein the instructions specify that the subject is to be administered an anti-PD-L1 antibody or antigen-binding fragment thereof after the administration of T cells, wherein the instructions specify:
(i) the administration of the anti-PD-L1 antibody or antigen-binding fragment is to be carried out for at least two 28-day cycles, each of said at least two 28-day cycles comprising administering a total dosage amount of 750 mg to 2000 mg of the antibody or antigen-binding fragment; and
(ii) in at least the first of said at least two 28-day cycles, the administration of the total dosage amount of the anti-PD-L1 antibody or antigen-binding fragment is carried out by administering the antibody or fragment more than one time.

92. A kit comprising:

(a) an anti-PD-L1 antibody or antigen-binding fragment thereof, optionally wherein the anti-PD-L1 antibody or fragment thereof is formulated in one or more unit doses; and

(b) instructions for administering the anti-PD-L1 antibody or antigen-binding fragment to a subject having a B cell malignancy, wherein the instructions specify that the anti-PD-L1 antibody or fragment is administered after initiation of administration of a T cell therapy, the T cell therapy comprising a dose of genetically engineered T cells expressing a recombinant receptor, wherein the instructions specify:
(i) the administration of the anti-PD-L1 antibody or antigen-binding fragment is to be carried out at least two 28-day cycles, each of said at least two 28-day cycles comprising administering a total dosage amount of 750 mg to 2000 mg of the antibody or antigen-binding fragment thereof; and
(ii) in at least the first of said at least two 28-day cycles, the administration of the total dosage amount of the anti-PD-L1 antibody or antigen-binding fragment is carried out by administering the antibody or fragment more than one time.

93. The kit of any one of embodiments 90-92, wherein the instructions further specify that in a first of said at least two 28-day cycles, the administration of the total dosage amount of the anti-PD-L1 antibody or antigen-binding fragment is carried out by administering the antibody or fragment a greater number of times as compared to a second and/or a subsequent 28-day cycle.

94. The kit of any one of embodiments 90, 92, and 93, wherein the total amount of the anti-PD-L1 antibody or antigen-binding fragment is or is about 225 mg to 2000 mg.

95. The kit of embodiment 94, wherein the total amount of the anti-PD-L1 antibody or antigen-binding fragment is or is about 750-1500 mg.

96. The kit of embodiment 94 or 95, wherein the anti-PD-L1 antibody or antigen-binding fragment is formulated in two or more unit doses, wherein each unit dose is or is about 225 mg to 2000 mg.

97. The kit of embodiment 96, wherein each unit dose is or is about 225 mg to 1500 mg.

98. The kit of any one of embodiments 90-97, wherein the anti-PD-L1 antibody or antigen-binding fragment thereof specifically binds to an extracellular domain of PD-L1.

99. The kit of any one of embodiments 90-98, wherein the anti-PD-L1 antibody or antigen-binding fragment thereof is MEDI4736 (durvalumab), MDPL3280A (atezolizumab), YW243.55.S70, MDX-1105 (BMS-936559), LY3300054, or MSB0010718C (avelumab), or is an antigen-binding fragment thereof.

100. The kit of embodiment 99, wherein the anti-PD-L1 antibody or antigen-binding fragment thereof is MEDI4736 (durvalumab) or is an antigen-binding fragment thereof.

101. The kit of any one of embodiments 90-100, wherein the recombinant receptor specifically binds to a target antigen expressed by the B cell malignancy.

102. The kit of embodiment 101, wherein the target antigen is a B cell antigen, optionally CD19.

103. The kit of any of embodiments 90-102, wherein the recombinant receptor is a chimeric antigen receptor (CAR).

104. The kit of embodiment 103, wherein the chimeric antigen receptor (CAR) comprises an extracellular antigen-recognition domain that specifically binds to the antigen and an intracellular signaling domain comprising an ITAM.

105. The kit of embodiment 104, wherein the intracellular signaling domain comprises an intracellular domain of a CD3-zeta (CD3) chain.

106. The kit of embodiment 103 or embodiment 104, wherein the chimeric antigen receptor (CAR) further comprises a costimulatory signaling region.

107. The kit of embodiment 106, wherein the costimulatory signaling region comprises a signaling domain of CD28 or 4-1BB.

108. The kit of embodiment 106 or embodiment 107, wherein the costimulatory domain is a domain of 4-1BB.

109. The kit of any one of embodiments 103-108, wherein:

the CAR comprises an scFv specific for the antigen, a transmembrane domain, a cytoplasmic signaling domain derived from a costimulatory molecule, which optionally is or comprises a 4-1BB, and a cytoplasmic signaling domain derived from a primary signaling ITAM-containing molecule, which optionally is or comprises a CD3zeta signaling domain and optionally further comprises a spacer between the transmembrane domain and the scFv;

the CAR comprises, in order, an scFv specific for the antigen, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is a CD3zeta signaling domain; or

the CAR comprises, in order, an scFv specific for the antigen, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain.

110. The kit of embodiment 109, wherein:

the spacer is optionally a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID O:N 34, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine; and/or

the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or

the primary signaling domain comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto; and/or

the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and/or a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any of the foregoing, and optionally wherein the scFv comprises, in order, a VH, a linker, optionally comprising SEQ ID NO: 41, and a VL, and/or the scFv comprises a flexible linker and/or comprises the amino acid sequence set forth as SEQ ID NO: 42.

111. The kit of any one of embodiments 90-110, where the T cell therapy comprises from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, 5×10⁷ to 1×10⁸ total CAR-expressing T cells, each inclusive.

112. The kit of any one of embodiments 90-111, wherein the T cell therapy comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

113. The kit of any one of embodiments 90-112, wherein the T cell therapy comprises at or about 5×10⁷ CAR-expressing cells.

114. The kit of any one of embodiments 90-113, wherein the T cell therapy comprises at or about 1×10⁸ CAR-expressing cells.

115. The kit of any one of embodiments 90-114, wherein the T cell therapy comprises primary T cells obtained from the subject.

116. The kit of any one of embodiments 90-115, wherein the T cell therapy comprises cells that are autologous to the subject.

117. The kit of any one of embodiments 90-116, wherein the T cell therapy comprises cells are allogeneic to the subject.

118. The kit of any one of embodiments 90-117, wherein the T cell therapy comprise CD4+ T cells expressing the CAR and CD8+ T cells expressing the CAR and the administration comprises administering a plurality of separate compositions, said plurality of separate compositions comprising a first composition comprising one of the CD4+ T cells and the CD8+ T cells and the second composition comprising the other of the CD4+ T cells or the CD8+ T cells.

119. The kit of embodiment 118, wherein:

the first composition and second composition are administered 0 to 12 hours apart, 0 to 6 hours apart or 0 to 2 hours apart or wherein the administration of the first composition and the administration of the second composition are carried out on the same day, are carried out between about 0 and about 12 hours apart, between about 0 and about 6 hours apart or between about 0 and 2 hours apart; and/or

the initiation of administration of the first composition and the initiation of administration of the second composition are carried out between about 1 minute and about 1 hour apart or between about 5 minutes and about 30 minutes apart.

120. The kit of embodiment 118 or embodiment 119, wherein the instructions specify that the first composition and second composition is administered no more than 2 hours, no more than 1 hour, no more than 30 minutes, no more than 15 minutes, no more than 10 minutes or no more than 5 minutes apart.

121. The kit of any one of embodiments 118-120, wherein the first composition comprises the CD4+ T cells.

122. The kit of any one of embodiments 118-121, wherein the first composition comprises the CD8+ T cells.

123. The kit of any one of embodiments 118-122, wherein the instructions specify that the first composition is administered prior to the second composition.

124. The kit of any one of embodiments 90-123, further comprising instructions for administering a lymphodepleting therapy comprising fludarabine and/or cyclophosphamide.

125. The kit of any one of embodiments 90-124, wherein the instructions specify that the lymphodepleting therapy is administered prior to the administration of the T cell therapy and/or the anti-PD-L1 antibody or fragment thereof.

126. The kit of any one of embodiments 90-125, wherein the instructions specify that the total dosage amount of the anti-PD-L1 antibody or antigen-binding fragment in each 28-day cycle independently is a dosage or a range of dosage in the range of about 750 mg to about 1500 mg.

127. The kit of any one of embodiments 90-126, wherein the instructions specify that the total dosage amount of the anti-PD-L1 antibody or antigen-binding fragment in at least one 28-day cycle is or is about 750 mg.

128. The kit of any one of embodiments 90-126, wherein the instructions specify that the total dosage amount of the anti-PD-L1 antibody or antigen-binding fragment in at least one 28-day cycle is or is about 1200 mg.

129. The kit of any one of embodiments 90-126, wherein the instructions specify that the total dosage amount of the anti-PD-L1 antibody or antigen-binding fragment in at least one 28-day cycle is or is about 1500 mg.

130. The kit of any one of embodiments 90-126 and embodiment 129, wherein the instructions specify that the total dosage amount of the anti-PD-L1 antibody or antigen-binding fragment in each 28-day cycle is or is about 1500 mg.

131. The kit of any one of embodiments 90-130, wherein the instructions specify that the total dosage amount of the anti-PD-L1 antibody or antigen-binding fragment in said at least two 28-day cycles is the same.

132. The kit of any one of embodiments 90-130, wherein the instructions specify that the total dosage amount of the anti-PD-L1 antibody or antigen-binding fragment in said at least two 28-day cycles is different.

133. The kit of any one of embodiments 90-130 and embodiment 132, wherein the instructions specify that the total dosage amount of the anti-PD-L1 antibody or antigen-binding fragment in the first 28-day cycle is lower than the second and/or a subsequent 28-day cycle.

134. The kit of any one of embodiments 90-133, wherein the instructions specify that the first 28-day cycle is carried out by administering 2, 3 or 4 doses of the anti-PD-L1 antibody or antigen-binding fragment thereof.

135. The kit of any one of embodiments 90-134, wherein the instructions specify that the first 28-day cycle is carried out by a dosing schedule selected from (i) once-weekly (Q1W) for two doses, optionally on days 15 and 22; (ii) once-weekly (Q1W) for four doses, optionally on days 1, 8, 15 and 22; (iii) Q1W for two consecutive doses, optionally on days 1 and 8, followed by every two weeks (Q2W) for one dose, optionally on day 15; or (iv) every two weeks (Q2W) for two doses, optionally on days 1 and 15.

136. The kit of any one of embodiments 135, wherein the instructions specify that:

each Q1W dose of the first 28-day cycle is independently from or from about 18% to 32% of the total dosage amount, optionally is or is about 25% of the total dosage amount in the cycle; and/or

each Q2W dose of the first 28-day cycle is independently from or from about 40% to 62.5% of the total dosage amount, optionally is or is about 50% of the total dosage amount in the cycle.

137. The kit of any one of embodiments 135 or embodiment 136, wherein the instructions specify that:

the first 28-day cycle is carried out by administering the anti-PD-L1 antibody or antigen-binding fragment thereof Q1W for two consecutive doses in an amount of or about 375 mg followed by Q2W for one dose in an amount of or about 750 mg;

the first 28-day cycle is carried out by administering the anti-PD-L1 antibody or antigen-binding fragment thereof Q1W for four doses, said four doses comprising two consecutive doses of or about 225 mg followed by two consecutive doses of or about 375 mg; or

the first 28-day cycle is carried out by administering the anti-PD-L1 antibody or antigen-binding fragment thereof Q1W for two consecutive doses in an amount of or about 375 mg.

138. The kit of any one of embodiments 90-137, wherein the instructions specify that the second and/or a subsequent 28-day cycle is carried out by administering 1 or 2 does of the anti-PD-L1 antibody or antigen-binding fragment thereof.

139. The kit of any one of embodiments 90-138, wherein the instructions specify that the second and/or a subsequent 28-day cycle is carried out with a dosing schedule selected from (i) every two weeks (Q2W) for two doses, optionally on days 1 and 15; or (ii) every four weeks (Q4W) for one dose, optionally on day 1.

140. The kit of embodiment 139, wherein the instructions specify that:

each Q2W dose of the second and/or a subsequent 28-day cycle is or is about 50% of the total dosage amount; and/or

the Q4W dose of the second and/or a subsequent 28-day cycle is or is about the total dosage amount.

141. The kit of embodiment 139 or embodiment 140, wherein the instructions specify that:

the second and/or a subsequent dose is carried out by administering the anti-PD-L1 antibody or antigen-binding fragment thereof Q2W for two doses in an amount of or about 750 mg; or

the second and/or a subsequent dose is carried out by administering the anti-PD-L1 antibody or antigen-binding fragment thereof Q4W for one dose in an amount of or about 1500 mg.

142. The kit of any one of embodiments 90-141, wherein the instructions specify that:

the first 28-day cycle is carried out by administering the anti-PD-L1 antibody or antigen-binding fragment thereof once-weekly (Q1W) for two consecutive doses in an amount of or about 375 mg followed by every two weeks (Q2W) for one dose in an amount of or about 750 mg; and

the second and/or a subsequent 28-day cycle is carried out by administering the anti-PD-L1 antibody or antigen-binding fragment thereof Q4W for one dose in an amount of or about 1500 mg.

143. The kit of any one of embodiments 90-141, wherein the instructions specify that:

the first 28-day cycle is carried out by administering the anti-PD-L1 antibody or antigen-binding fragment thereof once-weekly (Q1W) for four doses, said four doses have two consecutive doses of or about 225 mg followed by two consecutive doses of or about 375 mg; and

the second and/or a subsequent 28-day cycle is carried out by administering the anti-PD-L1 antibody or antigen-binding fragment thereof every two weeks (Q2W) for two doses in an amount of or about 750 mg.

144. The kit of any one of embodiments 90-141, wherein the instructions specify that:

the first 28-day cycle is carried out by administering the anti-PD-L1 antibody or antigen-binding fragment thereof once-weekly (Q1W) for two doses, each of said doses is or is about an amount of or about 375 mg, optionally wherein the doses are consecutive doses, optionally wherein the doses are carried out on days 15 and 22 in the 28-day cycle; and

the second and/or a subsequent 28-day cycle is carried out by administering the anti-PD-L1 antibody or antigen-binding fragment thereof Q4W for one dose in an amount of or about 1500 mg.

145. The kit of any one of embodiments 90-144, wherein the instructions specify that the administration of the administration of the anti-PD-L1 antibody or antigen-binding fragment carries out at least three 28-day cycles.

146. The kit of embodiment 145, wherein the instructions specify that the total dosage amount of the anti-PD-L1 antibody or antigen-binding fragment in the third 28-day cycle is the same as the first and/or second 28-day cycle.

147. The kit of embodiment 145 or embodiment 146, wherein the instructions specify the total dosage amount of the anti-PD-L1 antibody or antigen-binding fragment in the third 28-day cycle is or is about 1500 mg,

148. The kit of any one of embodiments 145-147, wherein the instructions specify that in the third 28-day cycle, the administration of the total dosage amount of the anti-PD-L1 antibody or antigen-binding fragment is carried out by administering the antibody or fragment a greater number of times as compared to the first and/or second 28-day cycle.

149. The kit of any one of embodiments 145-148, wherein the instructions specify that in the third 28-day cycle, the administration of the total dosage amount of the anti-PD-L1 antibody or antigen-binding fragment is carried out by administering the antibody or fragment the same number of times as compared to the second 28-day cycle.

150. The kit of any one of embodiments 145-149, wherein the instructions specify that the third 28-day cycle is carried out with a dosing schedule every four weeks (Q4W) for one dose, optionally on day 1.

151. The kit of any one of embodiments 90-150, wherein the instructions specify that the administration of the anti-PD-L1 antibody or antigen-binding fragment is carried out by no more than three 28-day cycles after initiation of the T cell therapy.

152. The kit of any one of embodiments 90-151, wherein the instructions specify that each 28-day cycle is independently carried out with a dosing schedule selected from (i) once-weekly (Q1W) for four doses, optionally on days 1, 8, 15 and 22; (ii) Q1W for two consecutive doses, optionally on days 1 and 8, followed by every two weeks (Q2W) for one dose, optionally on day 15; (iii) every two weeks (Q2W) for two doses, optionally on days 1 and 15; or (iv) every four weeks (Q4W) for one dose, optionally on day 1.

153. The kit of any one of embodiments 90-152, wherein the instructions specify that the anti-PD-L1 antibody or antigen-binding fragment is administered on day 1, 8 and 15 in a first 28-day cycle, on day 1 in a second 28-day cycle, and on day 1 in a third 28-day cycle.

154. The kit of any one of embodiments 90-152, wherein the instructions specify that the anti-PD-L1 antibody or antigen-binding fragment is administered on day 1, 8, 15 and 22 in a first 28-day cycle, on day 1 and 15 in a second 28-day cycle, and on day 1 in a third 28-day cycle.

155. The kit of any one of embodiments 90-152, wherein the instructions specify that the anti-PD-L1 antibody or antigen-binding fragment is administered on day 1 in each 28-day cycle.

156. The kit of any one of embodiments 90-155, wherein the instructions specify that the administration of the anti-PD-L1 antibody or antigen-binding fragment is carried out by one or more further 28-day cycle if the subject exhibits a partial response (PR) following the treatment.

157. The kit of any one of embodiments 90-156, wherein the instructions specify that the administration of the anti-PD-L1 antibody or antigen-binding fragment is carried out for a total duration of about 12 months or less than about 12 months.

158. The kit of any one of embodiments 90-157, wherein the instructions specify that the administration of the anti-PD-L1 antibody or antigen-binding fragment is initiated at a time greater than 21 days (e.g., at about 29 days, within 22-36 days) after initiation of administration of the T cell therapy.

159. The kit of any one of embodiment 90-157, wherein the instructions specify that the administration of the anti-PD-L1 antibody or antigen-binding fragment is initiated at or within about 29 days, 36 days, 43 days or 50 days after initiation of administration of the T cell therapy.

160. The kit of any one of embodiments 90-159, wherein the instructions instructs that anti-PD-L1 antibody or antigen-binding fragment should not be administered when the subject exhibits a severe toxicity.

161. The kit of embodiment 160, wherein the instructions specify that:

the severe toxicity is severe cytokine release syndrome (CRS), optionally grade 3 or higher, prolonged grade 3 or higher or grade 4 or 5 CRS; and/or

the severe toxicity is severe neurotoxicity, optionally grade 3 or higher, prolonged grade 3 or higher or grade 4 or 5 neurotoxicity.

162. The kit of any one of embodiments 90-161, wherein the instructions specify that:

the administration of the anti-PD-L1 antibody or antigen-binding fragment is initiated at a time at or after, optionally immediately after or within 1 to 3 days after:

(i) peak or maximum level of the cells of the T cell therapy are detectable in the blood of the subject;

(ii) the number of cells of the T cell therapy detectable in the blood, after having been detectable in the blood, is not detectable or is reduced, optionally reduced compared to a preceding time point after administration of the T cell therapy;

(iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy;

(iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject;

(v) the subject exhibits disease progression and/or has relapsed following remission after treatment with the T cell therapy; and/or

(iv) the subject exhibits increased tumor burden as compared to tumor burden at a time prior to or after administration of the cells and prior to initiation of administration of the anti-PD-L1 antibody.

163. The kit of any one of embodiments 90-162, wherein the subject is human.

164. The kit of any one of embodiments 90-163, wherein the instructions specify that the administration of the T cell therapy or the anti-PD-L1 antibody or antigen-binding fragment is for treating a non-Hodgkin lymphoma (NHL).

165. The kit of embodiment 164, wherein the instructions specify that the administration of the T cell therapy or the anti-PD-L1 antibody or antigen-binding fragment is for treating a non-Hodgkin lymphoma (NHL) in the subject, wherein the subject has relapsed following remission after treatment with, or become refractory to, one or more prior therapies for the NHL, optionally one or two prior therapies other than another dose of cells expressing the CAR, optionally wherein the prior therapy is or comprises a CD20-targeted agent or anthracycline.

166. The kit of embodiment 164 or embodiment 165, wherein the instructions specify the NHL as any one of aggressive NHL, diffuse large B cell lymphoma (DLBCL), DLBCL-NOS, optionally transformed indolent; EBV-positive DLBCL-NOS; T cell/histiocyte-rich large B-cell lymphoma; primary mediastinal large B cell lymphoma (PMBCL); follicular lymphoma (FL), optionally, follicular lymphoma Grade 3B (FL3B); and/or high-grade B-cell lymphoma, with MYC and BCL2 and/or BCL6 rearrangements with DLBCL histology (double/triple hit).

167. The kit of any one of embodiments 90-166, wherein the instructions specify that the subject must be identified as having an Eastern Cooperative Oncology Group Performance Status (ECOG) status of less than or equal to 1 to qualify for being a candidate who is or will be subject to the use of this kit.

168. The kit of any one of embodiments 90 and 92-167, wherein the cells are suitable for being administered parenterally, optionally intravenously.

169. The kit of any one of embodiments 90, 91, and 93-168, wherein the anti-PD-L1 antibody or antigen-binding fragment is suitable for being administered parenterally, optionally intravenously.

171. A method of treatment, the method comprising:

(a) administering a T cell therapy to a subject having a B cell malignancy, said cell therapy comprising a dose of genetically engineered T cells expressing a chimeric antigen receptor, wherein the chimeric antigen receptor specifically binds to a target antigen expressed by the B cell malignancy; and

(b) subsequently administering to the subject a checkpoint inhibitor that is an antibody or antigen-binding fragment thereof capable of blocking an immune checkpoint pathway protein, wherein a total dosage amount of the checkpoint inhibitor is administered in each of at least two dosage cycles, wherein the total dosage amount of the checkpoint inhibitor in the first of the at least two dosage cycles:

is the same as or less than the total dosage amount administered in the second and/or a subsequent dosage cycle; and

is administered in more than one individual dose over the course of the first dosage cycle, wherein the number of individual doses is greater than the number of individual doses administered in the second and/or a subsequent dosage cycle.

172. A method of treatment, the method comprising administering, to a subject having a B cell malignancy a checkpoint inhibitor that is an antibody or antigen-binding fragment thereof capable of blocking an immune checkpoint pathway protein, said subject having been administered a T cell therapy comprising a dose of genetically engineered T cells expressing a chimeric antigen receptor that specifically binds to a target antigen expressed by the B cell malignancy, wherein a total dosage amount of the checkpoint inhibitor is administered in each of at least two dosage cycles, wherein the total dosage amount of the checkpoint inhibitor in the first of the at least two dosage cycles:

is the same as or less than the total dosage amount administered in the second and/or a subsequent dosage cycle; and

is administered in more than one individual dose over the course of the first dosage cycle, wherein the number of individual doses is greater than the number of individual doses administered in the second and/or a subsequent dosage cycle.

173. The method of embodiment 171 or embodiment 172, wherein the dosage cycle is a 21-day cycle.

174. The method of embodiment 171 or embodiment 172, wherein the dosage cycle is a 28-day cycle.

175. The method of any of embodiments 171-174, wherein the total dosage amount in the first of the at least two dosage cycles is the same as the total dosage amount in the second of the at least two dosage cycles.

176. The method of any of embodiments 171-175, wherein the first of the at least two dosage cycles comprises 2, 3, 4 or more individual doses.

177. The method of embodiment 176, wherein the dosage cycle is a 28-day cycle and the individual doses of the first of the at least two 28-day cycles are administered as four doses each once every week (Q1W), two doses each as Q1W doses for two consecutive weeks, or two doses each as Q1W doses for two consecutive weeks and followed by one dose once in two weeks (Q2W).

178. The method of any of embodiments 171-177, wherein each of said at least two dosage cycles comprises administering independently a total dosage amount of at or about 400 mg to at or about 2000 mg of the checkpoint inhibitor.

179. The method of any of embodiments 171-178, wherein the checkpoint inhibitor blocks an immune checkpoint pathway protein selected from among PD-L1, PD-L2, PD-1 and CTLA-4.

180. The method of any of embodiments 171-179, wherein the checkpoint pathway is PD-1/PD-L1 and the checkpoint inhibitor is an anti-PD-1 antibody.

181. The method of embodiment 180, wherein the checkpoint inibitior is nivolumab, pembrolizumab, orcemiplimab.

182. The method of any of embodiments 171-181, wherein each of said at least two dosage cycle comprises administering independently a total dosage amount of at or about 400 mg to at or about 600 mg, optionally at or about 480 mg.

183. The method of any of embodiments 171-179, wherein the checkpoint pathway is PD-1/PD-L1 and the checkpoint inhibitor is an anti-PD-L1 antibody.

184. The method of any of embodiments 171-181 and 183, wherein each of said at least two dosage cycle comprises administering independently a total dosage amount of 750 mg to 2000 mg, optionally at or about 1500 mg.

185. The method of any of embodiments 171-184, wherein administration of the checkpoint inhibitor and/or the start of the first dosage cycle is initiated at a time at or after, optionally immediately after or within 1 to 3 days after:

(i) peak or maximum level of the cells of the T cell therapy are detectable in the blood of the subject;

(ii) the number of cells of the T cell therapy detectable in the blood, after having been detectable in the blood, is not detectable or is reduced, optionally reduced compared to a preceding time point after administration of the T cell therapy;

(iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy;

(iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject;

(v) the subject exhibits disease progression and/or has relapsed following remission after treatment with the T cell therapy; and/or

(iv) the subject exhibits increased tumor burden as compared to tumor burden at a time prior to or after administration of the cells and prior to initiation of administration of the checkpoint inhibitor.

186. The method of any of embodiments 171-185, wherein administration of the checkpoint inhibitor and/or the start of the first dosage cycle is initiated at or within 29 days, 36 days, 43 days or 50 days after initiation of administration of the T cell therapy.

187. The method of any of embodiments 171-186, wherein administration of the checkpoint inhibitor and/or the start of the first dosage cycle is initiated from or from about 22 days to 36 days after initiation of administration of the T cell therapy.

188. The method of any of embodiments 171-187, wherein administration of the checkpoint inhibitor and/or the start of the first dosage cycle is initiated at or about 29 days after initiation of administration of the T cell therapy.

189. The method of any of embodiments 171-188, wherein administration of the checkpoint inhibitor and/or the start of the first dosage cycle is initiated at or about 43 days after initiation of administration of the T cell therapy.

190. The method of any of embodiments 171-189, wherein at the time of administering the checkpoint inhibitor and/or the start of the first dosage cycle, the subject does not exhibit a severe toxicity following administration of the T cell therapy.

191. The method of embodiment 190, wherein:

the severe toxicity is severe cytokine release syndrome (CRS), optionally grade 3 or higher, prolonged grade 3 or higher or grade 4 or 5 CRS; and/or

the severe toxicity is severe neurotoxicity, optionally grade 3 or higher, prolonged grade 3 or higher or grade 4 or 5 neurotoxicity.

192. A method of treatment, the method comprising:

(a) administering a T cell therapy to a subject having a B cell malignancy, said T cell therapy comprising a dose of genetically engineered T cells expressing a chimeric antigen receptor, wherein the chimeric antigen receptor specifically binds to a target antigen expressed by cells of the B cell malignancy; and

(b) subsequently administering to the subject an anti-PD-L1 antibody or antigen-binding fragment thereof, wherein said administration comprises carrying out at least two 28-day cycles, wherein:

the first 28-day cycle comprises administering the anti-PD-L1 antibody or antigen-binding fragment thereof as two individual doses each once-weekly (Q1W) for two consecutive weeks of the 28-day cycle, each of said individual doses in an amount of or about 375 mg, followed by one dose once in two weeks (Q2W) of the 28-day cycle in an amount of or about 750 mg; and

the second and/or a subsequent 28-day cycle comprises administering the anti-PD-L1 antibody or antigen-binding fragment thereof as one dose every four weeks (Q4W) for in an amount of or about 1500 mg.

193. A method of treatment, the method comprising administering an anti-PD-L1 antibody or antigen-binding fragment thereof to a subject having a B cell malignancy, said subject having been administered a T cell therapy comprising a dose of genetically engineered T cells expressing a chimeric antigen receptor that specifically binds to a target antigen expressed by the B cell malignancy wherein the administration of the anti-PD-L1 antibody or antigen-binding fragment thereof comprises carrying out at least two 28-day cycles, wherein:

the first 28-day cycle comprises administering the anti-PD-L1 antibody or antigen-binding fragment thereof as two individual doses each once-weekly (Q1W) for two consecutive weeks of the 28-day cycle, each of said individual doses in an amount of or about 375 mg, followed by one dose once in two weeks (Q2W) of the 28-day cycle in an amount of or about 750 mg; and

the second and/or a subsequent 28-day cycle comprises administering the anti-PD-L1 antibody or antigen-binding fragment thereof as one dose every four weeks (Q4W) for in an amount of or about 1500 mg.

194. A method of treatment, the method comprising:

(a) administering a T cell therapy to a subject having a B cell malignancy, said T cell therapy comprising a dose of genetically engineered T cells expressing a chimeric antigen receptor, wherein the chimeric antigen receptor specifically binds to a target antigen expressed by the B cell maligancy; and

(b) subsequently administering to the subject an anti-PD-L1 antibody or antigen-binding fragment thereof, said administration comprises carrying out at least two 28-day cycles, wherein:
the first 28-day cycle comprises administering the anti-PD-L1 antibody or antigen-binding fragment thereof as four individual doses each once-weekly (Q1W) for the 28-day cycle, wherein, said four doses comprises two consecutive Q1W doses each independently of or of about 225 mg followed by two consecutive Q1W doses each independently of or of about 375 mg; and
the second and/or a subsequent 28-day cycle comprises administering the anti-PD-L1 antibody or antigen-binding fragment thereof as two doses each every two weeks (Q2W) of the 28-day cycle, wherein each Q2W administration is each independently in an amount of or of about 750 mg.

195. A method of treatment, the method comprising administering an anti-PD-L1 antibody or antigen-binding fragment thereof to a subject having a B cell malignancy, said subject having been administered a T cell therapy comprising a dose of genetically engineered T cells expressing a chimeric antigen receptor that specifically binds to a target antigen expressed on the B cell malignancy, wherein the anti-PD-L1 antibody or antigen-binding fragment thereof comprises carrying out at least two 28-day cycles, wherein:

the first 28-day cycle comprises administering the anti-PD-L1 antibody or antigen-binding fragment thereof as four individual doses each once-weekly (Q1W) for the 28-day cycle, wherein the four individual doses comprises two consecutive Q1W doses each independently of or of about 225 mg followed by two consecutive Q1W doses each independently of or about 375 mg; and

the second and/or a subsequent 28-day cycle comprises administering the anti-PD-L1 antibody or antigen-binding fragment thereof as two individual doses every two weeks (Q2W) for the second and/or subsequent 28-day cycle, wherein each dose independently isin an amount of or about 750 mg.

196. A method of treatment, the method comprising:

(a) administering a T cell therapy to a subject having a B cell malignancy, said T cell therapy comprising a dose of genetically engineered T cells expressing a chimeric antigen receptor, wherein the chimeric antigen receptor specifically binds to a target antigen expressed by the B cell malignancy; and

(b) subsequently administering to the subject an anti-PD-L1 antibody or antigen-binding fragment thereof, said administration comprises carrying out at least two 28-day cycles, wherein:
the first 28-day cycle comprises administering the anti-PD-L1 antibody or antigen-binding fragment thereof as two individual doses each once-weekly (Q1W), wherein each of said two doses independently comprises an amount of or of about 375 mg, optionally wherein the two doses are consecutive Q1W doses, optionally wherein the two doses are administered on days 15 and 22 in the 28-day cycle; and
the second and/or a subsequent 28-day cycle comprises administering the anti-PD-L1 antibody or antigen-binding fragment thereof Q4W for one dose in the second and/or subsequent 28-day cycle in an amount of or about 1500 mg.

197. A method of treatment, the method comprising administering an anti-PD-L1 antibody or antigen-binding fragment thereof to a subject having a B cell malignancy, said subject having been administered a T cell therapy comprising a dose of genetically engineered T cells expressing a chimeric antigen receptor that specifically binds to a target antigen expressed on the B cell malignancy, wherein the administration of the anti-PD-L1 antibody or antigen-binding fragment comprises carrying out at least two 28-day cycles, wherein:
the first 28-day cycle comprises administering the anti-PD-L1 antibody or antigen-binding fragment thereof as two individual doses each once-weekly (Q1W), wherein each of said two doses independently comprises an amount of or of about 375 mg, optionally wherein the two doses are consecutive Q1W doses, optionally wherein the two doses are administered on days 15 and 22 in the 28-day cycle; and
the second and/or a subsequent 28-day cycle comprises administering the anti-PD-L1 antibody or antigen-binding fragment thereof Q4W for one dose in the second and/or subsequent 28-day cycle in an amount of or about 1500 mg.

198. A method of treatment, the method comprising:

(a) administering a T cell therapy to a subject having a B cell malignancy, said cell therapy comprising a dose of genetically engineered T cells expressing a chimeric antigen receptor, wherein the chimeric antigen receptor specifically binds to a target antigen expressed by the B cell malignancy; and

(b) subsequently administering to the subject an anti-PD-L1 antibody or antigen-binding fragment thereof, wherein:
the administration of the anti-PD-L1 antibody or antigen-binding fragment comprises carrying out at least two 28-day cycles, each of said at least two 28-day cycles, comprising administering a total dosage amount of 750 mg to 2000 mg of the antibody or antigen-binding fragment; and
in at least one of said at least two 28-day cycles, the administration of the total dosage amount of the anti-PD-L1 antibody or antigen-binding fragment is carried out by administering more than one individual doses of the antibody or fragment over the course of the at least one 28-day cycle.

199. A method of treatment, the method comprising administering an anti-PD-L1 antibody or antigen-binding fragment thereof to a subject having a B cell malignancy, said subject having been administered a T cell therapy comprising a dose of genetically engineered T cells expressing a chimeric antigen receptor, wherein the chimeric antigen receptor specifically binds to a target antigen expressed by the B cell malignancy, wherein:

the administration of the anti-PD-L1 antibody or antigen-binding fragment comprises carrying out at least two 28-day cycles, each of said at least two 28-day cycles, independently, comprising administering a total dosage amount of 750 mg to 2000 mg of the antibody or antigen-binding fragment; and
in at least one of said at least two 28-day cycles, the administration of the total dosage amount of the anti-PD-L1 antibody or antigen-binding fragment is carried out by administering more than one individual dose of the antibody or fragment over the course of the at least one 28-day cycle.

200. The method of embodiment 198 or embodiment 199, wherein in a first of said at least two 28-day cycles, the administration of the total dosage amount of the anti-PD-L1 antibody or antigen-binding fragment is carried out by administering a greater number of individual doses of the antibody or fragment as compared to the administration in the second and/or a subsequent 28-day cycle.

201. The method of any of embodiments 198-200, wherein the total dosage amount of the anti-PD-L1 antibody or antigen-binding fragment in each 28-day cycle independently is between at or about 750 mg and at or about 1500 mg.

202. The method of any of embodiments 198-201, wherein the total dosage amount of the anti-PD-L1 antibody or antigen-binding fragment in at least one of the 28-day cycles is at or about 750 mg.

203. The method of any of embodiments 198-202, wherein the total dosage amount of the anti-PD-L1 antibody or antigen-binding fragment in at least one of the 28-day cycles is at or about 1200 mg.

204. The method of any of embodiments 198-201, wherein the total dosage amount of the anti-PD-L1 antibody or antigen-binding fragment in at least one of the 28-day cycles is at or about 1500 mg.

205. The method of any of embodiments 198-201 and 204, wherein the total dosage amount of the anti-PD-L1 antibody or antigen-binding fragment in each 28-day cycle, independently, is at or about 1500 mg.

206. The method of any of embodiments 198-205, wherein the total dosage amount of the anti-PD-L1 antibody or antigen-binding fragment in at least two of said at least two, and optionally in said at least two, 28-day cycles is the same total dosage amount.

207. The method of any of embodiments 198-205, wherein the total dosage amount of the anti-PD-L1 antibody or antigen-binding fragment is different in at least two of, or is different in each of, said at least two 28-day cycles.

208. The method of any of embodiments 198-205 and 207, wherein the total dosage amount of the anti-PD-L1 antibody or antigen-binding fragment in the first of said at least two 28-day cycles is lower than the second and/or a subsequent of said at least two 28-day cycle.

209. The method of any of embodiments 198-208, wherein the administration of the total dosage amount in the first of said at least two 28-day cycles comprises administering 2, 3 or 4 individual doses of the anti-PD-L1 antibody or antigen-binding fragment thereof.

210. The method of any of embodiments 198-209, wherein the administration of the total dosage amount in the first of said at least two 28-day cycles comprises administering the anti-PD-L1 antibody or antigen-binding fragment thereof as individual doses according to a dosing schedule selected from (i) two individual doses each once-weekly (Q1W) within the 28-day cycle, optionally on days 15 and 22 of the 28-day cycle; (ii) four individual doses each once-weekly (Q1W) for the 28-day cycle, optionally on days 1, 8, 15 and 22 of the 28-day cycle; (iii) two individual doses each Q1W for two consecutive weeks of the 28-day cycle, optionally on days 1 and 8 of the cycle, followed by one dose once in two weeks (Q2W) of the 28-day cycle, optionally on day 15 of the cycle; or (iv) two individual doses each every two weeks (Q2W) for the 28-day cycle, optionally on days 1 and 15 of the 28-day cycle.

211. The method of embodiment 210, wherein:

each Q1W dose administered in the first 28-day cycle is independently from or from at or about 18% to at or about 32% of the total dosage amount administered in the first 28-day cycle, optionally is at or about 25% of the total dosage amount administered in the first 28-day cycle; and/or

each Q2W dose administered in the first 28-day cycle is independently from or from at or about 40% to at or about 62.5% of the total dosage amount in the first 28-day cycle, optionally is at or about 50% of the total dosage amount administered in the first 28-day cycle.

212. The method of embodiment 210 or embodiment 211, wherein:

the administration of the total dosage amount in the first 28-day cycle comprises administering the anti-PD-L1 antibody or antigen-binding fragment thereof according to dosing schedule (iii), wherein each of the two individual doses Q1W for two consecutive weeks is, each independently, in an amount of or of about 375 mg followed by one dose once Q2W in an amount of or of about 750 mg;

the administration of the total dosage amount in the first 28-day cycle comprises administering the anti-PD-L1 antibody or antigen-binding fragment thereof according to dosing schedule set forth in (ii), wherein the four individual doses Q1W comprise two consecutive Q1W doses in an amount of or of about 225 mg followed by two consecutive Q1W doses in an amount of or of about 375 mg; or

the administration of the total dosage amount in the first 28-day cycle comprises administering the anti-PD-L1 antibody or antigen-binding fragment according to dosing schedule set forth in (i), wherein each of the two individual doses Q1W are carried out for two consecutive Q1W doses in an amount of or of about 375 mg.

213. The method of any of embodiments 198-210, wherein the administration of the total dosage amount in the first of said at least two 28-day cycles comprises administering individual doses according to a dosing schedule selected from (i) two individual doses on or about day 15 and on or about day 22 of the 28-day cycle; (ii) four individual doses on or about day 1, on or about day 8, on or about day 15 and on or about day 22 of the 28-day cycle; (iii) two individual doses on or about day 1 and on or about day 8 of the 28-day cycle, followed by one dose on or about day 15 of the cycle; or (iv) two doses on or about day 1 of the 28-day cycle and on or about day 15 of the 28-day cycle.

214. The method of any of embodiments 198-213, wherein:

the administration of the total dosage amount in the first 28-day cycle comprises administering the anti-PD-L1 antibody or antigen-binding fragment thereof according to dosing schedule (iii), wherein each of the two individual doses comprise an amount of or of about 375 mg on or about day 1 and on or about day 8 of the 28-day cycle, followed by one dose in an amount of or of about 750 mg on or about day 15 of the cycle

the administration of the total dosage amount in the first 28-day cycle comprises administering the anti-PD-L1 antibody or antigen-binding fragment thereof according to dosing schedule set forth in (ii), wherein the four individual doses comprise two consecutive doses in an amount of or of about 225 mg on or about day 1 and on or about day 8, followed by two consecutive doses in an amount of or of about 375 mg on or about day 15 and on or about day 22 of the 28-day cycle; or

the administration of the total dosage amount in the first 28-day cycle comprises administering the anti-PD-L1 antibody or antigen-binding fragment according to dosing schedule set forth in (i), wherein each of the two individual doses comprise two consecutive in an amount of or of about 375 mg on or about day 15 and on or about day 22 of the 28-day cycle.

215. The method of any of embodiments 200-214, wherein the administration of the total dosage amount in the second and/or a subsequent 28-day cycle, independently, comprises administering 1 or 2 doses of the anti-PD-L1 antibody or antigen-binding fragment thereof.

216. The method of any of embodiments 200-215, wherein the administration of the total dosage amount in the second and/or a subsequent 28-day cycle, independently, comprises a dosing schedule selected from (i) two individual doses each every two weeks (Q2W) for the second and/or subsequent 28-day cycle, optionally on days 1 and 15 of the second and/or subsequent cycle; or (ii) one dose every four weeks (Q4W) of the second and/or subsequent 28-day cycle, optionally on day 1 of the second and/or subsequent cycle.

217. The method of embodiment 216, wherein:

each Q2W dose of the second and/or subsequent 28-day cycle is or is about 50% of the total dosage amount of the second and/or subsequent 28 day cycle; and/or

the Q4W dose of the second and/or subsequent 28-day cycle is or is about the total dosage amount of the second and/or subsequent 28 day cycle.

218. The method of embodiment 216 or embodiment 217, wherein:

the second and/or a subsequent dose comprises administering the anti-PD-L1 antibody or antigen-binding fragment thereof Q2W for two doses in an amount of or of about 750 mg; or

the second and/or a subsequent dose comprises administering the anti-PD-L1 antibody or antigen-binding fragment thereof Q4W for one dose in an amount of or of about 1500 mg.

219. The method of any of embodiments 174-218, wherein at least two 28-day cycles further comprises a third 28-day cycle and/or wherein the subsequent 28-day cycle is a third 28-day cycle.

220. The method of embodiment 219, wherein the total dosage amount of the anti-PD-L1 antibody or antigen-binding fragment in the third 28-day cycle is the same as the total dosage amount administered in the first and/or in the second 28-day cycle.

221. The method of embodiment 219 or embodiment 220, wherein the total dosage amount of the anti-PD-L1 antibody or antigen-binding fragment in the third 28-day cycle is or is about 1500 mg.

222. The method of any of embodiments 219-221, wherein:

(a) in the third 28-day cycle, the administration of the total dosage amount of the anti-PD-L1 antibody or antigen-binding fragment is carried out by administering the antibody or fragment in a greater number of individual doses as compared to in the first and/or second 28-day cycle; or

(b) in the third 28-day cycle, the administration of the total dosage amount of the anti-PD-L1 antibody or antigen-binding fragment is carried out by administering the same number of doses of the antibody or fragment as compared to the second 28-day cycle.

223. The method of any of embodiments 219-222, wherein the administration of the total dosage amount in the third 28-day cycle comprises administration of one dose every four weeks (Q4W) of the third 28-day cycle, optionally on day 1 of the third 28-day cycle.

224. The method of any one of embodiments 192-223, wherein the first of said at least two 28-day cycles is initiated at a time:

(a) between day 22 and day 36 of initiation of the administration of the T cell therapy; or

(b) at or after, optionally immediately after or within 1 to 3 days after:
(i) peak or maximum level of the cells of the T cell therapy are detectable in the blood of the subject;
(ii) the number of cells of the T cell therapy detectable in the blood, after having been detectable in the blood, is not detectable or is reduced, optionally reduced compared to a preceding time point after administration of the T cell therapy;
(iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy;
(iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject;
(v) the subject exhibits disease progression and/or has relapsed following remission after treatment with the T cell therapy; and/or
(iv) the subject exhibits increased tumor burden as compared to tumor burden at a time prior to or after administration of the cells and prior to initiation of administration of the anti-PD-L1 antibody.

225. The method of any of embodiments 192-224, wherein the first of said at least two 28-day cycles is initiated at a time between day 22 and day 36 of initiation of the administration of the T cell therapy.

226. The method of any of embodiments 192-225, wherein the at least two 28-day cycles comprise no more than three 28-day cycles, optionally wherein the first of said at least two 28-day cycles is initiated between at or about day 22 and at or about day 36.

227. The method of any of embodiments 192-226, wherein the first of said at least two 28-day cycle is initiated at or about day 29 after initiation of the administration of the T cell therapy.

228. The method of any of embodiments 192-227, wherein the first of said at least two 28-day cycle is initiated at or about day 43 after initiation of administration of the T cell therapy.

229. A method of treatment, the method comprising:

(a) administering a T cell therapy to a subject having a B cell malignancy, said T cell therapy comprising a dose of genetically engineered T cells expressing a chimeric antigen receptor, wherein the chimeric antigen receptor specifically binds to a target antigen expressed by the B cell malignancy; and

(b) subsequently administering to the subject an anti-PD-L1 antibody or antigen-binding fragment thereof, wherein the administration of antibody or antigen-binding fragment comprises carrying out between one and three 28-day cycles, each cycle comprising administering a total dosage amount of 750 mg to 2000 mg of the antibody or fragment, optionally wherein the first of said between one and three 28-day cycle begins between at or about day 22 and at or about day 36, optionally at day 29, after initiation of the T cell therapy.

230. A method of treatment, the method comprising administering an anti-PD-L1 antibody or antigen-binding fragment thereof to a subject having a B cell malignancy, said subject having been administered a T cell therapy comprising a dose of genetically engineered T cells expressing a chimeric antigen receptor, wherein the chimeric antigen receptor specifically binds to a target antigen expressed by the B cell malignancy, wherein the administration of the antibody or antigen-binding fragment comprises carrying out between one and three 28-day cycles, each cycle comprises administering a total dosage amount of 900 mg to 2000 mg of the antibody or fragment, optionally wherein the first of said between one and three 28-day cycles begins between at or about day 22 and at or about day 36, optionally at about day 29, after initiation of the T cell therapy.

231. The method of embodiment 229 or embodiment 230, wherein the total dosage amount of the anti-PD-L1 antibody or antigen-binding fragment in each 28-day cycle independently is or is about 1200 mg to 1500 mg.

232. The method of any of embodiments 229-231, wherein the total dosage amount of the anti-PD-L1 antibody or antigen-binding fragment in at least one 28-day cycle is or is about 1200 mg.

233. The method of any of embodiments 229-231, wherein the total dosage amount of the anti-PD-L1 antibody or antigen-binding fragment in at least one 28-day cycle is or is about 1500 mg.

234. The method of any of embodiments 229-23 land 233, wherein the total dosage amount of the anti-PD-L1 antibody or antigen-binding fragment in each 28-day cycle is or is about 1500 mg.

235. The method of any of embodiments 229-234, wherein the total dosage amount in each 28-day cycle comprises administering 1, 2, 3 or 4 doses of the anti-PD-L1 antibody or antigen-binding fragment thereof.

236. The method of any of embodiments 229-235, wherein each 28-day cycle independently comprises a dosing schedule selected from (i) four doses each once-weekly (Q1W), optionally on days 1, 8, 15 and 22 of the 28-day cycle; (ii) two consecutive doses each Q1W, optionally on days 1 and 8, followed by one dose once in two weeks (Q2W) for one dose, optionally on day 15, of the 28-day cycle; (iii) two doses each every two weeks (Q2W), optionally on days 1 and 15 of the 28-day cycle; or (iv) one dose every four weeks (Q4W), optionally on day 1, of the 28-day cycle.

237. The method of any of embodiments 229-236 wherein the anti-PD-L1 antibody or antigen-binding fragment is administered on day 1, 8 and 15 in a first 28-day cycle, on day 1 in a second 28-day cycle, and on day 1 in a third 28-day cycle.

238. The method of any of embodiments 229-237, wherein the anti-PD-L1 antibody or antigen-binding fragment is administered on day 1, 8, 15 and 22 in a first 28-day cycle, on day 1 and 15 in a second 28-day cycle, and on day 1 in a third 28-day cycle.

239. The method of any of embodiments 229-238, wherein the anti-PD-L1 antibody or antigen-binding fragment is administered on day 1 in each 28-day cycle.

240. The method of any of embodiments 229-239, further comprising administering the anti-PD-L1 antibody or antigen-binding fragment in one or more further 28-day cycle if the subject exhibits no more than a partial response (PR) following the treatment and/or exhibits no more than a PR at three-months following initiation of administration of the T cell therapy and/or of the anti-PD-L1 antibody or fragment.

241. The method of embodiment 240, wherein the anti-PD-L1 antibody or antigen-binding fragment is administered in a total dosage amount of 900 mg to 2000 mg in each of the one or more further 28-day cycle, optionally in a total dosage amount of at or about 1500 mg.

242. The method of any of embodiments 183-241, wherein the anti-PD-L1 antibody or antigen-binding fragment is administered for a total duration of no more than 12 months.

243. The method of embodiment any of embodiments 183-242, wherein the administration of the anti-PD-L1 antibody or antigen-binding fragment and/or the start of the first 28-day cycle is initiated greater than 21 days after initiation of administration of the T cell therapy.

244. The method of any of embodiments 173-243, wherein administration of the anti-PD-L1 antibody or antigen-binding fragment and/or the start of the first 28-day cycle is initiated at a time at or after, optionally immediately after or within 1 to 3 days after:

(i) peak or maximum level of the cells of the T cell therapy are detectable in the blood of the subject;

(ii) the number of cells of the T cell therapy detectable in the blood, after having been detectable in the blood, is not detectable or is reduced, optionally reduced compared to a preceding time point after administration of the T cell therapy;

(iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy;

(iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject;

(v) the subject exhibits disease progression and/or has relapsed following remission after treatment with the T cell therapy; and/or

(iv) the subject exhibits increased tumor burden as compared to tumor burden at a time prior to or after administration of the cells and prior to initiation of administration of the anti-PD-L1 antibody.

245. The method of any of embodiments 183-244, wherein administration of the anti-PD-L1 antibody or antigen-binding fragment and/or the start of the first 28-day cycle is initiated at or within 29 days, 36 days, 43 days or 50 days after initiation of administration of the T cell therapy.

246. The method of any of embodiments 183-245, wherein administration of the anti-PD-L1 antibody or antigen-binding fragment and/or the start of the first 28-day cycle is initiated from or from about 22 days to 36 days after initiation of administration of the T cell therapy.

247. The method of any of embodiments 183-246, wherein administration of the anti-PD-L1 antibody or antigen-binding fragment and/or the start of the first 28-day cycle is initiated at or about 29 days after initiation of administration of the T cell therapy.

248. The method of any of embodiments 183-247, wherein administration of the checkpoint inhibitor and/or the start of the first dosage cycle is initiated at or about 43 days after initiation of administration of the T cell therapy.

249. The method of any of embodiments 183-248, wherein at the time of administering the anti-PD-L1 antibody or antigen-binding fragment and/or the start of the first 28-day cycle, the subject does not exhibit a severe toxicity following administration of the T cell therapy.

250. The method of embodiment 249, wherein:

the severe toxicity is severe cytokine release syndrome (CRS), optionally grade 3 or higher, prolonged grade 3 or higher or grade 4 or 5 CRS; and/or

the severe toxicity is severe neurotoxicity, optionally grade 3 or higher, prolonged grade 3 or higher or grade 4 or 5 neurotoxicity.

251. The method of any of embodiments 183-250, wherein the anti-PD-L1 antibody or antigen-binding fragment thereof specifically binds to an extracellular domain of PD-L1.

252. The method of any of embodiments 183-251, wherein the anti-PD-L1 antibody or antigen-binding fragment thereof is MEDI4736 (durvalumab), MDPL3280A (atezolizumab), YW243.55.S70, MDX-1105 (BMS-936559), LY3300054, or MSB0010718C (avelumab), or is or comprises an antigen-binding fragment or region of any of the foregoing.

253. The method of any of embodiments 183-252, wherein the anti-PD-L1 antibody or antigen-binding fragment thereof is MEDI4736 (durvalumab) or is or comprises an antigen-binding fragment or region thereof.

254. The method of any of embodiments 183-253, wherein the anti-PD-L1 antibody antibody or antigen binding fragment thereof of MEDI4736 (durvalumab).

255. The method of any of embodiments 171-254, wherein the B cell malignancy is a non-Hodgkin lymphoma (NHL).

256. The method of embodiment 255, wherein, at or immediately prior to the time of the administration of the T cell therapy the subject has relapsed following remission after treatment with, or become refractory to, one or more prior therapies for the NHL, optionally one or two prior therapies other than another dose of cells expressing the CAR, optionally wherein the one or more prior therapy is or comprises a CD20-targeted agent or anthracycline.

257. The method of embodiment 255 or embodiment 256, wherein the NHL comprises aggressive NHL; diffuse large B cell lymphoma (DLBCL); DLBCL-NOS, optionally transformed indolent; EBV-positive DLBCL-NOS; T cell/histiocyte-rich large B-cell lymphoma; primary mediastinal large B cell lymphoma (PMBCL); follicular lymphoma (FL), optionally, follicular lymphoma Grade 3B (FL3B); and/or high-grade B-cell lymphoma, with MYC and BCL2 and/or BCL6 rearrangements with DLBCL histology (double/triple hit).

258. The method of any of embodiments 255-257, wherein the NHL comprises diffuse large B cell lymphoma (DLBCL); DLBCL-NOS; DLBCL-NOS transformed indolent; follicular lymphoma Grade 3B (FL3B); and/or high-grade B-cell lymphoma, with MYC and BCL2 and/or BCL6 rearrangements with DLBCL histology (double/triple hit).

259. The method of any of embodiments 171-258, wherein the subject is or has been identified as having an Eastern Cooperative Oncology Group Performance Status (ECOG) status of less than or equal to 1.

260. The method of any of embodiments 171-199, wherein the target antigen is a B cell antigen.

261. The method of any of embodiments 171-260, wherein the target antigen is CD19.

262. The method of embodiment 261, wherein the chimeric antigen receptor (CAR) comprises an extracellular antigen-recognition domain that specifically binds to a target antigen and an intracellular signaling domain comprising an ITAM.

263. The method of embodiment 262, wherein the intracellular signaling domain comprises a signaling domain of a CD3-zeta (CD3) chain.

264. The method of embodiment 262 or embodiment 263, wherein the chimeric antigen receptor (CAR) further comprises a costimulatory signaling region comprising a cytoplasmic signaling domain of a costimulatory molecule.

265. The method of embodiment 264, wherein the costimulatory signaling region comprises a cytoplasmic signaling domain of CD28 or 4-1BB.

266. The method of embodiment 264 or embodiment 265, wherein the costimulatory domain is or comprises a cytoplasmic signaling domain of 4-1BB.

267. The method of any of embodiments 171-266, wherein:

the CAR comprises an scFv specific for CD19, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is or comprises a CD3zeta signaling domain, and optionally further comprises a spacer between the transmembrane domain and the scFv.

268. The method of any of embodiments 171-266, wherein the CAR comprises, in order, an scFv specific for CD19, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is a CD3zeta signaling domain.

269. The method of any of embodiments 171-266, wherin the CAR comprises, in order, an scFv specific for CD19, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain.

270. The method of embodiment 267 or embodiment 269, wherein:

the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof.

271. The method of any of embodiments 267, 269 and 270, wherein the spacer comprises or consists of the formula X₁PPX₂P (SEQ ID NO:58), where X₁ is glycine, cysteine or arginine and X₂ is cysteine or threonine.

272. The method of any of embodiments 267 and 269-271, wherein the spacer comprises or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto.

273. The method of any of embodiments 267 and 269-272, wherein the spacer comprises the sequence of SEQ ID NO: 1.

274. The method of any of embodiments 264-273, wherein the cytoplasmic signaling domain of a costimulatory molecule comprises SEQ ID NO: 182 or a variant thereof having at least 85%, 86%, 87%, 88%, 89%, 90%, 91%, 92%, 93%, 94%, 95%, 96%, 97%, 98%, 99% or more sequence identity thereto.

275. The method of any of embodiments 262-274, wherein the cytoplasmic signaling domain derived from a primary signaling ITAM-containing molecule comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto.

276. The method of any of embodiments 262-275, wherein the extracellular antigen-recognition domain is an scFv and the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40).

277. The method of any of embodiments 262-276, wherein the extracellular antigen-recognition domain is an scFv and the scFv comprises a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63.

278. The method of any of embodiments 262-277, wherein the extracellular antigen-recognition domain is an scFv and the scFv comprises a variable heavy chain region of FMC63 and a variable light chain region of FMC63.

279. The method of any of embodiments 262-278, wherein the wherein the extracellular antigen-recognition domain is an scFv and the scFv comprises a V_{H} region comprising an amino acid sequence set forth in SEQ ID NO:41.

280. The method of any of embodiments 262-279, wherein the wherein the extracellular antigen-recognition domain is an scFv and the scFv comprises a V_{L} region comprising an amino acid sequence set forth in SEQ ID NO:42.

281. The method of any of embodiments 262-270, wherein the extracellular antigen-recognition domain is an scFv and the scFv comprises, in order, a V_{H}, optionally comprising the amino acid sequence set forth in SEQ ID NO:41, a linker, optionally comprising SEQ ID NO: 59, and a V_{L}, optionally comprising the amino acid sequence set forth in SEQ ID NO:42, and/or the scFv comprises a flexible linker and/or comprises the amino acid sequence set forth as SEQ ID NO: 43.

282. The method of any of embodiments 262-281, wherein the wherein the extracellular antigen-recognition domain is an scFv and the scFv comprise an amino acid sequence set forth in SEQ ID NO:43.

283. The method of any of embodiments 171-282, wherein the dose of genetically engineered T cells comprises from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, 5×10⁷ to 1×10⁸ total CAR-expressing T cells, each inclusive.

284. The method of any of embodiments 171-283, wherein the dose of genetically engineered T cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

285. The method of any of embodiments 171-284, wherein the dose of genetically engineered T cells comprises at or about 5×10⁷ CAR-expressing cells.

286. The method of any of embodiments 171-284, wherein the dose of genetically engineered T cells comprises at or about 1×10⁸ CAR-expressing cells.

287. The method of any of embodiments 171-284, wherein the dose of genetically engineered T cells comprises at or about 1.5×10⁸ CAR-expressing cells.

288. The method of any of embodiments 171-287, wherein the dose of genetically engineered T cells is administered parenterally, optionally intravenously.

289. The method of embodiment 288, wherein the T cells are primary T cells obtained from a subject.

290. The method of any of embodiments 171-289, wherein the T cells are autologous to the subject.

291. The method of any of embodiments 171-289, wherein the T cells are allogeneic to the subject.

292. The method of any of embodiments 171-291, wherein the dose of genetically engineered T cells comprises CD4+ T cells expressing the CAR and CD8+ T cells expressing the CAR and the administration of the dose comprises administering a plurality of separate compositions, said plurality of separate compositions comprising a first composition comprising one of the CD4+ T cells and the CD8+ T cells and the second composition comprising the other of the CD4+ T cells or the CD8+ T cells.

293. The method of embodiment 292, wherein:

the first composition and second composition are administered 0 to 12 hours apart, 0 to 6 hours apart or 0 to 2 hours apart or wherein the administration of the first composition and the administration of the second composition are carried out on the same day, are carried out between about 0 and about 12 hours apart, between about 0 and about 6 hours apart or between about 0 and 2 hours apart; and/or

the initiation of administration of the first composition and the initiation of administration of the second composition are carried out between about 1 minute and about 1 hour apart or between about 5 minutes and about 30 minutes apart.

294. The method of embodiment 292 or embodiment 293, wherein the first composition and second composition are administered no more than 2 hours, no more than 1 hour, no more than 30 minutes, no more than 15 minutes, no more than 10 minutes or no more than 5 minutes apart.

295. The method of any of embodiments 122-294, wherein the first composition comprises the CD4+ T cells.

296. The method of any of embodiments 122-294, wherein the first composition comprises the CD8+ T cells.

297. The method of any of embodiments 122-296, wherein the first composition is administered prior to the second composition.

299. The method of any of embodiments 171-297, wherein, prior to the administration, the subject has been preconditioned with a lymphodepleting therapy comprising the administration of fludarabine and/or cyclophosphamide.

300. The method of any of embodiments 171-299, further comprising, immediately prior to the administration, administering a lymphodepleting therapy to the subject comprising the administration of fludarabine and/or cyclophosphamide.

301. The method of embodiment 299 or embodiment 300, wherein the lymphodepleting therapy comprises administration of cyclophosphamide at about 200-400 mg/m², optionally at or about 300 mg/m², inclusive, and/or fludarabine at about 20-40 mg/m², optionally 30 mg/m², daily for 2-4 days, optionally for 3 days, or wherein the lymphodepleting therapy comprises administration of cyclophosphamide at about 500 mg/m².

302. The method of any of embodiments 299-301, wherein:

the lymphodepleting therapy comprises administration of cyclophosphamide at or about 300 mg/m² and fludarabine at about 30 mg/m² daily for 3 days; and/or

the lymphodepleting therapy comprises administration of cyclophosphamide at or about 500 mg/m² and fludarabine at about 30 mg/m² daily for 3 days.

303. The method of any of embodiments 171-302, wherein the subject is a human.

304. A kit comprising:

(a) a T cell therapy comprising a dose of genetically engineered T cells expressing a chimeric antigen receptor, wherein the chimeric antigen receptor specifically binds to a target antigen expressed by the B cell malignancy;

(b) a checkpoint inhibitor that is an antibody or antigen-binding fragment thereof capable of blocking an immune checkpoint pathway protein, optionally wherein the checkpoint inhibitor thereof is formulated in one or more individual doses; and

(c) instructions for administering the T cell therapy and/or the checkpoint inhibitor to a subject having a B cell malignancy, wherein the instructions specify administration of the T cell therapy and/or the checkpoint inhibitor according to the method of any of embodiments 171-303.

305. A kit comprising:

(a) a T cell therapy comprising a dose of genetically engineered T cells expressing a chimeric antigen receptor, wherein the chimeric antigen receptor specifically binds to a target antigen expressed by the B cell malignancy; and

(b) instructions for administering the T cell therapy to a subject having a B cell malignancy, wherein the instructions specify that the subject is to be administered a checkpoint inhibitor that is an antibody or antigen-binding fragment thereof capable of blocking an immune checkpoint pathway protein, after the administration of the T cell therapy, wherein the instructions specify administration of the T cell therapy and/or the checkpoint inhibitor according to the method of any of embodiments 171-303.

306. A kit comprising:

(a) a checkpoint inhibitor that is an antibody or antigen-binding fragment thereof capable of blocking an immune checkpoint pathway protein, optionally wherein the checkpoint inhibitor thereof is formulated in one or more individual doses; and

(b) instructions for administering the checkpoint inhibitor to a subject having a B cell malignancy, wherein the instructions specify that the checkpoint inhibitor is administered after initiation of administration of a T cell therapy, the T cell therapy comprising a dose of genetically engineered T cells expressing a chimeric antigen receptor, wherein the chimeric antigen receptor specifically binds to a target antigen expressed by the B cell malignancy, wherein the instructions specify administration of the T cell therapy and/or the checkpoint inhibitor according to the method of any of embodiments 171-303.

### VIII. EXAMPLES

The following examples are included for illustrative purposes only and are not intended to limit the scope of the invention.

### Example 1 Assessment of Antigen-Specific Stimulation of PD-1 Expression in CAR-Expressing T Cells in the Presence of PD-L1-Expressing Cells

T cell compositions containing T cells engineered to express a chimeric antigen receptor (CAR) were assessed for expression of PD-1 after incubation in the presence of stimulatory conditions.

T cell compositions containing anti-CD19 CAR-expressing T cells were generated from leukapheresis samples from three healthy human adult donors by a process including immunoaffinity-based selection of T cells (including CD4+ and CD8+ cells) from the samples, for enrichment for CD4+ and CD8+ T cells, resulting in two compositions, enriched for CD8+ and CD4+ cells, respectively.

Cells of the enriched CD4+ and CD8+ compositions were separately activated with anti-CD3/anti-CD28 beads and subjected to lentiviral transduction with a vector encoding an anti-CD19 CAR. The anti-CD19 CAR contained an anti-CD19 scFv derived from a murine antibody (variable region derived from FMC63), an immunoglobulin-derived spacer, a transmembrane domain derived from CD28, a costimulatory region derived from 4-1BB, and a CD3-zeta intracellular signaling domain. The expression construct in the viral vector further contained sequences encoding a truncated receptor, which served as a surrogate marker for CAR expression; separated from the CAR sequence by a T2A ribosome skip sequence. Transduced populations then were separately incubated in the presence of stimulating reagents for cell expansion. Expanded CD8+ and CD4+ cells were formulated and cryopreserved separately and stored.

The cryopreserved CD4+ and CD8+ anti-CD19 CAR-expressing cells from each donor were thawed, combined at approximately a 1:1 CAR+CD4+:CD8+ ratio, and 5×10⁴ cells of the generated CAR+ T cell composition were plated in a 96-well flat bottom plate. Target K562 cells transduced with human CD19 (K562.CD19) were co-incubated overnight at 37° C., 5% CO2 with the CAR+ T cell composition at 3 different effector to target (E:T) ratios: 5:1 (1×10⁴ cells), 2.5:1 (2×10⁴ cells), and 1.25:1 (4×10⁴ cells).

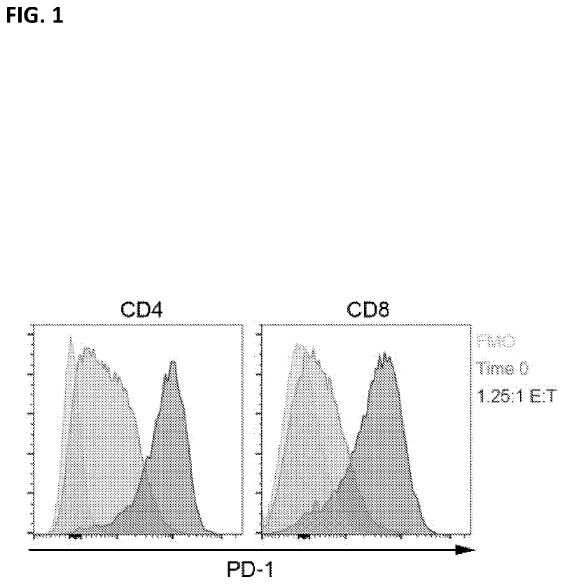

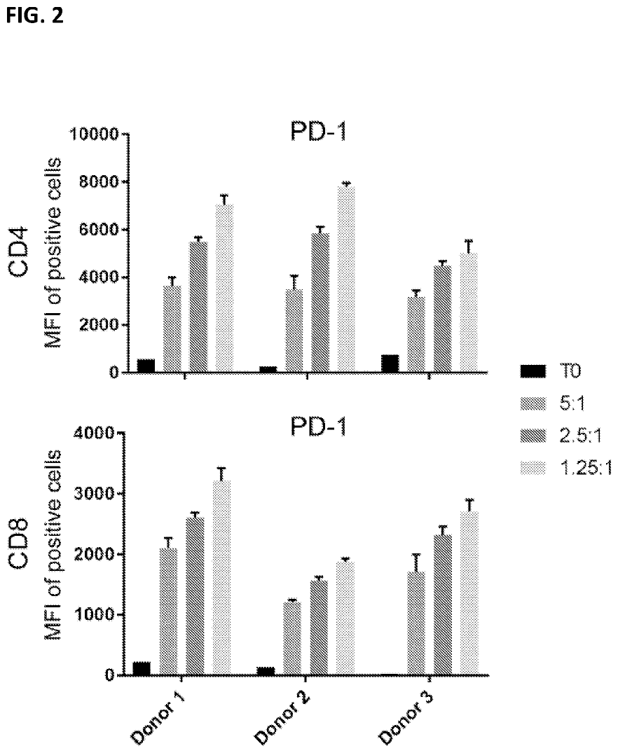

Following incubation, CAR+ T cells from all conditions were analyzed by flow cytometry for CAR expression using an antibody recognizing transduced cells, and antibodies specific for CD3, CD4, CD8, and PD-1. The percentage of T cells deemed CAR+(transduced) by this assay varied between donors and between CD4+ and CD8+ T cells. CD4+ T cells ranged from 70.4-80.5% CAR+, and CD8+ T cells ranged from 47.5-52.9% CAR+. Immediately post thaw, CD3+CD4+ and CD3+CD8+ anti-CD19 CAR-expressing cells demonstrated cell surface expression of PD-1 (time 0), which was upregulated after 24 hours of antigen exposure, by way of exposure to K562.CD19 cells (FIG. 1). The magnitude of PD-1 upregulation in both the CD4+ and CD8+ anti-CD19 CAR-expressing cells in response to culture with K562.CD19 cells was observed to correlate to the relative number of target cells present in culture, as demonstrated by increased PD-1 expression (MFI of positive cells) for the 1.25:1 effector to target ratios compared to 5:1 (FIG. 2).

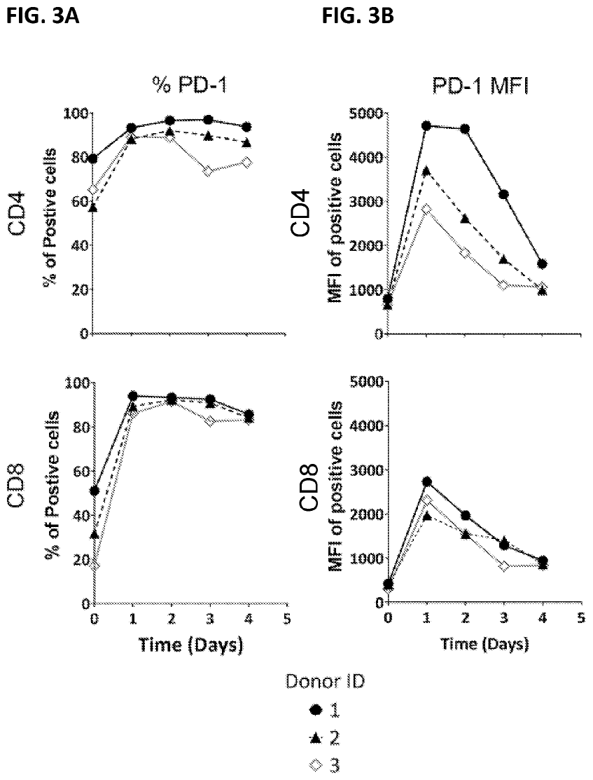

In addition, the 2.5:1 E:T (CAR-T:K562.CD19) ratio cell cultures were analyzed for PD-1 expression daily for 4 days after stimulation to monitor the kinetics of PD-1 expression. As shown in FIG. 3A, the percentage of anti-CD19 CAR-expressing cells that were positive for PD-1 expression was stable over a period of 4 days following a single stimulation with antigen-expressing (K562.CD19) cells. However, the amount of PD-1 expressed on the surface of both CD4+ and CD8+ cells, as determined by mean fluorescence intensity for PD-1 staining, decreased rapidly one day after stimulation, with surface expression levels returning to baseline by day 4 (FIG. 3B).

### Example 2 Evaluation of the Presence or Absence of an Anti-PD-L1 Antibody on Activity of Anti-CD19 CAR-Expressing Cells

Studies were undertaken to assess whether PD-1/PD-L1 engagement inhibited various readouts indicative of anti-CD19 CAR-expressing cell activity, and whether such effects could be blocked in the presence of an exemplary anti-PD-L1 antibody, durvalumab. Cytokine production and expression of T cell activation markers were assessed in anti-CD19 CAR-expressing T cell compositions generated from three healthy donors, following co-culture, essentially as described in Example 1, for 24 hours, with the target cells either being K562.CD19 cells or K562 cells transduced to express human CD19 and PD-L1 (K562.CD19.PDL1). The incubation was carried out in the presence or absence of durvalumab (20 μg/mL). An isotype control was used as a control.

A. Cytokine Production

Following incubation, supernatants from each condition were harvested and analyzed for cytokine production.

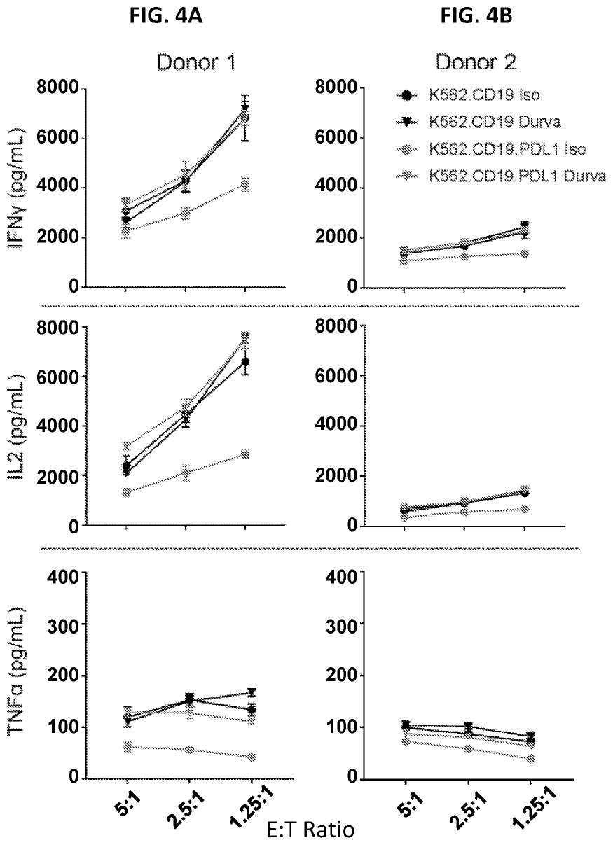

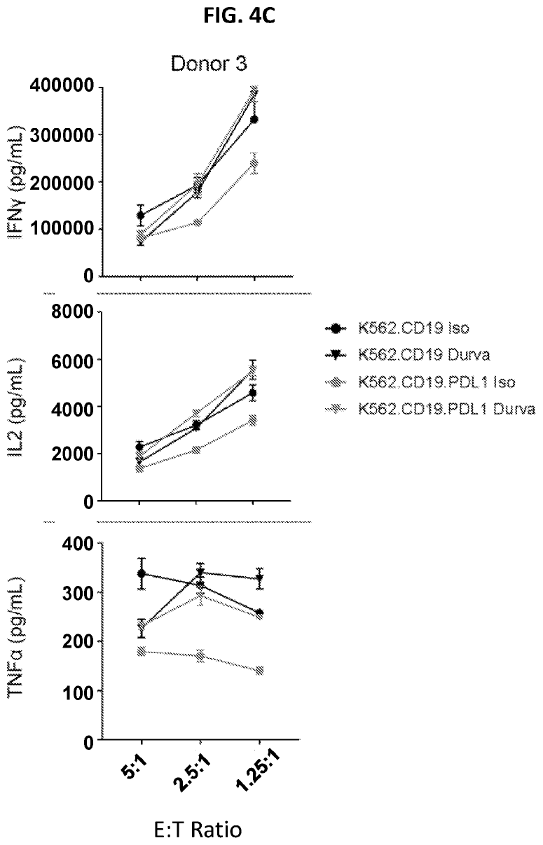

As shown in FIGS. 4A-4C, IFNγ, IL-2, and TNF-α cytokine production were reduced in the presence of K562.CD19.PDL1 cells as compared to K562.CD19 cells lacking PD-L1 expression, at E:T ratios of 5:1 through 1.25:1 across all three anti-CD19 CAR-expressing donor cell preparations. The presence of durvalumab was observed to restore cytokine production levels across cell compositions derived from different donors (FIGS. 4A, 4B and 4C), indicating the PD-L1-mediated inhibitory effect on antigen-induced IFN-γ, IL-2, and TNF-α production was restored in the presence of durvalumab.

B. Expression of T cell Activation Markers

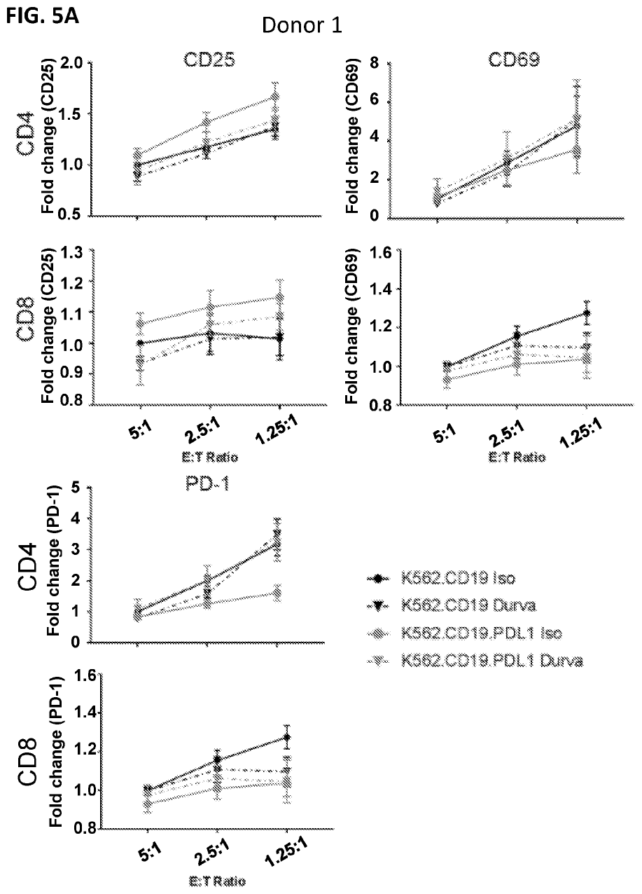

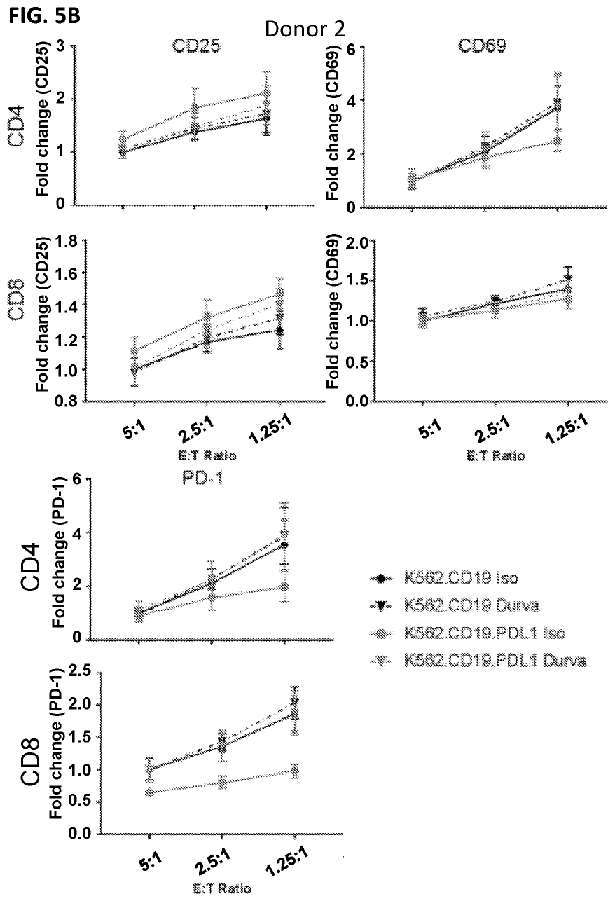

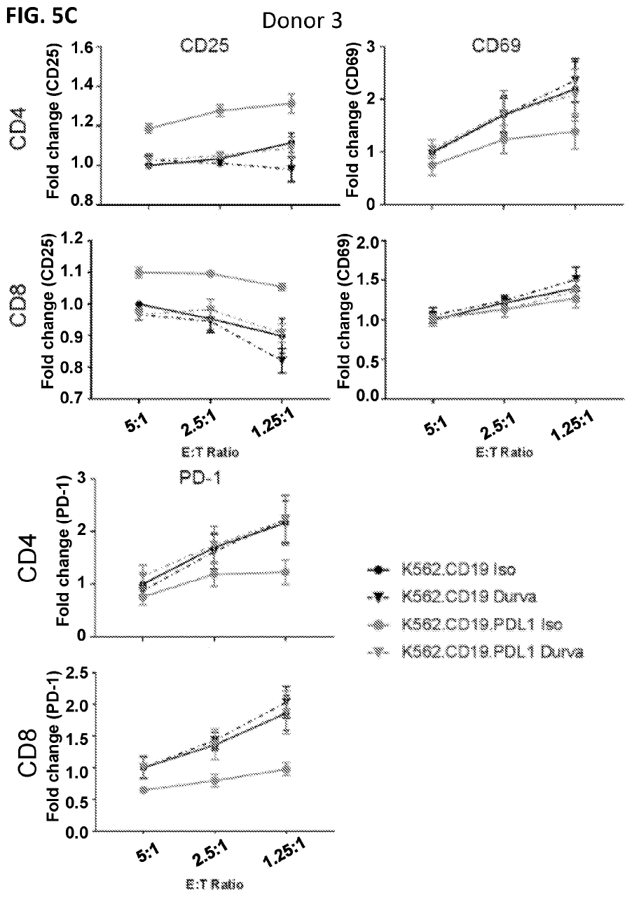

Expression of PD-1 and T cell activation markers CD25 and CD69 were assessed by flow cytometry on CD4+ and CD8+ anti-CD19 CAR-expressing cells that had been incubated for 24 hours with CD19+ target cells (K562.CD19 and K562.CD19.PDL1), in the presence or absence of durvalumab. When, the increase in PD-1 expression observed for CAR-expressing cells cultured for 24 hours with antigen-expressing K562.CD19 cells was not observed in cultures in which the target cells expressed PD-L1 (K562.CD19.PDL1 target cells), indicating that the antigen-induced increased PD-1 expression on anti-CD19 CAR-expressing cells was inhibited by the presence of PD-L1 on target cells (K562.CD19.PDL1). See FIGS. 5A-5C. As shown, the addition of durvalumab was observed to result in an increase in PD-1 expression levels on anti-CD19 CAR-expressing cells in co-cultures with PD-L1-expressing (K562.CD19.PDL1) target cells. In some embodiments, reduction of (or inhibition of increased) PD-1 expression in the presence of PD-L1-expressing target cells may be due to or related to PD-L1-mediated inhibition of activation response and/or shedding or internalization of the receptor upon engagement with its ligand.

CD25 expression on anti-CD19 CAR-expressing cells was observed to increase when anti-CD19 CAR-expressing cells were co-cultured with PD-L1-expressing (K562.CD19.PDL1) target cells compared to co-culture with K562.CD19 target cells not expressing PD-L1. CD4+, but not CD8+, anti-CD19 CAR-expressing T cells exhibited decreased expression of the early T cell activation marker, CD69, particularly at the 1.25:1 E:T ratio, when incubated with K562.CD19.PDL1 target cells compared to co-culture with K562.CD19 target cells not expressing PD-L1. The presence of durvalumab during co-culture of anti-CD19 CAR-expressing cells with K562.CD19.PDL1 was observed to reverse the observed decrease in CD69 expression in the presence of PD-L1-expressing target cells. The data indicates that effects of PD-L1 on surface expression of activation markers could be blocked by antagonizing PD-L1, such as by using an anti-PD-L1 antibody.

### Example 3 Re-expansion of Anti-CD19 CAR-Expressing Cells In Vivo

Anti-CD19 CAR-expressing cell compositions were produced substantially as described in Example 1 and were administered to subjects with Non-Hodgkin Lymphoma (NHL). Prior to administration of the CAR-expressing T cells (d=0), subjects were treated with 30 mg/m² fludarabine daily for 3 days and 300 mg/m² cyclophosphamide daily for 3 days. The cryopreserved cell compositions were thawed prior to intravenous administration. The therapeutic T cell dose was administered as a defined cell composition by administering a formulated CD4+ CAR-expressing cell population and a formulated CD8+ CAR-expressing population administered at a target ratio of approximately 1:1.

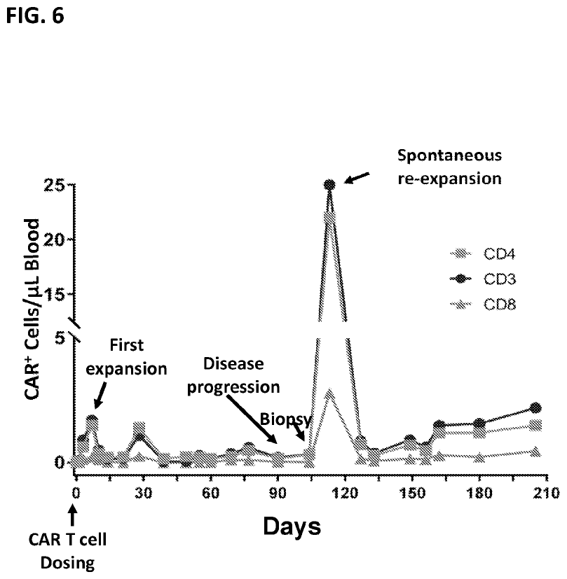

At d=0, treatment of a subject with chemorefractory transformed Diffuse Large B-Cell Lymphoma (DLBCL) (germinal center subtype with a BCL2 rearrangement and multiple copies of MYC and BCL6) was initiated, with a single-dose schedule by intravenous infusion. Each dose administered included 5×10⁷ CAR-expressing T cells (target 1:1 CD4+:CD8+ ratio). The numbers of CD3+/CAR+, CD4+/CAR+, CD8+/CAR+ T cells in peripheral blood, measured at certain time points, are shown in FIG. 6. The subject had previously been treated with, and was refractory to, five prior lines of therapy including dose-adjusted etoposide, doxorubicin, and cyclophosphamide with vincristine and prednisone plus rituximab (DA-EPOCH-R) and intermediate-intensity allogenic stem-cell transplantation from an 8/8 HLA-matched unrelated donor. Following allogeneic stem cell transplantation and prior to receiving anti-CD19 CAR-expressing T cells, the subject showed 100% donor chimerism in all blood lineages, had ceased taking immunosuppressive therapy, and did not have graft versus host disease (GVHD). Prior to administration of anti-CD19 CAR-expressing T cells, the subject had a periauricular mass and right-temporal lobe brain lesion observed by positron-emission tomography and computed tomography (PET-CT) and confirmed by magnetic resonance imaging (MRI).

After receiving anti-CD19 CAR-expressing T cell treatment, the subject achieved complete response (CR) 28 days post-infusion, as shown by PET-CT and brain MRI, with no observed signs of neurotoxicity or CRS. Three months post-infusion of the CAR-expressing T cells, relapse of the periauricular mass was noted in this subject, and an incisional biopsy was performed. Following biopsy, the visible tumor receded with no further therapy. As shown in FIG. 6, pharmacokinetic analysis showed a marked re-expansion of the CAR-T cells in peripheral blood (to a level higher than initial expansion observed, with peak levels observed at about 113 days post-infusion), which coincided with tumor regression. The subject then went on to achieve a second CR, as confirmed by restaging PET-CT one month following the biopsy, and remained in CR at 6 months post CAR-expressing T cell infusion. Further assessment of the subject showed that the CNS response was durable and the subject remained in CR at 12 months.

The results are consistent with a conclusion that re-expansion and activation of CAR-expressing T cells can be initiated in vivo following reduction or loss of functional or active CAR+ T cells and/or relapse following anti-tumor response to CAR-T cell therapy. Further, following re-expansion in vivo late after initial CAR+ T cell infusion, the CAR+ T cells are able to re-exert anti-tumor activity. This result supports that CAR+ T cell re-expansion and activation can be triggered in vivo and that methods of reactivating or boosting CAR+ T cells may further augment their efficacy.

### Example 4 Assessment of PD-L1 Expression on Biopsy Samples Post-Treatment with Anti-CD19 CAR-Expressing Cells

Expression of PD-L1 was assessed in tumor biopsies collected from subjects before and/or after administration of CAR-expressing cells.

Anti-CD19 CAR-expressing cell compositions were produced substantially as described in Example 1. Tumor biopsies were collected from selected subjects with relapsed or refractory (R/R) diffuse large B-cell lymphoma (DLBCL) or mantle cell lymphoma (MCL) with therapeutic CAR-expressing T cell compositions. Tumor biopsies were obtained prior to administration of the CAR-expressing T cells (pre-treatment) and at 7 to 20 days after administration (post-treatment). Results from 43 biopsies (26 pre-treatment; 17 post-treatment and 15 matched pairs) from 28 total subjects (25 DLBCL and 3 MCL) were examined.

Infiltration of CAR-expressing T cell in the tumor biopsy was quantified using in situ hybridization (ISH) probes specific to the mRNA encoding the anti-CD19 CAR. CAR-expressing T cells, non-CAR T cells and B cells were enumerated using multiplex immunofluorescence (IF) assays detecting for a cell surface surrogate marker for CAR-expressing cells, and for CD4, CD8, CD19, CD20, and PD-L1. Tumor biopsy sections were stained with hematoxylin and eosin (H&E) and assessed for tissue quality and tumor identification. Immunofluorescence images were analyzed using an image analysis software.

Both CD4⁺ and CD8⁺ CAR T cells were observed to have infiltrated the tumor area at the post-treatment time point (7 to 20 days after administration).

Expression of PD-L1 varied among subjects at pre-treatment (0.16%; 0-56%)) and post-treatment (3.3%; 0-65%)). Post-treatment increases in CD8⁺ cells in matched biopsies were observed to be associated with post-treatment increases in PD-L1 (R²=0.61) expression. This result is consistent with a conclusion that infiltration of CD8+ CAR+ cells at the time assessed may indicate that the presence and/or activity of such cells may result in upregulation of tumor microenvironment (TME) factors. In some embodiments, therapies targeting these factors, e.g. targeting PD-L1, such as those administered at the time of or following administration of the CAR-T cells, may enhance one or more therapeutic outcomes or duration thereof following CAR+ T cell administration.

### Example 5 Administration of Anti-CD19 CAR-Expressing Cells in Combination with an Immune Checkpoint Inhibitor (e.g., an Anti-PD-L1 Antibody) to Subjects with Relapsed and Refractory Non-Hodgkin's Lymphoma (NHL)

Anti-CD19 CAR-expressing T cell compositions were produced substantially as described in Example 1, and are administered to subjects with relapsed/refractory (R/R) B cell non-Hodgkin lymphoma (NHL). In some aspects, such compositions are administered to such subjects in combination with an anti-PD-L1 antibody, administered subsequently to the administration of the CAR-expressing T cell compositions. Groups of subjects selected for treatment include subjects with diffuse large B-cell lymphoma (DLBCL); de novo or transformed from indolent lymphoma (NOS); high-grade B-cell lymphoma, with MYC and BCL2 and/or BCL6 rearrangements with DLBCL histology (double/triple hit lymphoma); follicular lymphoma grade 3b (FLG3B); T cell/histiocyte-rich large B-cell lymphoma; Epstein-Barr virus (EBV) positive DLBCL, NOS; and primary mediastinal (thymic) large B-cell lymphoma (PMBCL). Subjects treated have relapsed following or are refractory to at least two prior lines of therapy, including a CD20-targeted agent and an anthracycline, and have an Eastern Cooperative Oncology Group (ECOG) score of less than or equal to 1 at screening.

Prior to CAR+ T cell infusion, subjects receive a lymphodepleting chemotherapy with fludarabine (flu, 30 mg/m²) and cyclophosphamide (Cy, 300 mg/m²) for three (3) days. The subjects receive CAR-expressing T cells 2-7 days after lymphodepletion. Subjects are administered a dose of CAR-expressing T cells (each single dose via separate infusions at a 1:1 ratio of CD4+ CAR-expressing T cells and CD8+ CAR-expressing T cells, respectively) as follows: a single dose of 5×10⁷ total CAR-expressing T cells (DL1) or a single dose of 1×10⁸ CAR-expressing T cells (DL2).

The exemplary anti-PD-L1 antibody durvalumab is administered to subjects post-CAR-T cell infusion as an intravenous (IV) infusion at a dosing schedule including one or more 28-day cycle, to result in the same or similar exposure levels as achieved by administration of durvalumab at a dose of 1500 mg Q4W (such as 10 mg/kg Q2W or 20 mg/kg Q4W), such as based on the area under the curves steady state. It was determined that, in a 28 day cycle, a dose of 375 mg every week (Q1W) is expected to be equivalent to a dose of 5 mg/kg Q1W, and that such dose and a dose of 750 mg every two weeks (Q2W), are comparable to a dose of 10 mg/kg Q2W and 20 mg/kg Q4W. Specifically, it was observed that dosing approaches with lower doses given more frequently during one or more 28 day cycle (such as a dose of 375 mg Q1W for two weeks, a dose of 750 mg Q2W, and a dose of 1500 mg Q4W, as compared to two doses of 1500 mg Q4W), resulted in similar biological PD-L1 occupancy but shorter half-life. Without wishing to be bound by theory, in some embodiments, such shorter half-life may result in a lower risk of adverse or unwanted effects such as toxicity following administration.

In some subjects receiving either DL1 or DL2 of anti-CD19 CAR+ T cells, durvalumab is administered beginning at day 29 (±7 days) post-CAR-T cell infusion at a dose of 375 mg Q1W for 2 weeks (e.g. at days 29 and 36), then one dose of 750 mg Q2W (e.g. at day 43), followed by 1500 mg Q4W for two doses (e.g. at days 57 and 85).

In some cases, an alternative dosing regimen involving administration of durvalumab at a lower dose and/or or at a delayed dose is given in subjects having received anti-CD19 CAR+ T cells, e.g. at DL1. An exemplary lower dosing schedule of durvalumab includes administration beginning at day 29 (±7 days) post-CAR-T cell infusion at a dose of 225 mg Q1W for 2 weeks (e.g. day 29 and 36), then 375 mg Q1W for 2 weeks (e.g. days 43 and 50), then two doses of 750 mg Q2W (e.g. at days 57 and 71), followed by 1500 mg Q4W for one dose (e.g. at day 85). An exemplary delayed dosing schedule of durvalumab includes administration beginning at day 43 (±7 days) post-CAR-T cell infusion at a dose of 375 mg Q1W for 2 weeks (e.g. at day 43 and 50), then two doses of 750 mg Q2W (e.g. at day 57 and 71), followed by 1500 mg Q4W for one dose (e.g. at day 85).

Response to treatment is assessed based on radiographic tumor assessment by positron emission tomography (PET) and/or computed tomography (CT) or magnetic resonance imaging (MRI) scans at baseline prior to treatment and at various times following treatment (e.g. based on Lugano classification, see, e.g., Cheson et al., (2014) JCO 32(27):3059-3067). The presence or absence of treatment-emergent adverse events (TEAE) following treatment also is assessed. Subjects also are assessed and monitored for neurotoxicity (neurological complications including symptoms of confusion, aphasia, encephalopathy, myoclonus seizures, convulsions, lethargy, and/or altered mental status), graded on a 1-5 scale, according to the National Cancer Institute—Common Toxicity Criteria (CTCAE) scale, version 4.03 (NCI-CTCAE v4.03). Common Toxicity Criteria (CTCAE) scale, version 4.03 (NCI-CTCAE v4.03). See Common Terminology for Adverse Events (CTCAE) Version 4, U.S. Department of Health and Human Services, Published: May 28, 2009 (v4.03: Jun. 14, 2010); and Guido Cavaletti & Paola Marmiroli Nature Reviews Neurology 6, 657-666 (December 2010). Cytokine release syndrome (CRS) also is determined and monitored, graded based on severity. See Lee et al, Blood. 2014; 124(2):188-95. Subjects also are assessed for PK of anti-CD19 CAR+ T cells pre- and post-treatment with durvalumab and for PK of durvalumab.

The dosing of durvalumab is stopped after three 28 day cycles (e.g. after 3 months), unless the subject achieves a partial response (PR) in which case further cycles of durvalumab may continue until disease progression, for a total duration of up to 12 months.

### Example 6 Assessment of Response, Safety and Pharmacokinetics in Subjects with Relapsed and Refractory Non-Hodgkin's Lymphoma (NHL) after Administration of Anti-CD19 CAR-Expressing Cells in Combination with an Immune Checkpoint Inhibitor (e.g., an Anti-PD-L1 Antibody)

Therapeutic CAR⁺ T cell compositions containing autologous T cells expressing a chimeric antigen-receptor (CAR) specific for CD19 and an exemplary anti-PD-L1 antibody durvalumab were administered to subjects with relapsed/refractory (R/R) B cell non-Hodgkin lymphoma (NHL), generally as described in Example 5 above. Results are described through a particular time-point in an ongoing clinical study administering such combination therapy to subjects with R/R NHL.

### A. SUBJECTS AND TREATMENT

Adult subjects, including those with DLBCL NOS including transformed indolent NHL; high grade B-cell lymphoma with MYC and BCL2 and/or BCL6 rearrangements with DLBCL histology (double/triple-hit lymphoma); FLG3B; T cell/histiocyte-rich large B-cell lymphoma; EBV-positive DLBCL, NOS; or PMBCL, as observed by positron-emission tomography (PET), that have relapsed following or were refractory to at least two prior lines of therapy, including a CD20-targeted agent and an anthracycline, and have an ECOG score of less than or equal to 1, were included in the study. Among the subjects enrolled in the study, some had DLBCL, NOS; and some had relapsed disease. Subjects were not excluded based on previous allogeneic hematopoietic stem cell transplant (HSCT) if the HSCT occurred more than 90 days before leukapheresis, or based on receiving a prior CD19-targeted CAR+ T cell therapy or an anti-PD-L1 antibody.

Subjects received a lymphodepleting chemotherapy with fludarabine and cyclophosphamide for three (3) days, then administered of a single dose of 5×10⁷ total CAR+ T cells (DL1; each single dose via separate administration at a 1:1 ratio of CD4+ CAR+ T cells and CD8+ CAR+ T cells, respectively) or a single dose of 1×10⁸ CAR+ T cells (DL2). The subjects were administered durvalumab, at a dose of 375 mg Q1W for two doses beginning at day 29 (±7 days) post-CAR+ T cell administration (e.g., at days 29 and 36), then one dose of 750 mg Q2W (e.g., at day 43), followed by 1500 mg Q4W for two doses (e.g. at days 57 and 85), with the exception of one subject who received the first dose of durvalumab at day 43, and 3 subjects whose last 1500 mg dose was delayed by 8, 9 or 44 days. Response, treatment-emergent adverse events (TEAEs) and incidence cytokine release syndrome (CRS) or neurological events (NE) were assessed as described in Example 5 above. Pharmacokinetic parameters based on the number of CAR+ T cells in peripheral blood was assessed at various time points post-administration of CAR+ T cells (e.g., at days 1, 2, 4, 8, 11, 15, 22, 29, 36, 43, 50, 57, 71, 85, 180, 270 or 365) by flow cytometry.

At the time point of analysis, eleven (11) total subjects had received CAR+ T cells and at least one dose of durvalumab. Eight (8) subjects received DL1 of the CAR+ T cells, and six (6) of those subjects had received all 5 doses of durvalumab; three (3) subjects received DL2 of the CAR+ T cells, and two (2) of those subjects had received all 5 doses of durvalumab.

### B. RESPONSE AND SAFETY

At the time point of analysis, no dose-limiting toxicities were observed following administration with either the CAR+ T cells alone or in combination with durvalumab.

At one month after administration of the CAR+ T cells (at or about the time of the first administration of durvalumab), the overall response rate (ORR) was 91% (10/11; DL1, 7/8 and DL2, 3/3), and 64% of the subject (7/11) achieved complete response (CR) (DL1, 5/8; DL2, 2/3). At month 3 after administration of CAR+ T cells, one subject who had a PR at month 1 converted to a CR at month 3. Among the subjects treated, 6 subjects were evaluated at month 6 after administration of CAR+ T cells, and four (4) of those subjects achieved complete response at 6 months. One such subject had a PR at month 1 and month 3, but converted to CR at month 6.

### C. PHARMACOKINETIC ASSESSMENT AND RESPONSE

Pharmacokinetics were assessed based on the number of CAR+ T cells in the peripheral blood of the subjects measured at various time points after administration of the CAR+ T cells. In three exemplary subjects, the number of CAR+ T cells in the blood was observed to decrease from the initial expansion around day 57, but an increase was observed after the first 1500 mg dose of durvalumab (e.g., between about day 57 and day 85). In one of the three subjects, the number of CAR+ T cells in the blood was undetectable at day 57, but increased at day 85, such that the number was greater than the number of CAR+ T cells in the blood at day 8 or 22 in this subject. In another of the three subjects, the CAR+ T cell expansion was observed until month 6 (in this subject, the final 1500 mg dose of durvalumab was delayed until day 129), at which time point the number was more than 10-fold greater than that at month 2 (e.g., day 57). In a different exemplary subject, a high number of CAR+ T cells was observed starting day 22, without a substantial decrease around day 57, and the high number was generally maintained at day 85. These four exemplary subjects who exhibited an increase in CAR+ T cells in the blood or maintenance of high CAR+ T cells after the final dose of durvalumab, were observed to have at least a partial response at month 3, and these subjects were the 4 subjects that achieved a complete response at month 6, including a subject that converted from PR to CR.

### D. CONCLUSION

At this time point in the ongoing study, administration of an exemplary immune checkpoint inhibitor, the anti-PD-L1 antibody durvalumab, in combination with CD19-specific CAR+ T cells as described was observed to exhibit safety profile with a low incidence of Grade 3 or higher CRS and neurological events, and favorable response outcomes. In some subjects, improved pharmacokinetic profile was observed after administration of durvalumab, and this improvement was also associated with prolonged CR. The results were consistent with the acceptable safety profile of the particular dosing schedule of an exemplary immune checkpoint inhibitor, the anti-PD-L1 antibody durvalumab, in combination with anti-CD19 CAR+ T cells. The results were also consistent with improved pharmacokinetics of the CAR+ T cells, including prolonged persistence of cells, and clinical response, as shown in this group of subjects.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | ANNOTATION |
|---|---|---|
| 1 | ESKYGPPCPPCP | spacer |
| | | (IgG4hinge) |
| | | (aa) |
| 2 | GAATCTAAGTACGGACCGCCCTGCCCCCCTTGCCCT | spacer |
| | | (IgG4hinge) |
| | | (nt) |
| 3 | ESKYGPPCPPCPGQPREPQVYTLPPSQEEMTKNQVSLTCLVKGFYPSD | Hingc-CH3 |
| | IAVEWESNGQPENNYKTTPPVLDSDGSFFLYSRLTVDKSRWQEGNVFS | spacer |
| | CSVMHEALHNHYTQKSLSLSLGK | |
| 4 | ESKYGPPCPPCPAPEFLGGPSVFLFPPKPKDTLMISRTPEVTCVVVDV | Hinge-CH2-CH3 |
| | SQEDPEVQFNWYVDGVEVHNAKTKPREEQFNSTYRVVSVLTVLHQDWL | spacer |
| | NGKEYKCKVSNKGLPSSIEKTISKAKGQPREPQVYTLPPSQEEMTKNQ | |
| | VSLTCLVKGFYPSDIAVEWESNGQPENNYKTTPPVLDSDGSFFLYSRL | |
| | TVDKSRWQEGNVFSCSVMHEALHNHYTQKSLSLSLGK | |
| 5 | RWPESPKAQASSVPTAQPQAEGSLAKATTAPATTRNTGRGGEEKKKEK | IgD-hingc-Fc |
| | EKEEQEERETKTPECPSHTQPLGVYLLTPAVQDLWLRDKATFTCFVVG | |
| | SDLKDAHLTWEVAGKVPTGGVEEGLLERHSNGSQSQHSRLTLPRSLWN | |
| | AGTSVTCTLNHPSLPPQRLMALREPAAQAPVKLSLNLLASSDPPEAAS | |
| | WLLCEVSGFSPPNILLMWLEDQREVNTSGFAPARPPPQPGSTTFWAWS | |
| | VLRVPAPPSPQPATYTCVVSHEDSRTLLNASRSLEVSYVTDH | |
| 6 | LEGGGEGRGSLLTCGDVEENPGPR | T2A |
| 7 | MLLLVTSLLLCELPHPAFLLIPRKVCNGIGIGEFKDSLSINATNIKHF | tEGFR |
| | KNCTSISGDLHILPVAFRGDSFTHTPPLDPQELDILKTVKEITGFLLI | |
| | QAWPENRTDLHAFENLEIIRGRTKQHGQFSLAVVSLNITSLGLRSLKE | |
| | ISDGDVIISGNKNLCYANTINWKKLFGTSGQKTKIISNRGENSCKATG | |
| | QVCHALCSPEGCWGPEPRDCVSCRNVSRGRECVDKCNLLEGEPREFVE | |
| | NSECIQCHPECLPQAMNITCTGRGPDNCIQCAHYIDGPHCVKTCPAGV | |
| | MGENNTLVWKYADAGHVCHLCHPNCTYGCTGPGLEGCPTNGPKIPSIA | |
| | TGMVGALLLLLWALGIGLFM | |
| 8 | FWVLWVGGVLACYSLLVTVAFIIFWV | CD28 |
| | | (amino acids |
| | | 153-179 of |
| | | Accession |
| | | No. P10747) |
| 9 | IEVMYPPPYLDNEKSNGTIIHVKGKHLCPSPLFPGPSKPFWVLVVVGG | CD28 |
| | VLACYSLLVTVAFIIFWV | (amino acids |
| | | 114-179 |
| | | of |
| | | Accession |
| | | No. P10747) |
| 10 | RSKRSRLLHSDYMNMTPRRPGPTRKHYQPYAPPRDFAAYRS | CD28 |
| | | (amino acids |
| | | 180-220 |
| | | of P10747) |
| 11 | RSKRSRGGHSDYMNMTPRRPGPTRKHYQPYAPPRDFAAYRS | CD28 (LL to GG) |
| 12 | KRGRKKLLYIFKQPFMRPVQTTQEEDGCSCRFPEEEEGGCEL | 4-1BB (amino acids |
| | | 214-255 of Q07011.1) |
| 13 | RVKFSRSADAPAYQQGQNQLYNELNLGRREEYDVLDKRRGRDPEMGGK | CD3 zcta |
| | PRRKNPQEGLYNELQKDKMAEAYSEIGMKGERRRGKGHDGLYQGLSTA | |
| | TKDTYDALHMQALPPR | |
| 14 | RVKFSRSAEPPAYQQGQNQLYNELNLGRREEYDVLDKRRGRDPEMGGK | CD3 zeta |
| | PRRKNPQEGLYNELQKDKMAEAYSEIGMKGERRRGKGHDGLYQGLSTA | |
| | TKDTYDALHMQALPPR | |
| 15 | RVKFSRSADAPAYKQGQNQLYNELNLGRREEYDVLDKRRGRDPEMGGK | CD3 zeta |
| | PRRKNPQEGLYNELQKDKMAEAYSEIGMKGERRRGKGHDGLYQGLSTA | |
| | TKDTYDALHMQALPPR | |
| 16 | RKVCNGIGIGEFKDSLSINATNIKHFKNCTSISGDLHILPVAFRGDSF | tEGFR |
| | THTPPLDPQELDILKTVKEITGFLLIQAWPENRTDLHAFENLEIIRGR | |
| | TKQHGQFSLAVVSLNITSLGLRSLKEISDGDVIISGNKNLCYANTINW | |
| | KKLFGTSGQKTKIISNRGENSCKATGQVCHALCSPEGCWGPEPRDCVS | |
| | CRNVSRGRECVDKCNLLEGEPREFVENSECIQCHPECLPQAMNITCTG | |
| | RGPDNCIQCAHYIDGPHCVKTCPAGVMGENNTLVWKYADAGHVCHLCH | |
| | PNCTYGCTGPGLEGCPTNGPKIPSIATGMVGALLLLLVVALGIGLFM | |
| 17 | EGRGSLLTCGDVEENPGP | T2A |
| 18 | GSGATNFSLLKQAGDVEENPGP | P2A |
| 19 | ATNFSLLKQAGDVEENPGP | P2A |
| 20 | QCTNYALLKLAGDVESNPGP | E2A |
| 21 | VKQTLNFDLLKLAGDVESNPGP | F2A |
| 22 | -PGGG-(SGGGG)5-P- wherein P is proline, G is | Linker |
| | glycine and S is serine | |
| 23 | GSADDAKKDAAKKDGKS | Linker |
| 24 | atgcttctcctggtgacaagccttctgctctgtgagttaccacaccca | GMCSFR alpha chain |
| | gcattcctcctgatccca | signal sequence |
| 25 | MLLLVTSLLLCELPHPAFLLIP | GMCSFR alpha chain |
| | | signal sequence |
| 26 | MALPVTALLLPLALLLHA | CD8 alpha signal |
| | | peptide |
| 27 | Glu Pro Lys Ser Cys Asp Lys Thr His Thr Cys Pro | Hinge |
| | Pro Cys Pro | |
| 28 | Glu Arg Lys Cys Cys Val Glu Cys Pro Pro Cys Pro | Hinge |
| 29 | ELKTPLGDTHTCPRCPEPKSCDTPPPCPRCPEPKSCDTPPPCPRCPEP | Hinge |
| | KSCDTPPPCPRCP | |
| 30 | Glu Ser Lys Tyr Gly Pro Pro Cys Pro Ser Cys Pro | Hinge |
| 31 | Glu Ser Lys Tyr Gly Pro Pro Cys Pro Pro Cys Pro | Hinge |
| 32 | Tyr Gly Pro Pro Cys Pro Pro Cys Pro | Hinge |
| 33 | Lys Tyr Gly Pro Pro Cys Pro Pro Cys Pro | Hinge |
| 34 | Glu Val Val Val Lys Tyr Gly Pro Pro Cys Pro Pro | Hinge |
| | Cys Pro | |
| 35 | RASQDISKYLN | CDR L1 |
| 36 | SRLHSGV | CDR L2 |
| 37 | GNTLPYTFG | CDR L3 |
| 38 | DYGVS | CDR H1 |
| 39 | VIWGSETTYYNSALKS | CDR H2 |
| 40 | YAMDYWG | CDR H3 |
| 41 | EVKLQESGPGLVAPSQSLSVTCTVSGVSLPDYGVSWIRQPPRKGLEWL | VH |
| | GVIWGSETTYYNSALKSRLTIIKDNSKSQVFLKMNSLQTDDTAIYYCA | |
| | KHYYYGGSYAMDYWGQGTSVTVSS | |
| 42 | DIQMTQTTSSLSASLGDRVTISCRASQDISKYLNWYQQKPDGTVKLLI | VL |
| | YHTSRLHSGVPSRFSGSGSGTDYSLTISNLEQEDIATYFCQQGNTLPY | |
| | TFGGGTKLEIT | |
| 43 | DIQMTQTTSSLSASLGDRVTISCRASQDISKYLNWYQQKPDGTVKLLI | scFv |
| | YHTSRLHSGVPSRFSGSGSGTDYSLTISNLEQEDIATYFCQQGNTLPY | |
| | TFGGGTKLEITGSTSGSGKPGSGEGSTKGEVKLQESGPGLVAPSQSLS | |
| | VTCTVSGVSLPDYGVSWIRQPPRKGLEWLGVIWGSETTYYNSALKSRL | |
| | TIIKDNSKSQVFLKMNSLQTDDTAIYYCAKHYYYGGSYAMDYWGQGTS | |
| | VTVSS | |
| 44 | KASQNVGTNVA | CDR L1 |
| 45 | SATYRNS | CDR L2 |
| 46 | QQYNRYPYT | CDR L3 |
| 47 | SYWMN | CDR H1 |
| 48 | QIYPGDGDTNYNGKFKG | CDR H2 |
| 49 | KTISSVVDFYFDY | CDR H3 |
| 50 | EVKLQQSGAELVRPGSSVKISCKASGYAFSSYWMNWVKQRPGQGLEWI | VH |
| | GQIYPGDGDTNYNGKFKGQATLTADKSSSTAYMQLSGLTSEDSAVYFC | |
| | ARKTISSWDFYFDYWGQGTTVTVSS | |
| 51 | DIELTQSPKFMSTSVGDRVSVTCKASQNVGTNVAWYQQKPGQSPKPLI | VL |
| | YSATYRNSGVPDRFTGSGSGTDFTLTITNVQSKDLADYFCQQYNRYPY | |
| | TSGGGTKLEIKR | |
| 52 | GGGGSGGGGSGGGGS | Linker |
| 53 | EVKLQQSGAELVRPGSSVKISCKASGYAFSSYWMNWVKQRPGQGLEWI | scFv |
| | GQIYPGDGDTNYNGKFKGQATLTADKSSSTAYMQLSGLTSEDSAVYFC | |
| | ARKTISSVVDFYFDYWGQGTTVTVSSGGGGSGGGGSGGGGSDIELTQS | |
| | PKFMSTSVGDRVSVTCKASQNVGTNVAWYQQKPGQSPKPLIYSATYRN | |
| | SGVPDRFTGSGSGTDFTLTITNVQSKDLADYFCQQYNRYPYTSGGGTK | |
| | LEIKR | |
| 54 | HYYYGGSYAMDY | HC-CDR3 |
| 55 | HTSRLHS | LC-CDR2 |
| 56 | QQGNTLPYT | LC-CDR3 |
| 57 | gacatccagatgacccagaccacctccagcctgagcgccagcctgggc | Sequence |
| | gaccgggtgaccatcagctgccgggccagccaggacatcagcaagtac | encoding |
| | ctgaactggtatcagcagaagcccgacggcaccgtcaagctgctgatc | scFv |
| | taccacaccagccggctgcacagcggcgtgcccagccggtttagcggc | |
| | agcggctccggcaccgactacagcctgaccatctccaacctggaacag | |
| | gaagatatcgccacctacttttgccagcagggcaacacactgccctac | |
| | acctttggcggcggaacaaagctggaaatcaccggcagcacctccggc | |
| | agcggcaagcctggcagcggcgagggcagcaccaagggcgaggtgaag | |
| | ctgcaggaaagcggccctggcctggtggcccccagccagagcctgagc | |
| | gtgacctgcaccgtgagcggcgtgagcctgcccgactacggcgtgagc | |
| | tggatccggcagccccccaggaagggcctggaatggctgggcgtgatc | |
| | tggggcagcgagaccacctactacaacagcgccctgaagagccggctg | |
| | accatcatcaaggacaacagcaagagccaggtgttcctgaagatgaac | |
| | agcctgcagaccgacgacaccgccatctactactgcgccaagcactac | |
| | tactacggcggcagctacgccatggactactggggccagggcaccagc | |
| | gtgaccgtgagcagc | |
| 58 | X₁PPX₂P | Hinge |
| | X₁ is glycine, cysteine or arginine | |
| | X₂ is cysteine or threonine | |
| 59 | GSTSGSGKPGSGEGSTKG | Linker |
| 60 | EVQLVESGGGLVQPGGSLRLSCAASGFTFSRYWMSWVRQAPGKGLEWV | heavy chain |
| | ANIKQDGSEKYYVDSVKGRFTISRDNAKNSLYLQMNSLRAEDTAVYYC | |
| | AREGGWFGELAFDYWGQGTLVTVSSASTKGPSVFPLAPSSKSTSGGTA | |
| | ALGCLVKDYFPEPVTVSWNSGALTSGVHTFPAVLQSSGLYSLSSVVTV | |
| | PSSSLGTQTYICNVNHKPSNTKVDKRVEPKSCDKTHTCPPCPAPEFEG | |
| | GPSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALPASI | |
| | EKTISKAKGQPREPQVYTLPPSREEMTKNQVSLTCLVKGFYPSDIAVE | |
| | WESNGQPENNYKTTPPVLDSDGSFFLYSKLTVDKSRWQQGNVFSCSVM | |
| | HEALHNHYTQKSLSLSPGK | |
| 61 | EIVLTQSPGTLSLSPGERATLSCRASQRVSSSYLAWYQQKPGQAPRLL | light chain |
| | IYDASSRATGIPDRFSGSGSGTDFTLTISRLEPEDFAVYYCQQYGSLP | |
| | WTFGQGTKVEIKRTVAAPSVFIFPPSDEQLKSGTASVVCLLNNFYPRE | |
| | AKVQWKVDNALQSGNSQESVTEQDSKDSTYSLSSTLTLSKADYEKHKV | |
| | YACEVTHQGLSSPVTKSFNRGEC | |

1. A method of treatment, the method comprising:
(a) administering a T cell therapy to a subject having a B cell malignancy, said cell therapy comprising a dose of genetically engineered T cells expressing a chimeric antigen receptor, wherein the chimeric antigen receptor specifically binds to a target antigen expressed by the B cell malignancy; and
(b) subsequently administering to the subject a checkpoint inhibitor that is an antibody or antigen-binding fragment thereof capable of blocking an immune checkpoint pathway protein, wherein a total dosage amount of the checkpoint inhibitor is administered in each of at least two dosage cycles, wherein the total dosage amount of the checkpoint inhibitor in the first of the at least two dosage cycles:
is the same as or less than the total dosage amount administered in the second and/or a subsequent dosage cycle; and
is administered in more than one individual dose over the course of the first dosage cycle, wherein the number of individual doses is greater than the number of individual doses administered in the second and/or a subsequent dosage cycle.

2. A method of treatment, the method comprising administering, to a subject having a B cell malignancy a checkpoint inhibitor that is an antibody or antigen-binding fragment thereof capable of blocking an immune checkpoint pathway protein, said subject having been administered a T cell therapy comprising a dose of genetically engineered T cells expressing a chimeric antigen receptor that specifically binds to a target antigen expressed by the B cell malignancy, wherein a total dosage amount of the checkpoint inhibitor is administered in each of at least two dosage cycles, wherein the total dosage amount of the checkpoint inhibitor in the first of the at least two dosage cycles:
is the same as or less than the total dosage amount administered in the second and/or a subsequent dosage cycle; and
is administered in more than one individual dose over the course of the first dosage cycle, wherein the number of individual doses is greater than the number of individual doses administered in the second and/or a subsequent dosage cycle.

3. The method of claim 1 or claim 2, wherein the dosage cycle is a 21-day cycle.

4. The method of claim 1 or claim 2, wherein the dosage cycle is a 28-day cycle.

5. The method of any of claims 1-4, wherein the total dosage amount in the first of the at least two dosage cycles is the same as the total dosage amount in the second of the at least two dosage cycles.

6. The method of any of claims 1-5, wherein the first of the at least two dosage cycles comprises 2, 3, 4 or more individual doses.

7. The method of claim 6, wherein the dosage cycle is a 28-day cycle and the individual doses of the first of the at least two 28-day cycles are administered as four doses each once every week (Q1W), two doses each as Q1W doses for two consecutive weeks, or two doses each as Q1W doses for two consecutive weeks and followed by one dose once in two weeks (Q2W).

8. The method of any of claims 1-7, wherein each of said at least two dosage cycles comprises administering independently a total dosage amount of at or about 400 mg to at or about 2000 mg of the checkpoint inhibitor.

9. The method of any of claims 1-8, wherein the checkpoint inhibitor blocks an immune checkpoint pathway protein selected from among PD-L1, PD-L2, PD-1 and CTLA-4.

10. The method of any of claims 1-9, wherein the checkpoint pathway is PD-1/PD-L1 and the checkpoint inhibitor is an anti-PD-1 antibody.

11. The method of claim 10, wherein the checkpoint inibitior is nivolumab, pembrolizumab, or cemiplimab.

12. The method of any of claims 1-11, wherein each of said at least two dosage cycle comprises administering independently a total dosage amount of at or about 400 mg to at or about 600 mg, optionally at or about 480 mg.

13. The method of any of claims 1-9, wherein the checkpoint pathway is PD-1/PD-L1 and the checkpoint inhibitor is an anti-PD-L1 antibody.

14. The method of any of claims 1-11 and 13, wherein each of said at least two dosage cycle comprises administering independently a total dosage amount of 750 mg to 2000 mg, optionally at or about 1500 mg.

15. The method of any of claims 1-14, wherein administration of the checkpoint inhibitor and/or the start of the first dosage cycle is initiated at a time at or after, optionally immediately after or within 1 to 3 days after:
(i) peak or maximum level of the cells of the T cell therapy are detectable in the blood of the subject;
(ii) the number of cells of the T cell therapy detectable in the blood, after having been detectable in the blood, is not detectable or is reduced, optionally reduced compared to a preceding time point after administration of the T cell therapy;
(iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy;
(iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject;
(v) the subject exhibits disease progression and/or has relapsed following remission after treatment with the T cell therapy; and/or
(iv) the subject exhibits increased tumor burden as compared to tumor burden at a time prior to or after administration of the cells and prior to initiation of administration of the checkpoint inhibitor.

16. The method of any of claims 1-15, wherein administration of the checkpoint inhibitor and/or the start of the first dosage cycle is initiated at or within 29 days, 36 days, 43 days or 50 days after initiation of administration of the T cell therapy.

17. The method of any of claims 1-16, wherein administration of the checkpoint inhibitor and/or the start of the first dosage cycle is initiated from or from about 22 days to 36 days after initiation of administration of the T cell therapy.

18. The method of any of claims 1-17, wherein administration of the checkpoint inhibitor and/or the start of the first dosage cycle is initiated at or about 29 days after initiation of administration of the T cell therapy.

19. The method of any of claims 1-18, wherein administration of the checkpoint inhibitor and/or the start of the first dosage cycle is initiated at or about 43 days after initiation of administration of the T cell therapy.

20. The method of any of claims 1-19, wherein at the time of administering the checkpoint inhibitor and/or the start of the first dosage cycle, the subject does not exhibit a severe toxicity following administration of the T cell therapy.

21. The method of claim 20, wherein:
the severe toxicity is severe cytokine release syndrome (CRS), optionally grade 3 or higher, prolonged grade 3 or higher or grade 4 or 5 CRS; and/or
the severe toxicity is severe neurotoxicity, optionally grade 3 or higher, prolonged grade 3 or higher or grade 4 or 5 neurotoxicity.

22. A method of treatment, the method comprising:
(a) administering a T cell therapy to a subject having a B cell malignancy, said T cell therapy comprising a dose of genetically engineered T cells expressing a chimeric antigen receptor, wherein the chimeric antigen receptor specifically binds to a target antigen expressed by cells of the B cell malignancy; and
(b) subsequently administering to the subject an anti-PD-L1 antibody or antigen-binding fragment thereof, wherein said administration comprises carrying out at least two 28-day cycles, wherein:
the first 28-day cycle comprises administering the anti-PD-L1 antibody or antigen-binding fragment thereof as two individual doses each once-weekly (Q1W) for two consecutive weeks of the 28-day cycle, each of said individual doses in an amount of or about 375 mg, followed by one dose once in two weeks (Q2W) of the 28-day cycle in an amount of or about 750 mg; and
the second and/or a subsequent 28-day cycle comprises administering the anti-PD-L1 antibody or antigen-binding fragment thereof as one dose every four weeks (Q4W) for in an amount of or about 1500 mg.

23. A method of treatment, the method comprising administering an anti-PD-L1 antibody or antigen-binding fragment thereof to a subject having a B cell malignancy, said subject having been administered a T cell therapy comprising a dose of genetically engineered T cells expressing a chimeric antigen receptor that specifically binds to a target antigen expressed by the B cell malignancy wherein the administration of the anti-PD-L1 antibody or antigen-binding fragment thereof comprises carrying out at least two 28-day cycles, wherein:
the first 28-day cycle comprises administering the anti-PD-L1 antibody or antigen-binding fragment thereof as two individual doses each once-weekly (Q1W) for two consecutive weeks of the 28-day cycle, each of said individual doses in an amount of or about 375 mg, followed by one dose once in two weeks (Q2W) of the 28-day cycle in an amount of or about 750 mg; and
the second and/or a subsequent 28-day cycle comprises administering the anti-PD-L1 antibody or antigen-binding fragment thereof as one dose every four weeks (Q4W) for in an amount of or about 1500 mg.

24. A method of treatment, the method comprising:
(a) administering a T cell therapy to a subject having a B cell malignancy, said T cell therapy comprising a dose of genetically engineered T cells expressing a chimeric antigen receptor, wherein the chimeric antigen receptor specifically binds to a target antigen expressed by the B cell maligancy; and
(b) subsequently administering to the subject an anti-PD-L1 antibody or antigen-binding fragment thereof, said administration comprises carrying out at least two 28-day cycles, wherein:
the first 28-day cycle comprises administering the anti-PD-L1 antibody or antigen-binding fragment thereof as four individual doses each once-weekly (Q1W) for the 28-day cycle, wherein, said four doses comprises two consecutive Q1W doses each independently of or of about 225 mg followed by two consecutive Q1W doses each independently of or of about 375 mg; and
the second and/or a subsequent 28-day cycle comprises administering the anti-PD-L1 antibody or antigen-binding fragment thereof as two doses each every two weeks (Q2W) of the 28-day cycle, wherein each Q2W administration is each independently in an amount of or of about 750 mg.

25. A method of treatment, the method comprising administering an anti-PD-L1 antibody or antigen-binding fragment thereof to a subject having a B cell malignancy, said subject having been administered a T cell therapy comprising a dose of genetically engineered T cells expressing a chimeric antigen receptor that specifically binds to a target antigen expressed on the B cell malignancy, wherein the anti-PD-L1 antibody or antigen-binding fragment thereof comprises carrying out at least two 28-day cycles, wherein:
the first 28-day cycle comprises administering the anti-PD-L1 antibody or antigen-binding fragment thereof as four individual doses each once-weekly (Q1W) for the 28-day cycle, wherein the four individual doses comprises two consecutive Q1W doses each independently of or of about 225 mg followed by two consecutive Q1W doses each independently of or about 375 mg; and
the second and/or a subsequent 28-day cycle comprises administering the anti-PD-L1 antibody or antigen-binding fragment thereof as two individual doses every two weeks (Q2W) for the second and/or subsequent 28-day cycle, wherein each dose independently is in an amount of or about 750 mg.

26. A method of treatment, the method comprising:
(a) administering a T cell therapy to a subject having a B cell malignancy, said T cell therapy comprising a dose of genetically engineered T cells expressing a chimeric antigen receptor, wherein the chimeric antigen receptor specifically binds to a target antigen expressed by the B cell malignancy; and
(b) subsequently administering to the subject an anti-PD-L1 antibody or antigen-binding fragment thereof, said administration comprises carrying out at least two 28-day cycles, wherein:
the first 28-day cycle comprises administering the anti-PD-L1 antibody or antigen-binding fragment thereof as two individual doses each once-weekly (Q1W), wherein each of said two doses independently comprises an amount of or of about 375 mg, optionally wherein the two doses are consecutive Q1W doses, optionally wherein the two doses are administered on days 15 and 22 in the 28-day cycle; and
the second and/or a subsequent 28-day cycle comprises administering the anti-PD-L1 antibody or antigen-binding fragment thereof Q4W for one dose in the second and/or subsequent 28-day cycle in an amount of or about 1500 mg.

27. A method of treatment, the method comprising administering an anti-PD-L1 antibody or antigen-binding fragment thereof to a subject having a B cell malignancy, said subject having been administered a T cell therapy comprising a dose of genetically engineered T cells expressing a chimeric antigen receptor that specifically binds to a target antigen expressed on the B cell malignancy, wherein the administration of the anti-PD-L1 antibody or antigen-binding fragment comprises carrying out at least two 28-day cycles, wherein:
the first 28-day cycle comprises administering the anti-PD-L1 antibody or antigen-binding fragment thereof as two individual doses each once-weekly (Q1W), wherein each of said two doses independently comprises an amount of or of about 375 mg, optionally wherein the two doses are consecutive Q1W doses, optionally wherein the two doses are administered on days 15 and 22 in the 28-day cycle; and
the second and/or a subsequent 28-day cycle comprises administering the anti-PD-L1 antibody or antigen-binding fragment thereof Q4W for one dose in the second and/or subsequent 28-day cycle in an amount of or about 1500 mg.

28. A method of treatment, the method comprising:
(a) administering a T cell therapy to a subject having a B cell malignancy, said cell therapy comprising a dose of genetically engineered T cells expressing a chimeric antigen receptor, wherein the chimeric antigen receptor specifically binds to a target antigen expressed by the B cell malignancy; and
(b) subsequently administering to the subject an anti-PD-L1 antibody or antigen-binding fragment thereof, wherein:
the administration of the anti-PD-L1 antibody or antigen-binding fragment comprises carrying out at least two 28-day cycles, each of said at least two 28-day cycles, comprising administering a total dosage amount of 750 mg to 2000 mg of the antibody or antigen-binding fragment; and
in at least one of said at least two 28-day cycles, the administration of the total dosage amount of the anti-PD-L1 antibody or antigen-binding fragment is carried out by administering more than one individual doses of the antibody or fragment over the course of the at least one 28-day cycle.

29. A method of treatment, the method comprising administering an anti-PD-L1 antibody or antigen-binding fragment thereof to a subject having a B cell malignancy, said subject having been administered a T cell therapy comprising a dose of genetically engineered T cells expressing a chimeric antigen receptor, wherein the chimeric antigen receptor specifically binds to a target antigen expressed by the B cell malignancy, wherein:
the administration of the anti-PD-L1 antibody or antigen-binding fragment comprises carrying out at least two 28-day cycles, each of said at least two 28-day cycles, independently, comprising administering a total dosage amount of 750 mg to 2000 mg of the antibody or antigen-binding fragment; and
in at least one of said at least two 28-day cycles, the administration of the total dosage amount of the anti-PD-L1 antibody or antigen-binding fragment is carried out by administering more than one individual dose of the antibody or fragment over the course of the at least one 28-day cycle.

30. The method of claim 28 or claim 29, wherein in a first of said at least two 28-day cycles, the administration of the total dosage amount of the anti-PD-L1 antibody or antigen-binding fragment is carried out by administering a greater number of individual doses of the antibody or fragment as compared to the administration in the second and/or a subsequent 28-day cycle.

31. The method of any of claims 28-30, wherein the total dosage amount of the anti-PD-L1 antibody or antigen-binding fragment in each 28-day cycle independently is between at or about 750 mg and at or about 1500 mg.

32. The method of any of claims 28-31, wherein the total dosage amount of the anti-PD-L1 antibody or antigen-binding fragment in at least one of the 28-day cycles is at or about 750 mg.

33. The method of any of claims 28-32, wherein the total dosage amount of the anti-PD-L1 antibody or antigen-binding fragment in at least one of the 28-day cycles is at or about 1200 mg.

34. The method of any of claims 28-31, wherein the total dosage amount of the anti-PD-L1 antibody or antigen-binding fragment in at least one of the 28-day cycles is at or about 1500 mg.

35. The method of any of claims 28-31 and 34, wherein the total dosage amount of the anti-PD-L1 antibody or antigen-binding fragment in each 28-day cycle, independently, is at or about 1500 mg.

36. The method of any of claims 28-35, wherein the total dosage amount of the anti-PD-L1 antibody or antigen-binding fragment in at least two of said at least two, and optionally in said at least two, 28-day cycles is the same total dosage amount.

37. The method of any of claims 28-35, wherein the total dosage amount of the anti-PD-L1 antibody or antigen-binding fragment is different in at least two of, or is different in each of, said at least two 28-day cycles.

38. The method of any of claims 28-35 and 37, wherein the total dosage amount of the anti-PD-L1 antibody or antigen-binding fragment in the first of said at least two 28-day cycles is lower than the second and/or a subsequent of said at least two 28-day cycle.

39. The method of any of claims 28-38, wherein the administration of the total dosage amount in the first of said at least two 28-day cycles comprises administering 2, 3 or 4 individual doses of the anti-PD-L1 antibody or antigen-binding fragment thereof.

40. The method of any of claims 28-39, wherein the administration of the total dosage amount in the first of said at least two 28-day cycles comprises administering the anti-PD-L1 antibody or antigen-binding fragment thereof as individual doses according to a dosing schedule selected from (i) two individual doses each once-weekly (Q1W) within the 28-day cycle, optionally on days 15 and 22 of the 28-day cycle; (ii) four individual doses each once-weekly (Q1W) for the 28-day cycle, optionally on days 1, 8, 15 and 22 of the 28-day cycle; (iii) two individual doses each Q1W for two consecutive weeks of the 28-day cycle, optionally on days 1 and 8 of the cycle, followed by one dose once in two weeks (Q2W) of the 28-day cycle, optionally on day 15 of the cycle; or (iv) two individual doses each every two weeks (Q2W) for the 28-day cycle, optionally on days 1 and 15 of the 28-day cycle.

41. The method of claim 40, wherein:
each Q1W dose administered in the first 28-day cycle is independently from or from at or about 18% to at or about 32% of the total dosage amount administered in the first 28-day cycle, optionally is at or about 25% of the total dosage amount administered in the first 28-day cycle; and/or
each Q2W dose administered in the first 28-day cycle is independently from or from at or about 40% to at or about 62.5% of the total dosage amount in the first 28-day cycle, optionally is at or about 50% of the total dosage amount administered in the first 28-day cycle.

42. The method of claim 40 or claim 41, wherein:
the administration of the total dosage amount in the first 28-day cycle comprises administering the anti-PD-L1 antibody or antigen-binding fragment thereof according to dosing schedule (iii), wherein each of the two individual doses Q1W for two consecutive weeks is, each independently, in an amount of or of about 375 mg followed by one dose once Q2W in an amount of or of about 750 mg;
the administration of the total dosage amount in the first 28-day cycle comprises administering the anti-PD-L1 antibody or antigen-binding fragment thereof according to dosing schedule set forth in (ii), wherein the four individual doses Q1W comprise two consecutive Q1W doses in an amount of or of about 225 mg followed by two consecutive Q1W doses in an amount of or of about 375 mg; or
the administration of the total dosage amount in the first 28-day cycle comprises administering the anti-PD-L1 antibody or antigen-binding fragment according to dosing schedule set forth in (i), wherein each of the two individual doses Q1W are carried out for two consecutive Q1W doses in an amount of or of about 375 mg.

43. The method of any of claims 28-40, wherein the administration of the total dosage amount in the first of said at least two 28-day cycles comprises administering individual doses according to a dosing schedule selected from (i) two individual doses on or about day 15 and on or about day 22 of the 28-day cycle; (ii) four individual doses on or about day 1, on or about day 8, on or about day 15 and on or about day 22 of the 28-day cycle; (iii) two individual doses on or about day 1 and on or about day 8 of the 28-day cycle, followed by one dose on or about day 15 of the cycle; or (iv) two doses on or about day 1 of the 28-day cycle and on or about day 15 of the 28-day cycle.

44. The method of any of claims 28-43, wherein:
the administration of the total dosage amount in the first 28-day cycle comprises administering the anti-PD-L1 antibody or antigen-binding fragment thereof according to dosing schedule (iii), wherein each of the two individual doses comprise an amount of or of about 375 mg on or about day 1 and on or about day 8 of the 28-day cycle, followed by one dose in an amount of or of about 750 mg on or about day 15 of the cycle the administration of the total dosage amount in the first 28-day cycle comprises administering the anti-PD-L1 antibody or antigen-binding fragment thereof according to dosing schedule set forth in (ii), wherein the four individual doses comprise two consecutive doses in an amount of or of about 225 mg on or about day 1 and on or about day 8, followed by two consecutive doses in an amount of or of about 375 mg on or about day 15 and on or about day 22 of the 28-day cycle; or
the administration of the total dosage amount in the first 28-day cycle comprises administering the anti-PD-L1 antibody or antigen-binding fragment according to dosing schedule set forth in (i), wherein each of the two individual doses comprise two consecutive in an amount of or of about 375 mg on or about day 15 and on or about day 22 of the 28-day cycle.

45. The method of any of claims 30-44, wherein the administration of the total dosage amount in the second and/or a subsequent 28-day cycle, independently, comprises administering 1 or 2 doses of the anti-PD-L1 antibody or antigen-binding fragment thereof.

46. The method of any of claims 30-45, wherein the administration of the total dosage amount in the second and/or a subsequent 28-day cycle, independently, comprises a dosing schedule selected from (i) two individual doses each every two weeks (Q2W) for the second and/or subsequent 28-day cycle, optionally on days 1 and 15 of the second and/or subsequent cycle; or (ii) one dose every four weeks (Q4W) of the second and/or subsequent 28-day cycle, optionally on day 1 of the second and/or subsequent cycle.

47. The method of claim 46, wherein:
each Q2W dose of the second and/or subsequent 28-day cycle is or is about 50% of the total dosage amount of the second and/or subsequent 28 day cycle; and/or
the Q4W dose of the second and/or subsequent 28-day cycle is or is about the total dosage amount of the second and/or subsequent 28 day cycle.

48. The method of claim 46 or claim 47, wherein:
the second and/or a subsequent dose comprises administering the anti-PD-L1 antibody or antigen-binding fragment thereof Q2W for two doses in an amount of or of about 750 mg; or
the second and/or a subsequent dose comprises administering the anti-PD-L1 antibody or antigen-binding fragment thereof Q4W for one dose in an amount of or of about 1500 mg.

49. The method of any of claims 4-48, wherein at least two 28-day cycles further comprises a third 28-day cycle and/or wherein the subsequent 28-day cycle is a third 28-day cycle.

50. The method of claim 49, wherein the total dosage amount of the anti-PD-L1 antibody or antigen-binding fragment in the third 28-day cycle is the same as the total dosage amount administered in the first and/or in the second 28-day cycle.

51. The method of claim 49 or claim 50, wherein the total dosage amount of the anti-PD-L1 antibody or antigen-binding fragment in the third 28-day cycle is or is about 1500 mg.

52. The method of any of claims 49-51, wherein:
(a) in the third 28-day cycle, the administration of the total dosage amount of the anti-PD-L1 antibody or antigen-binding fragment is carried out by administering the antibody or fragment in a greater number of individual doses as compared to in the first and/or second 28-day cycle; or
(b) in the third 28-day cycle, the administration of the total dosage amount of the anti-PD-L1 antibody or antigen-binding fragment is carried out by administering the same number of doses of the antibody or fragment as compared to the second 28-day cycle.

53. The method of any of claims 49-52, wherein the administration of the total dosage amount in the third 28-day cycle comprises administration of one dose every four weeks (Q4W) of the third 28-day cycle, optionally on day 1 of the third 28-day cycle.

54. The method of any of claims 22-53, wherein the first of said at least two 28-day cycles is initiated at a time:
(a) between day 22 and day 36 of initiation of the administration of the T cell therapy; or
(b) at or after, optionally immediately after or within 1 to 3 days after:
(i) peak or maximum level of the cells of the T cell therapy are detectable in the blood of the subject;
(ii) the number of cells of the T cell therapy detectable in the blood, after having been detectable in the blood, is not detectable or is reduced, optionally reduced compared to a preceding time point after administration of the T cell therapy;
(iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy;
(iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject;
(v) the subject exhibits disease progression and/or has relapsed following remission after treatment with the T cell therapy; and/or
(iv) the subject exhibits increased tumor burden as compared to tumor burden at a time prior to or after administration of the cells and prior to initiation of administration of the anti-PD-L1 antibody.

55. The method of any of claims 22-54, wherein the first of said at least two 28-day cycles is initiated at a time between day 22 and day 36 of initiation of the administration of the T cell therapy.

56. The method of any of claims 22-55, wherein the at least two 28-day cycles comprise no more than three 28-day cycles, optionally wherein the first of said at least two 28-day cycles is initiated between at or about day 22 and at or about day 36.

57. The method of any of claims 22-56, wherein the first of said at least two 28-day cycle is initiated at or about day 29 after initiation of the administration of the T cell therapy.

58. The method of any of claims 22-57, wherein the first of said at least two 28-day cycle is initiated at or about day 43 after initiation of administration of the T cell therapy.

59. A method of treatment, the method comprising:
(a) administering a T cell therapy to a subject having a B cell malignancy, said T cell therapy comprising a dose of genetically engineered T cells expressing a chimeric antigen receptor, wherein the chimeric antigen receptor specifically binds to a target antigen expressed by the B cell malignancy; and
(b) subsequently administering to the subject an anti-PD-L1 antibody or antigen-binding fragment thereof, wherein the administration of antibody or antigen-binding fragment comprises carrying out between one and three 28-day cycles, each cycle comprising administering a total dosage amount of 750 mg to 2000 mg of the antibody or fragment, optionally wherein the first of said between one and three 28-day cycle begins between at or about day 22 and at or about day 36, optionally at day 29, after initiation of the T cell therapy.

60. A method of treatment, the method comprising administering an anti-PD-L1 antibody or antigen-binding fragment thereof to a subject having a B cell malignancy, said subject having been administered a T cell therapy comprising a dose of genetically engineered T cells expressing a chimeric antigen receptor, wherein the chimeric antigen receptor specifically binds to a target antigen expressed by the B cell malignancy, wherein the administration of the antibody or antigen-binding fragment comprises carrying out between one and three 28-day cycles, each cycle comprises administering a total dosage amount of 900 mg to 2000 mg of the antibody or fragment, optionally wherein the first of said between one and three 28-day cycles begins between at or about day 22 and at or about day 36, optionally at about day 29, after initiation of the T cell therapy.

61. The method of claim 59 or claim 60, wherein the total dosage amount of the anti-PD-L1 antibody or antigen-binding fragment in each 28-day cycle independently is or is about 1200 mg to 1500 mg.

62. The method of any of claims 59-61, wherein the total dosage amount of the anti-PD-L1 antibody or antigen-binding fragment in at least one 28-day cycle is or is about 1200 mg.

63. The method of any of claims 59-61, wherein the total dosage amount of the anti-PD-L1 antibody or antigen-binding fragment in at least one 28-day cycle is or is about 1500 mg.

64. The method of any of claims 59-61 and 63, wherein the total dosage amount of the anti-PD-L1 antibody or antigen-binding fragment in each 28-day cycle is or is about 1500 mg.

65. The method of any of claims 59-64, wherein the total dosage amount in each 28-day cycle comprises administering 1, 2, 3 or 4 doses of the anti-PD-L1 antibody or antigen-binding fragment thereof.

66. The method of any of claims 59-65, wherein each 28-day cycle independently comprises a dosing schedule selected from (i) four doses each once-weekly (Q1W), optionally on days 1, 8, 15 and 22 of the 28-day cycle; (ii) two consecutive doses each Q1W, optionally on days 1 and 8, followed by one dose once in two weeks (Q2W) for one dose, optionally on day 15, of the 28-day cycle; (iii) two doses each every two weeks (Q2W), optionally on days 1 and 15 of the 28-day cycle; or (iv) one dose every four weeks (Q4W), optionally on day 1, of the 28-day cycle.

67. The method of any of claims 59-66 wherein the anti-PD-L1 antibody or antigen-binding fragment is administered on day 1, 8 and 15 in a first 28-day cycle, on day 1 in a second 28-day cycle, and on day 1 in a third 28-day cycle.

68. The method of any of claims 59-67, wherein the anti-PD-L1 antibody or antigen-binding fragment is administered on day 1, 8, 15 and 22 in a first 28-day cycle, on day 1 and 15 in a second 28-day cycle, and on day 1 in a third 28-day cycle.

69. The method of any of claims 59-68, wherein the anti-PD-L1 antibody or antigen-binding fragment is administered on day 1 in each 28-day cycle.

70. The method of any of claims 59-69, further comprising administering the anti-PD-L1 antibody or antigen-binding fragment in one or more further 28-day cycle if the subject exhibits no more than a partial response (PR) following the treatment and/or exhibits no more than a PR at three-months following initiation of administration of the T cell therapy and/or of the anti-PD-L1 antibody or fragment.

71. The method of claim 70, wherein the anti-PD-L1 antibody or antigen-binding fragment is administered in a total dosage amount of 900 mg to 2000 mg in each of the one or more further 28-day cycle, optionally in a total dosage amount of at or about 1500 mg.

72. The method of any of claims 13-71, wherein the anti-PD-L1 antibody or antigen-binding fragment is administered for a total duration of no more than 12 months.

73. The method of any of claims 13-72, wherein the administration of the anti-PD-L1 antibody or antigen-binding fragment and/or the start of the first 28-day cycle is initiated greater than 21 days after initiation of administration of the T cell therapy.

74. The method of any of claims 13-73, wherein administration of the anti-PD-L1 antibody or antigen-binding fragment and/or the start of the first 28-day cycle is initiated at a time at or after, optionally immediately after or within 1 to 3 days after:
(i) peak or maximum level of the cells of the T cell therapy are detectable in the blood of the subject;
(ii) the number of cells of the T cell therapy detectable in the blood, after having been detectable in the blood, is not detectable or is reduced, optionally reduced compared to a preceding time point after administration of the T cell therapy;
(iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy;
(iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject;
(v) the subject exhibits disease progression and/or has relapsed following remission after treatment with the T cell therapy; and/or
(iv) the subject exhibits increased tumor burden as compared to tumor burden at a time prior to or after administration of the cells and prior to initiation of administration of the anti-PD-L1 antibody.

75. The method of any of claims 13-74, wherein administration of the anti-PD-L1 antibody or antigen-binding fragment and/or the start of the first 28-day cycle is initiated at or within 29 days, 36 days, 43 days or 50 days after initiation of administration of the T cell therapy.

76. The method of any of claims 13-75, wherein administration of the anti-PD-L1 antibody or antigen-binding fragment and/or the start of the first 28-day cycle is initiated from or from about 22 days to 36 days after initiation of administration of the T cell therapy.

77. The method of any of claims 13-76, wherein administration of the anti-PD-L1 antibody or antigen-binding fragment and/or the start of the first 28-day cycle is initiated at or about 29 days after initiation of administration of the T cell therapy.

78. The method of any of claims 13-77, wherein administration of the checkpoint inhibitor and/or the start of the first dosage cycle is initiated at or about 43 days after initiation of administration of the T cell therapy.

79. The method of any of claims 13-78, wherein at the time of administering the anti-PD-L1 antibody or antigen-binding fragment and/or the start of the first 28-day cycle, the subject does not exhibit a severe toxicity following administration of the T cell therapy.

80. The method of claim 79, wherein:
the severe toxicity is severe cytokine release syndrome (CRS), optionally grade 3 or higher, prolonged grade 3 or higher or grade 4 or 5 CRS; and/or
the severe toxicity is severe neurotoxicity, optionally grade 3 or higher, prolonged grade 3 or higher or grade 4 or 5 neurotoxicity.

81. The method of any of claims 13-80, wherein the anti-PD-L1 antibody or antigen-binding fragment thereof specifically binds to an extracellular domain of PD-L1.

82. The method of any of claims 13-81, wherein the anti-PD-L1 antibody or antigen-binding fragment thereof is MEDI4736 (durvalumab), MDPL3280A (atezolizumab), YW243.55.S70, MDX-1105 (BMS-936559), LY3300054, or MSB0010718C (avelumab), or is or comprises an antigen-binding fragment or region of any of the foregoing.

83. The method of any of claims 13-82, wherein the anti-PD-L1 antibody or antigen-binding fragment thereof is MEDI4736 (durvalumab) or is or comprises an antigen-binding fragment or region thereof.

84. The method of any of claims 13-83, wherein the anti-PD-L1 antibody antibody or antigen binding fragment thereof of MEDI4736 (durvalumab).

85. The method of any of claims 1-84, wherein the B cell malignancy is a non-Hodgkin lymphoma (NHL).

86. The method of claim 85, wherein, at or immediately prior to the time of the administration of the T cell therapy the subject has relapsed following remission after treatment with, or become refractory to, one or more prior therapies for the NHL, optionally one or two prior therapies other than another dose of cells expressing the CAR, optionally wherein the one or more prior therapy is or comprises a CD20-targeted agent or anthracycline.

87. The method of claim 85 or claim 86, wherein the NHL comprises aggressive NHL; diffuse large B cell lymphoma (DLBCL); DLBCL-NOS, optionally transformed indolent; EBV-positive DLBCL-NOS; T cell/histiocyte-rich large B-cell lymphoma; primary mediastinal large B cell lymphoma (PMBCL); follicular lymphoma (FL), optionally, follicular lymphoma Grade 3B (FL3B); and/or high-grade B-cell lymphoma, with MYC and BCL2 and/or BCL6 rearrangements with DLBCL histology (double/triple hit).

88. The method of any of claims 85-87, wherein the NHL comprises diffuse large B cell lymphoma (DLBCL); DLBCL-NOS; DLBCL-NOS transformed indolent; follicular lymphoma Grade 3B (FL3B); and/or high-grade B-cell lymphoma, with MYC and BCL2 and/or BCL6 rearrangements with DLBCL histology (double/triple hit).

89. The method of any of claims 1-88, wherein the subject is or has been identified as having an Eastern Cooperative Oncology Group Performance Status (ECOG) status of less than or equal to 1.

90. The method of any of claims 1-89, wherein the target antigen is a B cell antigen.

91. The method of any of claims 1-90, wherein the target antigen is CD19.

92. The method of claim 91, wherein the chimeric antigen receptor (CAR) comprises an extracellular antigen-recognition domain that specifically binds to a target antigen and an intracellular signaling domain comprising an ITAM.

93. The method of claim 92, wherein the intracellular signaling domain comprises a signaling domain of a CD3-zeta (CD3) chain.

94. The method of claim 92 or claim 93, wherein the chimeric antigen receptor (CAR) further comprises a costimulatory signaling region comprising a cytoplasmic signaling domain of a costimulatory molecule.

95. The method of claim 94, wherein the costimulatory signaling region comprises a cytoplasmic signaling domain of CD28 or 4-1BB.

96. The method of claim 94 or claim 95, wherein the costimulatory domain is or comprises a cytoplasmic signaling domain of 4-1BB.

97. The method of any of claims 1-96, wherein:
the CAR comprises an scFv specific for CD19, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is or comprises a CD3zeta signaling domain, and optionally further comprises a spacer between the transmembrane domain and the scFv.

98. The method of any of claims 1-96, wherein the CAR comprises, in order, an scFv specific for CD19, a transmembrane domain, a cytoplasmic signaling domain derived from a costimulatory molecule, which optionally is or comprises a 4-1BB signaling domain, and a cytoplasmic signaling domain derived from a primary signaling ITAM-containing molecule, which optionally is a CD3zeta signaling domain.

99. The method of any of claims 1-96, wherein the CAR comprises, in order, an scFv specific for CD19, a spacer, a transmembrane domain, a cytoplasmic signaling domain derived from a costimulatory molecule, which optionally is a 4-1BB signaling domain, and a cytoplasmic signaling domain derived from a primary signaling ITAM-containing molecule, which optionally is or comprises a CD3zeta signaling domain.

100. The method of claim 97 or claim 99, wherein:
the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof.

101. The method of any of claims 97, 99 and 100, wherein the spacer comprises or consists of the formula X₁PPX₂P (SEQ ID NO:58), where X₁ is glycine, cysteine or arginine and X₂ is cysteine or threonine.

102. The method of any of claims 97 and 99-101, wherein the spacer comprises or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any of the foregoing having at least 85%, 86%, 87%, 88%, 89%, 90%, 91%, 92%, 93%, 94%, 95%, 96%, 97%, 98%, 99% or more sequence identity thereto.

103. The method of any of claims 97 and 99-102, wherein the spacer comprises the sequence of SEQ ID NO: 1.

104. The method of any of claims 94-103, wherein the cytoplasmic signaling domain of a costimulatory molecule comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto.

105. The method of any of claims 92-104, wherein the cytoplasmic signaling domain derived from a primary signaling ITAM-containing molecule comprises SEQ ID NO: 13 or 14 or 15 having at least 85%, 86%, 87%, 88%, 89%, 90%, 91%, 92%, 93%, 94%, 95%, 96%, 97%, 98%, 99% or more sequence identity thereto.

106. The method of any of claims 92-105, wherein the extracellular antigen-recognition domain is an scFv and the scFv comprises a CDRL1 sequence of RASQDISKYLN (SEQ ID NO: 35), a CDRL2 sequence of SRLHSGV (SEQ ID NO: 36), and/or a CDRL3 sequence of GNTLPYTFG (SEQ ID NO: 37) and a CDRH1 sequence of DYGVS (SEQ ID NO: 38), a CDRH2 sequence of VIWGSETTYYNSALKS (SEQ ID NO: 39), and/or a CDRH3 sequence of YAMDYWG (SEQ ID NO: 40).

107. The method of any of claims 92-106, wherein the extracellular antigen-recognition domain is an scFv and the scFv comprises a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63.

108. The method of any of claims 92-107, wherein the extracellular antigen-recognition domain is an scFv and the scFv comprises a variable heavy chain region of FMC63 and a variable light chain region of FMC63.

109. The method of any of claims 92-108, wherein the wherein the extracellular antigen-recognition domain is an scFv and the scFv comprises a V_{H} region comprising an amino acid sequence set forth in SEQ ID NO:41.

110. The method of any of claims 92-109, wherein the wherein the extracellular antigen-recognition domain is an scFv and the scFv comprises a V_{L} region comprising an amino acid sequence set forth in SEQ ID NO:42.

111. The method of any of claims 92-110, wherein the extracellular antigen-recognition domain is an scFv and the scFv comprises, in order, a V_{H}, optionally comprising the amino acid sequence set forth in SEQ ID NO:41, a linker, optionally comprising SEQ ID NO: 59, and a V_{L}, optionally comprising the amino acid sequence set forth in SEQ ID NO:42, and/or the scFv comprises a flexible linker and/or comprises the amino acid sequence set forth as SEQ ID NO: 43.

112. The method of any of claims 92-111, wherein the wherein the extracellular antigen-recognition domain is an scFv and the scFv comprise an amino acid sequence set forth in SEQ ID NO:43.

113. The method of any of claims 1-112, wherein the dose of genetically engineered T cells comprises from or from about 1×10⁵ to 5×10⁸ total CAR-expressing T cells, 1×10⁶ to 2.5×10⁸ total CAR-expressing T cells, 5×10⁶ to 1×10⁸ total CAR-expressing T cells, 1×10⁷ to 2.5×10⁸ total CAR-expressing T cells, 5×10⁷ to 1×10⁸ total CAR-expressing T cells, each inclusive.

114. The method of any of claims 1-113, wherein the dose of genetically engineered T cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

115. The method of any of claims 1-114, wherein the dose of genetically engineered T cells comprises at or about 5×10⁷ CAR-expressing cells.

116. The method of any of claims 1-114, wherein the dose of genetically engineered T cells comprises at or about 1×10⁸ CAR-expressing cells.

117. The method of any of claims 1-114, wherein the dose of genetically engineered T cells comprises at or about 1.5×10⁸ CAR-expressing cells.

118. The method of any of claims 1-117, wherein the dose of genetically engineered T cells is administered parenterally, optionally intravenously.

119. The method of claim 118, wherein the T cells are primary T cells obtained from a subject.

120. The method of any of claims 1-119, wherein the T cells are autologous to the subject.

121. The method of any of claims 1-119, wherein the T cells are allogeneic to the subject.

122. The method of any of claims 1-121, wherein the dose of genetically engineered T cells comprises CD4+ T cells expressing the CAR and CD8+ T cells expressing the CAR and the administration of the dose comprises administering a plurality of separate compositions, said plurality of separate compositions comprising a first composition comprising one of the CD4+ T cells and the CD8+ T cells and the second composition comprising the other of the CD4+ T cells or the CD8+ T cells.

123. The method of claim 122, wherein:
the first composition and second composition are administered 0 to 12 hours apart, 0 to 6 hours apart or 0 to 2 hours apart or wherein the administration of the first composition and the administration of the second composition are carried out on the same day, are carried out between about 0 and about 12 hours apart, between about 0 and about 6 hours apart or between about 0 and 2 hours apart; and/or
the initiation of administration of the first composition and the initiation of administration of the second composition are carried out between about 1 minute and about 1 hour apart or between about 5 minutes and about 30 minutes apart.

124. The method of claim 122 or claim 123, wherein the first composition and second composition are administered no more than 2 hours, no more than 1 hour, no more than 30 minutes, no more than 15 minutes, no more than 10 minutes or no more than 5 minutes apart.

125. The method of any of claims 122-124, wherein the first composition comprises the CD4+ T cells.

126. The method of any of claims 122-124, wherein the first composition comprises the CD8+ T cells.

127. The method of any of claims 122-126, wherein the first composition is administered prior to the second composition.

129. The method of any of claims 1-127, wherein, prior to the administration, the subject has been preconditioned with a lymphodepleting therapy comprising the administration of fludarabine and/or cyclophosphamide.

130. The method of any of claims 1-129, further comprising, immediately prior to the administration, administering a lymphodepleting therapy to the subject comprising the administration of fludarabine and/or cyclophosphamide.

131. The method of claim 129 or claim 130, wherein the lymphodepleting therapy comprises administration of cyclophosphamide at about 200-400 mg/m², optionally at or about 300 mg/m², inclusive, and/or fludarabine at about 20-40 mg/m², optionally 30 mg/m², daily for 2-4 days, optionally for 3 days, or wherein the lymphodepleting therapy comprises administration of cyclophosphamide at about 500 mg/m².

132. The method of any of claims 129-131, wherein:
the lymphodepleting therapy comprises administration of cyclophosphamide at or about 300 mg/m² and fludarabine at about 30 mg/m² daily for 3 days; and/or
the lymphodepleting therapy comprises administration of cyclophosphamide at or about 500 mg/m² and fludarabine at about 30 mg/m² daily for 3 days.

133. The method of any of claims 1-132, wherein the subject is a human.

134. A kit comprising:
(a) a T cell therapy comprising a dose of genetically engineered T cells expressing a chimeric antigen receptor, wherein the chimeric antigen receptor specifically binds to a target antigen expressed by the B cell malignancy;
(b) a checkpoint inhibitor that is an antibody or antigen-binding fragment thereof capable of blocking an immune checkpoint pathway protein, optionally wherein the checkpoint inhibitor thereof is formulated in one or more individual doses; and
(c) instructions for administering the T cell therapy and/or the checkpoint inhibitor to a subject having a B cell malignancy, wherein the instructions specify administration of the T cell therapy and/or the checkpoint inhibitor according to the method of any of claims 1-133.

135. A kit comprising:
(a) a T cell therapy comprising a dose of genetically engineered T cells expressing a chimeric antigen receptor, wherein the chimeric antigen receptor specifically binds to a target antigen expressed by the B cell malignancy; and
(b) instructions for administering the T cell therapy to a subject having a B cell malignancy, wherein the instructions specify that the subject is to be administered a checkpoint inhibitor that is an antibody or antigen-binding fragment thereof capable of blocking an immune checkpoint pathway protein, after the administration of the T cell therapy, wherein the instructions specify administration of the T cell therapy and/or the checkpoint inhibitor according to the method of any of claims 1-133.

136. A kit comprising:
(a) a checkpoint inhibitor that is an antibody or antigen-binding fragment thereof capable of blocking an immune checkpoint pathway protein, optionally wherein the checkpoint inhibitor thereof is formulated in one or more individual doses; and
(b) instructions for administering the checkpoint inhibitor to a subject having a B cell malignancy, wherein the instructions specify that the checkpoint inhibitor is administered after initiation of administration of a T cell therapy, the T cell therapy comprising a dose of genetically engineered T cells expressing a chimeric antigen receptor, wherein the chimeric antigen receptor specifically binds to a target antigen expressed by the B cell malignancy, wherein the instructions specify administration of the T cell therapy and/or the checkpoint inhibitor according to the method of any of claims 1-133.
